(19) United States
(12) Patent Application Publication
(10) Pub. No.: US 2014/0046407 A1
(43) Pub. Date: Feb. 13, 2014

(54) NERVE STIMULATION TECHNIQUES

(71) Applicant: BIO CONTROL MEDICAL (B.C.M.) LTD., Yehud (IL)

(72) Inventors: Omry BEN-EZRA, Tel Aviv (IL); Tamir BEN-DAVID, Tel Aviv (IL); Ehud COHEN, Ganei Tikva (IL); Shai AYAL, Shoham (IL)

(73) Assignee: BIO CONTROL MEDICAL (B.C.M.) LTD., Yehud (IL)

(21) Appl. No.: 14/059,104

(22) Filed: Oct. 21, 2013

Related U.S. Application Data
(63) Continuation-in-part of application No. 12/952,058, filed on Nov. 22, 2010, now Pat. No. 8,565,896.
Application No. 12/952,058 is a continuation-in-part of application No. 12/228,630, filed on Aug. 13, 2008, now Pat. No. 8,615,294.
Application No. 12/228,630 is a continuation-in-part of application No. 13/022,199, filed on Feb. 7, 2011, now Pat. No. 8,571,651.
Application No. 13/022,199 is a division of application No. 11/517,888, filed on Sep. 7, 2006, now Pat. No. 7,904,176.
Application No. 11/517,888 is a continuation-in-part of application No. 12/012,366, filed on Feb. 1, 2008.
Application No. 12/012,366 is a continuation-in-part of application No. 13/022,279, filed on Feb. 7, 2011, now Pat. No. 8,571,653.
Application No. 13/022,279 is a continuation-in-part of application No. 11/234,877, filed on Sep. 22, 2005, now Pat. No. 7,885,709.
Application No. 13/022,279 is a continuation-in-part of application No. 11/977,923, filed on Oct. 25, 2007, now abandoned.
Application No. 11/977,923 is a continuation-in-part of application No. 11/064,446, filed on Feb. 22, 2005, now Pat. No. 7,974,693.
Application No. 11/064,446 is a continuation-in-part of application No. 11/062,324, filed on Feb. 18, 2005, now Pat. No. 7,634,317.
Application No. 11/062,324 is a continuation-in-part of application No. 10/719,659, filed on Nov. 20, 2003, now Pat. No. 7,778,711.
Application No. 10/719,659 is a continuation-in-part of application No. PCT/IL03/00431, filed on May 23, 2003.
Application No. PCT/IL03/00431 is a continuation-in-part of application No. 10/205,475, filed on Jul. 24, 2002, now Pat. No. 7,778,703.
Application No. 10/205,475 is a continuation-in-part of application No. PCT/IL02/00068.
Application No. PCT/IL02/00068 is a continuation-in-part of application No. 09/944,913, filed on Aug. 31, 2001, now Pat. No. 6,684,105.
(60) Provisional application No. 60/937,351, filed on Jun. 26, 2007.
(60) Provisional application No. 60/965,731, filed on Aug. 21, 2007.
(60) Provisional application No. 60/612,428, filed on Sep. 23, 2004.
(60) Provisional application No. 60/668,275, filed on Apr. 4, 2005.

(51) Int. Cl. A61N 1/36

(52) U.S. Cl. CPC A61N 1/36189; A61N 1/36139
USPC 607/62; 607/72

(57) ABSTRACT

An electrode device is configured to be coupled to a parasympathetic site of a subject. A control unit is configured to drive the electrode device to apply a current in bursts of one or more pulses, during “on” periods that alternate with low stimulation periods, wherein at least one of the low stimulation periods immediately following the at least one of the “on” periods has a low stimulation duration equal to at least 50% of the “on” duration; set the current applied on average during the low stimulation periods to be less than 20% of the current applied on average during the “on” periods; and ramp a number of pulses per burst during a commencement of the at least one of the “on” periods and/or a conclusion of the at least one of the “on” periods.

### CROSS-REFERENCE TO RELATED APPLICATIONS

The present application is a continuation-in-part of:

(a) U.S. patent application Ser. No. 12/952,058, filed Nov. 22, 2010;

(b) U.S. patent application Ser. No. 12/228,630, filed Aug. 13, 2008;

(c) U.S. patent application Ser. No. 13/022,199, filed Feb. 7, 2011, which is a divisional of U.S. patent application Ser. No. 11/517,888, filed Sep. 7, 2006, now U.S. Pat. No. 7,904,176;

(d) U.S. patent application Ser. No. 12/012,366, filed Feb. 1, 2008, which claims the benefit of (i) U.S. Provisional Application 60/937,351, filed Jun. 26, 2007, and (ii) U.S. Provisional Application 60/965,731, filed Aug. 21, 2007; and

(e) U.S. patent application Ser. No. 13/022,279, filed Feb. 7, 2011, which is a continuation-in-part of:
(i) U.S. patent application Ser. No. 11/234,877, filed Sep. 22, 2005, now U.S. Pat. No. 7,885,709, which claims the benefit of (1) U.S. Provisional Patent Application 60/612,428, filed Sep. 23, 2004, and (2) U.S. Provisional Patent Application 60/668,275, filed Apr. 4, 2005;
(ii) U.S. patent application Ser. No. 11/977,923, filed Oct. 25, 2007, now abandoned;
(iii) U.S. patent application Ser. No. 11/064,446, filed Feb. 22, 2005, now U.S. Pat. No. 7,974,693, which is a continuation-in-part of U.S. patent application Ser. No. 11/062,324, filed Feb. 18, 2005, now U.S. Pat. No. 7,634,317, which is a continuation-in-part of U.S. patent application Ser. No. 10/719,659, filed Nov. 20, 2003, now U.S. Pat. No. 7,778,711, which is a continuation-in-part of PCT Patent Application PCT/IL03/00431, filed May 23, 2003, which: (1) is a continuation-in-part of U.S. patent application Ser. No. 10/205,475, filed Jul. 24, 2002, now U.S. Pat. No. 7,778,703, which is a continuation-in-part of PCT Patent Application PCT/IL02/00068, filed Jan. 23, 2002, which is a continuation-in-part of U.S. patent application Ser. No. 09/944,913, filed Aug. 31, 2001, now U.S. Pat. No. 6,684,105; and (2) claims the benefit of U.S. Provisional Patent Application 60/383,157, filed May 23, 2002; and
(iv) U.S. patent application Ser. No. 10/722,589, filed Nov. 25, 2003, now U.S. Pat. No. 7,890,185, which is a continuation of U.S. patent application Ser. No. 09/944,913, filed Aug. 31, 2001, now U.S. Pat. No. 6,684,105.

All of the above-mentioned applications are assigned to the assignee of the present application and are incorporated herein by reference.

### FIELD OF THE APPLICATION

The present invention relates generally to electrical stimulation of and/or sensing signals of tissue, and specifically to methods and devices for regulating the stimulation of nerves or other tissue, including the vagus nerve, and/or sensing of electrical cardiac signals.

### BACKGROUND

A number of patents and articles describe methods and devices for stimulating nerves to achieve a desired effect. Often these techniques include a design for an electrode or electrode cuff.

The use of nerve stimulation for treating and controlling a variety of medical, psychiatric, and neurological disorders has seen significant growth over the last several decades. In particular, stimulation of the vagus nerve (the tenth cranial nerve, and part of the parasympathetic nervous system) has been the subject of considerable research. The vagus nerve is composed of somatic and visceral afferents (inward conducting nerve fibers, which convey impulses toward the brain) and efferents (outward conducting nerve fibers, which convey impulses to an effector to regulate activity such as muscle contraction or glandular secretion).

The rate of the heart is restrained in part by parasympathetic stimulation from the right and left vagus nerves. Low vagal nerve activity is considered to be related to various arrhythmias, including tachycardia, ventricular accelerated rhythm, and rapid atrial fibrillation. By artificially stimulating the vagus nerves, it is possible to slow the heart, allowing the heart to more completely relax and the ventricles to experience increased filling. With larger diastolic volumes, the heart may beat more efficiently because it may expend less energy to overcome the myocardial viscosity and elastic forces of the heart with each beat.

Stimulation of the vagus nerve has been proposed as a method for treating various heart conditions, including heart failure and atrial fibrillation. Heart failure is a cardiac condition characterized by a deficiency in the ability of the heart to pump blood throughout the body and/or to prevent blood from backing up in the lungs. Customary treatment of heart failure includes medication and lifestyle changes. It is often desirable to lower the heart rates of patients suffering from faster than normal heart rates. The effectiveness of beta blockers in treating heart disease is attributed in part to their heart-rate-lowering effect.

Bilgutay et al., in “Vagal tuning: a new concept in the treatment of supraventricular arrhythmias, angina pectoris, and heart failure,” J. Thoracic Cardiovas. Surg. 56(1):71-82, July, 1968, which is incorporated herein by reference, studied the use of a permanently-implanted device with electrodes to stimulate the right vagus nerve for treatment of supraventricular arrhythmias, angina pectoris, and heart failure. Experiments were conducted to determine amplitudes, frequencies, wave shapes and pulse lengths of the stimulating current to achieve slowing of the heart rate. The authors additionally studied an external device, triggered by the R-wave of the electrocardiogram (ECG) of the subject to provide stimulation only upon an achievement of a certain heart rate. They found that when a pulsatile current with a frequency of ten pulses per second and 0.2 milliseconds pulse duration was applied to the vagus nerve, the heart rate could be decreased to half the resting rate while still preserving sinus rhythm. Low amplitude vagal stimulation was employed to control induced tachycardias and ectopic beats. The authors further studied the use of the implanted device in conjunction with the administration of Isuprel, a sympathomimetic drug. They found that Isuprel retained its inotropic effect of increasing contractility, while its chronotropic effect was controlled by the vagal stimulation: “An increased end diastolic volume brought about by slowing of the heart rate by vagal tuning, coupled with increased contractility of the heart induced by the inotropic effect of Isuprel, appeared to increase the efficiency of cardiac performance” (p. 79).

US Patent Application Publications 2005/0197675 and 2005/0267542 to Ben-David, which are assigned to the assignee of the present application and are incorporated herein by reference, describe apparatus including an electrode device, adapted to be coupled to a site of a subject; and a control unit, adapted to drive the electrode device to apply a current to the site intermittently during alternating “on” and “off” periods, each of the “on” periods having an “on” duration equal to between 1 and 10 seconds, and each of the “off” periods having an “off” duration equal to at least 50% of the “on” duration. In some embodiments, the control unit is configured to gradually ramp the commencement and/or termination of stimulation. In order to achieve the gradual ramp, the control unit is typically configured to gradually modify one or more stimulation parameters, such as those described hereinabove, e.g., pulse amplitude, pulses per trigger (PPT), pulse frequency, pulse width, “on” time, and/or “off” time. As appropriate, one or more of these parameters are varied by less than 50% of a pre-termination value per heart beat, in order to achieve the gradual ramp. For example, stimulation at 5 PPT may be gradually terminated by reducing the PPT by 1 pulse per hour. Alternatively, one or more of the parameters are varied by less than 5% per heart beat, in order to achieve the gradual ramp.

U.S. Pat. No. 6,167,304 to Loos, which is incorporated herein by reference, describes techniques for manipulating the nervous system of a subject by applying to the skin a pulsing external electric field which, although too weak to cause classical nerve stimulation, modulates the normal spontaneous spiking patterns of certain kinds of afferent nerves. For certain pulse frequencies the electric field stimulation can excite in the nervous system resonances with observable physiological consequences. Pulse variability is introduced for the purpose of thwarting habituation of the nervous system to the repetitive stimulation, or to alleviate the need for precise tuning to a resonance frequency, or to control pathological oscillatory neural activities such as tremors or seizures. Pulse generators with stochastic and deterministic pulse variability are described, and the output of a generator of the latter type is characterized. Techniques for achieving pulse variability include ramping the pulse frequency in time, or switching the pulses on and off according to a certain schedule determined by dedicated digital circuitry or by a programmable microprocessor.

US Patent Application Publication 2005/0222644 to Killian et al., which is incorporated herein by reference, describes a method for stimulating nerve or tissue fibers and a prosthetic hearing device implementing same. The method comprises: generating a stimulation signal comprising a plurality of pulse bursts each comprising a plurality of pulses; and distributing said plurality of pulse bursts across one or more electrodes each operatively coupled to nerve or tissue fibers such that each of said plurality of pulse bursts delivers a charge to said nerve or tissue fibers to cause dispersed firing in said nerve or tissue fibers. In an embodiment, individual pulses of a pulse burst are non-repeatedly interleaved on three channels. Multiple pulses may be repeated on one channel.

U.S. Pat. No. 5,562,718 to Palermo, which is incorporated herein by reference, describes an electronic neuromuscular stimulation device that is operated by a control unit. The control unit includes at least two output channels to which are connected to a corresponding set of electrode output cables. Each cable has attached a positive electrode and a negative electrode that are attached to selected areas of a patient's anatomy. The control unit also includes controls, indicators, and circuitry that produce nerve stimulation pulses that are applied to the patient through the electrodes. The nerve stimulation pulses consist of individual pulses that are arranged into pulse trains and pulse train patterns. The pulse train patterns, whose selection depends on the type of ailment being treated, includes sequential patterns, delayed overlapping patterns, triple-phase overlapping patterns, reciprocal pulse trains, and delayed sequenced “sprint interval” patterns. The overlapping patterns are described as being particularly timed to take advantage of neurological enhancement. In an embodiment, the pulse trains operate at a pulse rate interval of between 10 and 20 milliseconds which corresponds to a frequency of between 50 Hz and 100 Hz respectively. If a ramp frequency is used, it is applied just prior to the application of a long pulse train. The ramp frequency varies between 18 and 50 Hz and progresses over a 0.5 to 2.0 second period.

U.S. Pat. No. 5,707,400 to Terry, Jr. et al., which is incorporated herein by reference, describes a method for treating patients suffering from refractory hypertension, which includes identifying a patient suffering from the disorder and applying a stimulating electrical signal to the patient's vagus nerve predetermined to modulate the electrical activity of the nerve and to alleviate the hypertension. The stimulating signal is a pulse waveform with programmable signal parameter values including pulse width, output current, frequency, on time and off time. Patient discomfort may be alleviated by a ramping up the pulses during the first two seconds of stimulation, rather than abrupt application at the programmed level.

U.S. Pat. No. 6,928,320 to King, which is incorporated herein by reference, describes techniques for producing a desired effect by therapeutically activating tissue at a first site within a patient's body, and reducing a corresponding undesired side effect by blocking activation of tissue or conduction of action potentials at a second site within the patient's body by applying high frequency stimulation and/or direct current pulses at or near the second site. Time-varying DC pulses may be used before or after a high frequency blocking signal. The high frequency stimulation may begin before and continue during the therapeutic activation. The high frequency stimulation may begin with a relatively low amplitude, and the amplitude may be gradually increased. The desired effect may be promotion of micturition or defecation and the undesired side effect may be sphincter contraction. The desired effect may be defibrillation of the patient's atria or defibrillation of the patient's ventricles, and the undesired side effect may be pain. In an embodiment, the amplitude of the pulse waveform is ramped up or gradually increased at the beginning of the waveform, and ramped down or gradually decreased at the end of the waveform, respectively. Such ramping may be used in order to minimize creation of any action potentials that may be caused by more abruptly starting and/or more abruptly stopping the high frequency blocking stimulation.

US Patent Application Publication 2006/0129205 to Boveja et al., which is incorporated herein by reference, describes techniques for providing rectangular and/or complex electrical pulses to cortical tissues of a patient for at least one of providing therapy or alleviating symptoms of neurological disorders including Parkinson's disease, or for providing improvement of functional recovery following stroke. The intracranial electrodes may be implanted epidurally, or subdurally on the pia mater of the cortical surface. In an embodiment, a microcontroller is configured to deliver a pulse train by “ramping up” of the pulse train. The purpose of the ramping-up is to avoid sudden changes in stimulation when the pulse train begins.

U.S. Pat. No. 6,895,280 to Meadows et al., which is incorporated herein by reference, describes a spinal cord stimulation (SCS) system that includes multiple electrodes, multiple, independently programmable, stimulation channels within an implantable pulse generator (IPG) which channels can provide concurrent, but unique stimulation fields, permitting virtual electrodes to be realized. If slow start/end is enabled, the stimulation intensity is ramped up gradually when the IPG is first turned ON. If slow start/end is enabled, the stimulation intensity may be ramped down gradually rather than abruptly turned off. In an embodiment, a pulse ramping control technique for providing a slow turn-on of the stimulation burst includes modulating pulse amplitude at the beginning of a stimulation burst, while maintaining the pulse width as wide as possible, e.g., as wide as the final pulse duration.

US Patent Application Publication 2006/0015153A1 to Gliner et al., which is incorporated herein by reference, describes techniques for enhancing or affecting neural stimulation efficiency and/or efficacy. In one embodiment, electromagnetic stimulation is applied to a patient's nervous system over a first time domain according to a first set of stimulation parameters, and over a second time domain according to a second set of stimulation parameters. The first and second time domains may be sequential, simultaneous, or nested. Stimulation parameters may vary in accordance with one or more types of duty cycle, amplitude, pulse repetition frequency, pulse width, spatiotemporal, and/or polarity variations. Stimulation may be applied at subthreshold, threshold, and/or suprathreshold levels in one or more periodic, aperiodic (e.g., chaotic), and/or pseudo-random manners. In some embodiments stimulation may comprise a burst pattern having an interburst frequency corresponding to an intrinsic brainwave frequency, and regular and/or varying intraburst stimulation parameters. In an embodiment, within a time interval under consideration (e.g., 250 milliseconds), an interpulse interval of 8 milliseconds may occur 5 times; an interpulse interval of 10 milliseconds may occur 8 times; an interpulse interval of 12 milliseconds may occur 6 times; an interpulse interval of 14 milliseconds may occur 2 times; and interpulse intervals of 16 milliseconds and 18 milliseconds may each occur once.

U.S. Pat. No. 5,330,507 to Schwartz, which is incorporated herein by reference, describes techniques for stimulating the right or left vagus nerve with continuous and/or phasic electrical pulses, the latter in a specific relationship with the R-wave of the patient's electrogram. The automatic detection of the need for vagal stimulation is responsive to increases in the heart rate greater than a predetermined threshold, the occurrence of frequent or complex ventricular arrhythmias, and/or a change in the ST segment elevation greater than a predetermined or programmed threshold.

US Patent Application Publication 2003/0040774 to Terry et al., which is incorporated herein by reference, describes a device for treating patients suffering from congestive heart failure that includes an implantable neurostimulator for stimulating the patient's vagus nerve at or above the cardiac branch with an electrical pulse waveform at a stimulating rate sufficient to maintain the patient's heart beat at a rate well below the patient's normal resting heart rate, thereby allowing rest and recovery of the heart muscle, to increase in coronary blood flow, and/or growth of coronary capillaries. A metabolic need sensor detects the patient's current physical state and concomitantly supplies a control signal to the neurostimulator to vary the stimulating rate. If the detection indicates a state of rest, the neurostimulator rate reduces the patient's heart rate below the patient's normal resting rate. If the detection indicates physical exertion, the neurostimulator rate increases the patient's heart rate above the normal resting rate.

U.S. Pat. No. 5,203,326 to Collins, which is incorporated herein by reference, describes an antiarrhythmia pacemaker which detects a cardiac abnormality and responds with electrical stimulation of the heart combined with vagus nerve stimulation. The pacemaker controls electrical stimulation of the heart in terms of timing, frequency, amplitude, duration and other operational parameters, to provide such pacing therapies as antitachycardia pacing, cardioversion, and defibrillation. The vagal stimulation frequency is progressively increased in one-minute intervals, and, for the pulse delivery rate selected, the heart rate is described as being slowed to a desired, stable level by increasing the pulse current.

An article by Nickel C H et al., “The role of copeptin as a diagnostic and prognostic biomarker for risk stratification in the emergency department,” BMC Medicine 10:7 (2012), is incorporated herein by reference.

The following references, all of which are incorporated herein by reference, may be of interest:
Bilgutay et al., “Vagal tuning: a new concept in the treatment of supraventricular arrhythmias, angina pectoris, and heart failure,” J. Thoracic Cardiovas. Surg. 56(1):71-82, July, 1968
U.S. Pat. No. 6,473,644 to Terry, Jr. et al.
US Patent Application Publication 2003/0040774 to Terry et al.
PCT Publication WO 04/043494 to Paterson et al.
US Patent Application Publication 2005/0131467 to Boveja
US Patent Application Publication 2003/0045909 to Gross et al.
US Patent Application Publication 2005/0197675
US Patent Application Publication 2004/0193231
PCT Publication WO 03/099377 to Ayal et al.
PCT Publication WO 03/018113 to Cohen et al.
U.S. Pat. No. 6,684,105 to Cohen et al.
U.S. Pat. No. 6,610,713 to Tracey

The effect of vagal stimulation on heart rate and other aspects of heart function, including the relationship between the timing of vagal stimulation within the cardiac cycle and the induced effect on heart rate, has been studied in animals. For example, Zhang Y et al., in “Optimal ventricular rate slowing during atrial fibrillation by feedback AV nodal-selective vagal stimulation,” Am J Physiol Heart Circ Physiol 282:H1102-H1110 (2002), describe the application of selective vagal stimulation by varying the nerve stimulation intensity, in order to achieve graded slowing of heart rate. This article is incorporated herein by reference.

The following articles and book, which are incorporated herein by reference, may be of interest:
Levy M N et al., in “Parasympathetic Control of the Heart,” Nervous Control of Vascular Function, Randall W C ed., Oxford University Press (1984)
Levy M N et al. ed., Vagal Control of the Heart: Experimental Basis and Clinical Implications (The Bakken Research Center Series Volume 7), Futura Publishing Company, Inc., Armonk, N.Y. (1993)
Randall W C ed., Neural Regulation of the Heart, Oxford University Press (1977), particularly pages 100-106.
Armour J A et al. eds., Neurocardiology, Oxford University Press (1994)
Perez M G et al., “Effect of stimulating non-myelinated vagal axon on atrioventricular conduction and left ventricular function in anaesthetized rabbits,” Auton Neurosco 86 (2001)
Jones, J F X et al., “Heart rate responses to selective stimulation of cardiac vagal C fibres in anaesthetized cats, rats and rabbits,” J Physiol 489 (Pt 1):203-14 (1995)
Wallick D W et al., “Effects of ouabain and vagal stimulation on heart rate in the dog,” Cardiovasc. Res., 18(2):75-9 (1984)
Martin P J et al., “Phasic effects of repetitive vagal stimulation on atrial contraction,” Circ. Res. 52(6):657-63 (1983)
Wallick D W et al., “Effects of repetitive bursts of vagal activity on atrioventricular junctional rate in dogs,” Am J Physiol 237(3):H275-81 (1979)
Fuster V and Ryden L E et al., “ACC/AHA/ESC Practice Guidelines—Executive Summary,” J Am Coll Cardiol 38(4):1231-65 (2001)
Fuster V and Ryden L E et al., “ACC/AHA/ESC Practice Guidelines—Full Text,” J Am Coll Cardiol 38(4):1266i-12661xx (2001)
Morady F et al., “Effects of resting vagal tone on accessory atrioventricular connections,” Circulation 81(1):86-90 (1990)
Waninger M S et al., “Electrophysiological control of ventricular rate during atrial fibrillation,” PACE 23:1239-1244 (2000)
Wijffels M C et al., “Electrical remodeling due to atrial fibrillation in chronically instrumented conscious goats: roles of neurohumoral changes, ischemia, atrial stretch, and high rate of electrical activation,” Circulation 96(10):3710-20 (1997)
Wijffels M C et al., “Atrial fibrillation begets atrial fibrillation,” Circulation 92:1954-1968 (1995)
Goldberger A L et al., “Vagally-mediated atrial fibrillation in dogs: conversion with bretylium tosylate,” Int J Cardiol 13(1):47-55 (1986)
Takei M et al., “Vagal stimulation prior to atrial rapid pacing protects the atrium from electrical remodeling in anesthetized dogs,” Jpn Circ J 65(12):1077-81 (2001)
Friedrichs G S, “Experimental models of atrial fibrillation/flutter,” J Pharmacological and Toxicological Methods 43:117-123 (2000)
Hayashi H et al., “Different effects of class Ic and III antiarrhythmic drugs on vagotonic atrial fibrillation in the canine heart,” Journal of Cardiovascular Pharmacology 31:101-107 (1998)
Morillo C A et al., “Chronic rapid atrial pacing. Structural, functional, and electrophysiological characteristics of a new model of sustained atrial fibrillation,” Circulation 91:1588-1595 (1995)
Lew S J et al., “Stroke prevention in elderly patients with atrial fibrillation,” Singapore Med J 43(4):198-201 (2002)
Higgins C B, “Parasympathetic control of the heart,” Pharmacol. Rev. 25:120-155 (1973)
Hunt R, “Experiments on the relations of the inhibitory to the accelerator nerves of the heart,” J. Exptl. Med. 2:151-179 (1897)
Billette J et al., “Roles of the AV junction in determining the ventricular response to atrial fibrillation,” Can J Physiol Pharamacol 53(4)575-85 (1975)
Stramba-Badiale M et al., “Sympathetic-Parasympathetic Interaction and Accentuated Antagonism in Conscious Dogs,” American Journal of Physiology 260 (2Pt 2):H335-340 (1991)
Garrigue S et al., “Post-ganglionic vagal stimulation of the atrioventricular node reduces ventricular rate during atrial fibrillation,” PACE 21(4), 878 (Part II) (1998)
Kwan H et al., “Cardiovascular adverse drug reactions during initiation of antiarrhythmic therapy for atrial fibrillation,” Can J Hosp Pharm 54:10-14 (2001)
Jidéus L, “Atrial fibrillation after coronary artery bypass surgery: A study of causes and risk factors,” Acta Universitatis Upsaliensis, Uppsala, Sweden (2001)
Borovikova L V et al., “Vagus nerve stimulation attenuates the systemic inflammatory response to endotoxin,” Nature 405(6785):458-62 (2000)
Wang H et al., “Nicotinic acetylcholine receptor alpha-7 subunit is an essential regulator of inflammation,” Nature 421:384-388 (2003)
Vanoli E et al., “Vagal stimulation and prevention of sudden death in conscious dogs with a healed myocardial infarction,” Circ Res 68(5):1471-81 (1991)
De Ferrari G M, “Vagal reflexes and survival during acute myocardial ischemia in conscious dogs with healed myocardial infarction,” Am J Physiol 261(1 Pt 2):H63-9 (1991)
Li D et al., “Promotion of Atrial Fibrillation by Heart Failure in Dogs: Atrial Remodeling of a Different Sort,” Circulation 100(1):87-95 (1999)
Feliciano L et al., “Vagal nerve stimulation during muscarinic and beta-adrenergic blockade causes significant coronary artery dilation,” Cardiovasc Res 40(1):45-55 (1998)
Sabbah H N et al., “A canine model of chronic heart failure produced by multiple sequential coronary microembolizations,” Am J Physiol 260:H1379-1384 (1991)
Sabbah H N et al., “Effects of long-term monotherapy with enalapril, metoprolol, and digoxin on the progression of left ventricular dysfunction and dilation in dogs with reduced ejection fraction,” Circulation 89:2852-2859 (1994)
Dodge H T et al., “Usefulness and limitations of radiographic methods for determining left ventricular volume,” Am J Cardiol 18:10-24 (1966)
Sabbah H N et al., “Left ventricular shape: A factor in the etiology of functional mitral regurgitation in heart failure,” Am Heart J 123: 961-966 (1992)

Heart rate variability is considered an important determinant of cardiac function. Heart rate normally fluctuates within a normal range in order to accommodate constantly changing physiological needs. For example, heart rate increases during waking hours, exertion, and inspiration, and decreases during sleeping, relaxation, and expiration. Two representations of heart rate variability are commonly used: (a) the standard deviation of beat-to-beat RR interval differences within a certain time window (i.e., variability in the time domain), and (b) the magnitude of variability as a function of frequency (i.e., variability in the frequency domain).

Short-term (beat-to-beat) variability in heart rate represents fast, high-frequency (HF) changes in heart rate. For example, the changes in heart rate associated with breathing are characterized by a frequency of between about 0.15 and about 0.4 Hz (corresponding to a time constant between about 2.5 and 7 seconds). Low-frequency (LF) changes in heart rate (for example, blood pressure variations) are characterized by a frequency of between about 0.04 and about 0.15 Hz (corresponding to a time constant between about 7 and 25 seconds). Very-low-frequency (VLF) changes in heart rate are characterized by a frequency of between about 0.003 and about 0.04 Hz (0.5 to 5 minutes). Ultra-low-frequency (ULF) changes in heart rate are characterized by a frequency of between about 0.0001 and about 0.003 Hz (5 minutes to 2.75 hours). A commonly used indicator of heart rate variability is the ratio of HF power to LF power.

High heart rate variability (especially in the high frequency range, as described hereinabove) is generally correlated with a good prognosis in conditions such as ischemic heart disease and heart failure. In other conditions, such as atrial fibrillation, increased heart rate variability in an even higher frequency range can cause a reduction in cardiac efficiency by producing beats that arrive too quickly (when the ventricle is not optimally filled) and beats that arrive too late (when the ventricle is fully filled and the pressure is too high).

Kamath et al., in “Effect of vagal nerve electrostimulation on the power spectrum of heart rate variability in man,” Pacing Clin Electrophysiol 15:235-43 (1992), describe an increase in the ratio of low frequency to high frequency components of the peak power spectrum of heart rate variability during a period without vagal stimulation, compared to periods with vagal stimulation. Iwao et al., in “Effect of constant and intermittent vagal stimulation on the heart rate and heart rate variability in rabbits,” Jpn J Physiol 50:33-9 (2000), describe no change in heart rate variability caused by respiration in all modes of stimulation with respect to baseline data. Each of these articles is incorporated herein by reference.

The following articles, which are incorporated herein by reference, may be of interest:
Kleiger R E et al., “Decreased heart rate variability and its association with increased mortality after myocardial infarction,” Am J Cardiol 59: 256-262 (1987)
Akselrod S et al., “Power spectrum analysis of heart rate fluctuation: a quantitative probe of beat-to-beat cardiovascular control,” Science 213: 220-222 (1981)

A number of patents describe techniques for treating arrhythmias and/or ischemia by, at least in part, stimulating the vagus nerve. Arrhythmias in which the heart rate is too fast include fibrillation, flutter and tachycardia. Arrhythmia in which the heart rate is too slow is known as bradyarrhythmia. U.S. Pat. No. 5,700,282 to Zabara, which is incorporated herein by reference, describes techniques for stabilizing the heart rhythm of a patient by detecting arrhythmias and then electronically stimulating the vagus and cardiac sympathetic nerves of the patient. The stimulation of vagus efferents directly causes the heart rate to slow down, while the stimulation of cardiac sympathetic nerve efferents causes the heart rate to quicken.

The following references, all of which are incorporated herein by reference, may be of interest:
U.S. Pat. No. 5,330,507 to Schwartz
European Patent Application EP 0 688 577 to Holmström et al.
U.S. Pat. Nos. 5,690,681 and 5,916,239 to Geddes et al.
U.S. Pat. No. 5,203,326 to Collins
U.S. Pat. No. 6,511,500 to Rahme
U.S. Pat. No. 5,199,428 to Obel et al.
U.S. Pat. Nos. 5,334,221 to Bardy and 5,356,425 to Bardy et al.
U.S. Pat. No. 5,522,854 to Ideker et al.
U.S. Pat. No. 6,434,424 to Igel et al.
US Patent Application Publication 2002/0120304 to Mest
U.S. Pat. Nos. 6,006,134 and 6,266,564 to Hill et al.
PCT Publication WO 02/085448 to Foreman et al.
U.S. Pat. No. 5,243,980 to Mehra
U.S. Pat. No. 5,658,318 to Stroetmann et al.
U.S. Pat. No. 6,292,695 to Webster, Jr. et al.
U.S. Pat. No. 6,134,470 to Hartlaub

The use of nerve stimulation for treating and controlling a variety of medical, psychiatric, and neurological disorders has experienced significant growth over the last several decades, including for treatment of heart conditions. In particular, stimulation of the vagus nerve (the tenth cranial nerve, and part of the parasympathetic nervous system) has been the subject of considerable research. The vagus nerve is composed of somatic and visceral afferents (inward conducting nerve fibers, which convey impulses toward the brain) and efferents (outward conducting nerve fibers, which convey impulses to an effector to regulate activity such as muscle contraction or glandular secretion).

The rate of the heart is restrained in part by parasympathetic stimulation from the right and left vagus nerves. Low vagal nerve activity is considered to be related to various arrhythmias, including tachycardia, ventricular accelerated rhythm, and rapid atrial fibrillation. Stimulation of the vagus nerve has been proposed as a method for treating various heart conditions, including atrial fibrillation and heart failure. By artificially stimulating the vagus nerves, it is possible to slow the heart, allowing the heart to more completely relax and the ventricles to experience increased filling. With larger diastolic volumes, the heart may beat more efficiently because it may expend less energy to overcome the myocardial viscosity and elastic forces of the heart with each beat.

Atrial fibrillation is a condition in which the atria of the heart fail to continuously contract in synchrony with the ventricles of the heart. During fibrillation, the atria undergo rapid and unorganized electrical depolarization, so that no contractile force is produced. The ventricles, which normally receive contraction signals from the atria (through the atrioventricular (AV) node), are inundated with signals, typically resulting in a rapid and irregular ventricular rate. Because of this rapid and irregular rate, the patient suffers from reduced cardiac output, a feeling of palpitations, and/or increased risk of thromboembolic events.

Current therapy for atrial fibrillation includes cardioversion and rate control. Cardioversion is the conversion of the abnormal atrial rhythm into normal sinus rhythm. This conversion is generally achieved pharmacologically or electrically. An atrial defibrillator applies an electrical shock when an episode of arrhythmia is detected. Such a device has not shown widespread clinical applicability because of the pain that is often associated with such electrical shocks. Atrial override pacing (the delivery of rapid atrial pacing to override abnormal atrial rhythms) has not shown sufficient clinical benefit to justify clinical use. Rate control therapy is used to control the ventricular rate, while allowing the atria to continue fibrillation. This is generally achieved by slowing the conduction of signals through the AV node from the atria to the ventricles.

Current treatment techniques have generally not demonstrated long-term efficacy in preventing the recurrence of episodes of atrial fibrillation. Because of the high frequency of recurrences (up to several times each day), and a lack of effective preventive measures, many patients live in a constant state of atrial arrhythmia, which is associated with increased morbidity and mortality.

An article by Vincenzi et al., entitled, “Release of autonomic mediators in cardiac tissue by direct subthreshold electrical stimulation,” J Pharmacol Exp Ther. 1963 August; 141:185-94, which is incorporated herein by reference, describes subthreshold electrical stimuli for myocardial excitation. Such excitation was described as being effective in causing the release of autonomic mediators in several types of cardiac tissue derived from rabbit, guinea pig, dog, and cat.

U.S. Pat. No. 5,411,531 to Hill et al., which is incorporated herein by reference, describes a device for controlling the duration of A-V conduction intervals in the heart. Stimulation of the AV nodal fat pad is employed to maintain the durations of the A-V conduction intervals within a desired interval range, which may vary as a function of sensed heart rate or other physiologic parameter. AV nodal fat pad stimulation may also be triggered in response to defined heart rhythms such as a rapid rate or the occurrence of premature ventricular depolarizations (PVCs), to terminate or prevent induction of arrhythmias.

Cooper T B et al., in “Neural effects on sinus rate and atrioventricular conduction produced by electrical stimulation from a transveous electrode catheter in the canine right pulmonary artery,” Circulation Research 46:48-57 (1980), which is incorporated herein by reference, studied the effects on sinus rate and atrioventricular (AV) conduction of electrical stimulation from a 12-polar electrode catheter advanced into the right pulmonary artery of 21 anesthetized dogs. In each experiment, the distal tip of the electrode catheter was positioned at a standard fluoroscopic site, and a sequence of bipolar electrograms was recorded during sinus rhythm from the 11 adjacent catheter electrode pairs using a standardized technique. Stimulus-strength response testing was performed from each catheter electrode pair during spontaneous sinus rhythm and during atrial fibrillation sustained by rapid atrial pacing. Negative chronotropic and negative dromotropic effects persisted throughout 5-minute periods of stimulation from the optimal stimulation site and could be modulated by varying stimulus parameters. Using neurophysiological and neuropharmacological techniques, they demonstrated that these effects were produced by stimulation of preganglionic parasympathetic efferent nerve fibers.

Quan K J et al., in “Endocardial Stimulation of Efferent Parasympathetic Nerves to the Atrioventricular Node in Humans: Optimal Stimulation Sites and the Effects of Digoxin,” Journal of Interventional Cardiac Electrophysiology 5:145-152 (2001), which is incorporated herein by reference, describe a study to identify optimal sites of stimulation of efferent parasympathetic nerve fibers to the human atrioventricular node via an endocardial catheter and to investigate the interaction between digoxin and vagal activation at the end organ.

Bluemel K M et al., in “Parasympathetic postganglionic pathways to the sinoatrial node,” Am J Physiol 259(5 Pt 2):H1504-10 (1990), which is incorporated herein by reference, describes the mapping of the ventral epicardial surface of the right atrium in dogs. A concentric bipolar exploring electrode was used to stimulate (during the atrial refractory period and using trains of five to eight stimuli per beat) systematically in the epicardial regions between the right pulmonary vein complex and the SA node. The authors report that the primary vagal postganglionic pathways to the SA nodal region are subepicardial and adjacent to the SA node artery along the sulcus terminalis.

U.S. Pat. No. 6,298,268 to Ben-Haim et al., which is incorporated herein by reference, describes apparatus for modifying cardiac output of the heart of a subject, including one or more sensors which sense signals responsive to cardiac activity, and a stimulation probe including one or more stimulation electrodes which apply non-excitatory stimulation pulses to a cardiac muscle segment. Signal generation circuitry is coupled to the one or more sensors and the stimulation probe. The circuitry receives the signals from the one or more sensors and generates the non-excitatory stimulation pulses responsive to the signals.

U.S. Pat. No. 6,292,695 to Webster, Jr. et al., which is incorporated herein by reference, describes a method of controlling cardiac fibrillation, tachycardia, or cardiac arrhythmia by the use of an electrophysiology catheter having a tip section that contains at least one stimulating electrode, the electrode being stably placed at a selected intravascular location. The electrode is connected to a stimulating means, and stimulation is applied across the wall of the vessel, transvascularly, to a sympathetic or parasympathetic nerve that innervates the heart at a strength sufficient to depolarize the nerve and effect the control of the heart.

US Statutory Invention Registration H1,905 to Hill, which is incorporated herein by reference, describes an endocardial pacing and/or cardioversion/defibrillation lead having a plurality of electrodes and a mechanism for adjusting the exposed surface area of one or more electrode and/or the position and/or angular orientation of an electrode along a lead body. In an embodiment, movable electrodes may be positioned to facilitate delivery of electrical stimulation through the atrial wall or the superior vena cava wall to autonomic nerves to influence sinus heart rate, the A-V interval, and blood pressure or the like. For example, vagal nerve stimulation may be effected through the atrial wall by an electrode that is oriented towards the vagal nerves. The vagal stimulation may be delivered during an episode of atrial fibrillation or tachycardia in order to slow the ventricular heart rate response to the atrial heart rate.

U.S. Pat. No. 7,269,457 to Shafer et al., which is incorporated herein by reference, describes a medical procedure including stimulation of a patient's heart while stimulating a nerve of the patient in order to modulate the patient's inflammatory process. More particularly, the medical procedure includes pacing the ventricles of the patient's heart while stimulating the vagal nerve of the patient.

U.S. Pat. No. 6,937,897 to Min et al., which is incorporated herein by reference, describes an electrical lead equipped with cathode and anode active succession electrodes for positioning in the vicinity of the His bundle tissue. The lead includes a lead body for carrying conductors coupled between electrodes located at or near the distal lead end and a connector assembly located at the proximal lead end for connecting to an implantable pacemaker. The electrode is shaped, at the distal end, for positioning and attachment in the His bundle and branches thereof, cathode and anode electrodes co-extensive with the lead body. The cathode and anode electrodes may be helical screw-in type or equivalent electrodes adapted for secure fixation deep within the His bundle tissue or the tissue in the vicinity of the His bundle.

PCT Publication WO 02/22206 to Lee, which is incorporated herein by reference, describes a pacing lead characterized by a screw-in tip that is longer than conventional tips and is provided with an electrically active distal electrode, which is insulated from the proximal part of the screw tip of the pacemaker lead. This electrically active distal screw-in tip is extended from the right ventricular septal endocardium into the left side of the interventricular septum and is used for left ventricular pacing with optional properly synchronized right ventricular pacing.

U.S. Pat. No. 6,611,713 to Schauerte, which is incorporated herein by reference, describes an implantable device for diagnosing and distinguishing supraventricular and ventricular tachycardias includes electrodes for stimulating parasympathetic nerves of the atrioventricular and/or sinus node; electrodes for stimulating the atria and ventricles and/or for ventricular cardioversion/defibrillation; a device for producing electrical parasympathetic stimulation pulses passed to the electrodes; a device for detecting the atrial and/or ventricular rate, by ascertaining a time interval between atrial and/or ventricular depolarization; a device for programming a frequency limit above which a rate of the ventricles is recognized as tachycardia; a comparison device for comparing the measured heart rate during parasympathetic stimulation to the heart rate prior to or without parasympathetic stimulation and/or to the frequency limit, which delivers an output signal when with parasympathetic stimulation the heart rate falls below the comparison value by more than a predetermined amount; and an inhibition unit which responds to the output signal to inhibit ventricular myocardial over-stimulation therapy.

U.S. Pat. No. 7,212,870 to Helland, which is incorporated herein by reference, describes an implantable lead for use with an implantable medical device, which includes a lead body with first and second electrical conductors extending between its proximal and distal ends. An electrical connector at the proximal end of the lead body includes terminals electrically connected to the first and second conductors. First and second coaxial active fixation helices are coupled to the lead body's distal end, one being an anode, the other an electrically isolated cathode. Each helix has an outer peripheral surface with alternating insulated and un-insulated portions along its length with about a half of the surface area being insulated. The un-insulated portions of the helices may be formed as a plurality of islands in the insulated portions, or as rings spaced by insulative rings, or as longitudinally extending strips spaced by longitudinally extending insulative strips.

US Patent Application Publication 2006/0206159 to Moffitt et al., which is incorporated herein by reference, describes techniques for applying neural stimulation to first and second neural stimulation sites of a heart. Nerve endings in an IVC-LA fat pad are stimulated in some embodiments using an electrode screwed into the fat pad using either an epicardial or intravascular lead, and are transvascularly stimulated in some embodiments using an intravascular electrode proximately positioned to the fat pad in a vessel such as the inferior vena cava or coronary sinus, or a lead in the left atrium. Some embodiments use an intravascularly-fed lead adapted to puncture through a vessel wall to place an electrode proximate to a target neural stimulation site.

US Patent Application Publication 2006/0217772 to Libbus et al., which is incorporated herein by reference, describes a stimulation platform, including a sensing circuit configured to sense an intrinsic cardiac signal, and a stimulation circuit configured to deliver a stimulation signal for both neural stimulation therapy and cardiac rhythm management (CRM) therapy. Neural targets in a fat pad are stimulated in some embodiments using an electrode screwed into the fat pad, and are stimulated in some embodiments using an intravenously-fed lead proximately positioned to the fat pad in a vessel such as the right pulmonary artery, right pulmonary vein, the inferior vena cava, coronary sinus, or a lead in the left atrium, for example.

US Patent Application Publication 2006/0241725 to Libbus et al., which is incorporated herein by reference, describes a presentation device such as a display screen or a printer that provides for simultaneous presentation of temporally aligned cardiac and neural signals. At least one cardiac signal in the form of a cardiac signal trace or cardiac event markers and at least one neural signal in the form of a neural signal trace or neural event markers are simultaneously presented. The cardiac signal indicates sensed cardiac electrical activities and/or cardiac stimulation pulse deliveries. The neural signal indicates sensed neural electrical activities and/or neural stimulation pulse deliveries. In one embodiment, the presentation device is part of an external system communicating with an implantable system that senses cardiac and/or neural signals and delivers cardiac and/or neural stimulation pulses.

US Patent Application Publication 2006/0271108 to Libbus et al., which is incorporated herein by reference, describes a neural stimulation system that includes a safety control system that prevents delivery of neural stimulation pulses from causing potentially harmful effects. The neural stimulation pulses are delivered to one or more nerves to control the physiological functions regulated by the one or more nerves. Examples of such harmful effects include unintended effects in physiological functions associated with autonomic neural stimulation and nerve injuries caused by excessive delivery of the neural stimulation pulses.

US Patent Application Publication 2006/0206153 to Libbus et al., which is incorporated herein by reference, describes a main lead assembly having a proximal portion adapted for connection to a device and a distal portion adapted for placement in a coronary sinus, the distal portion terminating in a distal end for placement proximal a left ventricle. Additionally, the main lead assembly includes a left ventricular electrode located at its distal end which is adapted to deliver cardiac resynchronization therapy to reduce ventricular wall stress. The main lead assembly also includes a fat pad electrode disposed along the main lead assembly a distance from the distal end to position the fat pad electrode proximal to at least one parasympathetic ganglia located in a fat pad bounded by an inferior vena cava and a left atrium. The fat pad electrode is adapted to stimulate the parasympathetic ganglia to reduce ventricular wall stress.

U.S. Pat. No. 5,334,221 to Bardy, which is incorporated herein by reference, describes a stimulator for providing stimulus pulses to the SA nodal fat pad, in response to heart rate exceeding a predetermined level, in order to reduce the ventricular rate. The device is also provided with a cardiac pacemaker to pace the ventricle in the event that the stimulus pulses reduce the heart rate below a predetermined value. The device is also provided with a feedback regulation mechanism for controlling the parameters of the stimulation pulses applied to the AV nodal fat pad, as a result of their determined effect on heart rate.

U.S. Pat. No. 7,020,530 to Ideker et al., which is incorporated herein by reference, describes a passive conductor assembly for use with an implanted device having an intra-cavitarily or trans-venously disposed electrode. The assembly can include electrical components in electrical communication therewith which provide for the manipulation, and/or modification of the electrical stimulus or waveform generated by the implanted stimulus generator, which can be designed, for example, to selectively stimulate only neural tissue, not cardiac tissue or vice versa through the same passive conductor assembly. The uninsulated portions (electrodes) of at least one conductive element are disposed in contact with the heart and/or other tissues such as neural tissue, fat pads containing post-ganglionic neural fibers, cardiac veins adjacent to neural fibers, or other electrically excitable tissues such as the stellate ganglia and the vagus. The conductive element can also run circumferentially along the atrial-ventricular groove of the heart such that the sympathetic and the parasympathetic innervation, running parallel to cardiac vasculature, can be directly stimulated or inhibited.

U.S. Pat. No. 4,161,952 to Kinney et al., which is incorporated herein by reference, describes an implantable catheter-type cardioverting electrode whose conductive discharge surface is comprised of coils of wound spring wire. An electrically conductive lead extends through the wound wire section of the electrode and has its distal end connected to the discharge coil at two locations. The proximal end of the conductive lead is adapted for connection to an implanted pulse generator.

U.S. Pat. No. 6,934,583 to Weinberg et al., which is incorporated herein by reference, describes techniques for stimulating the right vagal nerve by positioning an electrode portion of a lead proximate to the portion of the vagus nerve where the right cardiac branch is located (e.g., near or within an azygos vein, or the superior vena cava near the opening of the azygos vein) and delivering an electrical signal to an electrode portion adapted to be implanted therein. Stimulation of the right vagus nerve and/or the cardiac branch thereof act to slow the atrial heart rate. Exemplary embodiments include deploying an expandable or self-oriented electrode (e.g., a basket, an electrode umbrella, and/or an electrode spiral electrode, electrode pairs, etc).

U.S. Pat. No. 7,027,876 to Casavant et al., which is incorporated herein by reference, describes methods and endocardial screw-in leads for enabling provision of electrical stimulation to the heart, particularly the His Bundle in the intraventricular septal wall. An endocardial screw-in lead having a distal end coupled to a retractable fixation helix wherein a distal portion of the fixation helix extends beyond the lead distal end when the fixation helix is fully retracted or partially extended is positioned in proximity to the His Bundle in the septal wall. The lead body is rotated to attach the distal portion of the fixation helix into the septal wall. The fixation helix is rotated with respect to the lead body to fully extend the fixation helix so that a portion of the fixation helix is in proximity to the His Bundle, enabling provision of electrical stimulation to the His Bundle and/or to sense electrical signals of the heart traversing the His Bundle through the fixation helix.

US Patent Application Publication 2006/0241733 to Zhang et al., which is incorporated herein by reference, describes a lead that includes a lead body having an expandable section. A plurality of electrodes are disposed on the expandable section. The expandable section is adapted to expand against an inner surface of a heart so as to position at least one of the plurality of electrodes at or near an SA node of the heart.

U.S. Pat. No. 6,850,801 to Kieval et al., which is incorporated herein by reference, describes techniques for selectively and controllably reducing blood pressure, nervous system activity, and neurohormonal activity by activating baroreceptors. A baroreceptor activation device is positioned near a baroreceptor, preferably in the carotid sinus. A mapping method permits the baroreceptor activation device to be precisely located to maximize therapeutic efficacy.

An article by Lemery R et al., entitled, “Feasibility study of endocardial mapping of ganglionated plexuses during catheter ablation of atrial fibrillation,” Heart Rhythm 3:387-396 (2006), which is incorporated herein by reference, describes methods of assessing the safety and efficacy of high-frequency stimulation at mapping cardiac ganglionated plexuses in patients undergoing catheter ablation of AF. In their study, fourteen patients with a history of symptomatic AF underwent a single transseptal approach and electroanatomic mapping of the left atrium, right atrium, and coronary sinus. Using high-frequency stimulation with patients under general anesthesia (20-50 Hz, 5-15 V, pulse width 10 ms), mapping of ganglionated plexuses was performed. Radiofrequency (RF) ablation was performed during AF guided by complex fractionated atrial electrograms. Lesions were mostly delivered circumferentially in the antral area of the PVs, predominantly over and adjacent to regions of ganglionated plexuses. There was a mean of 4+/−1 (range 2-6) ganglionated plexuses per patient, and a mean total of 3+/−1 RF applications were delivered over positive vagal sites. Although a vagal response occurred infrequently during ablation (0.9%), postablation high-frequency stimulation failed to provoke a vagal response in 30 (88%) of 34 previously positive vagal sites that underwent ablation. Thus, it was concluded that ganglionated plexuses can be precisely mapped using high-frequency stimulation and are located predominantly in the path of lesions delivered during ablation of AF. Objective documentation of modification of autonomic tone can be documented in the majority of patients. Future studies were described as being required to determine the specific role of mapping and targeting of ganglionated plexuses in patients undergoing catheter ablation of AF.

The following references, all of which are incorporated herein by reference, may be of interest:
U.S. Pat. No. 4,010,755 to Preston
U.S. Pat. No. 5,170,802 to Mehra
U.S. Pat. No. 5,224,491 to Mehra
U.S. Pat. No. 5,243,980 to Mehra
U.S. Pat. No. 5,203,326 to Collins
U.S. Pat. No. 5,356,425 to Bardy et al.
U.S. Pat. No. 5,507,784 to Hill et al.
U.S. Pat. No. 6,006,134 to Hill et al.
U.S. Pat. No. 6,542,774 to Hill et al.
U.S. Pat. No. 5,690,681 to Geddes et al.
U.S. Pat. No. 5,916,239 to Geddes et al.
U.S. Pat. No. 5,700,282 to Zabara
U.S. Pat. No. 6,073,048 to Kieval et al.
U.S. Pat. No. 6,161,029 to Spreigl et al.
U.S. Pat. No. 6,317,631 to Ben-Haim et al.
U.S. Pat. No. 6,363,279 to Ben-Haim et al.
U.S. Pat. No. 6,463,324 to Ben-Haim et al.
U.S. Pat. No. 6,445,953 to Bulkes et al.
U.S. Pat. No. 7,167,748 to Ben-Haim et al.
U.S. Pat. No. 6,564,096 to Mest
U.S. Pat. No. 6,985,774 to Kieval et al.
U.S. Pat. No. 6,865,416 to Dev et al.
U.S. Pat. No. 7,087,053 to Vanney
U.S. Pat. No. 7,123,961 to Kroll et al.
U.S. Pat. No. 7,139,607 to Shelchuk
U.S. Pat. No. 7,236,821 to Cates et al.
U.S. Pat. No. 7,245,967 to Shelchuk
U.S. Pat. No. RE38,705 to Hill et al.
US Patent Application Publication 2004/0199210 to Shelchuk
US Patent Application Publication 2005/0131467 to Boveja
US Patent Application Publication 2005/0187584 to Denker et al.
US Patent Application Publication 2006/0074449 to Denker et al.
US Patent Application Publication 2005/0261672 to Deem et al.
US Patent Application Publication 2005/0273138 to To et al.
US Patent Application Publication 2006/0052831 to Fukui
US Patent Application Publication 2006/0206154 to Moffitt et al.
US Patent Application Publication 2006/0229677 to Moffitt et al.
US Patent Application Publication 2006/0247607 to Cornelius et al.
PCT Publication WO 04/52444 to Vaingast et al.
U.S. Pat. No. 6,628,987 to Hill et al.
PCT Publication WO 07/053,065 to Eckerdal et al.
US Patent Application Publication 2007/0162079 to Shemer et al.
U.S. Pat. No. 5,893,881 to Elsberry et al.
U.S. Pat. No. 5,800,470 to Stein et al.
Goldberger J J et al., “New technique for vagal nerve stimulation,” J Neurosci Methods 91(1-2):109-14 (1999)
Wallick D W et al., “Selective AV nodal vagal stimulation improves hemodynamics during acute atrial fibrillation in dogs,” Am J Physiol Heart Circ Physiol 281:H1490-H1497 (2001)
Zhang Y et al., “Optimal ventricular rate slowing during atrial fibrillation by feedback AV nodal-selective vagal stimulation,” Am J Physiol Heart Circ Physiol 282:H1102-H1110 (2002)
Zhang Y et al., “Chronic atrioventricular nodal vagal stimulation: First evidence for long-term ventricular rate control in canine atrial fibrillation model,” Circulation 112:2904-2911 (2005)
Schauerte P et al., “Ventricular rate control during atrial fibrillation by cardiac parasympathetic nerve stimulation: A transveous approach,” Journal of the American College of Cardiology 34(7):2043-2050 (1999)
Schauerte P et al., “Catheter stimulation of cardiac parasympathetic nerves in humans: A novel approach to the cardiac autonomic nervous system,” Circulation 104:2430-2435 (2001)
Schauerte P et al., “Transvenous parasympathetic cardiac nerve stimulation for treatment of tachycardic atrial fibrillation,” Tachycarde Rhythmusstorungen 89:766-773 (2000)
Lazzara R et al., “Selective in situ parasympathetic control of the canine sinuatrial and atrioventricular node,” Circulation Research 32:393-401 (1973)
Chen S A, et al., “Intracardiac stimulation of human parasympathetic nerve fibers induces negative dromotropic effects: implication with the lesions of radiofrequency catheter ablation,” Journal of Cardiovascular Electrophysiology 9(3):245-52 (1998)
Tarver W B et al., “Clinical experience with a helical bipolar stimulating lead,” Pace, Vol. 15, October, Part 11 (1992)

A number of patents and articles describe other methods and devices for stimulating nerves to achieve a desired effect. Often these techniques include a design for an electrode or electrode cuff.

The following references, all of which are incorporated herein by reference, may be of interest:
US Patent Application Publication 2003/0050677 to Gross et al.
U.S. Pat. Nos. 4,608,985 to Crish et al. and 4,649,936 to Ungar et al.
PCT Patent Publication WO 01/10375 to Felsen et al.
U.S. Pat. No. 5,755,750 to Petruska et al.

The following articles, which are incorporated herein by reference, may be of interest:
Ungar IJ et al., “Generation of unidirectionally propagating action potentials using a monopolar electrode cuff,” Annals of Biomedical Engineering, 14:437-450 (1986)
Sweeney J D et al., “An asymmetric two electrode cuff for generation of unidirectionally propagated action potentials,” IEEE Transactions on Biomedical Engineering, vol. BME-33(6) (1986)
Sweeney J D et al., “A nerve cuff technique for selective excitation of peripheral nerve trunk regions,” IEEE Transactions on Biomedical Engineering, 37(7) (1990)
Naples G G et al., “A spiral nerve cuff electrode for peripheral nerve stimulation,” by IEEE Transactions on Biomedical Engineering, 35(11) (1988)
van den Honert C et al., “Generation of unidirectionally propagated action potentials in a peripheral nerve by brief stimuli,” Science, 206:1311-1312 (1979)
van den Honert C et al., “A technique for collision block of peripheral nerve: Single stimulus analysis,” MP-11, IEEE Trans. Biomed. Eng. 28:373-378 (1981)
van den Honert C et al., “A technique for collision block of peripheral nerve: Frequency dependence,” MP-12, IEEE Trans. Biomed. Eng. 28:379-382 (1981)
Rijkhoff N J et al., “Acute animal studies on the use of anodal block to reduce urethral resistance in sacral root stimulation,” IEEE Transactions on Rehabilitation Engineering, 2(2):92 (1994)
Mushahwar V K et al., “Muscle recruitment through electrical stimulation of the lumbo-sacral spinal cord,” IEEE Trans Rehabil Eng, 8(1):22-9 (2000)
Deurloo K E et al., “Transverse tripolar stimulation of peripheral nerve: a modelling study of spatial selectivity,” Med Biol Eng Comput, 36(1):66-74 (1998)
Tarver W B et al., “Clinical experience with a helical bipolar stimulating lead,” Pace, Vol. 15, October, Part II (1992)
Manfredi M, “Differential block of conduction of larger fibers in peripheral nerve by direct current,” Arch. Ital. Biol., 108:52-71 (1970)

In physiological muscle contraction, nerve fibers are recruited in the order of increasing size, from smaller-diameter fibers to progressively larger-diameter fibers. In contrast, artificial electrical stimulation of nerves using standard techniques recruits fibers in a larger- to smaller-diameter order, because larger-diameter fibers have a lower excitation threshold. This unnatural recruitment order causes muscle fatigue and poor force gradation. Techniques have been explored to mimic the natural order of recruitment when performing artificial stimulation of nerves to stimulate muscles.

Fitzpatrick et al., in “A nerve cuff design for the selective activation and blocking of myelinated nerve fibers,” Ann. Conf. of the IEEE Eng. in Medicine and Biology Soc, 13(2), 906 (1991), which is incorporated herein by reference, describe a tripolar electrode used for muscle control. The electrode includes a central cathode flanked on its opposite sides by two anodes. The central cathode generates action potentials in the motor nerve fiber by cathodic stimulation. One of the anodes produces a complete anodal block in one direction so that the action potential produced by the cathode is unidirectional. The other anode produces a selective anodal block to permit passage of the action potential in the opposite direction through selected motor nerve fibers to produce the desired muscle stimulation or suppression.

The following articles, which are incorporated herein by reference, may be of interest:
Rijkhoff N J et al., “Orderly recruitment of motoneurons in an acute rabbit model,” Ann. Conf. of the IEEE Eng., Medicine and Biology Soc., 20(5):2564 (1998)
Rijkhoff N J et al., “Selective stimulation of small diameter nerve fibers in a mixed bundle,” Proceedings of the Annual Project Meeting Sensations/Neuros and Mid-Term Review Meeting on the TMR-Network Neuros, Apr. 21-23, 1999, pp. 20-21 (1999)
Baratta R et al., “Orderly stimulation of skeletal muscle motor units with tripolar nerve cuff electrode,” IEEE Transactions on Biomedical Engineering, 36(8):836-43 (1989)
Levy M N, Blattberg B., “Effect of vagal stimulation on the overflow of norepinephrine into the coronary sinus during sympathetic nerve stimulation in the dog,” Circ Res 1976 February; 38(2):81-4
Lavallee et al. “Muscarinic inhibition of endogenous myocardial catecholamine liberation in the dog,” Can J Physiol Pharmacol 1978 August; 56(4):642-9
Mann D L, Kent R L, Parsons B, Cooper G, “Adrenergic effects on the biology of the adult mammalian cardiocyte,” Circulation 1992 February; 85(2):790-804
Mann D L, “Basic mechanisms of disease progression in the failing heart: role of excessive adrenergic drive,” Prog Cardiovasc Dis 1998 July-August; 41(1suppl 1):1-8
Barzilai A, Daily D, Zilkha-Falb R, Ziv I, Offen D, Melamed E, Siry A, “The molecular mechanisms of dopamine toxicity,” Adv Neurol 2003; 91:73-82

The following articles, which are incorporated herein by reference, describe techniques using point electrodes to selectively excite peripheral nerve fibers:
Grill W M et al., “Inversion of the current-distance relationship by transient depolarization,” IEEE Trans Biomed Eng, 44(1):1-9 (1997)
Goodall E V et al., “Position-selective activation of peripheral nerve fibers with a cuff electrode,” IEEE Trans Biomed Eng, 43(8):851-6 (1996)
Veraart C et al., “Selective control of muscle activation with a multipolar nerve cuff electrode,” IEEE Trans Biomed Eng, 40(7):640-53 (1993)

As defined by Rattay, in the article, “Analysis of models for extracellular fiber stimulation,” IEEE Transactions on Biomedical Engineering, Vol. 36, no. 2, p. 676, 1989, which is incorporated herein by reference, the activation function (AF) is the second spatial derivative of the electric potential along an axon. In the region where the activation function is positive, the axon depolarizes, and in the region where the activation function is negative, the axon hyperpolarizes. If the activation function is sufficiently positive, then the depolarization will cause the axon to generate an action potential; similarly, if the activation function is sufficiently negative, then local blocking of action potentials transmission occurs. The activation function depends on the current applied, as well as the geometry of the electrodes and of the axon.

For a given electrode geometry, the equation governing the electrical potential is:

∇(σ∇U)=4πj,

where U is the potential, σ is the conductance tensor specifying the conductance of the various materials (electrode housing, axon, intracellular fluid, etc.), and j is a scalar function representing the current source density specifying the locations of current injection.

Nitric oxide is an important signaling molecule that acts in many tissues to regulate a diverse range of physiological processes, including: (a) vasodilation or vasoconstriction, with resulting changes in blood pressure and blood flow, (b) neurotransmission in the central and peripheral nervous system, including mediation of signals for normal gastrointestinal motility, and (c) defense against pathogens such as bacteria, fungus, and parasites due to the toxicity of high levels of NO to pathogenic organisms.

NO is synthesized within cells by three NO synthases (NOSs):
Neuronal NOS (nNOS), also known as NOS-1, which is regulated by calcium/calcium-calmodulin;
Inducible NOS (iNOS), also known as NOS-2, which is cytokine-inducible and calcium-independent; and
Endothelial NOS (eNOS), also known as NOS-3, which is regulated by calcium/calcium-calmodulin enzymes.

The major roles of nitric oxide include:
vasodilation or vasoconstriction, with resulting changes in blood pressure and blood flow;
neurotransmission in the central and peripheral nervous system, including mediation of signals for normal gastrointestinal motility; and
defense against pathogens such as bacteria, fungus, and parasites, because of the toxicity of high levels of NO to pathogenic organisms.

In blood vessels, NOS-3 mediates endothelium-dependent vasodilation in response to acetylcholine, bradykinin, and other mediators. NO also maintains basal vascular tone and regulates regional blood flow. NO levels increase in response to shear stress (Furchgott et al., and Ignarro (1989) (this and the following references are cited hereinbelow)).

In the nervous system, NOS-1 is localized to discrete populations of neurons in the cerebellum, olfactory bulb, hippocampus, cortex, striatum, basal forebrain, and brain stem. NO plays a role in nervous system morphogenesis and synaptic plasticity. NO is used as a neurotransmitter particularly for long-term potentiation, which is essential for learning and memory (Bishop et al.). The central nervous system immune cell counterparts, microglia and astrocytes, also synthesize NOS-2, which generates a burst of NO in response to injury. Upregulation of NOS expression is seen in many neurodegenerative diseases, and in injury. In the peripheral nervous system, NO mediates relaxation of smooth muscle. NOS-containing neurons also innervate the corpora cavernosa of the penis. Stimulation of these nerves can lead to penile erection and dilation of cerebral arteries, respectively (Snyder, Schmidt et al.).

In the immune system, NO is produced by cytokine-activated macrophages and neutrophils as a cytotoxic agent. High concentrations of NO produced in these cells kill target cells, such as tumor cells and pathogens. In inflammation, a number of factors upregulate NOS-2, including interleukins, interferon-gamma, TNF-alpha, and LPS (Nathan, Marletta (1993), Salvemini (1998)). NOS-2 also plays an important role in innate immunity (Bogdan et al.). A role for constitutive NOS (i.e., NOS expressed without stimulation) and NOS-2 has been demonstrated in an experimental model of bacterial component-induced joint inflammation and tissue degradation (Whal et al. (2003)).

NOSs exert a large number of biological effects in the cardiovascular system. NOSs modulate myocardial oxygen consumption, enhance perfusion-contraction matching and mechanical efficiency, influence cardiac substrate utilization, and prevent apoptosis (Massion et al.). A decrease in the expression of NOS-3 occurs in heart failure. NOS-3 produces low concentrations of NO which is believed necessary for good endothelial function and integrity, and is viewed as a protective agent in a variety of diseases including heart failure, because it plays an important role in the control of myocardial oxygen consumption. Mice deficient in NOS-3 develop postnatal heart failure. Lack of NOS-3 decreases vascular endothelial growth factor (VEGF) expression, and can impair angiogenesis and capillary development that can contribute to cardiac abnormalities. Increased expression of cytokines (in particular, tumor necrosis factor (TNF), such as in heart failure) can induce downregulation of NOS-3. Reduced NOS-3 in heart failure increases the activity of caspase 3, and thus can trigger cardiomyocytes' apoptosis or programmed cell death. (Ferreiro et al.)

Feron et al. showed that agonist binding to muscarinic acetylcholine (mAchRs) receptors on cardiomyocytes results in the activation of NOS-3. Balligand et al. showed that NOS inhibitors reduce the influence of muscarinic agonists on the spontaneous beating rate of rat cardiac myocytes. They also showed that NOS inhibitors increased the inotropic effect of the beta-adrenergic agonist isoproterenol on electrically stimulated adult rat ventricular myocytes. They thus concluded that NOS can protect the heart against excessive stimulation by catecholamines, just as an endogenous beta-blocker. Massion et al. confirmed that NOS-3 attenuates beta adrenergic activity by showing that overexpression of NOS-3 in mice increases the negative chronotropic effect of carbamylcholine as well as attenuated the b-adrenergic positive inotropic effect of isoproterenol. Bendall et al. demonstrated that cardiac NOS-1 expression significantly increased in failing hearts. Failing hearts exposed to NOS-1 inhibition demonstrated better left ventricular function.

Ziolo et al. showed that high levels of iNOS contribute to blunted beta-adrenergic response in failing human hearts by decreasing Ca2+ transients. The presence of systemic inflammation determined by elevations in C-reactive protein (CRP) has been associated with persistence of atrial fibrillation (AF). CRP measurement and cardiovascular assessment were performed at baseline in 5806 subjects. Elevated CRP predicted increased risk for developing future AF (Aviles et al.).

NOS enzymes play critical roles in the physiology and pathophysiology of neuronal, renal, pulmonary, gastrointestinal, skeletal muscle, reproductive, and cardiovascular biology.

All NOS isoforms are involved in promoting or inhibiting the etiology of cancer. NOS activity has been detected in tumor cells of various origins and has been associated with tumor grade, proliferation rate, and expression (Xu et al., Ignarro (1989), Jaiswal (2001)). NOS stimulates angiogenesis, and correlates with tumor growth and aggressiveness (Morbidelli).

Upregulation of NOS expression occurs in many neurodegenerative diseases, including Alzheimer's disease, dementia, stress, and depression (Togo et al., and McLeod et al.). NO mediates relaxation of smooth muscle in the gut, and peristalsis.

NO is an important neurohumoral modulator of renal hemodynamics. NO serves as a neurotransmitter in the lower urinary tract, affects relaxation of the bladder and urethra, and also affects overactive bladder, bladder outlet obstruction, diabetic cystopathy, interstitial cystitis, and bladder inflammation (Ho).

NOS has been reported to be expressed and to play a role in white adipose tissue (Fruhbeck).

NOS plays multiple roles in airway physiology and pathophysiology. In the respiratory tract, NO adduct molecules (nitrosothiols) have been shown to be modulators of bronchomotor tone. The concentration of this molecule in exhaled air is abnormal in activated states of different inflammatory airway diseases, and asthma (Ricciardolo et al.).

In diabetic mellitus, alterations in production of the NOS-3/NO system cause angiopathy and death. Hyperglycemia causes NOS uncoupling, which results in a perturbation of the physiological properties of NO. Abnormality in NO availability thus results in generalized accelerated atherosclerosis, hyperfiltration, glomerulosclerosis, tubulointerstitial fibrosis and progressive decline in glomerular filtration rate, and apoptosis and neovascularization in the retina (Santilli et al.).

Increased expression of NOS-1 has been found in both chronic and acute hepatic encephalopathy (Rao).

The following articles, which are incorporated herein by reference, may be of interest:
Furchgott R F et al., “Endothelium-derived relaxing and contracting factors,” FASEB J 3:2007-2018 (1989)
Ignarro L J, “Endothelium-derived nitric oxide: actions and properties,” FASEB J 3:31-36. (1989)
Ignarro L J, Introduction and overview, in Ignarro L J, Editors, Nitric Oxide: Biology and Pathobiology, Academic Press, San Diego, Calif. (2000), pp. 3-19.
Schmidt HHHW et al., “NO at Work,” Cell 78:919-925 (1994)
Snyder S H, “No endothelial NO,” Nature 377:196-197 (1995)
Jaiswal NF et al., “Nitric oxide in gastrointestinal epithelial cell carcinogenesis: linking inflammation to carcinogenesis,” Am J Physiol Gastrointest Liver Physiol 281:G626-G634 (2001)
Chinthalapally V et al., “Nitric oxide signaling in colon cancer chemoprevention,” Mutat Res 555(1-2):107-19 (2004)
Ho M H et al., “Physiologic role of nitric oxide and nitric oxide synthase in female lower urinary tract,” Curr Opin Obstet Gynecol 16(5):423-9 (2004)
Fruhbeck G, “The adipose tissue as a source of vasoactive factors,” Curr Med Chem Cardiovasc Hematol Agents 2(3):197-208 (2004)
Marletta M A, “Nitric Oxide Synthase Structure and Mechanism,” J Biol Chem 268:12231-12234 (1993)
Nathan C, “Nitric oxide as a secretory product of mammalian cells,” FASEB J 6:3051-3064 (1992)
Ricciardolo F L, et al., “Nitric oxide in health and disease of the respiratory system,” Physiol Rev 84(3):731-65 (2004)
Bishop A et al., “NO signaling in the CNS: from the physiological to the pathological,” Toxicology 208:193-205 (2005)
Togo T et al., “Nitric oxide pathways in Alzheimer's disease and other neurodegenerative dementias,” Neurol Res 26(5):563-6 (2004)
Santilli F et al., “The role of nitric oxide in the development of diabetic angiopathy,” Horm Metab Res 36(5):319-35 (2004)
Xu W et al., “The role of nitric oxide in cancer,” Cell Res 12(5-6):311-20 (2002)
Morbidelli L et al., “Role of nitric oxide in the modulation of angiogenesis,” Curr Pharm Des 9(7):521-30 (2003)
McLeod T et al., “Nitric oxide, stress, and depression,” Psychopharmacol Bull 35(1):24-41 (2001)
Whitworth J A et al., “The nitric oxide system in glucocorticoid-induced hypertension,” J Hypertens 20(6):1035-43 (2002)
Rao V L, “Nitric oxide in hepatic encephalopathy and hyperammonemia,” Neurochem Int 41(2-3):161-70 (2002)
Blantz R C et al., “The complex role of nitric oxide in the regulation of glomerular ultrafiltration,” Kidney Int 61(3):782-5 (2002)
Wahl S M et al., “Nitric oxide in experimental joint inflammation. Benefit or detriment?” Cells Tissues Organs 174(1-2):26-33 (2003)
Massion P B et al., “Regulation of the mammalian heart function by nitric oxide,” Comp Biochem Physiol A Mol Integr Physiol 2005 Jun. 25 [epublication ahead of print]
Bogdan C et al., “The role of nitric oxide in innate immunity,” Immunol Rev 173:17-26 (2000)
Salvemini D et al., “Inducible nitric oxide synthase and inflammation,” Expert Opin Investig Drugs 7(1):65-75 (1998)
Balligand J L et al., “Abnormal contractile function due to induction of nitric oxide synthesis in rat cardiac myocytes follows exposure to activated macrophage-conditioned medium,” J Clin Invest 91:2314-2319 (1993)
Feron O et al., “Dynamic targeting of the agonist-stimulated m2 muscarinic acetylcholine receptor to caveolae in cardiac myocytes,” J Biol Chem 272:17744-17748 (1997)
Bendall J K et al., “Role of myocardial neuronal nitric oxide synthase-derived nitric oxide in beta-adrenergic hyporesponsiveness after myocardial infarction-induced heart failure in rat,” Circulation 110(16):2368-75 (2004) Epub 2004 Oct. 4
Ziolo M T et al., “Myocyte nitric oxide synthase 2 contributes to blunted beta-adrenergic response in failing human hearts by decreasing Ca2+ transients,” Circulation 109(15):1886-91 (2004) Epub 2004 Mar. 22
Aviles RJ et al., “Inflammation as a Risk Factor for Atrial Fibrillation,” Circulation 108:3006-3010 (2003)
Bruins P et al., “Activation of the complement system during and after cardiopulmonary bypass surgery: postsurgery activation involves C-reactive protein and is associated with postoperative arrhythmia,” Circulation 96(10):3542-3548 (1997)
Frustaci A et al., “Histological substrate of atrial biopsies in patients with lone atrial fibrillation,” Circulation 96(4):1180-1184 (1997)
Mihm M J et al., “Impaired myofibrillar energetics and oxidative injury during human atrial fibrillation,” Circulation 104(2):174-180 (2001)
Marin F et al., “Factor XIII Val34Leu polymorphism modulates the prothrombotic and inflammatory state associated with atrial fibrillation,” J Mol Cell Cardiol 37:699-704 (2004)
Gaudino M et al., “The −174G/C interleukin-6 polymorphism influences postoperative interleukin-6 levels and postoperative atrial fibrillation. Is atrial fibrillation an inflammatory complication?” Circulation 108 (Suppl. 1): II195-199 (2003)
Chung M K et al., “C-reactive protein elevation in patients with atrial arrhythmias: inflammatory mechanisms and persistence of atrial fibrillation,” Circulation 104(24):2886-2891 (2001)
Conway D S et al., “Predictive value of indexes of inflammation and hypercoaguability on success of cardioversion of persistent atrial fibrillation,” Am J Cardiol 94:508-510 (2004)
Dernellis J et al., “C-reactive protein and paroxysmal atrial fibrillation: evidence of the implication of an inflammatory process in paroxysmal atrial fibrillation,” Acta Cardiol 56:375-380 (2001)
Korantzopoulos P et al., “Variation of inflammatory indexes after electrical cardioversion of persistent atrial fibrillation. Is there an association with early recurrence rates?” Int J Clin Pract 59(8):881-885 (2005)
Ferreiro C R et al., “Expression of inducible nitric oxide synthase is increased in patients with heart failure due to ischemic disease,” Braz J Med Biol Res. 37(9):1313-20 (2004) Epub 2004 Aug. 24
Mak B C et al., “Aberrant beta-catenin signaling in tuberous sclerosis,” Am J Pathol 167(1):107-16 (2005)
Gould V E et al., “The phosphorylated form of connexin43 is up-regulated in breast hyperplasias and carcinomas and in their neoformed capillaries,” Hum Pathol 36(5):536-45 (2005)
Haass N K et al., “The role of altered cell-cell communication in melanoma progression,” J Mol Histol 35(3):309-18 (2004)
Marino A A et al., Increased intercellular communication through gap junctions may contribute to progression of osteoarthritis,” Clin Orthop Relat Res (422):224-32 (2004)
Brandner J M et al., “Connexins 26, 30, and 43: differences among spontaneous, chronic, and accelerated human wound healing,” J Invest Dermatol 122(5):1310-20 (2004)
Gajda Z et al., “Involvement of gap junctions in the manifestation and control of the duration of seizures in rats in vivo,” Epilepsia 44(12):1596-600 (2003)
Christ G J et al., “Increased connexin43-mediated intercellular communication in a rat model of bladder overactivity in vivo,” Am J Physiol Regul Integr Comp Physiol 284(5):R1241-8 (2003)
Haefliger J A et al., “Connexins 43 and 26 are differentially increased after rat bladder outlet obstruction,” Exp Cell Res 274(2):216-25 (2002)
Vis J C et al., “Connexin expression in Huntington's diseased human brain,” Cell Biol Int 22(11-12):837-47 (1998)
Nagy J I et al., “Elevated connexin43 immunoreactivity at sites of amyloid plaques in Alzheimer's disease,” Brain Res 717(1-2):173-8 (1996)
Wahl S M et al., “Nitric oxide in experimental joint inflammation. Benefit or detriment?” Cells Tissues Organs 174(1-2):26-33 (2003)
Ilebekk et al., “Influence of endogenous neuropeptide Y (NPY) on the sympathetic-parasympathetic interaction in the canine heart,” Cardiovasc Pharmacol 46(4):474-480 (2005)
Danson et al., “Cardiac nitric oxide: Emerging role for nNOS in regulating physiological function,” Pharmacol Ther 106(1):57-74 (2005)

### SUMMARY OF THE APPLICATION

In some embodiments of the present invention, an electrode assembly for applying current to tubular body tissue, such as a nerve, comprises one or more electrode contact surfaces fixed to a cuff. The cuff is shaped so as to define a plurality of recesses that extend radially outwardly from an innermost surface of the cuff surrounding a longitudinal axis of the cuff. Typically, the cuff is recessed in at least one radially outward direction at every longitudinal location along its entire length. The recesses may serve to prevent damage to the nerve by allowing the nerve to swell in at least one radial direction into at least one of the recesses, along the entire length of the cuff. Providing the recesses generally does not have a material impact on the activation function achieved by the electrode assembly.

Typically, each of the recesses has a length along the cuff that is less than the entire length of the cuff, e.g., less than 50% or 25% of the length of the cuff. This design generally prevents migration of the nerve over time into the recesses, away from the center of the cuff, as might occur if any of the recesses extended along the entire length, or even most of the length, of the cuff. Holding the cuff in position around the nerve helps maintain good electrical contact between the electrical contact surfaces and the nerve. In addition, because longitudinally adjacent recesses extend in different radially directions, the recesses do not provide a continuous path for current applied by the electrode contact surfaces to pass through the cuff without entering the nerve.

The cuff is typically shaped such that each perpendicular cross section thereof includes one or more portions that coincide with the innermost surface of the cuff. These non-recessed portions serve in part to hold the cuff in position around the nerve. At the same time, the recesses provide space into which the nerve can swell in varying radial directions along the entire length of the cuff, thereby minimizing any damage the cuff may cause to the nerve. Some of these non-recessed portions further serve in part to electrically isolate longitudinally adjacent recesses from each other along the longitudinal axis of the cuff.

In the present application, including in the claims, a “perpendicular cross section” is a planar cross section perpendicular to the longitudinal axis of the cuff.

For some applications, at least two of the recesses extend radially outwardly in different radial directions, such as in opposite radial directions. Typically, at least two of the perpendicular cross sections of the cuff define respective inner closed curves having shapes that differ from one other, when orientation and position of the perpendicular cross sections with respect to the cuff are preserved. For some applications, the inner closed curves of the at least two of the perpendicular cross sections would cross, and not merely intersect, one another if superimposed while preserving orientation and position of the perpendicular cross sections with respect to the cuff. In contrast, in some other nerve cuffs having recesses, the inner curves of the perpendicular cross sections defining the recesses merely have a larger diameter than the inner curves of the non-recessed perpendicular cross sections, but have the same shape (e.g., circular shape).

For some applications, one or more of the recesses have respective electrode contact surfaces coupled therein, such that the electrode contact surfaces are not in physical contact with the nerve when the cuff is placed around the nerve. In addition, one or more of the recesses may not have an electrode contact surface coupled therein. Because the recesses typically do not extend along the entire length of the cuff, electrode contact surfaces coupled within different recesses are electrically isolated from one another along the longitudinal axis of the cuff. Alternatively or additionally, one or more of the electrode contact surfaces are coupled to respective portions of the innermost, non-recessed surface of the cuff, such that the electrode contact surfaces are in physical contact with the nerve when the cuff is placed around the nerve.

As mentioned above, the cuff may define a plurality of planar cross sections perpendicular to the longitudinal axis, which are distributed continuously along the entire length of the cuff. The perpendicular cross sections may define respective inner closed curves surrounding the longitudinal axis. These inner closed curves, if superimposed while preserving orientation and position of the perpendicular cross sections with respect to the cuff, would together define the innermost closed curve surrounding the longitudinal axis, which is mentioned above. For some applications, this innermost closed curve is elliptical, such as circular.

There is therefore provided, in accordance with an application of the present invention, apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of planar cross sections perpendicular to the longitudinal axis, distributed continuously along an entire length of the cuff along the longitudinal axis, such that the perpendicular cross sections define respective inner closed curves that together define an inner surface that defines and completely surrounds a volume that extends along the entire length of the cuff,

wherein the inner closed curves of at least two of the perpendicular cross sections would cross, and not merely intersect, one another if superimposed while preserving orientation and position of the perpendicular cross sections with respect to the cuff.

For some applications, all of the inner closed curves, if superimposed while preserving orientation and position of the perpendicular cross sections with respect to the cuff, would together define a combined innermost closed curve, and the inner closed curves respectively defined by the perpendicular cross sections enclose respective areas, each of which areas is greater than an area enclosed by the combined innermost closed curve.

For some applications, the entire length of the cuff is between 1 and 40 mm. Alternatively or additionally, for some applications, the volume has a volume of between 10 and 5000 mm3.

For some applications, the cuff is shaped so as to define a plurality of longitudinal segments, distributed continuously along the entire length of the cuff; the segments are shaped so as to define respective ones of the inner closed curves, such that the inner closed curve of each of the segments is of uniform shape along the segment; each of the inner closed curves of at least four of the longitudinal segments has a different shape, and not merely a different size, from the inner closed curve of at least one adjacent longitudinal segment, when orientation and position of the segments with respect to the cuff are preserved; and the at least three segments have respective lengths, measured in parallel with the longitudinal axis, each of which is at least 0.1 mm. For some applications, the inner closed curve of each of the at least four segments is of uniform size along the segment. Alternatively or additionally, for some applications, the inner closed curve of each of at least one of the at least four segments is of non-uniform size along the segment.

For some applications, a first set of a plurality of the perpendicular cross sections contiguous to one another define a first segment of the cuff, a second set of a plurality of the perpendicular cross sections contiguous to one another define a second segment of the cuff, the first and second segments have respective lengths, measured in parallel with the longitudinal axis, each of which is at least 0.1 mm, the first and second segments do not overlap each other lengthwise along the cuff, at least one of the electrode contact surfaces is fixed to the inner surface at the first segment, and none of the electrode contact surfaces is fixed to the inner surface at the second segment. For some applications, all of the inner closed curves defined by the perpendicular cross sections of the first set are identical to one another in shape and size, when orientation and position of the perpendicular cross sections with respect to the cuff are preserved. For some applications, all of the inner closed curves defined by the perpendicular cross sections of the second set are identical to one another in shape and size, when orientation and position of the perpendicular cross sections with respect to the cuff are preserved, and the inner closed curves defined by the perpendicular cross sections of the first set have different shapes, and not merely different sizes, from the inner closed curves defined by the perpendicular cross sections of the second set, when orientation and position of the perpendicular cross sections with respect to the cuff are preserved.

For some applications, the cuff is configured to assume the open and closed positions by defining a slit therethrough that extends along the entire length of the cuff.

For some applications, all of the inner closed curves, if superimposed while preserving orientation and position of the perpendicular cross sections with respect to the cuff, would together define a combined innermost closed curve, and at least a first one of the inner closed curves extends radially outwardly from the combined innermost closed curve in a first radial direction, and at least a second one of the inner closed curves, different from the first inner closed curve, extends radially outwardly from the combined innermost closed curve in a second radial direction different from the first radial direction. For example, the first and second radial directions may be opposite each other.

For some applications, all of the inner closed curves, if superimposed while preserving orientation and position of the perpendicular cross sections with respect to the cuff, would together define a combined innermost closed curve, and each of the inner closed curves partially coincides with the combined innermost closed curve.

There is further provided, in accordance with an application of the present invention, apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define:
(i) a plurality of planar cross sections perpendicular to the longitudinal axis, distributed continuously along an entire length of the cuff along the longitudinal axis, such that the perpendicular cross sections define respective inner closed curves surrounding the longitudinal axis, which inner closed curves define and enclose respective inner cross-sectional regions, wherein an intersection of the cross-sectional regions, if the cross-sectional regions were to be superimposed while preserving orientation and position of the cross-sectional regions with respect to the cuff, would define a combined inner cross-sectional region, which, if extended along the entire length of the cuff, would define a combined innermost volume, and
(ii) a plurality of recesses that are recessed radially outwardly from the combined innermost volume, each of which recesses extends along the longitudinal axis of the cuff and has a greatest length, measured in parallel with the longitudinal axis, that is less than 50% of the entire length of the cuff, wherein the inner closed curves enclose respective areas, each of which areas
   is greater than an area of the combined inner cross-sectional region.

For some applications, the entire length of the cuff is between 1 and 40 mm.

For some applications, the cuff is shaped such that the combined inner cross-sectional region is elliptical, for example, circular.

For some applications, a periphery of the combined inner cross-sectional region defines a combined innermost closed curve, and each of the inner closed curves coincides with the combined innermost closed curve at a portion of, but not all, angles with respect to the longitudinal axis.

For some applications, first and second ones of the recesses overlap each other lengthwise along the cuff, and do not overlap each other anglewise with respect to the longitudinal axis. For some applications, a length of the overlap between the first and second recesses, measured in parallel with the longitudinal axis of the cuff, is at least 0.1 mm.

For some applications, at least a first one of the inner closed curves extends radially outwardly from the combined innermost volume in a first radial direction, and at least a second one of the inner closed curves, different from the first inner closed curve, extends radially outwardly from the combined innermost volume in a second radial direction different from the first radial direction. For example, the first and second radial directions may be opposite each other.

For some applications, each of the recesses has a length, measured in parallel with the longitudinal axis, of at least 0.1 mm.

For some applications, the cuff is configured to assume the open and closed positions by defining a slit therethrough that extends along the entire length of the cuff.

For some applications, at least one of the electrode contact surfaces is fixed within one of the recesses.

For some applications, a first set of a plurality of the perpendicular cross sections contiguous to one another define a first segment of the cuff; a second set of a plurality of the perpendicular cross sections contiguous to one another define a second segment of the cuff; the first and second segments have respective lengths, measured in parallel with the longitudinal axis, each of which is at least 0.1 mm; the first and second segments do not overlap each other lengthwise along the cuff; at least one of the electrode contact surfaces is fixed to an inner surface of the first segment; and none of the electrode contact surfaces is fixed to an inner surface of the second segment.

For some applications:

13 sets of pluralities of the perpendicular cross sections define 13 segments of the cuff, respectively, such that the perpendicular cross sections are contiguous within each of the sets, and the sets are arranged in numerical order from a first set to a thirteenth set along the cuff, such that none of the segments overlap one other lengthwise along the cuff,

the 13 segments have respective first through thirteenth lengths, measured in parallel with the longitudinal axis, each of which is at least 0.1 mm,

the inner closed curves of the first, fifth, ninth, and thirteenth segments have the same shape as one another, while preserving orientation and position of the inner closed curves with respect to the cuff,

the inner closed curves of the second, fourth, sixth, tenth, and twelfth segments have the same shape as one another, while preserving orientation and position of the inner closed curves with respect to the cuff,

the inner closed curves of the third, seventh, and eleventh segments have the same shape as one another, while preserving orientation and position of the inner closed curves with respect to the cuff,

the inner closed curve of the eighth segment has a shape that is different from the shapes of the inner closed curves of the other segments, while preserving orientation and position of the inner closed curves with respect to the cuff, respective ones of the electrode contact surfaces are fixed within the recesses defined by the second, fourth, sixth, tenth, and twelfth segments, and

none of the electrode contact surfaces is fixed within the recesses defined by the first, third, fifth, seventh, eighth, ninth, eleventh, and thirteenth segments.

For some applications, the first, fifth, ninth, and thirteenth segments define respective ones of the recesses that extend generally in a first radial direction, and the third, seventh, and eleventh segments define respective ones of the recesses that extend generally in a second radial direction different from the first radial direction. Alternatively or additionally, for some applications, the first through thirteenth lengths are 0.8 mm, 0.7 mm, 0.8 mm, 0.7 mm, 1.6 mm, 1.1 mm, 0.8 mm, 1.4 mm, 0.8 mm, 0.7 mm, 1.2 mm, 0.7 mm, and 0.8 mm, respectively.

For some applications:

13 sets of pluralities of the perpendicular cross sections define 13 segments of the cuff, respectively, such that the perpendicular cross sections are contiguous within each of the sets, and the sets are arranged in numerical order from a first set to a thirteenth set along the cuff, such that none of the segments overlap one other lengthwise along the cuff,

the 13 segments have respective first through thirteenth lengths, measured in parallel with the longitudinal axis, each of which is at least 0.1 mm,

respective ones of the electrode contact surfaces are fixed within the recesses defined of the second, fourth, sixth, tenth, and twelfth segments,

none of the electrode contact surfaces is fixed within the recesses defined by the first, third, fifth, seventh, eighth, ninth, eleventh, and thirteenth segments,

the apparatus further includes a control unit, which configures the electrode contact surface fixed in the recess of the fourth segment to function as an anode, and the electrode contact surfaces fixed within the recesses of the sixth and tenth segments to function as cathodes, and

the electrode contact surfaces fixed within the recesses of the second and twelfth segments are electrically device-coupled to each other, and are electrically device-coupled to neither the control unit nor an energy source.

For some applications:

13 sets of pluralities of the perpendicular cross sections define 13 segments of the cuff, respectively, such that the perpendicular cross sections are contiguous within each of the sets, and the sets are arranged in numerical order from a first set to a thirteenth set along the cuff, such that none of the segments overlap one other lengthwise along the cuff,

the 13 segments have respective first through thirteenth lengths, measured in parallel with the longitudinal axis, each of which is at least 0.1 mm,

respective ones of the electrode contact surfaces are fixed within the recesses defined of the second, fourth, sixth, tenth, and twelfth segments,

none of the electrode contact surfaces is fixed within the recesses defined by the first, third, fifth, seventh, eighth, ninth, eleventh, and thirteenth segments,

the apparatus further includes a control unit, which configures the electrode contact surface fixed in the recess of the fourth segment to function as an cathode, and the electrode contact surfaces fixed within the recesses of the sixth and tenth segments to function as anodes, and

the electrode contact surfaces fixed within the recesses of the second and twelfth segments are electrically device-coupled to each other, and are electrically device-coupled to neither the control unit nor an energy source.

There is still further provided, in accordance with an application of the present invention, apparatus placeable around tubular body tissue, the apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of recesses that are recessed radially outwardly from the tubular body tissue if the cuff is placed therearound, such that the cuff is recessed at every longitudinal location along an entire length of the cuff along the longitudinal axis, and each of the recesses extends along the longitudinal axis of the cuff and has a greatest length, measured in parallel with the longitudinal axis, that is less than 50% of the entire length of the cuff.

For some applications, the entire length of the cuff is between 1 and 40 mm.

For some applications, the cuff is shaped so as to come in contact with the tubular body tissue at a portion of, but not all, angles with respect to the longitudinal axis, at every longitudinal location along the entire length of the cuff, if the cuff is placed around the tubular body tissue in the closed position.

For some applications, the tubular body tissue is a nerve, and the cuff is configured to be applied to the nerve.

For some applications, at least one of the electrode contact surfaces is fixed within one of the recesses.

There is additionally provided, in accordance with an application of the present invention, apparatus placeable around an elliptical cylinder having a major axis that is between 1 and 8 mm and a minor axis that is between 0.5 and 6 mm, the apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of recesses that are recessed radially outwardly from the cylinder if the cuff is placed therearound, such that the cuff is recessed at every longitudinal location along an entire length of the cuff along the longitudinal axis, and each of the recesses extends along the longitudinal axis of the cuff and has a greatest length, measured in parallel with the longitudinal axis, that is less than 50% of the entire length of the cuff.

For some applications, the entire length of the cuff is between 1 and 40 mm.

For some applications, the cuff is shaped so as to come in contact with the cylinder at a portion of, but not all, angles with respect to the longitudinal axis, at every longitudinal location along the entire length of the cuff, if the cuff is placed around the cylinder in the closed position.

For some applications, at least one of the electrode contact surfaces is fixed within one of the recesses.

There is yet additionally provided, in accordance with an application of the present invention, apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, which are (i) distributed continuously along an entire length of the cuff along the longitudinal axis, and (ii) shaped so as to define respective inner closed curves surrounding the longitudinal axis, such that the inner closed curve of each of the segments is of uniform shape along the segment,

wherein each of the inner closed curves of at least four of the longitudinal segments has a different shape, and not merely a different size, from the inner closed curve of at least one adjacent longitudinal segment, when orientation and position of the segments with respect to the cuff are preserved, the at least four segments having respective lengths, measured in parallel with the longitudinal axis, each of which is at least 0.1 mm.

For some applications, the inner closed curve of each of the at least four segments is of uniform size along the segment.

For some applications, the inner closed curve of each of at least one of the at least four segments is of non-uniform size along the segment.

There is also provided, in accordance with an application of the present invention, apparatus including an electrode assembly, which includes:

one or more electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, which are (i) distributed continuously along an entire length of the cuff along the longitudinal axis, and (ii) shaped so as to define respective inner closed curves surrounding the longitudinal axis, such that the inner closed curve of each of the segments is of uniform shape along the segment,

wherein each of the inner closed curves of at least three of the longitudinal segments has a different shape, and not merely a different size, from the inner closed curve of at least one adjacent longitudinal segment, when orientation and position of the segments with respect to the cuff are preserved, the at least three segments having respective lengths, measured in parallel with the longitudinal axis, each of which is at least 0.1 mm and no more than 50% of the entire length of the cuff.

For some applications, the inner closed curve of each of the at least three segments is of uniform size along the segment.

For some applications, the inner closed curve of each of at least one of the at least three segments is of non-uniform size along the segment.

For some applications, each of the inner closed curves of at least four of the longitudinal segments has a different shape, and not merely a different size, from the inner closed curve of at least one adjacent longitudinal segment, when the orientation and position of the segments with respect to the cuff are preserved, the at least four segments having respective lengths, measured in parallel with the longitudinal axis, each of which is at least 0.1 mm.

For some applications, the entire length of the cuff is between 1 and 40 mm.

For some applications, each of the inner closed curves of at least five (e.g., at least ten) of the longitudinal segments has a different shape, and not merely a different size, from the inner closed curve of at least one adjacent longitudinal segment, when the orientation and position of the segments with respect to the cuff are preserved, the at least five (e.g., at least ten) segments having respective lengths, measured in parallel with the longitudinal axis, each of which is at least 0.1 mm.

For some applications, the inner closed curves of at least two of the longitudinal segments that are not longitudinally adjacent to each other have the same shape, when the orientation and position of the segments with respect to the cuff are preserved.

For some applications, the one or more electrode contact surfaces are fixed to exactly one of the segments.

For some applications, at least one of the electrode contact surfaces is fixed to an inner surface of a first one of the segments, and none of the electrode contact surfaces is fixed to an inner surface of at least a second one of the segments. For some applications, at least one of the electrode contact surfaces is fixed to an inner surface of a third one of the segments, and the first and third segments are longitudinally separated by the at least a second one of the segments.

For some applications, all of the inner closed curves, if superimposed while preserving orientation and position of the inner closed curves with respect to the cuff, would together define a combined innermost closed curve, and the inner closed curves respectively defined by the inner closed curves enclose respective areas, each of which areas is greater than an area enclosed by the combined innermost closed curve.

For some applications, all of the inner closed curves, if superimposed while preserving orientation and position of the inner closed curves with respect to the cuff, would together define a combined innermost closed curve, and each of the inner closed curves coincides with the combined innermost closed curve at a portion of, but not all, angles with respect to the longitudinal axis.

For some applications, the cuff is configured to assume the open and closed positions by defining a slit therethrough that extends along the entire length of the cuff.

There is further provided, in accordance with an application of the present invention, apparatus including an electrode assembly, which includes:

a plurality of electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, distributed continuously along an entire length of the cuff along the longitudinal axis, the segments having respective planar cross sections perpendicular to the longitudinal axis, which perpendicular cross sections define respective inner closed curves surrounding the longitudinal axis, such that the inner closed curve of each of the segments is of uniform shape along the segment,

wherein the inner closed curves, if superimposed while preserving orientation and position of the inner closed curves with respect to the cuff, would together define a combined innermost closed curve surrounding the longitudinal axis, which combined innermost closed curve, if extended along the entire length of the cuff, would define a combined innermost volume,

wherein a first one of the segments is shaped so as to define one or more first recesses that are recessed radially outward from the combined innermost volume,

wherein a second one of the segments is shaped so as to define one or more second recesses that are recessed radially outward from the combined innermost volume,

wherein a first one of the electrode contact surfaces is fixed within one of the first recesses, the one of the first recesses being recessed radially outward from the combined innermost volume at a first range of angles with respect to the longitudinal axis,

wherein a second one of the electrode contact surfaces is fixed within one of the second recesses, the one of the second recesses being recessed radially outward from the combined innermost volume at a second range of angles with respect to the longitudinal axis,

wherein one or more third ones of the segments longitudinally separate the first segment from the second segment, and each of the respective inner closed curves of the third segments coincides with the combined innermost closed curve at both the first and second ranges of angles with respect to the longitudinal axis, and

wherein the inner closed curves of the third segments enclose respective areas, each of which areas is greater than an area enclosed by the combined innermost closed curve.

For some applications, the inner closed curve of each of the segments is of uniform size along the segment.

For some applications, the inner closed curve of each of at least one of the segments is of non-uniform size along the segment.

For some applications, the entire length of the cuff is between 1 and 40 mm.

For some applications, the first and second ranges of angles coincide.

For some applications, none of the electrode contact surfaces is fixed to the one or more third segments.

For some applications, the one or more first recesses include the one of the first recesses and at least one additional first recess. For some applications, none of the electrode contact surfaces is fixed in the at least one additional first recess. For some applications, at least one of the electrode contact surfaces is fixed in the at least one additional first recess.

For some applications, the segments have respective lengths, measured in parallel with the longitudinal axis, each of which is at least 0.1 mm.

For some applications, all of the electrode contact surfaces are recessed away from the combined innermost volume.

For some applications, the plurality of electrode contact surfaces includes at least three electrode contact surfaces.

For some applications, the inner closed curves enclose respective areas, each of which areas is greater than an area enclosed by the combined innermost closed curve.

For some applications, each of the inner closed curves coincides with the combined innermost closed curve at a portion of, but not all, angles with respect to the longitudinal axis.

For some applications, the cuff is shaped such that the combined innermost closed curve is elliptical, for example, circular.

For some applications, the cuff is configured to assume the open and closed positions by defining a slit therethrough that extends along the entire length of the cuff.

There is still further provided, in accordance with an application of the present invention, apparatus placeable around tubular body tissue, including an electrode assembly, which includes:

a plurality of electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, distributed continuously along an entire length of the cuff along the longitudinal axis, the segments having respective planar cross sections perpendicular to the longitudinal axis, which perpendicular cross sections define respective inner closed curves surrounding the longitudinal axis, such that the inner closed curve of each of the segments is of uniform shape along the segment,

wherein a first one of the segments is shaped so as to define one or more first recesses that are recessed radially outward from the tubular body tissue if the cuff is placed therearound,

wherein a second one of the segments is shaped so as to define one or more second recesses that are recessed radially outward from the tubular body tissue if the cuff is placed therearound,

wherein a first one of the electrode contact surfaces is fixed within one of the first recesses, the one of the first recesses being recessed radially outward from the tubular body tissue, if the cuff is placed therearound, at a first range of angles with respect to the longitudinal axis,

wherein a second one of the electrode contact surfaces is fixed within one of the second recesses, the one of the second recesses being recessed radially outward from the tubular body tissue, if the cuff is placed therearound, at a second range of angles with respect to the longitudinal axis,

wherein one or more third ones of the segments longitudinally separate the first segment from the second segment, and each of the respective inner closed curves of the third segments coincides with the combined innermost closed curve at both the first and second ranges of angles with respect to the longitudinal axis, and

wherein the inner closed curves of the third segments enclose respective areas, each of which areas is greater than a perpendicular cross-sectional area of the tubular body tissue.

For some applications, the inner closed curve of each of the segments is of uniform size along the segment.

For some applications, the inner closed curve of each of at least one of the segments is of non-uniform size along the segment.

For some applications, the entire length of the cuff is between 1 and 40 mm.

For some applications, the first and second ranges of angles coincide.

For some applications, none of the electrode contact surfaces is fixed to the one or more third segments.

For some applications, the segments have respective lengths, measured in parallel with the longitudinal axis, each of which is at least 0.1 mm.

For some applications, the tubular body tissue is a nerve, and the cuff is configured to be applied to the nerve.

For some applications, all of the electrode contact surfaces are recessed away from the tubular body tissue, if the cuff is placed therearound.

There is additionally provided, in accordance with an application of the present invention, apparatus placeable around an elliptical cylinder having a major axis that is between 1 and 8 mm and a minor axis that is between 0.5 and 6 mm, the apparatus including an electrode assembly, which includes:

a plurality of electrode contact surfaces; and

a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, distributed continuously along an entire length of the cuff along the longitudinal axis, the segments having respective planar cross sections perpendicular to the longitudinal axis, which perpendicular cross sections define respective inner closed curves surrounding the longitudinal axis, such that the inner closed curve of each of the segments is of uniform shape along the segment,

wherein a first one of the segments is shaped so as to define one or more first recesses that are recessed radially outward from the cylinder if the cuff is placed therearound,

wherein a second one of the segments is shaped so as to define one or more second recesses that are recessed radially outward from the cylinder if the cuff is placed therearound,

wherein a first one of the electrode contact surfaces is fixed within one of the first recesses, the one of the first recesses being recessed radially outward from the cylinder, if the cuff is placed therearound, at a first range of angles with respect to the longitudinal axis,

wherein a second one of the electrode contact surfaces is fixed within one of the second recesses, the one of the second recesses being recessed radially outward from the cylinder, if the cuff is placed therearound, at a second range of angles with respect to the longitudinal axis,

wherein one or more third ones of the segments longitudinally separate the first segment from the second segment, and each of the respective inner closed curves of the third segments coincides with the combined innermost closed curve at both the first and second ranges of angles with respect to the longitudinal axis, and, and

wherein the inner closed curves of the third segments enclose respective areas, each of which areas is greater than a perpendicular cross-sectional area of the cylinder.

For some applications, the inner closed curve of each of the segments is of uniform size along the segment.

For some applications, the inner closed curve of each of at least one of the segments is of non-uniform size along the segment.

For some applications, the entire length of the cuff is between 1 and 40 mm.

For some applications, the first and second ranges of angles coincide.

There is yet additionally provided, in accordance with an application of the present invention, a method including:

providing an electrode assembly that includes (1) one or more electrode contact surfaces, and (2) a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of planar cross sections perpendicular to the longitudinal axis, distributed continuously along an entire length of the cuff along the longitudinal axis, such that the perpendicular cross sections define respective inner closed curves that together define an inner surface that defines and completely surrounds a volume that extends along the entire length of the cuff, wherein the inner closed curves of at least two of the perpendicular cross sections would cross, and not merely intersect, one another if superimposed while preserving orientation and position of the perpendicular cross sections with respect to the cuff;

while the cuff is in the open position, placing the electrode assembly around tubular body tissue of a subject; and

coupling the cuff to the tubular body tissue by causing the cuff to assume the closed position.

For some applications, providing the electrode assembly includes providing the electrode assembly in which all of the inner closed curves, if superimposed while preserving orientation and position of the perpendicular cross sections with respect to the cuff, would together define a combined innermost closed curve, and the inner closed curves respectively defined by the perpendicular cross sections enclose respective areas, each of which areas is greater than an area enclosed by the combined innermost closed curve.

For some applications, placing includes placing the electrode assembly around the nerve such that the electrode contacts surfaces are not in physical contact with the nerve.

There is also provided, in accordance with an application of the present invention, a method including:

providing an electrode assembly that includes (1) one or more electrode contact surfaces, and (2) a cuff, to which the electrode contact surfaces are fixed, and which: (a) includes an electrically insulating material, (b) has a longitudinal axis, and (c) is configured to assume open and closed positions, and (d) when in the closed position, is shaped so as to define a plurality of recesses that are recessed radially outwardly from the tubular body tissue, such that the cuff is recessed at every longitudinal location along an entire length of the cuff along the longitudinal axis, and each of the recesses extends along the longitudinal axis of the cuff and has a greatest length, measured in parallel with the longitudinal axis, that is less than 50% of the entire length of the cuff;

while the cuff is in the open position, placing the electrode assembly around tubular body tissue of a subject; and

coupling the cuff to the tubular body tissue by causing the cuff to assume the closed position.

For some applications, placing including placing the cuff around a nerve of the subject.

For some applications, coupling includes coupling the cuff to the tubular body tissue such that the cuff comes in contact with the tubular body tissue at a portion of, but not all, angles with respect to the longitudinal axis, at every longitudinal location along the entire length of the cuff.

For some applications, providing includes providing the electrode assembly in which first and second ones of the recesses overlap each other lengthwise along the cuff, and do not overlap each other anglewise with respect to the longitudinal axis.

For some applications, providing includes providing the electrode assembly in which at least a first one of the inner closed curves extends radially outwardly from the combined innermost volume in a first radial direction, and at least a second one of the inner closed curves, different from the first inner closed curve, extends radially outwardly from the combined innermost volume in a second radial direction different from the first radial direction.

For some applications, placing includes placing the electrode assembly around the nerve such that the electrode contacts surfaces are not in physical contact with the nerve.

There is further provided, in accordance with an application of the present invention, a method including:

providing an electrode assembly that includes (1) one or more electrode contact surfaces, and (2) a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, which are (i) distributed continuously along an entire length of the cuff along the longitudinal axis, and (ii) shaped so as to define respective inner closed curves surrounding the longitudinal axis, such that the inner closed curve of each of the segments is of uniform shape along the segment, wherein each of the inner closed curves of at least four of the longitudinal segments has a different shape, and not merely a different size, from the inner closed curve of at least one adjacent longitudinal segment, when orientation and position of the segments with respect to the cuff are preserved, the at least four segments having respective lengths, measured in parallel with the longitudinal axis, each of which is at least 0.1 mm;

while the cuff is in the open position, placing the electrode assembly around tubular body tissue of a subject; and

coupling the cuff to the tubular body tissue by causing the cuff to assume the closed position.

For some applications, placing includes placing the electrode assembly around the nerve such that the electrode contacts surfaces are not in physical contact with the nerve.

There is still further provided, in accordance with an application of the present invention, a method including:

providing an electrode assembly that includes (1) one or more electrode contact surfaces, and (2) a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, which are (i) distributed continuously along an entire length of the cuff along the longitudinal axis, and (ii) shaped so as to define respective inner closed curves surrounding the longitudinal axis, such that the inner closed curve of each of the segments is of uniform shape along the segment, wherein each of the inner closed curves of at least three of the longitudinal segments has a different shape, and not merely a different size, from the inner closed curve of at least one adjacent longitudinal segment, when orientation and position of the segments with respect to the cuff are preserved, the at least three segments having respective lengths, measured in parallel with the longitudinal axis, each of which is at least 0.1 mm and no more than 50% of the entire length of the cuff;

while the cuff is in the open position, placing the electrode assembly around tubular body tissue of a subject; and

coupling the cuff to the tubular body tissue by causing the cuff to assume the closed position.

For some applications, placing includes placing the electrode assembly around the nerve such that the electrode contacts surfaces are not in physical contact with the nerve.

There is additionally provided, in accordance with an application of the present invention, a method including:

providing an electrode assembly that includes (1) a plurality of electrode contact surfaces, and (2) a cuff, to which the electrode contact surfaces are fixed, and which: (a) includes an electrically insulating material, (b) has a longitudinal axis, (c) is configured to assume open and closed positions, and (d) when in the closed position, is shaped so as to define a plurality of longitudinal segments, distributed continuously along an entire length of the cuff along the longitudinal axis, the segments having respective planar cross sections perpendicular to the longitudinal axis, which perpendicular cross sections define respective inner closed curves surrounding the longitudinal axis, such that the inner closed curve of each of the segments is of uniform shape along the segment;

while the cuff is in the open position, placing the electrode assembly around tubular body tissue of a subject; and

coupling the cuff to the tubular body tissue by causing the cuff to assume the closed position, such that:
a first one of the segments is shaped so as to define one or more first recesses that are recessed radially outward from the tubular body tissue,
a second one of the segments is shaped so as to define one or more second recesses that are recessed radially outward from the tubular body tissue,
a first one of the electrode contact surfaces is fixed within one of the first recesses, the one of the first recesses being recessed radially outward from the tubular body tissue at a first range of angles with respect to the longitudinal axis,
a second one of the electrode contact surfaces is fixed within one of the second recesses, the one of the second recesses being recessed radially outward from the tubular body tissue at a second range of angles with respect to the longitudinal axis,
one or more third ones of the segments longitudinally separate the first segment from the second segment, and each of the respective inner closed curves of the third segments coincides with the combined innermost closed curve at both the first and second ranges of angles with respect to the longitudinal axis, and
perpendicular cross-sectional areas respectively enclosed by the third segments are each greater than a perpendicular cross-sectional area of the tubular body tissue.

For some applications, placing includes placing the electrode assembly around the nerve such that the electrode contacts surfaces are not in physical contact with the nerve.

In some embodiments of the present invention, apparatus for treating a heart condition comprises a multipolar electrode device that is applied to a portion of a vagus nerve that innervates the heart of a patient. Typically, the system is configured to treat heart failure and/or heart arrhythmia, such as atrial fibrillation or tachycardia. A control unit typically drives the electrode device to (i) apply signals to induce the propagation of efferent action potentials towards the heart, and (ii) suppress artificially-induced afferent and efferent action potentials, in order to minimize any unintended side effect of the signal application. Alternatively, the control unit drives the electrode device to apply signals that induce symmetric or asymmetric bi-directional propagation of nerve impulses.

The control unit typically suppresses afferent action potentials induced by the cathodic current by inhibiting essentially all or a large fraction of fibers using anodal current (“afferent anodal current”) from a second set of one or more anodes (the “afferent anode set”). The afferent anode set is typically placed between the central cathode and the edge of the electrode device closer to the brain (the “afferent edge”), to block a large fraction of fibers from conveying signals in the direction of the brain during application of the afferent anodal current.

In some embodiments of the present invention, the cathodic current is applied with an amplitude sufficient to induce action potentials in large- and medium-diameter fibers (e.g., A- and B-fibers), but insufficient to induce action potentials in small-diameter fibers (e.g., C-fibers). Simultaneously, a small anodal current is applied in order to inhibit action potentials induced by the cathodic current in the large-diameter fibers (e.g., A-fibers). This combination of cathodic and anodal current generally results in the stimulation of medium-diameter fibers (e.g., B-fibers) only. At the same time, a portion of the afferent action potentials induced by the cathodic current are blocked, as described above. By not stimulating large-diameter fibers, such stimulation generally avoids adverse effects sometimes associated with recruitment of such large fibers, such as dyspnea and hoarseness. Stimulation of small-diameter fibers is avoided because these fibers transmit pain sensations and are important for regulation of reflexes such as respiratory reflexes. Alternatively, the control unit is configured to apply a current that does not select for fibers of particular diameters.

In some embodiments of the present invention, the efferent anode set comprises a plurality of anodes. Application of the efferent anodal current in appropriate ratios from the plurality of anodes in these embodiments generally minimizes the “virtual cathode effect,” whereby application of too large an anodal current creates a virtual cathode, which stimulates rather than blocks fibers. When such techniques are not used, the virtual cathode effect generally hinders blocking of smaller-diameter fibers, because a relatively large anodal current is typically necessary to block such fibers, and this same large anodal current induces the virtual cathode effect Likewise, the afferent anode set typically comprises a plurality of anodes in order to minimize the virtual cathode effect in the direction of the brain.

In some embodiments of the present invention, the efferent and afferent anode sets each comprise exactly one electrode, which are directly electrically coupled to each other. The cathodic current is applied with an amplitude sufficient to induce action potentials in large- and medium-diameter fibers (e.g., A- and B-fibers), but insufficient to induce action potentials in small-diameter fibers (e.g., C-fibers). Simultaneously, an anodal current is applied in order to inhibit action potentials induced by the cathodic current in the large-diameter fibers (e.g., A-fibers), but not in the small- and medium-diameter fibers (e.g., B- and C-fibers). This combination of cathodic and anodal current generally results in the stimulation of medium-diameter fibers (e.g., B-fibers) only.

Typically, parasympathetic stimulation of the vagus nerve is applied responsive to one or more sensed physiological parameters or other parameters, such as heart rate, electrocardiogram (ECG), blood pressure, indicators of cardiac contractility, cardiac output, norepinephrine concentration, baroreflex sensitivity, or motion of the patient. Typically, stimulation is applied in a closed-loop system in order to achieve and maintain a desired heart rate responsive to one or more such sensed parameters. For some applications, such stimulation is applied chronically, i.e., during a period having a duration of at least one week, e.g., at least one month.

In some embodiments of the present invention, vagal stimulation is applied in a burst (i.e., a series of pulses). The application of the burst in each cardiac cycle typically commences after a variable delay after a detected R-wave, P-wave, or other feature of an ECG. The delay is typically calculated in real time using a function, the inputs of which include one or more pre-programmed but updateable constants and one or more sensed parameters, such as the R-R interval between cardiac cycles and/or the P-R interval. Alternatively or additionally, a lookup table of delays is used to determine in real time the appropriate delay for each application of pulses, based on the one or more sensed parameters.

In some embodiments of the present invention, the control unit is configured to drive the electrode device to stimulate the vagus nerve so as to reduce the heart rate of the subject towards a target heart rate. Parameters of stimulation are varied in real time in order to vary the heart-rate-lowering effects of the stimulation. In embodiments of the present invention in which the stimulation is applied in a series of pulses that are synchronized with the cardiac cycle of the subject, such as described hereinabove, parameters of such pulse series typically include, but are not limited to: (a) timing of the stimulation within the cardiac cycle, (b) pulse duration (width), (c) pulse repetition interval, (d) pulse period, (e) number of pulses per burst, also referred to herein as “pulses per trigger” (PPT), (f) amplitude, (g) duty cycle, (h) choice of vagus nerve, and (i) “on”/“off” ratio and timing (i.e., during intermittent operation).

In some embodiments of the present invention, the control unit is configured to drive the electrode device to stimulate the vagus nerve so as to modify heart rate variability of the subject. For some applications, the control unit is configured to apply stimulation with parameters that tend to or that are selected to reduce heart rate variability, while for other applications parameters are used that tend to or that are selected to increase variability. For some applications, the parameters of the stimulation are selected to both reduce the heart rate of the subject and heart rate variability of the subject. For other applications, the parameters are selected to reduce heart rate variability while substantially not reducing the heart rate of the subject. For some applications, the control unit is configured to drive the electrode device to stimulate the vagus nerve so as to modify heart rate variability in order to treat a condition of the subject.

Advantageously, the techniques described herein generally enable relatively fine control of the level of stimulation of the vagus nerve, by imitating the natural physiological smaller-to-larger diameter recruitment order of nerve fibers. This recruitment order allows improved and more natural control over the heart rate. Such fine control is particularly advantageous when applied in a closed-loop system, wherein such control results in smaller changes in heart rate and lower latencies in the control loop, which generally contribute to greater loop stability and reduced loop stabilization time.

“Heart failure,” as used in the specification and the claims, is to be understood to include all forms of heart failure, including ischemic heart failure, non-ischemic heart failure, and diastolic heart failure.

“Vagus nerve,” and derivatives thereof, as used in the specification and the claims, is to be understood to include portions of the left vagus nerve, the right vagus nerve, the cervical vagus nerve, branches of the vagus nerve such as the superior cardiac nerve, superior cardiac branch, and inferior cardiac branch, and the vagus trunk. Similarly, stimulation of the vagus nerve is described herein by way of illustration and not limitation, and it is to be understood that in some embodiments of the present invention, other autonomic and/or parasympathetic nerves and/or parasympathetic tissue are stimulated, including sites where the vagus nerve innervates a target organ, vagal ganglions, nerves in the epicardial fat pads, a carotid artery, a jugular vein (e.g., an internal jugular vein), a carotid sinus, a coronary sinus, a vena cava vein, a pulmonary vein, and/or a right ventricle, for treatment of heart conditions or other conditions.

There is therefore provided, in accordance with an embodiment of the present invention, apparatus for treating a condition of a subject, including:

an electrode device, adapted to be coupled to an autonomic nerve of the subject; and

a control unit, adapted to:

drive the electrode device to apply to the nerve a stimulating current, which is capable of inducing action potentials in a therapeutic direction in a first set and a second set of nerve fibers of the nerve, and

drive the electrode device to apply to the nerve an inhibiting current, which is capable of inhibiting the induced action potentials traveling in the therapeutic direction in the second set of nerve fibers, the nerve fibers in the second set having generally larger diameters than the nerve fibers in the first set.

It is to be understood that for some applications the stimulating current may also be capable of inducing action potentials in a non-therapeutic direction opposite the therapeutic direction, and that this embodiment of the present invention is not limited to application of a stimulating current that is capable of inducing action potentials only in a therapeutic direction.

In an embodiment of the present invention, the electrode device is adapted to be coupled to parasympathetic nervous tissue of the subject, and the control unit is adapted to drive the electrode device to apply to the tissue a stimulating current that is not necessarily configured to stimulate only a subset of nerve fibers of the tissue.

In an embodiment, the autonomic nerve includes a parasympathetic nerve of the subject, and the electrode device is adapted to be coupled to the parasympathetic nerve.

In an embodiment, the control unit is adapted to configure the stimulating current to treat one or more of the following conditions of the subject: heart failure, atrial fibrillation, angina, cardiac arrest, arrhythmia, myocardial infarction, hypertension, endocarditis, myocarditis, asthma, an allergy, a neoplastic disorder, rheumatoid arthritis, septic shock, hepatitis, hypertension, diabetes mellitus, an autoimmune disease, a gastric ulcer, a neurological disorder, pain, a migraine headache, peripheral neuropathy, an addiction, a psychiatric disorder, obesity, an eating disorder, impotence, a skin disease, an infectious disease, a vascular disease, a kidney disorder, and a urinary tract disorder.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat heart failure of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat atrial fibrillation of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat angina of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat cardiac arrest of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat arrhythmia of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat myocardial infarction of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat hypertension of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat endocarditis of the subject.

In an embodiment, the nerve includes a vagus nerve of the subject, the electrode device is adapted to be coupled to the vagus nerve, and the control unit is adapted to configure the stimulating current to change a physiological parameter of the subject selected from the list consisting of: a hemodynamic parameter, and a cardiac geometry parameter, sufficiently to treat myocarditis of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat heart failure of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat atrial fibrillation of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat angina of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat cardiac arrest of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat arrhythmia of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat myocardial infarction of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat hypertension of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat endocarditis of the subject.

In an embodiment, the nerve includes a vagus nerve of the subject, the electrode device is adapted to be coupled to the vagus nerve, and the control unit is adapted to configure the stimulating current to change a myocardial cellular anatomy parameter of the subject sufficiently to treat myocarditis of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a stimulation-treatable condition of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a portion of a body of the subject selected from the list consisting of: a heart, a brain, lungs, an organ of a respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to attenuate muscle contractility of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat heart failure of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat atrial fibrillation of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat angina of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat cardiac arrest of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat arrhythmia of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat myocardial infarction of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat hypertension of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat endocarditis of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat myocarditis of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat asthma of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat an allergy of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a neoplastic disorder of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat rheumatoid arthritis of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat septic shock of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat hepatitis of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat hypertension of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat diabetes mellitus of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat an autoimmune disease of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a gastric ulcer of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a neurological disorder of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat pain of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a migraine headache of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat peripheral neuropathy of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat an addiction of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a psychiatric disorder of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat obesity of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat an eating disorder of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat impotence of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a skin disease of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat an infectious disease of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a vascular disease of the subject.

In an embodiment, the nerve includes a vagus nerve of the subject, the electrode device is adapted to be coupled to the vagus nerve, and the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein, sufficiently to treat a disorder of the subject selected from the list consisting of: a kidney disorder, and a urinary tract disorder.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat heart failure of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat atrial fibrillation of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat angina of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat cardiac arrest of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat arrhythmia of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat myocardial infarction of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat hypertension of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat endocarditis of the subject.

In an embodiment, the nerve includes a vagus nerve of the subject, the electrode device is adapted to be coupled to the vagus nerve, and the control unit is adapted to configure the stimulating current to change a level of a neurohormone peptide selected from the list consisting of: N-terminal pro-brain natriuretic peptide (NT-pro-BNP), and a catecholamine, sufficiently to treat myocarditis of the subject.

In an embodiment, the nerve includes a vagus nerve of the subject, the electrode device is adapted to be coupled to the vagus nerve, the subject has undergone a coronary artery bypass graft (CABG) procedure, and the control unit is adapted to configure the stimulating current to suppress at least one of: post-CABG inflammation and post-CABG atrial fibrillation.

In an embodiment, the apparatus includes an electrical cardioversion device, the subject is suffering from atrial fibrillation, and the control unit is adapted to configure the stimulating current to suppress inflammation of the subject, and, thereafter, drive the cardioversion device to apply cardioversion treatment to the subject.

In an embodiment, the inhibiting current includes a first inhibiting current, and the control unit is adapted to drive the electrode device to apply to the nerve a second inhibiting current, which is capable of inhibiting device-induced action potentials traveling in a non-therapeutic direction opposite the therapeutic direction in the first and second sets of nerve fibers.

In an embodiment, the electrode device includes a cathode, adapted to apply the stimulating current, and a primary set of anodes, adapted to apply the inhibiting current. For some applications, the primary set of anodes includes a primary anode and a secondary anode, adapted to be disposed so that the primary anode is located between the secondary anode and the cathode, and the secondary anode is adapted to apply a current with an amplitude less than about one half an amplitude of a current applied by the primary anode.

In an embodiment, the nerve includes a vagus nerve of the subject, the electrode device is adapted to be coupled to the vagus nerve, and the control unit is adapted to configure the stimulating current to change a level of at least one NO synthase of the subject selected from the list consisting of: NOS-1, NOS-2, and NOS-3. For some applications, the control unit is adapted to configure the stimulating current to reduce the level of NOS-1 and the level of NOS-2, and to increase the level of NOS-3. For some applications, the control unit is adapted to apply the stimulating and inhibiting currents during a period having a duration of at least one week.

For some applications, the control unit is adapted to configure the stimulating current to change the level of the at least one NO synthase by an amount sufficient to treat a stimulation-treatable condition of the subject.

For some applications, the control unit is adapted to configure the stimulating current to change the level of the at least one NO synthase by an amount sufficient to treat a portion of a body of the subject selected from the list consisting of: a brain, lungs, an organ of a respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex.

For some applications, the control unit is adapted to configure the stimulating current to change the level of the at least one NO synthase by an amount sufficient to attenuate muscle contractility of the subject.

For some applications, the control unit is adapted to configure the stimulating current to change the level of the at least one NO synthase of heart tissue of the subject. For some applications, the control unit is adapted to configure the stimulating current to change the level of the at least one NO synthase of the heart tissue by an amount sufficient to treat heart failure of the subject. For some applications, the control unit is adapted to apply the stimulating and inhibiting currents during a period having a duration of at least one week.

For some applications, the control unit is adapted to configure the stimulating current to change the level of the at least one NO synthase of the heart tissue by an amount sufficient to treat atrial fibrillation of the subject. For some applications, the control unit is adapted to apply the stimulating and inhibiting currents during a period having a duration of at least one week.

For some applications, the control unit is adapted to configure the stimulating current to change the level of the at least one NO synthase of the heart tissue by an amount sufficient to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

For some applications, the control unit is adapted to configure the stimulating current to change the level of the at least one NO synthase of the heart tissue by an amount sufficient to treat one or more of the following conditions of the subject: angina, cardiac arrest, arrhythmia, myocardial infarction, hypertension, endocarditis, myocarditis, asthma, an allergy, a neoplastic disorder, rheumatoid arthritis, septic shock, hepatitis, hypertension, diabetes mellitus, an autoimmune disease, a gastric ulcer, a neurological disorder, pain, a migraine headache, peripheral neuropathy, an addiction, a psychiatric disorder, obesity, an eating disorder, impotence, a skin disease, an infectious disease, a vascular disease, a kidney disorder, and a urinary tract disorder.

In an embodiment, the nerve includes a vagus nerve of the subject, the electrode device is adapted to be coupled to the vagus nerve, and the control unit is adapted to configure the stimulating current to suppress inflammation of the subject.

For some applications, the control unit is adapted to configure the stimulating current to suppress the inflammation sufficiently to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

For some applications, the control unit is adapted to configure the stimulating current to suppress the inflammation sufficiently to treat a stimulation-treatable condition of the subject.

For some applications, the control unit is adapted to configure the stimulating current to suppress inflammation sufficiently to treat heart failure of the subject.

For some applications, the control unit is adapted to configure the stimulating current to suppress inflammation sufficiently to treat atrial fibrillation of the subject. For some applications, the control unit is adapted to configure the stimulating current to reduce thromboembolism of the subject. For some applications, the control unit is adapted to configure the stimulating current to increase a likelihood of successful cardioversion.

In an embodiment, the nerve includes a vagus nerve of the subject, the electrode device is adapted to be coupled to the vagus nerve, and the control unit is adapted to configure the stimulating current to inhibit release of a proinflammatory cytokine.

For some applications, the control unit is adapted to configure the stimulating current to inhibit the release of the proinflammatory cytokine sufficiently to treat heart failure of the subject. For some applications, the control unit is adapted to configure the stimulating current to inhibit the release of the proinflammatory cytokine sufficiently to treat atrial fibrillation of the subject.

For some applications, the control unit is adapted to apply the stimulating and inhibiting currents during a period having a duration of at least one week. For some applications, the control unit is adapted to configure the stimulating current to inhibit the release of the proinflammatory cytokine sufficiently to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock. For some applications, the control unit is adapted to configure the stimulating current to inhibit the release of the proinflammatory cytokine sufficiently to treat a stimulation-treatable condition of the subject.

In an embodiment, the nerve includes a vagus nerve of the subject, the electrode device is adapted to be coupled to the vagus nerve, and the control unit is adapted to configure the stimulating current to inhibit release of C-reactive protein.

For some applications, the control unit is adapted to configure the stimulating current to inhibit the release of the C-reactive protein sufficiently to treat heart failure of the subject. For some applications, the control unit is adapted to configure the stimulating current to inhibit the release of the C-reactive protein sufficiently to treat atrial fibrillation of the subject. For some applications, the control unit is adapted to apply the stimulating and inhibiting currents during a period having a duration of at least one week.

In an embodiment, the nerve includes a vagus nerve of the subject, the electrode device is adapted to be coupled to the vagus nerve, and the control unit is adapted to configure the stimulating current to change a level of N-terminal pro-brain natriuretic peptide (NT-pro-BNP). For some applications, the control unit is adapted to configure the stimulating current to change the level of NT-pro-BNP by an amount sufficient to treat heart failure of the subject. For some applications, the control unit is adapted to configure the stimulating current to change the level of NT-pro-BNP by an amount sufficient to treat atrial fibrillation of the subject. For some applications, the control unit is adapted to apply the stimulating and inhibiting currents during a period having a duration of at least one week.

In an embodiment, the nerve includes a vagus nerve of the subject, the electrode device is adapted to be coupled to the vagus nerve, and the control unit is adapted to configure the stimulating current to change a level of Connexin 43. For some applications, the control unit is adapted to configure the stimulating current to change the level of Connexin 43 by an amount sufficient to treat one or more of the following conditions of the subject: heart failure, atrial fibrillation, angina, cardiac arrest, arrhythmia, myocardial infarction, hypertension, endocarditis, and myocarditis.

For some applications, the control unit is adapted to apply the stimulating and inhibiting currents during a period having a duration of at least one week.

For some applications, the control unit is adapted to configure the stimulating current to change the level of Connexin 43 by an amount sufficient to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

For some applications, the control unit is adapted to configure the stimulating current to change the level of Connexin 43 by an amount sufficient to treat a portion of a body of the subject selected from the list consisting of: a heart, a brain, lungs, an organ of a respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex.

For some applications, the control unit is adapted to configure the stimulating current to change the level of Connexin 43 by an amount sufficient to treat a condition of the subject selected from the list consisting of: tuberous sclerosis, breast cancer, carcinomas, melanoma, osteoarthritis, a wound, a seizure, bladder overactivity, bladder outlet obstruction, Huntington's disease, and Alzheimer's disease.

For some applications, the autonomic nerve includes a lacrimal nerve, and the control unit is adapted to drive the electrode device to apply the stimulating and inhibiting currents to the lacrimal nerve. For some applications, the autonomic nerve includes a salivary nerve, and the control unit is adapted to drive the electrode device to apply the stimulating and inhibiting currents to the salivary nerve. For some applications, the autonomic nerve includes a pelvic splanchnic nerve, and the control unit is adapted to drive the electrode device to apply the stimulating and inhibiting currents to the pelvic splanchnic nerve.

In an embodiment, the autonomic nerve includes a sympathetic nerve, and the control unit is adapted to drive the electrode device to apply the stimulating and inhibiting currents to the sympathetic nerve.

For some applications, the control unit is adapted to drive the electrode device to apply the stimulating and inhibiting currents to the nerve so as to affect behavior of one or more of the following organs of the subject, so as to treat the condition: a stomach, a pancreas, a small intestine, a liver, a spleen, a kidney, a bladder, a rectum, a large intestine, a reproductive organ, and an adrenal gland.

There is also provided, in accordance with an embodiment of the present invention, a method for treating a condition of a subject, including:

applying, to an autonomic nerve of the subject, a stimulating current which is capable of inducing action potentials in a therapeutic direction in a first set and a second set of nerve fibers of the nerve; and

applying to the nerve an inhibiting current which is capable of inhibiting the induced action potentials traveling in the therapeutic direction in the second set of nerve fibers, the nerve fibers in the second set having generally larger diameters than the nerve fibers in the first set.

In an embodiment, the autonomic nerve includes a parasympathetic nerve of the subject, and applying the stimulating current includes applying the stimulating current to the parasympathetic nerve.

In an embodiment, the method includes identifying a clinical benefit for the subject to experience a change in a level of at least one NO synthase of the subject selected from the list consisting of: NOS-1, NOS-2, and NOS-3; the nerve includes a vagus nerve of the subject; applying the stimulating and inhibiting currents includes applying the stimulating and inhibiting currents to the vagus nerve; and applying the stimulating current includes configuring the stimulating current to change the level of the at least one NO synthase.

There is further provided, in accordance with an embodiment of the present invention, apparatus for treating a condition of a subject, including:

an electrode device, adapted to be coupled to parasympathetic nervous tissue of the subject; and

a control unit, adapted to drive the electrode device to apply a stimulating current to the tissue, and to configure the stimulating current to change a level of at least one NO synthase of the subject selected from the list consisting of: NOS-1, NOS-2, and NOS-3.

For some applications, the control unit is adapted to configure the stimulating current to reduce the level of NOS-1 and the level of NOS-2, and to increase the level of NOS-3.

For some applications, the control unit is adapted to apply the stimulating current during a period having a duration of at least one week.

In an embodiment, the control unit is adapted to configure the stimulating current to change the level of the at least one NO synthase of heart tissue of the subject.

For some applications, the control unit is adapted to configure the stimulating current to change the level of the at least one NO synthase of the heart tissue by an amount sufficient to treat heart failure of the subject.

For some applications, the control unit is adapted to configure the stimulating current to change the level of the at least one NO synthase of the heart tissue by an amount sufficient to treat atrial fibrillation of the subject.

For some applications, the control unit is adapted to configure the stimulating current to change the level of the at least one NO synthase of the heart tissue by an amount sufficient to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is still further provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to change a physiological parameter of the subject selected from the list consisting of: a hemodynamic parameter, and a cardiac geometry parameter, sufficiently to treat a cardiac condition of the subject.

For some applications, the cardiac condition is selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock, and the control unit is adapted to configure the stimulating current to change the selected physiological parameter sufficiently to treat the selected cardiac condition.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is yet further provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to change a myocardial cellular anatomy parameter of the subject sufficiently to treat a cardiac condition of the subject.

For some applications, the cardiac condition is selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock, and the control unit is adapted to configure the stimulating current to change the myocardial cellular anatomy parameter sufficiently to treat the selected cardiac condition.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is also provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to suppress inflammation of the subject.

For some applications, the control unit is adapted to configure the stimulating current to suppress the inflammation sufficiently to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, cardiogenic shock, atrial fibrillation, and thromboembolism.

For some applications, the control unit is adapted to configure the stimulating current to suppress the inflammation sufficiently to treat a stimulation-treatable condition of the subject.

In an embodiment, the control unit is adapted to configure the stimulating current to change a level of an inflammatory marker selected from the list consisting of: tumor necrosis factor alpha, interleukin 6, activin A, transforming growth factor, interferon, interleukin 1 beta, interleukin 18, interleukin 12, and C-reactive protein. For some applications, the control unit is adapted to configure the stimulating current to change the level of the selected inflammatory marker sufficiently to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock. For some applications, the control unit is adapted to configure the stimulating current to change the level of the selected inflammatory marker sufficiently to treat a stimulation-treatable condition of the subject. For some applications, the control unit is adapted to configure the stimulating current to change the level of the selected inflammatory marker sufficiently to treat a portion of a body of the subject selected from the list consisting of: a heart, a brain, lungs, an organ of a respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex. For some applications, the control unit is adapted to configure the stimulating current to change the level of the selected inflammatory marker sufficiently to attenuate muscle contractility of the subject.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is further provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to change a level of a neurohormone peptide selected from the list consisting of: N-terminal pro-brain natriuretic peptide (NT-pro-BNP), and a catecholamine, sufficiently to treat a cardiac condition of the subject.

For some applications, the cardiac condition is selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock, and the control unit is adapted to configure the stimulating current to change the level of the selected neurohormone peptide sufficiently to treat the selected cardiac condition.

In an embodiment, the neurohormone peptide includes NT-pro-BNP, and the control unit is adapted to configure the stimulating current to change the level of NT-pro-BNP. For some applications, the control unit is adapted to configure the stimulating current to change the level of NT-pro-BNP by an amount sufficient to treat heart failure of the subject. For some applications, the control unit is adapted to configure the stimulating current to change the level of NT-pro-BNP by an amount sufficient to treat atrial fibrillation of the subject. For some applications, the control unit is adapted to apply the stimulating current during a period having a duration of at least one week.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is still further provided, in accordance with an embodiment of the present invention, apparatus for treating a subject who has undergone a coronary artery bypass graft (CABG) procedure, including:

an electrode device, adapted to be coupled to parasympathetic nervous tissue of the subject; and

a control unit, adapted to drive the electrode device to apply a stimulating current to the tissue, and to configure the stimulating current to suppress at least one of: post-CABG inflammation and post-CABG atrial fibrillation.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is yet further provided, in accordance with an embodiment of the present invention, apparatus for treating a subject suffering from atrial fibrillation, including:

an electrical cardioversion device;

an electrode device, adapted to be coupled to parasympathetic nervous tissue of the subject; and

a control unit, adapted to drive the electrode device to apply a stimulating current to the tissue, and to configure the stimulating current to suppress inflammation of the subject, and, thereafter, drive the cardioversion device to apply cardioversion treatment to the subject.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is also provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to inhibit release of a proinflammatory cytokine.

For some applications, the control unit is adapted to configure the stimulating current to inhibit the release of the proinflammatory cytokine sufficiently to treat heart failure of the subject. For some applications, the control unit is adapted to configure the stimulating current to inhibit the release of the proinflammatory cytokine sufficiently to treat atrial fibrillation of the subject.

For some applications, the control unit is adapted to apply the stimulating current during a period having a duration of at least one week. For some applications, the control unit is adapted to configure the stimulating current to inhibit the release of the proinflammatory cytokine sufficiently to treat a cardiac condition of the subject selected from the list consisting of: heart failure, congestive heart failure, diastolic heart failure, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock. For some applications, the control unit is adapted to configure the stimulating current to inhibit the release of the proinflammatory cytokine sufficiently to treat a stimulation-treatable condition of the subject.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is additionally provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to inhibit release of C-reactive protein.

For some applications, the control unit is adapted to configure the stimulating current to inhibit the release of the C-reactive protein sufficiently to treat heart failure of the subject. For some applications, the control unit is adapted to configure the stimulating current to inhibit the release of the C-reactive protein sufficiently to treat atrial fibrillation of the subject.

For some applications, the control unit is adapted to apply the stimulating current during a period having a duration of at least one week.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is yet additionally provided, in accordance with an embodiment of the present invention, apparatus for treating a subject, including:

an electrode device, adapted to be coupled to parasympathetic nervous tissue of the subject; and

a control unit, adapted to drive the electrode device to apply a stimulating current to the tissue, and to configure the stimulating current to change a level of Connexin 43.

For some applications, the control unit is adapted to configure the stimulating current to change the level of Connexin 43 by an amount sufficient to treat a cardiac condition of the subject selected from the list consisting of: heart failure, congestive heart failure, diastolic heart failure, atrial fibrillation, angina, cardiac arrest, arrhythmia, myocardial infarction, hypertension, endocarditis, myocarditis, atherosclerosis, restenosis, cardiomyopathy, post-myocardial infarct remodeling, arteritis, thrombophlebitis, pericarditis, myocardial ischemia, sick sinus syndrome, and cardiogenic shock.

For some applications, the control unit is adapted to configure the stimulating current to change the level of Connexin 43 by an amount sufficient to treat a portion of a body of the subject selected from the list consisting of: a heart, a brain, lungs, an organ of a respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex.

For some applications, the control unit is adapted to configure the stimulating current to change the level of Connexin 43 by an amount sufficient to treat a condition of the subject selected from the list consisting of: tuberous sclerosis, breast cancer, carcinomas, melanoma, osteoarthritis, a wound, a seizure, bladder overactivity, bladder outlet obstruction, Huntington's disease, and Alzheimer's disease.

For some applications, the control unit is adapted to apply the stimulating current during a period having a duration of at least one week.

In an embodiment, the parasympathetic tissue includes a vagus nerve of the subject, and the electrode device is adapted to be coupled to the vagus nerve. Alternatively, the parasympathetic tissue includes an epicardial fat pad of the subject, and the electrode device is adapted to be coupled to the epicardial fat pad. Further alternatively, the parasympathetic tissue is selected from the list consisting of: parasympathetic tissue of a pulmonary vein, parasympathetic tissue of a carotid artery, parasympathetic tissue of a carotid sinus, parasympathetic tissue of a coronary sinus, parasympathetic tissue of a vena cava vein, parasympathetic tissue of a right ventricle, and parasympathetic tissue of a jugular vein, and the electrode device is adapted to be coupled to the selected parasympathetic tissue.

There is still additionally provided, in accordance with an embodiment of the present invention, a method for treating a condition of a subject, including:

identifying a clinical benefit for the subject to experience a change in a level of at least one NO synthase of the subject selected from the list consisting of: NOS-1, NOS-2, and NOS-3;

applying a stimulating current to parasympathetic nervous tissue of the subject; and

configuring the stimulating current to change the level of the at least one NO synthase.

There is yet additionally provided, in accordance with an embodiment of the present invention, a method for treating a subject, including:

identifying a clinical benefit for the subject to experience a change in a physiological parameter of the subject selected from the list consisting of: a hemodynamic parameter, and a cardiac geometry parameter;

applying a stimulating current to parasympathetic nervous tissue of the subject; and

configuring the stimulating current to change the selected physiological parameter sufficiently to treat a cardiac condition of the subject.

There is still additionally provided, in accordance with an embodiment of the present invention, a method for treating a subject, including:

identifying a clinical benefit for the subject to experience a change in a myocardial cellular anatomy parameter of the subject;

applying a stimulating current to parasympathetic nervous tissue of the subject; and

configuring the stimulating current to change the myocardial cellular anatomy parameter sufficiently to treat a cardiac condition of the subject.

There is yet additionally provided, in accordance with an embodiment of the present invention, a method for treating a subject, including:

identifying a clinical benefit for the subject to experience a suppression of inflammation of the subject;

applying a stimulating current to parasympathetic nervous tissue of the subject; and

configuring the stimulating current to suppress the inflammation.

There is also provided, in accordance with an embodiment of the present invention, a method for treating a subject, including:

identifying a clinical benefit for the subject to experience a change in a level of a neurohormone peptide selected from the list consisting of: N-terminal pro-brain natriuretic peptide (NT-pro-BNP), and a catecholamine;

applying a stimulating current to parasympathetic nervous tissue of the subject; and

configuring the stimulating current to change the level of the selected neurohormone peptide sufficiently to treat a cardiac condition of the subject.

There is further provided, in accordance with an embodiment of the present invention, a method including:

selecting a subject who has undergone a coronary artery bypass graft (CABG) procedure;

applying a stimulating current to parasympathetic nervous tissue of the subject; and

configuring the stimulating current to suppress at least one of: post-CABG inflammation and post-CABG atrial fibrillation.

There is still further provided, in accordance with an embodiment of the present invention, a method including:

selecting a subject suffering from atrial fibrillation;

applying a stimulating current to parasympathetic nervous tissue of the subject;

configuring the stimulating current to suppress inflammation of the subject; and

after applying and configuring the stimulating current, applying electrical cardioversion treatment to the subject.

There is additionally provided, in accordance with an embodiment of the present invention, a method for treating a subject, including:

identifying a clinical benefit for the subject to experience inhibition of release of a proinflammatory cytokine;

applying a stimulating current to parasympathetic nervous tissue of the subject; and

configuring the stimulating current to inhibit release of the proinflammatory cytokine.

There is yet additionally provided, in accordance with an embodiment of the present invention, a method for treating a subject, including:

identifying a clinical benefit for the subject to experience inhibition of release of C-reactive protein;

applying a stimulating current to parasympathetic nervous tissue of the subject; and

configuring the stimulating current to inhibit release of the C-reactive protein.

There is still additionally provided, in accordance with an embodiment of the present invention, a method for treating a subject, including:

identifying a clinical benefit for the subject to experience a change in a level of Connexin 43;

applying a stimulating current to parasympathetic nervous tissue of the subject; and

configuring the stimulating current to change the level of Connexin 43.

In some embodiments of the present invention, the vagal stimulation system is configured to apply vagal stimulation in a series of bursts, at least one of which bursts includes a plurality of pulses. The control unit configures: (a) a pulse repetition interval (PRI) within each of the multi-pulse bursts (i.e., the time from the initiation of a pulse to the initiation of the following pulse within the same burst) to be on average at least 20 ms, such as at least 30 ms, e.g., at least 50 ms, and (b) the burst duration to be less than 75% of the interburst interval (i.e., the time from the initiation of a burst to the initiation of the following burst), such as less than 67% of the interburst interval, e.g., less than 50% or 33%. (“Burst duration,” as used in the present application, including in the claims, is the time from the initiation of the first pulse within a burst to the conclusion of the last pulse within the burst.) In experiments conducted on human subjects, the inventors found that increasing the PRI of the applied stimulation reduced sensations of acute pain experienced by the subjects.

For some applications, the control unit is configured to synchronize the bursts with a feature of the cardiac cycle of the subject. For example, each of the bursts may commence after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity.

In some embodiments of the present invention, the control unit is configured to apply the vagal stimulation during “on” periods alternating with “off” periods, during which no stimulation is applied (each set of a single “on” period followed by a single “off” period is referred to hereinbelow as a “cycle”). Typically, each cycle has a duration of between about 10 seconds and about 10 minutes, such as between about 20 seconds and about 5 minutes, e.g., about 30 seconds. The control unit is further configured to apply such intermittent stimulation during stimulation periods alternating with rest periods, during which no stimulation is applied. Each of the rest periods typically has a duration equal to at least the duration of one cycle, e.g., between one and 50 cycles, such as between two and four cycles, and each of the stimulation periods typically has a duration equal to at least 5 times the rest period duration, such as at least 10 times, e.g., at least 15 times. For example, each of the stimulation periods may have a duration of at least 30 cycles, e.g., at least 60 cycles or at least 120 cycles, and no greater than 2400 cycles, e.g., no greater than 1200 cycles. Alternatively, the duration of the stimulation and rest periods are expressed in units of time, and each of the rest periods has a duration of at least 30 seconds, e.g., such as at least one minute, at least two minutes, at least 5 minutes, or at least 25 minutes, and each of the stimulation periods has a duration of at least 10 minutes, e.g., at least 30 minutes, such as at least one hour, and less than 12 hours, e.g., less than six hours, such as less than two hours.

In human experiments conducted by the inventors, it was observed that application of continuous intermittent stimulation (i.e., without providing the rest periods described above) for long periods of time (e.g., several hours or several days) sometimes causes neuropathic pain. Providing a rest period of several minutes duration once every several hours eliminated this neuropathic pain and prevented its recurrence.

In some embodiments of the present invention, the vagal stimulation system is configured to apply vagal stimulation in a series of bursts, each of which includes one or more pulses (pulses per trigger, or PPT). The control unit is configured to apply the vagal stimulation during “on” periods alternating with “off” periods, during which no stimulation is applied. At the commencement of each “on” period, the control unit ramps up the PPT of successive bursts, and at the conclusion of each “on” period, the control unit ramps down the PPT of successive bursts. For example, the first four bursts of an “on” period may have respective PPTs of 1, 2, 3, and 3, or 1, 2, 3, and 4, and the last four bursts of an “on” period may have respective PPTs of 3, 3, 2, and 1, or 4, 3, 2, and 1. Use of such ramping generally prevents or reduces sudden drops and rebounds in heart rate at the beginning and end of each “on” period, respectively. Such sudden drops and rebounds are particularly undesirable in subjects suffering from heart disease, such as heart failure.

For some applications, the control unit is configured to synchronize the bursts with a feature of the cardiac cycle of the subject. For example, each of the bursts may commence after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity. For some applications, such ramping is applied only at the commencement of each “on” period, or only at the conclusion of each “on” period, rather than during both transitional periods. For some applications, such ramping techniques are combined with the extended PRI techniques described hereinabove, and/or with the rest period techniques described hereinabove.

In some embodiments of the present invention, for applications in which the control unit is configured to apply vagal stimulation intermittently, as described hereinabove, the control unit begins the stimulation with an “off” period, rather than with an “on” period. As a result, a delay having the duration of an “off” period occurs prior to beginning stimulation. Alternatively or additionally, whether or not configured to apply stimulation intermittently, the control unit is configured to delay beginning the application of stimulation for a certain time period after receiving an external command to apply the stimulation. For some applications, the length of the time period is determined responsive to the output of a pseudo-random number generator. The use of these delaying techniques generally reduces a subject's anticipation of any discomfort that he may associate with stimulation, and disassociates the sensations of stimulation from the physician and/or an external control device such as a wand.

There is therefore provided, in accordance with an embodiment of the present invention, an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and

a control unit, configured to:

drive the electrode device to apply to the site a current in at least first and second bursts, the first burst including a plurality of pulses, and the second burst including at least one pulse, and

set (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

In an embodiment, the control unit is configured to set the percentage to be less than 50%, such as less than 33%.

For some applications, the control unit is configured to set the average PRI of the first burst to be less than 200 ms. For some applications, the control unit is configured to set the interburst interval to be between 400 ms and 1500 ms.

For some applications, the control unit is configured to configure the first burst to include at least three pulses. Alternatively or additionally, the control unit is configured to set the first burst to include no more than six pulses.

For some applications, the control unit is configured to set an average duration of the pulses of the first burst to be less than 4 ms.

In an embodiment, the site includes the vagus nerve, and the electrode device is configured to be coupled to the vagus nerve.

For some applications, the control unit is configured to set the first burst to include a desired number of the pulses, and set the average PRI to be at least 75% of a maximum PRI possible given the interburst interval, the percentage, and the desired number of the pulses, but, in any event, no greater than 225 ms.

For some applications, the control unit is configured to withhold applying the current to the site when the pulses of the first and second bursts are not being applied.

For some applications, the control unit is configured to:

drive the electrode device to apply the current in at least the first and the second bursts, and in at least a third burst following the second burst, wherein the second burst includes a plurality of pulses, and wherein the third burst includes at least one pulse, and

set (a) a PRI of the second burst to be on average at least 20 ms, (b) an interburst interval between initiation of the second burst and initiation of the third burst to be less than 10 seconds, (c) an interburst gap between a conclusion of the second burst and the initiation of the third burst to have a duration greater than the average PRI of the second burst, and (d) a burst duration of the second burst to be less than 67% of the interburst interval between the initiation of the second burst and initiation of the third burst.

For some applications, the control unit is configured to set the average PRI to be at least 30 ms, or at least 50 ms.

For some applications, the control unit is configured to apply an interburst current to the site during at least a portion of the interburst gap, and to set the interburst current on average to be less than 50% of the current applied on average during the first burst. For some applications, the control unit is configured to apply an interburst current to the site during at least a portion of the interburst gap, and to set the interburst current on average to be less than 20% of the current applied on average during the first burst, such as less than 5% of the current applied on average during the pulses.

In an embodiment, the control unit is configured to:

drive the electrode device to apply the current during “on” periods that alternate with low stimulation periods, at least one of the “on” periods having an “on” duration of at least three seconds, and including at least three bursts, and at least one of the low stimulation periods immediately following the at least one of the “on” periods having a low stimulation duration equal to at least 50% of the “on” duration, wherein the at least three bursts of the at least one of the “on” periods include the first and second bursts,

set the current applied on average during the low stimulation periods to be less than 50% of the current applied on average during the “on” periods, and

during at least one transitional period of at the least one of the “on” periods, ramp a number of pulses per burst, the at least one transitional period selected from the group consisting of: a commencement of the at least one of the “on” periods, and a conclusion of the at least one of the “on” periods.

For some applications, the control unit is configured to set the current applied on average during the low stimulation periods to be less than 20% of the current applied on average during the “on” periods, such as less than 5% of the current applied on average during the “on” periods. For some applications, the control unit is configured to withhold applying the current during the low stimulation periods.

In an embodiment, the control unit is configured to:
drive the electrode device, during stimulation periods alternating with rest periods, to apply the current during “on” periods that alternate with low stimulation periods, the “on” periods having on average an “on” duration equal to at least 1 second, and the low stimulation periods having on average a low stimulation duration equal to at least 50% of the “on” duration, wherein at least one of the “on” periods includes the first and second bursts,
set the current applied on average during the low stimulation periods to be less than 20% of the current applied on average during the “on” periods, and
set the current applied on average during the rest periods to be less than 20% of the current applied on average during the “on” periods,

wherein the rest periods have on average a rest period duration equal to at least a cycle duration that equals a duration of a single “on” period plus a duration of a single low stimulation period, and

wherein the stimulation periods have on average a stimulation period duration equal to at least five times the rest period duration.

For some applications, the control unit is configured to set the current applied on average during the low stimulation periods to be less than 5% of the current applied on average during the “on” periods, and to set the current applied on average during the rest periods to be less than 5% of the current applied on average during the “on” periods. For some applications, the control unit is configured to withhold applying the current during the low stimulation periods and during the rest periods.

In an embodiment, the control unit is configured to set the first burst to include a desired number of the pulses, and set the average PRI to be at least 75% of a maximum PRI possible given the interburst interval, the percentage, and the desired number of the pulses. For some applications, the control unit is configured to set the average PRI to be at least 75% of (a) the interburst interval times (b) the percentage divided by (c) the difference between (i) the desired number of the pulses and (ii) one.

For some applications, the control unit is configured to set the average PRI of the first burst to be at least 30 ms, such as at least 50 ms, or at least 75 ms.

In an embodiment, the apparatus includes a sensor configured to sense a physiological parameter of the subject indicative of physiological activity of the subject, and the control unit is configured to synchronize the first and second bursts with the physiological activity. For some applications, the physiological activity is selected from the group consisting of: respiration of the subject, muscle contractions of the subject, and spontaneous nerve activity of the subject, and the sensor is configured to sense the physiological parameter indicative of the selected physiological activity. For some applications, the physiological activity includes cardiac activity of the subject, and the control unit is configured to synchronize the first and second bursts with a feature of a cardiac cycle of the subject. For example, the control unit may be configured to set the interburst interval to be equal to a sum of one or more sequential R-R intervals of the subject.

There is further provided, in accordance with an embodiment of the present invention, apparatus including:

an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and

a control unit, configured to:
drive the electrode device, during stimulation periods that alternate with rest periods, to apply to the site a current during “on” periods that alternate with low stimulation periods, the “on” periods having on average an “on” duration equal to at least 1 second, and the low stimulation periods having on average a low stimulation duration equal to at least 50% of the “on” duration,
set the current applied on average during the low stimulation periods to be less than 20% of the current applied on average during the “on” periods, and
set the current applied on average during the rest periods to be less than 20% of the current applied on average during the “on” periods,

wherein the rest periods have on average a rest period duration equal to at least a cycle duration that equals a duration of a single “on” period plus a duration of a single low stimulation period, and

wherein the stimulation periods have on average a stimulation period duration equal to at least five times the rest period duration.

For some applications, the control unit is configured to set the low stimulation duration to be at least 100% of the “on” duration. For some applications, the control unit is configured to set the rest period duration to be on average at least two times the cycle duration. For some applications, the control unit is configured to set the rest period duration to be on average at least 30 seconds.

For some applications, the control unit is configured to set the “on” duration to be on average at least 5 seconds.

For some applications, the control unit is configured to set the stimulation period duration to be on average at least 30 times the cycle duration. For some applications, the control unit is configured to set the stimulation period duration to be on average at least 30 minutes.

For some applications, the control unit is configured to:

drive the electrode device, during at least one of the “on” periods, to apply the current in at least first and second bursts, the first burst including a plurality of pulses, and the second burst including at least one pulse, and

set (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, the control unit is configured to:

set the “on” duration of at least one of the “on” periods to be at least three seconds,

configure the at least one of the “on” periods to include at least three bursts,

during at least one transitional period of the at least one of the “on” periods, ramp a number of pulses per burst, the at least one transitional period selected from the group consisting of: a commencement of the at least one of the “on” periods, and a conclusion of the at least one of the “on” periods.

For some applications, the control unit is configured to set the low stimulation duration to be less than 5 times the “on” duration.

For some applications, the control unit is configured to set the stimulation period duration to be on average at least 10 times the rest period duration, such as at least 15 times the rest period duration.

For some applications, the control unit is configured to set the current applied on average during the low stimulation periods to be less than 20% of the current applied on average during the “on” periods, and to set the current applied on average during the rest periods to be less than 20% of the current applied on average during the “on” periods. For example, the control unit may be configured to set the current applied on average during the low stimulation periods to be less than 5% of the current applied on average during the “on” periods, and to set the current applied on average during the rest periods to be less than 5% of the current applied on average during the “on” periods. For some applications, the control unit is configured to withhold applying the current during the low stimulation periods and during the rest periods.

In an embodiment, the control unit is configured to:

drive the electrode device to apply the current at least intermittently to the site for at least three hours, which at least three hours includes a period having a duration of three hours, which period is divided into a number of equal-duration sub-periods such that each of the sub-periods has a sub-period duration equal to three hours divided by the number, wherein the number is between 5 and 10,

configure the current to cause, during at least 20% of each of the sub-periods, an average reduction of at least 5% in a heart rate of the subject compared to a baseline heart rate of the subject, and

configure the current to not cause secondary neuropathic pain.

In an embodiment, the site includes the vagus nerve, and the electrode device is configured to be coupled to the vagus nerve.

In an embodiment, the control unit is configured to:

drive the electrode device to apply the current at least intermittently to the vagus nerve for at least three hours, which at least three hours includes a period having a duration of three hours,

configure the stimulation to include at least 3000 pulses during the period, the pulses having on average a pulse duration of at least 0.5 ms,

configure the stimulation to cause, on average during the pulses, at least 3 mA to enter tissue of the vagus nerve, and

configure the stimulation to not cause secondary neuropathic pain.

There is still further provided, in accordance with an embodiment of the present invention, apparatus including:

an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and

a control unit, configured to:

drive the electrode device to apply to the site a current in bursts of one or more pulses, during “on” periods that alternate with low stimulation periods, wherein at least one of the “on” periods has an “on” duration of at least three seconds, and including at least three bursts, and wherein at least one of the low stimulation periods immediately following the at least one of the “on” periods has a low stimulation duration equal to at least 50% of the “on” duration,

set the current applied on average during the low stimulation periods to be less than 20% of the current applied on average during the “on” periods, and

during at least one transitional period of the at least one of the “on” periods, ramp a number of pulses per burst, the at least one transitional period selected from the group consisting of: a commencement of the at least one of the “on” periods, and a conclusion of the at least one of the “on” periods.

For some applications, the control unit is configured to set the one or more pulses to have a characteristic pulse duration, at least one of the number of pulses includes a non-integer portion, and the control unit is configured to drive the electrode device to apply the non-integer portion by applying a pulse having a duration less than the characteristic pulse duration.

In an embodiment, the apparatus includes a sensor configured to sense a physiological parameter of the subject indicative of physiological activity of the subject, and the control unit is configured to synchronize the bursts with the physiological activity. For some applications, the physiological activity is selected from the group consisting of: respiration of the subject, muscle contractions of the subject, and spontaneous nerve activity of the subject, and the sensor is configured to sense the physiological parameter indicative of the selected physiological activity. For some applications, the physiological activity includes cardiac activity of the subject, and the control unit is configured to synchronize the bursts with a feature of a cardiac cycle of the subject. For example, the control unit may be configured to set the at least one of the “on” periods to include at least 10 bursts.

In an embodiment, the control unit is configured to:

drive the electrode device, during the at least one of the “on” periods, to apply the current in at least first and second bursts, the first burst including a plurality of pulses, and the second burst including at least one pulse, and

set (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, the control unit is configured to set the low stimulation duration of the at least one of the low stimulation periods immediately following the at least one of the “on” periods to be less than 5 times the “on” duration.

In an embodiment, the site includes the vagus nerve, and the electrode device is configured to be coupled to the vagus nerve.

In an embodiment, the control unit is configured to drive the electrode device to apply the current during stimulation periods alternating with rest periods, and to set the current applied on average during the rest periods to be less than 50% of the current applied on average during the “on” periods, wherein the rest periods have on average a rest period duration equal to at least a cycle duration that equals a duration of a single “on” period plus a duration of a single low stimulation period, and wherein the stimulation periods have on average a stimulation period duration equal to at least five times the rest period duration. For some applications, the control unit is configured to set the current applied on average during the rest periods to be less than 20% of the current applied on average during the “on” periods, such as less than 5% of the current applied on average during the “on” periods. For some applications, the control unit is configured to withhold applying the current during the rest periods.

For some applications, the at least one transitional period includes the commencement of the at least one of the “on” periods, and the control unit is configured to ramp up the number of pulses per burst during the commencement. For some applications, the control unit is configured to set the number of pulses of an initial burst of the at least one of the “on” periods and a second burst immediately subsequent to the initial burst to be equal to 1 and 2, respectively. For some applications, the control unit is configured to set the number of pulses of a third burst of the at least one of the “on” periods immediately subsequent to the second burst to be equal to 3.

For some applications, the at least one transitional period includes the conclusion of the at least one of the “on” periods, and the control unit is configured to ramp down the number of pulses per burst during the conclusion. For some applications, the control unit is configured to set the number of pulses of last and penultimate bursts of the at least one of the “on” periods to be equal to 1 and 2, respectively. For some applications, the control unit is configured to set the number of pulses of an antepenultimate burst of the at least one of the “on” periods to be equal to 3.

For some applications, the control unit is configured to set the current applied on average during the low stimulation periods to be less than 20% of the current applied on average during the “on” periods, such as less than 5% of the current applied on average during the “on” periods. For some applications, the control unit is configured to withhold applying the current during the low stimulation periods.

There is additionally provided, in accordance with an embodiment of the present invention, apparatus including:

an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and

a control unit, configured to:

drive the electrode device to apply electrical stimulation to the site for at least three hours, which at least three hours includes a period having a duration of three hours, which period is divided into a number of equal-duration sub-periods such that each of the sub-periods has a sub-period duration equal to three hours divided by the number, wherein the number is between 5 and 10,

configure the stimulation to cause, during at least 20% of each of the sub-periods, an average reduction of at least 5% in a heart rate of the subject compared to a baseline heart rate of the subject, and

configure the stimulation to not cause secondary neuropathic pain.

In an embodiment, the control unit is configured to configure the stimulation to not cause local pain in a vicinity of the site.

For some applications, the control unit is configured to configure the stimulation to cause the average reduction during at least 40% of each of the sub-periods.

For some applications, the number of sub-periods is 6, such that the sub-period duration equals 30 minutes. Alternatively, for some applications, the number of sub-periods is 9, such that the sub-period duration equals 20 minutes.

In an embodiment, the site includes the vagus nerve, and the electrode device is configured to be coupled to the vagus nerve.

There is yet additionally provided, in accordance with an embodiment of the present invention, apparatus including:

an electrode device, configured to be coupled to a site of a vagus nerve of a subject; and

a control unit, configured to:

drive the electrode device to apply electrical stimulation to the site for at least three hours, which at least three hours includes a period having a duration of three hours,

configure the stimulation to include at least 3000 pulses during the period, the pulses having on average a pulse duration of at least 0.5 ms,

configure the stimulation to cause, on average during the pulses, at least 3 mA to enter tissue of the vagus nerve, and

configure the stimulation to not cause secondary neuropathic pain.

In an embodiment, the control unit is configured to configure the stimulation to not cause local pain in a vicinity of the site.

For some applications, the control unit is configured to configure the stimulation to include at least 5000 pulses during the period. For some applications, the control unit is configured to configure the stimulation to cause, on average during the pulses, at least 4 mA to enter the tissue of the vagus nerve. For some applications, the control unit is configured to set the pulse duration to be at least 0.9 ms.

There is also provided, in accordance with an embodiment of the present invention, apparatus including:

an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein;

a sensor configured to sense a physiological parameter of the subject indicative of physiological activity of the subject; and

a control unit, configured to:

drive the electrode device to apply to the site a current in at least first and second bursts, the first burst including a plurality of pulses, and the second burst including at least one pulse,

set a pulse repetition interval (PRI) of the first burst to be on average at least 20 ms, and

synchronize the first and second bursts with the physiological activity.

For some applications, the physiological activity is selected from the group consisting of: respiration of the subject, muscle contractions of the subject, and spontaneous nerve activity of the subject, and the sensor is configured to sense the physiological parameter indicative of the selected physiological activity.

In an embodiment, the physiological activity includes cardiac activity of the subject, and the control unit is configured to synchronize the first and second bursts with a feature of a cardiac cycle of the subject. For some applications, the control unit is configured to set an interburst interval between initiation of the first burst and initiation of the second burst to be equal to a sum of one or more sequential R-R intervals of the subject.

In an embodiment, the site includes the vagus nerve, and the electrode device is configured to be coupled to the vagus nerve.

For some applications, the control unit is configured to set an interburst interval between initiation of the first burst and initiation of the second burst to be less than 10 seconds. Alternatively or additionally, the control unit is configured to set an interburst gap between a conclusion of the first burst and the initiation of the second burst to have a duration greater than the average PRI. Further alternatively or additionally, the control unit is configured to set a burst duration of the first burst to be less than a percentage of an interburst interval between initiation of the first burst and initiation of the second burst, the percentage being less than 67%.

There is also provided, in accordance with an embodiment of the present invention, a method including:

applying, to a site of a subject, a current in at least first and second bursts, the first burst including a plurality of pulses, and the second burst including at least one pulse, the site selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and

setting (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

There is further provided, in accordance with an embodiment of the present invention, a method including:

applying, to a site of a subject, during stimulation periods that alternate with rest periods, a current during “on” periods that alternate with low stimulation periods, the “on” periods having on average an “on” duration equal to at least 1 second, and the low stimulation periods having on average a low stimulation duration equal to at least 50% of the “on” duration, the site selected from the group consisting of: a vagus nerve, an epicardial fat pad, a pulmonary vein, a carotid artery, a carotid sinus, a coronary sinus, a vena cava vein, a right ventricle, a right atrium, and a jugular vein;

setting the current applied on average during the low stimulation periods to be less than 20% of the current applied on average during the “on” periods;

setting the current applied on average during the rest periods to be less than 20% of the current applied on average during the “on” periods; and

setting the rest periods to have on average a rest period duration equal to at least a cycle duration that equals a duration of a single “on” period plus a duration of a single low stimulation period, and the stimulation periods to have on average a stimulation period duration equal to at least five times the rest period duration.

There is still further provided, in accordance with an embodiment of the present invention, a method including:

applying, to a site of a subject, a current in bursts of one or more pulses, during “on” periods that alternate with low stimulation periods, at least one of the “on” periods having an “on” duration of at least three seconds, and including at least three bursts, and at least one of the low stimulation periods immediately following the at least one of the “on” periods having a low stimulation duration equal to at least 50% of the “on” duration, the site selected from the group consisting of: a vagus nerve, an epicardial fat pad, a pulmonary vein, a carotid artery, a carotid sinus, a coronary sinus, a vena cava vein, a right ventricle, a right atrium, and a jugular vein;

setting the current applied on average during the low stimulation periods to be less than 20% of the current applied on average during the “on” periods; and

during at least one transitional period of the at least one of the “on” periods, ramping a number of pulses per burst, the at least one transitional period selected from the group consisting of: a commencement of the at least one of the “on” periods, and a conclusion of the at least one of the “on” periods.

There is additionally provided, in accordance with an embodiment of the present invention, a method including:

applying electrical stimulation to a site of a subject for at least three hours, which at least three hours includes a period having a duration of three hours, which period is divided into a number of equal-duration sub-periods such that each of the sub-periods has a sub-period duration equal to three hours divided by the number, wherein the number is between 5 and 10, wherein the site is selected from the group consisting of: a vagus nerve, an epicardial fat pad, a pulmonary vein, a carotid artery, a carotid sinus, a coronary sinus, a vena cava vein, a right ventricle, a right atrium, and a jugular vein;

configuring the stimulation to cause, during at least 20% of each of the sub-periods, an average reduction of at least 5% in a heart rate of the subject compared to a baseline heart rate of the subject; and

configuring the stimulation to not cause secondary neuropathic pain.

There is yet additionally provided, in accordance with an embodiment of the present invention, a method including:

applying electrical stimulation to a site of vagus nerve of a subject for at least three hours, which at least three hours includes a period having a duration of three hours;

configuring the stimulation to include at least 3000 pulses during the period, the pulses having on average a pulse duration of at least 0.5 ms;

configuring the stimulation to cause, on average during the pulses, at least 3 mA to enter tissue of the vagus nerve; and

configuring the stimulation to not cause secondary neuropathic pain.

There is also provided, in accordance with an embodiment of the present invention, apparatus including:

an electrode device, configured to be coupled to tissue of a subject selected; and

a control unit, configured to:

drive the electrode device to apply to the tissue a current in at least first and second bursts, the first burst including a plurality of pulses, and the second burst including at least one pulse, and

set (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, the tissue includes nerve tissue of the subject, and the electrode device is configured to be coupled to the nerve tissue. For some applications, the tissue includes muscle tissue of the subject, and the electrode device is configured to be coupled to the muscle tissue.

For some applications, the electrode device is configured to be implantable in a body of the subject.

There is further provided, in accordance with an embodiment of the present invention, apparatus including:

an electrode device, configured to be coupled to tissue of a subject; and

a control unit, configured to:
drive the electrode device, during stimulation periods that alternate with rest periods, to apply to the tissue a current during “on” periods that alternate with low stimulation periods, the “on” periods having on average an “on” duration equal to at least 1 second, and the low stimulation periods having on average a low stimulation duration equal to at least 50% of the “on” duration,
set the current applied on average during the low stimulation periods to be less than 20% of the current applied on average during the “on” periods, and
set the current applied on average during the rest periods to be less than 20% of the current applied on average during the “on” periods,

wherein the rest periods have on average a rest period duration equal to at least a cycle duration that equals a duration of a single “on” period plus a duration of a single low stimulation period, and

wherein the stimulation periods have on average a stimulation period duration equal to at least five times the rest period duration.

For some applications, the tissue includes nerve tissue of the subject, and the electrode device is configured to be coupled to the nerve tissue. For some applications, the tissue includes muscle tissue of the subject, and the electrode device is configured to be coupled to the muscle tissue.

For some applications, the electrode device is configured to be implantable in a body of the subject.

There is also provided, in accordance with an embodiment of the present invention, a method including:

applying, to a site of a subject, a current in at least first and second bursts, the first burst including a plurality of pulses, and the second burst including at least one pulse, the site selected from the group consisting of: a vagus nerve, an epicardial fat pad, a pulmonary vein, a carotid artery, a carotid sinus, a coronary sinus, a vena cava vein, a right ventricle, a right atrium, and a jugular vein;

setting (a) a pulse repetition interval (PRI) of the first burst to be on average at least 20 ms;

sensing a physiological parameter of the subject indicative of physiological activity of the subject; and

synchronizing the first and second bursts with the physiological activity.

There is still further provided, in accordance with an embodiment of the present invention, a method including:

applying, to tissue of a subject, a current in at least first and second bursts, the first burst including a plurality of pulses, and the second burst including at least one pulse; and

setting (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

There is additionally provided, in accordance with an embodiment of the present invention, a method including:

applying, to tissue of a subject, during stimulation periods that alternate with rest periods, a current during “on” periods that alternate with low stimulation periods, the “on” periods having on average an “on” duration equal to at least 1 second, and the low stimulation periods having on average a low stimulation duration equal to at least 50% of the “on” duration;

setting the current applied on average during the low stimulation periods to be less than 20% of the current applied on average during the “on” periods;

setting the current applied on average during the rest periods to be less than 20% of the current applied on average during the “on” periods; and

setting the rest periods to have on average a rest period duration equal to at least a cycle duration that equals a duration of a single “on” period plus a duration of a single low stimulation period, and the stimulation periods to have on average a stimulation period duration equal to at least five times the rest period duration.

In some embodiments of the present invention, techniques are provided for applying intra-atrial parasympathetic stimulation. For some applications, the stimulation is applied to a site in an atrium of a subject, such as myocardium of the left atrium, myocardium of the right atrium, or myocardium of the interatrial septum.

In some embodiments of the present invention, a subject is identified as suffering from a cardiac condition, and intra-atrial stimulation of one or more parasympathetic epicardial fat pads is applied to treat the condition. The condition typically includes chronic heart failure (HF), atrial flutter, chronic atrial fibrillation (AF), chronic AF combined with HF, hypertension, angina, and/or an inflammatory condition of the heart. Alternatively or additionally, the techniques described herein are used post-myocardial infarct, post heart surgery, post heart transplant, during heart surgery, or during an otherwise indicated catheterization (such as PTCA). Further alternatively or additionally, the stimulation is applied to regular the production of nitric oxide (NO) (e.g., by changing the level of at least one NO synthase), such as in combination with techniques described in U.S. application Ser. No. 11/234,877, filed Sep. 22, 2005, entitled, “Selective nerve fiber stimulation,” which issued as U.S. Pat. No. 7,885,709 and is assigned to the assignee of the present application and is incorporated herein by reference.

For some applications, such implantation is used for applying stimulation for preventing and/or terminating atrial fibrillation, typically by applying the stimulation to the AV node fat pad. For some applications, techniques are used that are described in U.S. patent application Ser. No. 11/657,784, filed Jan. 24, 2007, which issued as U.S. Pat. No. 8,204,591, and/or U.S. patent application Ser. No. 10/560,654, filed May 1, 2006, which issued as U.S. Pat. No. 7,885,711, both of which are assigned to the assignee of the present application and are incorporated herein by reference. For some applications, such epicardial implantation is used for treating a subject suffering from both heart failure and atrial fibrillation. For some applications, such stimulation is applied only when a sensed heart rate of the subject exceeds a threshold heart rate, such as about 60 BMP.

In some embodiments of the present invention, the techniques of the present invention are performed using a parasympathetic stimulation system that comprises at least one electrode assembly, which is applied to a cardiac site containing parasympathetic nervous tissue, such as an atrial site, and an implantable or external control unit. The electrode assembly comprises a lead coupled to one or more electrode contacts. For some applications, the electrode contacts are configured to be implanted in a right atrium, typically in contact with atrial muscle tissue in a vicinity of a parasympathetic epicardial fat pad. For some applications, the electrode contacts are fixed within the atrium using active fixation techniques. For some applications, the parasympathetic epicardial fat pad comprises a sinoatrial (SA) fat pad, while for other applications, the parasympathetic epicardial fat pad comprises an atrioventricular (AV) fat pad. For some applications, separate electrode assemblies, or separate electrode contacts of a single electrode assembly, are implanted in the vicinity of both the SA node fat pad and the AV node fat pad, for activating both fat pads.

In some embodiments of the present invention, the electrode assembly comprises a rotational-engagement fixation element, typically a screw-in fixation element. For some applications, the fixation element is sized such that its proximal end extends to the surface of the atrial wall when fully implanted, while for other applications, the fixation mechanism is shorter, such that its proximal end does not reach the surface of the atrial wall when fully implanted, but instead terminates inside the atrial wall. The surface of a proximal portion of the fixation element is electrically insulated, e.g., comprises a non-conductive coating, such as Teflon or silicone, around a conductive core. A distal portion of the fixation element is conductive, and serves as one of the electrode contacts.

The insulated portion of the fixation element is configured to be chronically disposed at least partially within the atrial muscle tissue, and the electrode contacts are configured to be chronically disposed in contact with the parasympathetic epicardial fat pad, typically within the fat pad. Typically, but not necessarily, the electrode contacts are positioned entirely within the fat pad, such that no portion of the electrode contacts are in contact with the atrial muscle tissue. Avoidance of direct application of current to the atrial muscle tissue generally decreases the risk of undesired cardiac capture.

In some embodiments of the present invention, an electrode contact, e.g., part of a screw-in fixation element, is configured to implanted in the atrial muscle tissue, typically either in a vicinity of the SA node fat pad 46 or the AV node fat pad. A second electrode contact is disposed on a lead which passes through the superior vena cava, such that the second electrode contact is positioned in the superior vena cava. An electric field is generated, the magnitude of which is highest in the area generally between the electrode contacts. In this manner, current generated between the electrode contacts affects the fat pad to a greater extent than the muscle tissue. Alternatively, the second electrode contact is placed in another blood vessel, such as an inferior vena cava, a coronary sinus, a right pulmonary vein, a left pulmonary vein, or a right ventricular base. Alternatively, an electrode contact positioned outside of the heart and the circulatory system in a vicinity of the fat pad (but not in physical contact with the heart or the fat pad) serves as one of the electrode contacts.

In some embodiments of the present invention, at least one electrode contact is positioned at an atrial region within an atrium (typically the right atrium, or alternatively in the left atrium) in contact with the atrial wall, within the atrial wall, and/or through the atrial wall, in a vicinity of postganglionic fibers of a parasympathetic epicardial fat pad, such as the SA node fat pad and/or the AV node fat pad, but not at or in the fat pad itself (i.e., not in contact with, or within, tissue of the cardiac wall that underlies the fat pad). Typically, but not necessarily, the atrial region is located generally between the SA node fat pad and an SA node, or generally between the AV node fat pad and an AV node. The inventors believe that stimulation of the postganglionic fibers in this region has a greater heart-rate-reduction effect than stimulation at or in the fat pads. The inventors also hypothesize that such postganglionic stimulation generally causes less afferent activation than stimulation of the fat pads or preganglionic fibers, and is thus less likely to cause side effects.

In some embodiments of the present invention, the electrode assembly comprises one or more first electrode contacts which are configured to be placed in a coronary sinus. For some applications, the first electrode contacts comprise ring electrodes, or are incorporated into one or more baskets or coronary stents. A second electrode contact (which may comprise any of the fixation elements described herein, such as a screw-in fixation element) is configured to be implanted in a vicinity of the AV node fat pad, in contact with the atrial wall, within the atrial wall, and/or through the atrial wall into the fat pad. The control unit drives a current between the second electrode contact and each of the first electrode contacts in alternation. The alternation among the first electrode contacts generally reduces the likelihood of exhausting the ganglia within the AV node fat pad.

In some embodiments of the present invention, a method for implanting an electrode contact is provided, comprising placing the electrode contact within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base. The electrode contact is advanced into a wall of the organ; a property (e.g., impedance) of tissue in a vicinity of a distal tip of the electrode contact is monitored over time. A determination is made that the tip of the electrode contact has penetrated through the wall into the fat pad upon detecting a change in the property, upon which advancement of the electrode contact is ceased.

There is therefore provided, in accordance with an embodiment of the present invention, a method including:

implanting in an atrial wall of a subject, from within an atrium, a first electrode contact in a vicinity of a parasympathetic epicardial fat pad of the subject;

implanting a second electrode contact in a body of the subject outside of a heart and a circulatory system; and

driving a current between the first and second electrode contacts, and configuring the current to cause parasympathetic activation of the fat pad.

In an embodiment, implanting the first electrode contact includes implanting, from within the atrium, a fixation element including a screw that includes the first electrode contact.

For some applications, implanting the second electrode includes implanting the second electrode at a location that is not in physical contact with the heart or the fat pad.

For some applications, configuring the current includes configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

For some applications, implanting the second electrode contact includes implanting the second electrode contact in a vicinity of left sides of right ribs of the subject. Alternatively or additionally, implanting the second electrode contact includes implanting the second electrode contact under right ribs of the subject. Further alternatively or additionally, implanting the second electrode contact includes subcutaneously implanting the second electrode contact on a right side of a chest of the subject.

For some applications, implanting the fixation element and the second electrode contact includes implanting the fixation element and the second electrode contact such that a distance between the first and second electrode contacts is no more than 4 cm.

For some applications, driving the current includes configuring the current such that the first electrode contact serves as a cathode, and the second electrode contact as an anode.

For some applications, implanting the second electrode element includes implanting the second electrode element before implanting the fixation element, and implanting the fixation element includes: positioning the first electrode contact at a plurality of locations of in the vicinity of the fat pad; while the first electrode contact is positioned at each of the locations, driving the current between the first and second electrode contacts and sensing a vagomimetic effect; and implanting the fixation element such that the first electrode contact is positioned at the one of the locations at which a greatest vagomimetic effect was sensed.

There is further provided, in accordance with an embodiment of the present invention, apparatus including:

a first electrode contact, configured to be implanted, from within an atrium, in an atrial wall of a subject in a vicinity of a parasympathetic epicardial fat pad of the subject;

a second electrode contact, configured to be implanted in a body of the subject outside of a heart and a circulatory system; and

a control unit, configured to:

drive a current between the first and second electrode contacts, and

configure the current to cause parasympathetic activation of the fat pad.

There is still further provided, in accordance with an embodiment of the present invention, a method including:

implanting in an atrial wall of a subject, from within an atrium, a first electrode contact in a vicinity of a parasympathetic epicardial fat pad of the subject;

placing a second electrode contact within an organ of a circulatory system selected from the group consisting of: a superior vena cava, an inferior vena cava, a coronary sinus, a right pulmonary vein, a left pulmonary vein, and a right ventricular base; and

driving a current between the first and second electrode contacts, and configuring the current to cause parasympathetic activation of the fat pad.

In an embodiment, implanting the first electrode contact includes implanting, from within the atrium, a fixation element including a screw that includes the first electrode contact.

For some applications, configuring the current includes configuring the current such that a pulse frequency, an amplitude, and a pulse width thereof have a product that is less than 12 Hz*mA*ms, and such that the current reduces a heart rate of the subject by at least 10% compared to a baseline heart rate of the subject in the absence of the application of the current.

For some applications, the site includes the coronary sinus, the fat pad includes an atrioventricular (AV) node fat pad, placing includes placing the second electrode contact in the coronary sinus, and implanting includes implanting the first electrode contact in the vicinity of the AV node fat pad.

For some applications, implanting and placing include implanting the first electrode contact and placing the second electrode contact such that a distance between the first and second electrode contacts is no more than 2 cm.

For some applications, driving the current includes configuring the current such that the first electrode contact serves as a cathode, and the second electrode contact as an anode.

There is additionally provided, in accordance with an embodiment of the present invention, apparatus including:

a first electrode contact, configured to be implanted, from within an atrium, in an atrial wall of a subject in a vicinity of a parasympathetic epicardial fat pad of the subject;

a second electrode contact, configured to be placed within an organ of a circulatory system selected from the group consisting of: a superior vena cava, an inferior vena cava, a coronary sinus, a right pulmonary vein, a left pulmonary vein, and a right ventricular base; and

a control unit, configured to:

drive a current between the first and second electrode contacts, and

configure the current to cause parasympathetic activation of the fat pad.

There is yet additionally provided, in accordance with an embodiment of the present invention, a method including:

implanting in an atrial wall of a subject, from within an atrium, at least two fixation elements including respective screws that include respective electrode contacts, such that the electrode contacts are positioned in a vicinity of a parasympathetic epicardial fat pad of the subject; and

driving a current between the electrode contacts, and configuring the current to cause parasympathetic activation of the fat pad.

In an embodiment, implanting comprises implanting at least one of the fixation elements such that the electrode contact thereof is positioned entirely within the fat pad, and no other portion of the at least one of the fixation elements is in direct electrical contact with tissue of the atrial wall.

For some applications, at least one of the screws has a proximal portion having a non-conductive external surface, and a distal portion having a conductive external surface that serves as the electrode contact of the screw.

There is also provided, in accordance with an embodiment of the present invention, a method including:

placing at least three electrodes contacts at respective sites in a vicinity of a parasympathetic epicardial fat pad;

selecting a first set of at least two of the electrode contacts, and a second set of at least two of the electrode contacts, wherein the first and second sets are not identical;

during at least one stimulation period for each of 30 consecutive days, alternatingly (a) driving a current between the electrode contacts of the first set, and (b) driving the current between the electrode contacts of the second set; and

configuring the current to cause parasympathetic activation of the fat pad.

For some applications, selecting the first and second sets includes including at least one common electrode in both sets.

In an embodiment, placing includes implanting, in an atrial wall, from within an atrium, a fixation element including a screw that includes at least one of the electrode contacts, such that the at least one of the electrode contacts is positioned in the vicinity of the fat pad.

For some applications, driving the current includes configuring the current such that the at least one of the electrode contacts serves as a cathode.

For some applications, the least one of the electrode contacts includes a first one of the electrode contacts, placing includes placing second and third ones of the electrode contacts within a coronary sinus, the first set includes the first and the second ones of the electrode contacts, and the second set includes the first and the third ones of the electrode contacts.

For some applications, the least one of the electrode contacts includes a first one of the electrode contacts, and placing includes implanting second and third ones of the electrode contacts in a body of the subject outside of a heart and a circulatory system.

There is further provided, in accordance with an embodiment of the present invention, apparatus including:

an electrode assembly including an intracardiac lead and proximal and distal intracardiac electrode contacts coupled to the lead;

an intracardiac sheath sized so as to allow passage of the lead therethrough, and having a wall that includes a conducting portion through which electricity is conductible, wherein the electrode assembly and the sheath are configured such that the proximal electrode contact is aligned with the conducting portion when the distal electrode contact is advanced through the sheath at least to a distal opening of the sheath; and

a control unit, configured to drive a current between the proximal and distal electrode contacts when the proximal electrode contact is aligned with the conducting portion of the sheath.

In an embodiment of the present invention, the wall of the sheath is shaped so as to define a window that defines the conducting portion. Alternatively, the wall of the sheath includes a conductive element that serves as the conducting portion.

For some applications, the sheath is configured such that the conducing portion extends from the distal opening of the sheath for at least 1 cm in a proximal direction along the sheath. For some applications, at least a portion of the conducting portion is deflectable.

There is still further provided, in accordance with an embodiment of the present invention, a method including:

providing an electrode assembly including an intracardiac lead and proximal and distal intracardiac electrode contacts coupled to the lead;

positioning an intracardiac sheath such that at least a distal end thereof is in a heart, the sheath sized so as to allow passage of the lead therethrough, and having a wall that includes a conducting portion through which electricity is conductible;

advancing the distal electrode contact through the sheath at least to a distal opening of the sheath, such that the proximal electrode contact is aligned with the conducting portion; and

driving a current between the proximal and distal electrode contacts when the proximal electrode contact is aligned with the conducting portion of the sheath.

There is additionally provided, in accordance with an embodiment of the present invention, a method for implanting an electrode contact, including:

placing the electrode contact within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

advancing the electrode contact into a wall of the organ;

monitoring a property of tissue in a vicinity of a distal tip of the electrode contact over time;

making a determination that the tip of the electrode contact has penetrated through the wall into the fat pad upon detecting a change in the property; and

ceasing advancing the electrode contact responsively to the determination.

In an embodiment, the organ includes the right atrium, and placing includes implanting, within the right atrium, a fixation element including a screw that includes the electrode contact.

For some applications, the electrode contact includes a first electrode contact, and monitoring the property includes placing a second electrode contact in a vicinity of the first electrode contact, and monitoring impedance between the first and second electrode contacts.

For some applications, ceasing the advancing includes advancing the tip slightly further into the fat pad before ceasing the advancing, and monitoring the property includes continuing to monitor the property after making the determination, and further including: making a subsequent determination that the tip of the electrode contact has penetrated through the fat pad into a pericardial space beyond the fat pad upon detecting a subsequent change in the property; and withdrawing the tip of the electrode contact back into the fat pad responsively to the subsequent determination.

For some applications, ceasing advancing includes leaving the electrode contact in contact with the fat pad for at least one week.

There is yet additionally provided, in accordance with an embodiment of the present invention, apparatus including:

an electrode contact configured to penetrate, from with an atrium of a subject, an atrial wall at a penetration site;

a lead coupled to the electrode contact at a distal end of the lead, the lead including an element having a greater diameter than the electrode contact, and configured to surround the penetration site and reduce potential blood flow through the penetration site; and

a control unit configured to drive the electrode contact to apply stimulation to tissue of the subject, and configure the stimulation to cause parasympathetic activation.

For some applications, the apparatus further includes a sheath surrounding the electrode contact, and the element is connected to the sheath, and is configured to be pushed forward to press against the atrial wall after the electrode contact is implanted. For some applications, at least a portion of the electrode that penetrates the atrial wall has a proximal portion having a greater cross-sectional area than a distal portion thereof.

There is also provided, in accordance with an embodiment of the present invention, apparatus including:

a helically-shaped intracardiac screw-in fixation element, including, at a distal tip thereof, a bipolar electrode, which includes:
an outer electrode contact; and
and an inner electrode contact arranged coaxially within the outer electrode contact, and electrically isolated from the outer electrode contact; and

a control unit, configured to drive the bipolar electrode to apply cardiac stimulation.

There is further provided, in accordance with an embodiment of the present invention, a method including:

providing an electrode assembly including a lead and at least two electrode contacts coupled to the lead;

positioning the electrode assembly such that the at least two electrode contacts are within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

advancing the at least two electrode contacts into a wall of the organ.

There is still further provided, in accordance with an embodiment of the present invention, a method including:

coupling, from within an atrium of a subject, a distal portion of at least one electrode assembly to an atrial wall, such that at least one electrode contact of the electrode assembly is in contact with tissue in a vicinity of a parasympathetic epicardial fat pad;

driving the at least one electrode contact to apply electrical stimulation to the tissue; and

configuring the stimulation such that a pulse frequency, an amplitude, and a pulse width thereof have a product that is less than 12 Hz*mA*ms, and such that the stimulation reduces a heart rate of the subject by at least 10% compared to a baseline heart rate of the subject in the absence of the stimulation.

For some applications, configuring includes configuring the stimulation to reduce the heart rate by at least 20% compared to the baseline heart rate.

There is additionally provided, in accordance with an embodiment of the present invention, a method including:

chronically implanting at least one screw electrode within an atrium of a subject;

driving the at least one electrode to apply stimulation to tissue of the atrium;

configuring the stimulation to stimulate at least one vagal ganglion plexus (GP) of the subject; and

setting a duration of the stimulation to be between 1 and 10 microseconds.

There is yet additionally provided, in accordance with an embodiment of the present invention, a method including:

implanting, from within a right atrium, an electrode contact in an atrial wall at a site that is in a vicinity of postganglionic fibers of a parasympathetic epicardial fat pad, and that does not underlie the fat pad;

driving the electrode contact to apply a current to the postganglionic fibers; and

configuring the current to activate the postganglionic fibers.

For some applications, the fat pad includes a sinoatrial (SA) node fat pad, and the site is generally between the SA node fat pad and an SA node. For some applications, the site is at least 1 mm from the SA node.

In an embodiment, the fat pad includes a atrioventricular (AV) node fat pad, and the site is generally between the AV node fat pad and an AV node. For some applications, the site is at least 1 mm from the AV node.

For some applications, the site is at least 1 mm from a region of an interior surface of the atrial wall that underlies the fat pad.

There is also provided, in accordance with an embodiment of the present invention, a method including:

implanting in an atrial wall of a subject, from within an atrium, at least two fixation elements including respective screws that include respective electrode contacts, such that the electrode contacts are positioned in vicinities of respective vagomimetic sites; and

driving the electrode contacts to apply electrical stimulation to the respective vagomimetic sites, and configuring the stimulation to cause parasympathetic activation of the sites.

For some applications, the at least two fixation elements include at least three fixation elements, and implanting includes implanting the at least three fixation elements in the atrial wall, such that the respective electrode contacts are positioned in the vicinities of the respective vagomimetic sites.
For some applications, driving includes simultaneously driving all of the electrode contacts to apply the stimulation to the respective sites. Alternatively, driving includes driving each of the electrode contacts to apply the stimulation to its respective site during a local refractory period at the site.

For some applications, implanting includes implanting the fixation elements such that a first one of the electrode contacts is positioned in a vicinity of an SA node fat pad, and a second one of the electrode contacts is positioned in a vicinity of the AV node fat pad. For some applications, implanting includes identifying that the subject suffers from heart failure and paroxysmal atrial fibrillation (AF), and implanting responsively to the identifying. For some applications, driving includes: detecting whether the subject is currently in normal sinus rhythm (NSR) or experiencing an episode of the AF; upon detecting that the subject is experiencing the episode of the AF, driving the second one of the electrode contacts to apply the stimulation to the AV node fat pad; and upon detecting that the subject is currently in NSR, driving the first one of the electrode contacts to apply the stimulation to the SA node fat pad.

For some applications, configuring the stimulation includes: sensing a measure of cardiac performance of the subject; and responsively to the measure, configuring one or more parameters of the stimulation applied by the second one of the electrode contacts to the AV node fat pad to cause an improvement in the sensed measure of cardiac performance.

There is further provided, in accordance with an embodiment of the present invention, apparatus including:

first and second electrode contacts, configured to be implanted, from within a right atrium of a subject, in an atrial wall at respective sites that are in respective vicinities of an sinoatrial (SA) node fat pad and an atrioventricular (AV) node fat pad; and

a control unit, configured to:

detect an episode of non-sinus atrial tachycardia, and

responsively to the detection, restore normal sinus rhythm (NSR) of the subject by:

driving the first and second electrode contacts to apply respective parasympathetic stimulation signals to the sites, and

configuring the parasympathetic stimulation signals to activate parasympathetic nervous tissue of the SA node and AV node fat pads sufficiently to restore the NSR.

For some applications, the non-sinus atrial tachycardia includes atrial fibrillation (AF), and the control unit is configured to detect the episode of the AF. Alternatively or additionally, the non-sinus atrial tachycardia includes atrial flutter, and the control unit is configured to detect the episode of the atrial flutter.

There is still further provided, in accordance with an embodiment of the present invention, a method including:

implanting, from within a right atrium, first and second electrode contacts in an atrial wall of a subject at respective sites that are in respective vicinities of an sinoatrial (SA) node fat pad and an atrioventricular (AV) node fat pad; and

detecting an episode of non-sinus atrial tachycardia of the subject; and

responsively to the detection, restoring normal sinus rhythm (NSR) of the subject by:

driving the first and second electrode contacts to apply respective parasympathetic stimulation signals to the sites, and

configuring the parasympathetic stimulation signals to activate parasympathetic nervous tissue of the SA node and AV node fat pads sufficiently to restore the NSR.

There is additionally provided, in accordance with an embodiment of the present invention, apparatus including:

an electrode contact, configured to be implanted, from within a right atrium of a subject, in an atrial wall at a site that is in a vicinity of an atrioventricular (AV) node fat pad; and

a control unit, configured to:

detect whether the subject is experiencing atrial fibrillation or is in normal sinus rhythm (NSR),

responsively to detecting that the subject is experiencing AF, drive the electrode contact to applying stimulation to the site, and configure the stimulation to cause parasympathetic activation of the fat pad at a strength sufficient to reduce a heart rate of the subject; and

responsively to detecting that the subject is in NSR, converting the subject to AF by driving the electrode contact to apply a pacing signal to the site having a frequency of at least 1.5 Hz.

There is yet additionally provided, in accordance with an embodiment of the present invention, a method including:

implanting, from within a right atrium of a subject, an electrode contact in an atrial wall at a site that is in a vicinity of an atrioventricular (AV) node fat pad;

detecting whether the subject is experiencing atrial fibrillation or is in normal sinus rhythm (NSR);

responsively to detecting that the subject is experiencing AF, driving the electrode contact to applying stimulation to the site, and configuring the stimulation to cause parasympathetic activation of the fat pad at a strength sufficient to reduce a heart rate of the subject; and

responsively to detecting that the subject is in NSR, converting the subject to AF by driving the electrode contact to apply a pacing signal to the site having a frequency of at least 1.5 Hz.

There is also provided, in accordance with an embodiment of the present invention, a method including:

identifying that a subject suffers from at least one condition selected from the group consisting of: heart failure (HF) combined with atrial fibrillation, HF combined with atrial flutter, hypertension, and an inflammatory condition of the heart;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

treating the condition by driving the at least one electrode to apply electrical stimulation to the fat pad.

There is further provided, in accordance with an embodiment of the present invention, a method including:

identifying a clinical benefit for a subject of an increased eNOS level;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

increasing the eNOS level by driving the at least one electrode to apply electrical stimulation to the fat pad.

There is still further provided, in accordance with an embodiment of the present invention, a method including:

identifying a clinical benefit for a subject of a reduced iNOS level and a reduced nNOS level in cardiac tissue;

responsively to the identifying:

implanting in an atrial wall of the subject, from within an atrium, a fixation element including a screw that includes at least one electrode contact, such that the at least one electrode contact is positioned in a vicinity of a parasympathetic epicardial fat pad; and

reducing the iNOS and nNOS levels by driving the at least one electrode to apply electrical stimulation to the fat pad.

There is additionally provided, in accordance with an embodiment of the present invention, a method including:

implanting in an atrial wall of a subject, from within an atrium, at least one electrode contact at site in a vicinity of a parasympathetic epicardial fat pad;

driving the electrode contact to apply an electrical signal to the site; and

configuring the signal to both pace the heart and cause parasympathetic activation of the fat pad.

For some applications, configuring includes configuring the signal to include bursts, each of which includes a plurality of pulses, and configuring one or more initial pulses of each of the bursts to pace the heart.

There is yet additionally provided, apparatus including:

an intravascular lead;

a first electrode contact coupled to the lead at a distal end thereof, and configured to be positioned in a right atrium in a vicinity of a parasympathetic epicardial fat pad selected from the group consisting of: an atrioventricular (AV) node fat pad, and a sinoatrial (SA) node fat pad;

a second electrode contact coupled to the lead within 2 cm of the first lead;

a third electrode contact coupled to the lead such that the second electrode contact is between the first and third electrode contacts, the third electrode contact configured to be positioned within an organ selected from the group consisting of: a superior vena cava, and a right atrium in a vicinity of the superior vena cava; and

a control unit, configured to:

sense a commencement of a P-wave using the third electrode contact and at least one electrode contact selected from the group consisting of: the first electrode contact, and the second electrode contact, and

responsively to the sensing of the commencement of the P-wave, drive a current between the first and second electrode contacts, and configure the current to cause parasympathetic activation of the fat pad.

For some applications, the control unit is configured to drive the current within 30 ms of the sensing of the commencement of the P-wave.

There is also provided, in accordance with an embodiment of the present invention, a method including:

positioning a first electrode contact coupled to an intravascular lead at a distal of the lead in a right atrium in a vicinity of a parasympathetic epicardial fat pad selected from the group consisting of: an atrioventricular (AV) node fat pad, and a sinoatrial (SA) node fat pad, wherein a second electrode contact is coupled to the lead within 2 cm of the first lead;

positioning within an organ a third electrode contact coupled to the lead such that the second electrode contact is between the first and third electrode contacts, the organ selected from the group consisting of: a superior vena cava, and a right atrium in a vicinity of the superior vena cava;

sensing a commencement of a P-wave using the third electrode contact and at least one electrode contact selected from the group consisting of: the first electrode contact, and the second electrode contact; and

responsively to the sensing of the commencement of the P-wave, driving a current between the first and second electrode contacts, and configure the current to cause parasympathetic activation of the fat pad.

There is further provided, in accordance with an embodiment of the present invention, a method for implanting an electrode assembly having at least one electrode contact, including:

positioning the electrode assembly such that the at least one electrode contact is within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

advancing the electrode contact into a wall of the organ until the electrode contact is positioned entirely within the fat pad, and no other portion of the electrode assembly is in direct electrical contact with tissue of the wall.

For some applications, the at least one electrode contact includes at least two electrode contacts, and the electrode assembly includes a fixation element including a screw that includes the at least two electrode contacts.

For some applications, the electrode assembly includes a fixation element including a screw having a proximal portion having a non-conductive external surface, and a distal portion having a conductive external surface that serves as the at least one electrode contact.

There is still further provided, in accordance with an embodiment of the present invention, a method including:

implanting at least one electrode contact within an atrium of a subject in a vicinity of an interatrial groove; and

during at least one stimulation period per day over a thirty-day period, driving the electrode contact to apply stimulation to tissue of the subject, and configuring the stimulation to cause parasympathetic activation.

For some applications, chronically implanting the at least one electrode contact in the vicinity includes chronically implanting the at least one electrode contact within 2 mm of the groove. For some applications, chronically implanting the at least one electrode contact in the vicinity includes chronically implanting the at least one electrode contact in physical contact with the groove.

There is additionally provided, in accordance with an embodiment of the present invention, apparatus including:

one or more electrode contacts, configured to be placed within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

a control unit, configured to:

drive the electrode contacts to apply to the fat pad a burst of pulses including one or more initial pulses followed by one or more subsequent pulses,

set a preconditioning strength of the initial pulses to be insufficient to cause parasympathetic activation of the fat pad, and

set an activating strength of the subsequent pulses to be sufficient to cause the parasympathetic activation.

There is yet additionally provided, in accordance with an embodiment of the present invention, a method including:

placing one or more electrode contacts within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

driving the electrode contacts to apply to the fat pad a burst of pulses including one or more initial pulses followed by one or more subsequent pulses;

setting a preconditioning strength of the initial pulses to be insufficient to cause parasympathetic activation of the fat pad; and

setting an activating strength of the subsequent pulses to be sufficient to cause the parasympathetic activation.

For some applications, setting the strength of the initial and subsequent pulses includes: during a calibration procedure, driving the electrode contacts to apply a plurality of calibration bursts at respective calibration strengths; sensing whether the calibration bursts cause a vagomimetic effect; finding a minimal strength necessary to cause the vagomimetic effect; and setting the preconditioning strength to be less than the minimal strength, and the activating strength to be at least the minimal strength.

There is also provided, in accordance with an embodiment of the present invention, apparatus including:

one or more electrode contacts, configured to be placed within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base; and

a control unit, configured to:

drive the electrode contacts to apply a plurality of bursts to the fat pad,

set a frequency of the bursts to be less than or equal to 2.5 Hz, and

configure each of the bursts to include between 2 and 20 pulses, and to have a pulse repetition interval (PRI) of between 1 ms and 30 ms.

For some applications, the control unit is configured to set the burst frequency to be less than or equal to 2 Hz.

There is further provided, in accordance with an embodiment of the present invention, a method including:

placing one or more electrode contacts within an organ of a circulatory system in a vicinity of a parasympathetic epicardial fat pad, the organ selected from the group consisting of: a right atrium, a left atrium, a superior vena cava, an inferior vena cava, a coronary sinus, a right pulmonary vein, a left pulmonary vein, and a right ventricular base;

driving the electrode contacts to apply a plurality of bursts to the fat pad;

setting a frequency of the bursts to be less than or equal to 2.5 Hz; and

configuring each of the bursts to include between 2 and 20 pulses, and to have a pulse repetition interval (PRI) of between 1 ms and 30 ms.

There is still further provided, in accordance with an embodiment of the present invention, apparatus including:

at least one electrode contact, configured to be implanted, from within a right atrium of a subject, in an atrial wall in a vicinity of a parasympathetic epicardial fat pad; and

a control unit, configured to:

drive the at least one electrode contact to apply bursts of pulses without synchronizing the bursts with any feature of a cardiac cycle of the subject,

configure a burst frequency to be no more than 2.5 Hz, and

configure a pulse repetition interval (PRI) to be no more than 30 ms.

There is additionally provided, in accordance with an embodiment of the present invention, a method including:

implanting, from within a right atrium of a subject, at least one electrode contact in an atrial wall in a vicinity of a parasympathetic epicardial fat pad;

driving the at least one electrode contact to apply bursts of pulses without synchronizing the bursts with any feature of a cardiac cycle of the subject;

configuring a burst frequency to be no more than 2.5 Hz; and

configure a pulse repetition interval (PRI) to be no more than 30 ms.

There is yet additionally provided, in accordance with an embodiment of the present invention, apparatus including:

an intracardiac screw-in electrode assembly including:
an outer helical fixation element having a first radius, and including a first intracardiac electrode contact; and
an inner helical fixation element having a second radius less than the first radius, and including a second intracardiac electrode contact,
wherein the inner helical fixation element is positioned within the outer helical fixation element; and

a control unit, configured to drive a current between the first and second electrode contacts, and to configure the current to provide cardiac stimulation.

For some applications, the electrode assembly is configured such that the outer and inner helical fixation members are independently rotatable.

There is also provided, in accordance with an embodiment of the present invention, a method including:

chronically implanting at least one electrode contact within an atrium of a subject in a vicinity of a parasympathetic epicardial fat pad;

driving the at least one electrode contact to apply stimulation to tissue of the atrium;

determining whether the stimulation activates a phrenic nerve of the subject; and

responsively to finding that the stimulation activates the phrenic nerve, configuring at least one parameter of the stimulation so as to not activate the phrenic nerve.

For some applications, driving includes configuring the stimulation to cause parasympathetic activation.

There is further provided, in accordance with an embodiment of the present invention, a method including:

implanting in an atrial wall of a subject, from within an atrium, at least one electrode contact in a vicinity of a parasympathetic epicardial fat pad of the subject;

sensing, using the at least one electrode, an electrogram, and analyzing the electrogram;

upon finding that the electrogram is characteristic of atrial electrical activity, driving the at least one electrode contact to apply stimulation, and configuring the stimulation to cause parasympathetic activation of the fat pad; and

upon finding that the electrogram is not characteristic of the atrial electrical activity, withholding driving the at least one electrode contact to apply the stimulation.

For some applications, sensing includes withhold applying the stimulation during a sensing period having a duration of at least 2 seconds, and sensing during the sensing period.

There is still further provided, in accordance with an embodiment of the present invention, apparatus including:

at least one electrode contact, configured to be implanted, from within an atrium, in an atrial wall of a subject in a vicinity of a parasympathetic epicardial fat pad of the subject; and

a control unit, configured to:

sense, using the at least one electrode, an electrogram, and analyze the electrogram,

upon finding that the electrogram is characteristic of atrial electrical activity, drive the at least one electrode contact to apply stimulation, and configuring the stimulation to cause parasympathetic activation of the fat pad, and

upon finding that the electrogram is not characteristic of the atrial electrical activity, withhold driving the at least one electrode contact to apply the stimulation.

There is additionally provided, in accordance with an embodiment of the present invention, a method including:

positioning two electrode contacts within an atrium of a subject at respective locations against a wall of the atrium in a vicinity of a parasympathetic epicardial fat pad;

performing a plurality of times the steps of:
(a) separately driving the two electrode contacts to apply stimulation to the wall, and determining respective heart-rate-lowering effects of the stimulation applied by the two electrode contacts;
(b) repositioning at at least one other location against the wall whichever of the electrodes achieved a lesser heart-rate-lowering effect, while leaving the other of the electrode contacts at its location against the wall;

chronically implanting one of the electrode contacts at its location, and removing the other of the electrodes from the atrium.

For some applications, implanting includes again performing step (a), and implanting whichever of the electrode contacts achieved a greater heart-rate-lowering effect, at the location of the electrode.

For some applications, implanting includes identifying that the respective heart-rate-lowering effects have converged. Alternatively, implanting includes, upon finding that the respective heart-rate-lowering effects have not converged, implanting whichever of the electrode contacts achieved a greater heart-rate-lowering effect, at the location of the electrode, and removing the other of the electrodes from the atrium.

For some applications, performing the steps the plurality of times includes repositioning each of the electrode contacts at least once.

In an embodiment, positioning the electrode contacts includes placing at least one of the electrode contacts in a sheath that includes at least one conducting portion through which electricity is conductible, and driving the electrode contacts to apply the stimulation includes driving the at least one electrode contact to apply the stimulation through the at least one conducting portion. For some applications, the sheath is shaped so as to define at least one window that defines the at least one conducting portion. For some applications, the sheath includes a conductive element that serves as the at least one conducting portion.

A number of patents and articles describe methods and devices for stimulating nerves to achieve a desired effect. Often these techniques include a design for an electrode or electrode cuff.

The control unit of an implantable electronic device such as a pacemaker or a defibrillator typically has two portions: a metal can, which includes the circuitry of the device, and a non-metallic header, which provides connection points for leads.

U.S. Pat. No. 6,907,295 to Gross et al., which is assigned to the assignee of the present application and is incorporated herein by reference, describes apparatus for applying current to a nerve. A cathode is adapted to be placed in a vicinity of a cathodic longitudinal site of the nerve and to apply a cathodic current to the nerve. A primary inhibiting anode is adapted to be placed in a vicinity of a primary anodal longitudinal site of the nerve and to apply a primary anodal current to the nerve. A secondary inhibiting anode is adapted to be placed in a vicinity of a secondary anodal longitudinal site of the nerve and to apply a secondary anodal current to the nerve, the secondary anodal longitudinal site being closer to the primary anodal longitudinal site than to the cathodic longitudinal site.

US Patent Application Publication 2006/0106441 to Ayal et al., which is assigned to the assignee of the present application and is incorporated herein by reference, describes apparatus for applying current to a nerve, including a housing, adapted to be placed in a vicinity of the nerve, and at least one cathode and at least one anode, fixed to the housing. The apparatus further includes two or more passive electrodes, fixed to the housing, and a conducting element, which electrically couples the passive electrodes to one another.

The following patents, which are incorporated herein by reference, may be of interest:
U.S. Pat. No. 6,684,105 to Cohen et al.
U.S. Pat. No. 5,423,872 to Cigaina
U.S. Pat. No. 4,573,481 to Bullara
U.S. Pat. No. 6,230,061 to Hartung
U.S. Pat. No. 5,282,468 to Klepinski
U.S. Pat. No. 5,487,756 to Kallesoe et al.
U.S. Pat. No. 5,634,462 to Tyler et al.
U.S. Pat. No. 6,456,866 to Tyler et al.
U.S. Pat. No. 4,602,624 to Naples et al.
U.S. Pat. No. 6,600,956 to Maschino et al.
U.S. Pat. No. 5,199,430 to Fang et al.
U.S. Pat. No. 5,824,027 Hoffer et al.

The following articles, which are incorporated herein by reference, may be of interest:
Naples G G et al., “A spiral nerve cuff electrode for peripheral nerve stimulation,” by IEEE Transactions on Biomedical Engineering, 35(11) (1988)
Deurloo K E et al., “Transverse tripolar stimulation of peripheral nerve: a modelling study of spatial selectivity,” Med Biol Eng Comput, 36(1):66-74 (1998)
Tarver W B et al., “Clinical experience with a helical bipolar stimulating lead,” Pace, Vol. 15, October, Part II (1992)
Fitzpatrick et al., “A nerve cuff design for the selective activation and blocking of myelinated nerve fibers,” Ann. Conf. of the IEEE Eng. in Medicine and Biology Soc, 13(2), 906 (1991)
Baratta R et al., “Orderly stimulation of skeletal muscle motor units with tripolar nerve cuff electrode,” IEEE Transactions on Biomedical Engineering, 36(8):836-43 (1989)

In some embodiments of the present invention, a nerve stimulation and cardiac sensing system comprises at least one electrode device, which is applied to a nerve of a subject, such as a vagus nerve, at a location neither within nor in contact with a heart of the subject. The electrode device comprises one or more device first electrode contact surfaces that are configured to be placed in electrical contact with the nerve. The system further comprises a control unit, and at least one second sensing electrode contact surface which is not directly mechanically coupled to the electrode device, and which is configured to be positioned in the subject's body elsewhere than in the subject's heart, optionally at a location neither within nor in contact with the heart. The control unit uses the second sensing electrode contact surface and at least one of the device first electrode contact surfaces to sense a signal indicative of a parameter of a cardiac cycle of subject, such as one or more components of an electrocardiogram (ECG) of a heart of the subject. The control unit is typically configured to apply stimulation to the nerve, and/or configure the applied stimulation, at least in part responsively to the sensed cardiac parameter. For example, the control unit may configure the stimulation to regulate a heart rate of the subject. For some applications, the second sensing electrode contact surface is directly mechanically coupled to the control unit, while for other applications, the second sensing electrode contact surface is directly mechanically coupled to a lead that couples the control unit to the electrode device.

In some embodiments of the present invention, in addition to comprising a plurality of stimulating electrode contact surfaces within the electrode device, the electrode device comprises one or more external sensing electrode contact surfaces, which are fixed to an outer surface of the device. The control unit uses the external sensing electrode contact surfaces to sense a signal indicative of a parameter of a cardiac cycle of subject. In order to sense this property, the electrode device is typically configured to be implanted in a vicinity of a blood vessel of the subject. For some applications, the electrode device is implanted around a cervical vagus nerve in a vicinity of the carotid artery or the jugular vein. The control unit is typically configured to apply stimulation to the nerve, and/or configure the applied stimulation, at least in part responsively to the sensed cardiac parameter. For example, the control unit may configure the stimulation to regulate a heart rate of the subject.

As used in the present application, including in the claims, an “electrode” is an electrically conductive contact surface that is not electrically insulated, which is typically coupled to at least one other element by one or more leads, and an “electrode device” is a device which is configured to be positioned in a vicinity of a nerve, and which comprises at least one electrode that is configured to make electrical contact with tissue, in order to apply electrical stimulation to the tissue and/or sense an electrical property of the tissue.

There is therefore provided, in accordance with an embodiment of the present invention, apparatus for application to a nerve of a subject, the apparatus including an electrode device, which includes:

a housing, which is configured to be placed at least partially around the nerve, so as to define an outer surface of the electrode device and an inner surface that faces the nerve;

one or more first electrode contact surfaces, fixed to the inner surface of the housing; and

one or more second electrode contact surfaces, fixed to the outer surface of the housing.

In an embodiment, the apparatus further includes a control unit, which includes:

a driving unit, which is configured to drive the first electrode contact surfaces to apply electrical stimulation to the nerve;

a sensing unit, which is configured to sense an electrical signal, using at least one of the second electrode contact surfaces; and

an analysis unit, which is configured to analyze the signal to identify a parameter of a cardiac cycle of the subject.

For some applications, the nerve is the vagus nerve, and the electrode device is configured to be implanted such that the second electrode contact surfaces are in a vicinity of a blood vessel selected from the group consisting of: a carotid artery and a jugular vein.

There is further provided, in accordance with an embodiment of the present invention, apparatus including:

an electrode device, which includes one or more device first electrode contact surfaces, and which is configured to be placed in a vicinity of a nerve of a subject at a first location neither within nor in contact with a heart of the subject;

at least one second electrode contact surface, which is not directly mechanically coupled to the electrode device, and which is configured to be positioned in a body of the subject at a second location neither within nor in contact with the heart; and

a control unit, which includes:

a sensing unit, which is configured to sense, using at least one of the device first electrode contact surfaces and the at least one second electrode contact surface, an electrical signal; and

an analysis unit, which is configured to analyze the signal to identify a parameter of a cardiac cycle of the subject.

For some applications, the at least one second electrode contact surface is directly mechanically coupled to the control unit. For some applications, the control unit includes a metal can having an outer conductive surface, at least a portion of which serves as the at least one second electrode contact surface.

For some applications, the electrode device includes a housing, which is configured to be placed at least partially around the nerve, so as to define an outer surface of the electrode device and an inner surface that faces the nerve, and the one or more device first electrode contact surfaces are fixed to the inner surface of the housing. For some applications, the electrode device includes a housing, which is configured to be placed at least partially around the nerve, so as to define an outer surface of the electrode device and an inner surface that faces the nerve, and the one or more device first electrode contact surfaces are fixed to the outer surface of the housing.

For some applications, the nerve is the vagus nerve, and the electrode device is configured to be placed in the vicinity of the vagus nerve.

For some applications, the electrode device is configured to be placed at least partially around the nerve.

In an embodiment, the control unit includes a driving unit, which is configured to drive at least some of the device first electrode contact surfaces to apply electrical stimulation to the nerve, and configure the stimulation responsively to the parameter of the cardiac cycle. For some applications, the device first electrode contact surfaces include one or more stimulating device first electrode contact surfaces and one or more sensing stimulating device first electrode contact surfaces, the driving unit is configured to drive the stimulating device first electrode contact surfaces, and not the sensing stimulating device first electrode contact surfaces, to apply the stimulation, and the sensing unit is configured to sense the electrical signal using the sensing device first electrode contact surfaces, and not using the stimulating device first electrode contact surfaces. For some applications, the parameter of the cardiac cycle is indicative of ventricular contraction, and the driving unit is configured to drive the at least some of the device first electrode contact surfaces to apply the stimulation during at least one heart beat after a delay from the ventricular contraction, the delay having a duration of at least 20 ms.

In an embodiment, the apparatus further includes at least one lead coupled to the control unit, and the at least one second electrode contact surface is directly mechanically coupled to the lead. For some applications, the at least one lead couples the electrode device to the control unit, and the at least one second electrode contact surface is directly mechanically coupled to the lead at a position between the electrode device and the control unit.

There is still further provided, in accordance with an embodiment of the present invention, apparatus including:

an electrode device, which is configured to be placed in a vicinity of a nerve of a subject at a location neither within nor in contact with a heart of the subject;

a control unit;

at least one lead that couples the electrode device to the control unit;

at least one first electrode contact surface that is directly mechanically coupled to the lead at a location between the electrode device and the control unit; and

at least one second electrode contact surface that is directly mechanically coupled to the control unit, and

the control unit includes:

a sensing unit, which is configured to sense, using the at least one first electrode contact surface and the at least one second electrode contact surface, an electrical signal; and

an analysis unit, which is configured to analyze the signal to identify a parameter of a cardiac cycle of the subject.

For some applications, the control unit includes a metal can having an outer conductive surface, at least a portion of which serves as the at least one second electrode contact surface.

For some applications, the electrode device is configured to be placed at least partially around the nerve.

There is additionally provided, in accordance with an embodiment of the present invention, a method including:

placing an electrode device in a vicinity of a nerve and in a vicinity of a blood vessel of a subject, at a location neither within nor in contact with a heart of the subject;

applying electrical stimulation to the nerve using the electrode device; and

sensing, using the electrode device, a signal from the blood vessel indicative of a parameter of a cardiac cycle of the subject.

For some applications, the nerve is cervical vagus nerve, the blood vessel is selected from the group consisting of: a carotid artery and a jugular vein, and placing includes placing the electrode device in the vicinity of the cervical vagus nerve and in the vicinity of the selected blood vessel.

There is yet additionally provided, in accordance with an embodiment of the present invention, a method including:

placing an electrode device in a vicinity of a nerve of a subject at a first location neither within nor in contact with a heart of the subject; and

sensing, between the electrode device and a second location within a body of the subject neither within nor in contact with the heart, a signal indicative of a parameter of a cardiac cycle of the subject.

There is also provided, in accordance with an embodiment of the present invention, a method including:

placing an electrode device in a vicinity of a nerve of a subject at location neither within nor in contact with a heart of the subject; and

using an implanted control unit coupled to the electrode device by at least one lead, sensing, between the control unit and a location along the lead, a signal indicative of a parameter of a cardiac cycle of the subject.

The present invention will be more fully understood from the following detailed description of embodiments thereof, taken together with the drawings, in which:

### BRIEF DESCRIPTION OF THE DRAWINGS

FIG. 1A is a schematic cut-away illustration of an electrode assembly, and

FIGS. 1B and 1C are schematic cut-away illustrations of a cuff of the electrode assembly, in accordance with an embodiment of the present invention;

FIGS. 2A and 2B are schematic illustrations of the cuff of FIGS. 1A-C in open and closed positions, respectively, in accordance with an application of the present invention;

FIGS. 3 and 4A-D are schematic longitudinal cut-away and perpendicular cross-sectional illustrations of the cuff of FIGS. 1A-C, respectively, in accordance with an application of the present invention;

FIGS. 5A and 5B are symbolic perpendicular cross-sectional illustrations of the superimposition of inner closed curves of the cuff of FIGS. 1A-C, in accordance with an application of the present invention;

FIG. 6 is a schematic illustration of the crossing and intersection of two of the inner closed curves of FIGS. 4A-D, in accordance with an application of the present invention;

FIGS. 7A and 7B are schematic illustrations of the intersection, but not crossing, of two sets of inner closed curves;

FIG. 8 is a schematic longitudinal cut-away illustration of the cuff of FIGS. 1A-C, in accordance with an application of the present invention;

FIG. 9 is a schematic longitudinal cut-away illustration of an alternative configuration of the cuff of FIGS. 1A-C, in accordance with an application of the present invention;

FIGS. 10A-C are perpendicular cross-sectional illustrations of the cuff of FIGS. 1A-C, in accordance with an application of the present invention;

FIG. 11 is a block diagram that schematically illustrates a vagal stimulation system applied to a vagus nerve of a patient, in accordance with an embodiment of the present invention;

FIG. 12A is a simplified cross-sectional illustration of a multipolar electrode device applied to a vagus nerve, in accordance with an embodiment of the present invention;

FIG. 12B is a simplified cross-sectional illustration of a generally-cylindrical electrode device applied to a vagus nerve, in accordance with an embodiment of the present invention;

FIG. 12C is a simplified perspective illustration of the electrode device of FIG. 12A, in accordance with an embodiment of the present invention;

FIG. 13 is a simplified perspective illustration of a multipolar point electrode device applied to a vagus nerve, in accordance with an embodiment of the present invention;

FIG. 14 is a conceptual illustration of the application of current to a vagus nerve, in accordance with an embodiment of the present invention;

FIG. 15 is a simplified illustration of an electrocardiogram (ECG) recording and of example timelines showing the timing of the application of a series of stimulation pulses, in accordance with an embodiment of the present invention;

FIG. 16 is a graph showing in vivo experimental results, measured in accordance with an embodiment of the present invention;

FIGS. 17 and 18 are tables showing hemodynamic, angiographic, echocardiographic, and Doppler measurements made during an in vivo experiment conducted on 19 dogs, measured in accordance with an embodiment of the present invention;

FIG. 19 is a table showing histomorphometric measurements made during the experiment of FIGS. 17 and 18, measured in accordance with an embodiment of the present invention;

FIGS. 20-23 are graphs showing densitometry measurements of mRNA expression for TNF-alpha, IL-6, Activin-A, and TGF-beta, respectively, made during the experiment of FIGS. 17 and 18, measured in accordance with an embodiment of the present invention;

FIGS. 24A, 24B, and 24C are graphs showing densitometry measurements of protein expression of NOS-1, NOS-2, and NOS-3, respectively, made during the experiment of FIGS. 17 and 18, measured in accordance with an embodiment of the present invention;

FIG. 25 is a graph showing densitometry measurements of protein expression of Connexin 43, made during the experiment of FIGS. 17 and 18, measured in accordance with an embodiment of the present invention;

FIG. 26 is a graph showing N-terminal pro-brain natriuretic peptide (NT-pro-BNP) serum levels in two human subjects, measured in accordance with an embodiment of the present invention;

FIG. 27 is a schematic illustration of a nerve, showing the placement of electrode devices thereon, in accordance with a preferred embodiment of the present invention;

FIG. 28 illustrates the construction and mode of operation of a tripolar electrode device particularly useful in the present invention;

FIG. 29 diagrammatically illustrates an array of tripolar electrode devices constructed in accordance with the present invention for selectively blocking the propagation through certain nerve-fibers of body-generated action potentials;

FIG. 30 is a block diagram illustrating the stimulator in the apparatus of FIG. 13;

FIG. 31 is a block diagram illustrating the operation of the apparatus of FIGS. 29 and 30 for suppressing pain sensations;

FIGS. 32A and 32B are block diagrams illustrating how the apparatus of FIGS. 29 and 30 may also be used for suppressing selected muscular or glandular activities controlled by the motor nerves;

FIGS. 33A and 33B are block diagrams illustrating how the apparatus of FIGS. 29 and 30 may also be used for stimulating selected motor or glandular activities upon the failure of the body to generate the required action potentials;

FIGS. 34A and 34B are diagrams helpful in explaining the manner of calibrating the apparatus of FIGS. 29 and 30;

FIG. 35 is a schematic illustration of a nerve and experimental apparatus applied thereto, in accordance with a preferred embodiment of the present invention;

FIGS. 36A, 36B, and 36C are graphs showing data measured using the experimental apparatus of FIG. 27;

FIG. 37 is a schematic illustration of a series of bursts, in accordance with an embodiment of the present invention;

FIG. 38 is a graph showing experimental results obtained in an experiment performed on human subjects, in accordance with an embodiment of the present invention;

FIG. 39 is a schematic illustration of a stimulation regimen, in accordance with an embodiment of the present invention;

FIG. 40 is a schematic illustration of another stimulation regimen, in accordance with an embodiment of the present invention;

FIG. 41 is a graph showing experimental results obtained in an animal experiment, in accordance with an embodiment of the present invention;

FIG. 42 is a schematic illustration of a parasympathetic stimulation system for stimulating autonomic nervous tissue from at least partially within a heart, in accordance with an embodiment of the present invention;

FIGS. 43A-C are schematic illustrations of configurations of an electrode assembly of the system of FIG. 42, in accordance with respective embodiments of the present invention;

FIGS. 44A-C are schematic illustrations of a screw-in fixation elements of the system of FIG. 42, in accordance with respective embodiments of the present invention;

FIGS. 45A-B are schematic illustrations of electrode assemblies configured to minimize the risk of bleeding, in accordance with respective embodiments of the present invention;

FIG. 46A is a schematic illustration of another parasympathetic stimulation system, in accordance with an embodiment of the present invention;

FIG. 46B is a schematic illustration of an alternative configuration of the system of FIG. 46A, in accordance with another embodiment of the present invention;

FIG. 47 is a schematic illustration of yet another parasympathetic stimulation system, in accordance with an embodiment of the present invention;

FIG. 48 is a schematic illustration of a sheath, in accordance with an embodiment of the present invention;

FIG. 49 is a schematic illustration of a parasympathetic stimulation system for stimulation of postganglionic fibers, in accordance with an embodiment of the present invention;

FIG. 50 is a schematic illustration of tripolar ganglion plexus (GP) electrode assembly, in accordance with an embodiment of the present invention;

FIG. 51 is a schematic illustration of an atrial region for stimulation of postganglionic fibers, in accordance with an embodiment of the present invention;

FIG. 52 is a schematic illustration of yet another configuration of the stimulation system of FIG. 42, in accordance with an embodiment of the present invention;

FIG. 53 is a flow chart schematically illustrating a method for implanting an electrode assembly at a desired position in an atrial fat pad, in accordance with an embodiment of the present invention;

FIGS. 54A-G are graphs illustrating electrical data recorded and/or analyzed in accordance with respective embodiments of the present invention;

FIG. 55 is a schematic illustration of a nerve stimulation and cardiac sensing system, in accordance with an embodiment of the present invention;

FIG. 56 is a schematic illustration of an electrode device, in accordance with an embodiment of the present invention;

FIG. 57 is a schematic, cross-sectional illustration of an electrode cuff for applying current to a nerve, in accordance with an embodiment of the present invention;

FIG. 58 is a schematic, cross-sectional illustration of an electrode cuff for sensing a cardiac signal at a blood vessel, in accordance with an embodiment of the present invention; and

FIGS. 59-61 are graphs illustrating experimental results measured in accordance with respective embodiments of the present invention.

### DETAILED DESCRIPTION OF APPLICATIONS

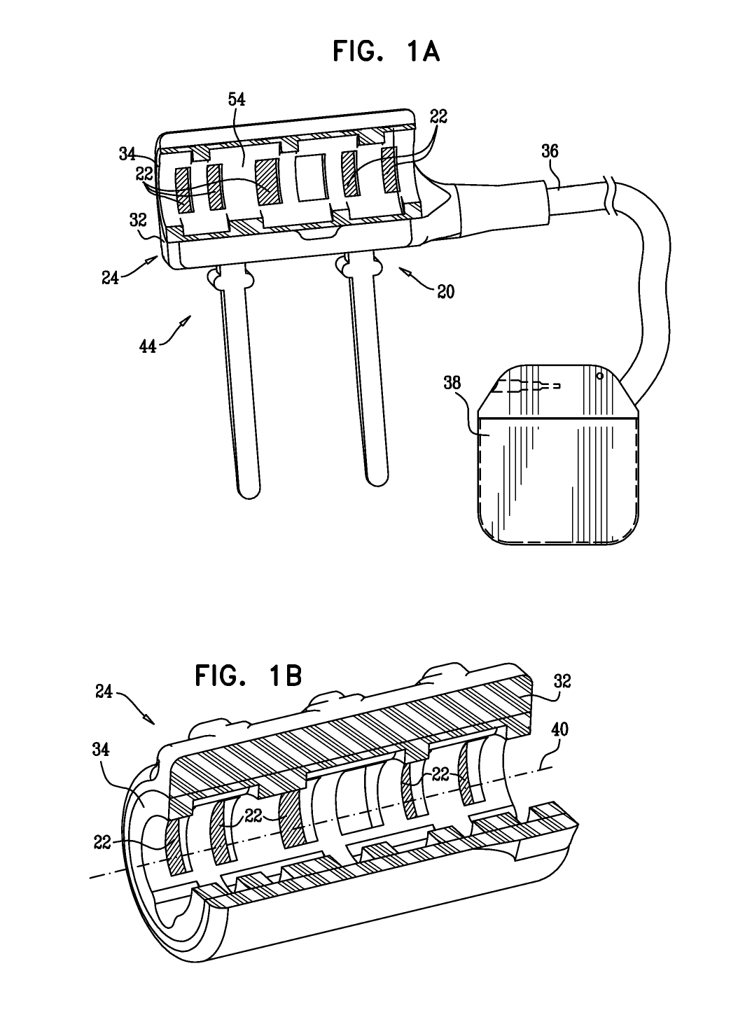

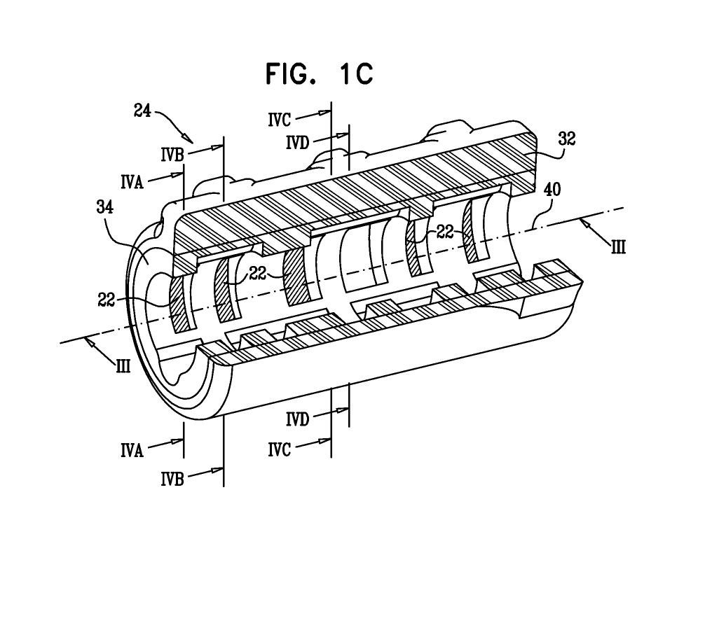

FIG. 1A is a schematic cut-away illustration of an electrode assembly 20, and FIGS. 1B and 1C are schematic cut-away illustrations of a cuff 24 of electrode assembly 20, in accordance with an embodiment of the present invention. Electrode assembly 20 comprises cuff 24 and one or more electrode contact surfaces 22. Cuff 24 is configured to be placed at least partially around (typically entirely around) a nerve or other tubular body tissue, such as a blood vessel, a muscle, a tendon, a ligament, an esophagus, intestine, a fallopian tube, a neck of a gall bladder, a cystic duct, a hepatic duct, a common hepatic duct, a bile duct, and/or a common bile duct. Cuff 24 defines and at least partially surrounds (typically entirely surrounds) a longitudinal axis 40. The cross section of FIG. 1A shows 180 degrees of a circumference of cuff 24 (i.e., 50% of the cuff; the cuff actually completely surrounds 360 degrees of axis 40), while the cross section of FIG. 1B shows 270 degrees of the circumference of the cuff (i.e., 75% of the cuff; the cuff actually completely surrounds 360 degrees of axis 40). Typically, electrode contact surfaces 22 are fixed to cuff 24 such that the contact surfaces are electrically exposed to and face axis 40.

For some applications, as shown in FIGS. 1A-C, cuff 24 comprises an outer housing 32 and an inner insulating tube 34. Outer housing 32 is fixed around inner insulating tube 34, and defines an outer surface of the cuff. Providing these inner and outer layers may facilitate manufacturing of the cuff, including placement of electrode contact surfaces 22 within recesses of the cuff, as described hereinbelow. Housing 32 typically comprises an elastic, electrically-insulating material such as silicone or polyurethane, which may have, for example, a hardness of between about 40 Shore A and about 80 Shore A, such as about 40 Shore A. Inner insulating tube 34 typically comprises an elastic, electrically-insulating material such as silicone or silicone copolymer, which, for some applications, is softer than that of housing 32, for example, having a hardness of between about 1 Shore A and about 40 Shore A, such as about 10 Shore A.

For other applications, cuff 24 comprises a single integrated element, rather than a separate outer housing and inner insulating tube (configuration not shown). The element typically comprises an elastic, electrically-insulating material such as silicone or polyurethane, which may have, for example, a hardness of between about 5 Shore A and about 40 Shore A, such as about 10 Shore A. Alternatively, cuff 24 comprises more than two elements that are fixed to one another.

Electrode assembly 20 optionally further comprises a lead assembly 36, which comprises one or more electrical leads, as is known in the art. The leads are coupled to all or a portion of electrode contact surfaces 22. Lead assembly 36 couples electrode assembly 20 to an implanted or external control unit 38, which comprises appropriate circuitry for driving current between two or more of electrode contact surfaces 22, as is known in the art. Typically, the control unit configures the current such that one or more of the contact surfaces function as cathodes, and one or more function as anodes.

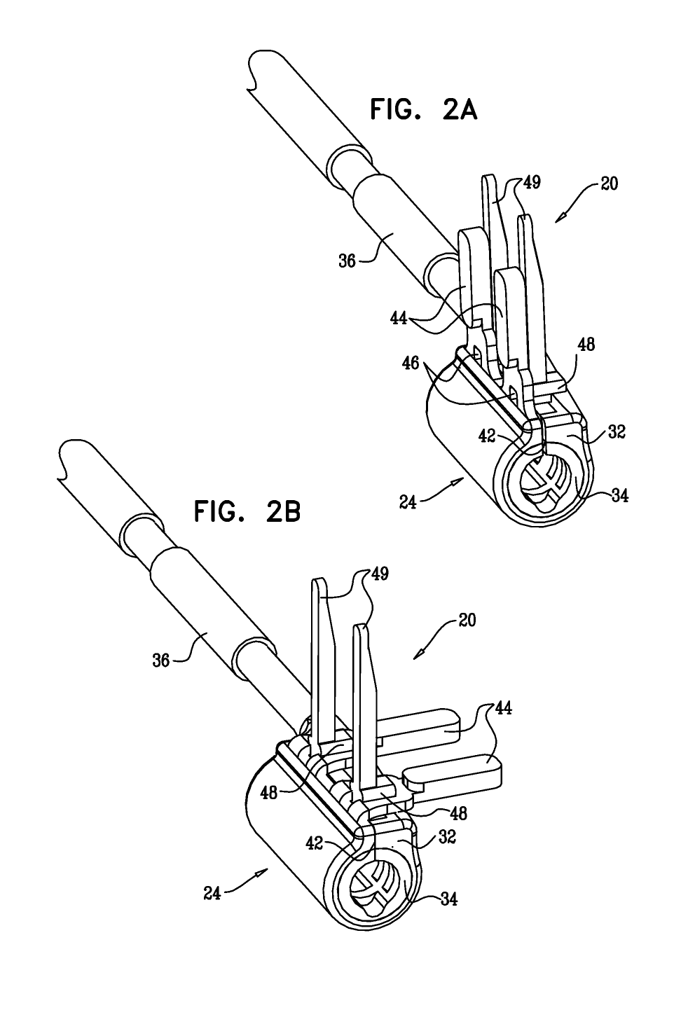

Reference is made to FIGS. 2A and 2B, which are schematic illustrations of cuff 24 in open and closed positions, respectively, in accordance with an application of the present invention. Typically, cuff 24 is shaped so as to define a longitudinal slit 42 along the entire length of the cuff. The cuff assumes the open position when the edges of the slit do not touch each other. The cuff is placed around the tubular body tissue, such as the nerve, by passing the tubular body tissue through the slit. The edges of the slit are brought together to bring the cuff into the closed position.

For some applications, electrode assembly 20 further comprises one or more closing elements 44, which are configured to hold the edges of slit 42 together. For some applications, each of the closing elements comprises an opening 46 near one edge of slit 42 and a corresponding protrusion 48 on the other edge of the slit. To close the cuff, each of the protrusions is inserted into the corresponding slit. Optionally, each of the closing elements further comprises a tab 49, which the surgeon implanting the cuff may grasp to help pull protrusion 48 through opening 46.

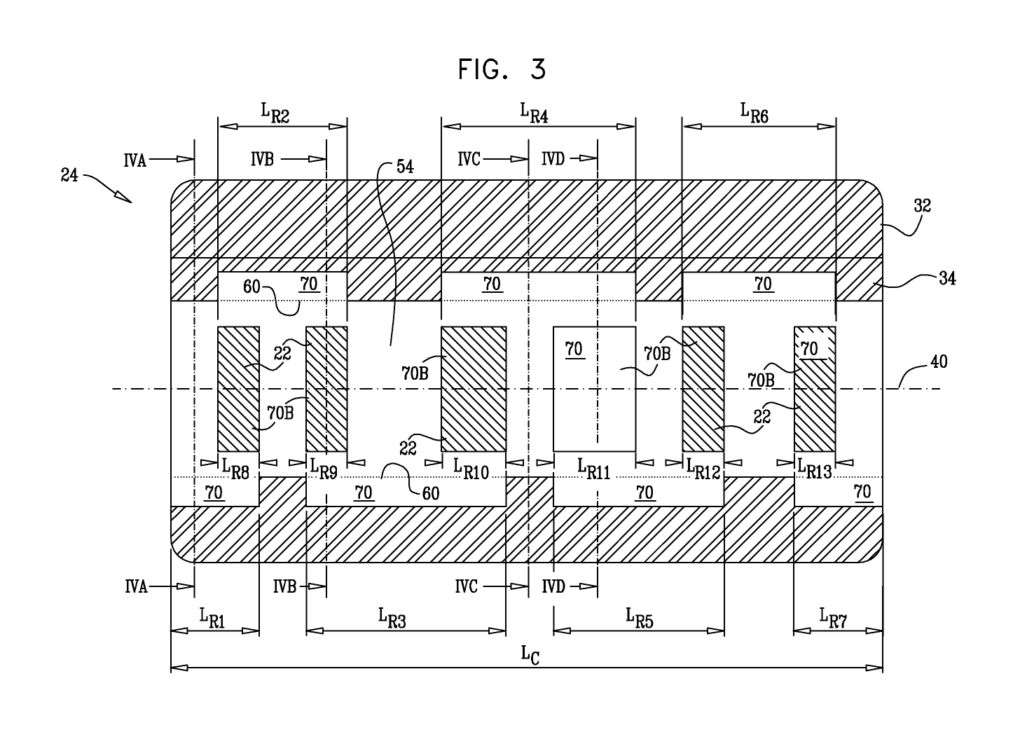

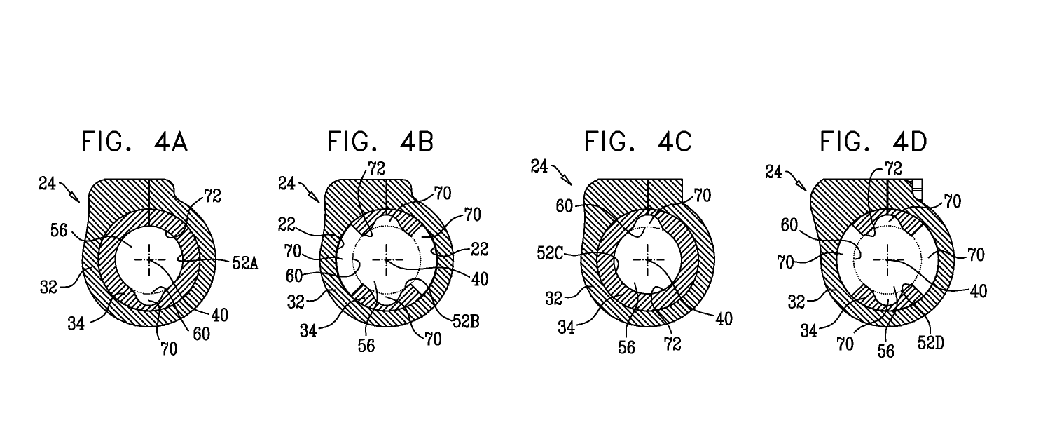

Reference is made to FIGS. 3 and 4A-D, which are schematic longitudinal cut-away and perpendicular cross-sectional illustrations of cuff 24, respectively, in accordance with an application of the present invention. In FIG. 3, the recesses labeled 70B extend in a direction perpendicular to the plane of the page, into the page. When cuff 24 is in the closed position, such as described hereinabove with reference to FIG. 2B, the cuff is shaped so as to define a plurality of planar cross sections perpendicular to longitudinal axis 40, distributed continuously along an entire length L_{C} of the cuff. Perpendicular cross sections IVA-IVA, IVB-IVB, IVC-IVC, and IVD-IVD, indicated in FIGS. 1C and 3, are three of these perpendicular planar cross sections. The plurality of perpendicular cross sections define respective inner closed curves 52 surrounding longitudinal axis 40. For example, perpendicular cross sections IVA-IVA, IVB-IVB, IVC-IVC, and IVD-IVD, shown in FIGS. 4A, 4B, 4C, and 4D, respectively, define respective inner closed curves 52A, 52B, 52C, and 52D, respectively. Inner closed curves 52 together define an inner surface 54 that defines and completely surrounds a combined innermost volume that extends along entire length L_{C} of the cuff. Because not all of the inner closed curves have the same shape, the perpendicular cross-sectional shape of volume 56 varies along the length of the cuff. In addition, inner closed curves 52 define and enclose respective inner cross-sectional regions 56.

Entire length L_{C} of cuff 24, measured along longitudinal axis 40, is typically at least 1 mm, no more than 40 mm, and/or between 1 and 40 mm. Typically, the combined innermost volume has a volume of at least 10 mm3, no more than 5000 mm3, and/or between 10 and 5000 mm3, such as at least 15 mm3, no more than 200 mm3, and/or between 15 and 200 mm3.

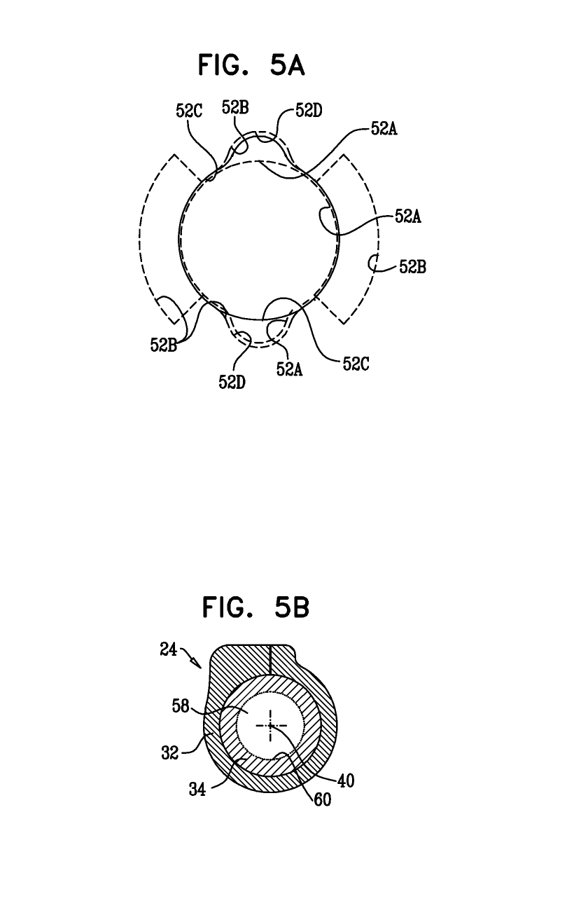

Reference is made to FIGS. 5A and 5B, which are symbolic perpendicular cross-sectional illustrations of the superimposition of inner closed curves 52 of cuff 24, in accordance with an application of the present invention. FIG. 5B additionally shows outer housing 32 and inner insulating tube 34. All of inner closed curves 52, if superimposed while preserving orientation and position of the perpendicular cross sections and the inner closed curves with respect to the cuff, would together define a combined innermost closed curve 60 surrounding longitudinal axis 40. For example, FIG. 5A shows inner curves 52A, 52B, 52C, and 52D superimposed while preserving orientation and position of the perpendicular cross sections and the inner closed curves with respect to cuff 24. (In order to better illustrate the curves, coinciding portions are shown slightly offset from one another, even though they actually coincide.) Curves 52A, 52B, 52C, and 52D, if thus superimposed, would together define a combined innermost closed curve 60 surrounding longitudinal axis 40, as shown in FIG. 5B. (In this example, combined innermost closed curve 60 is a complete circle.) In addition, an intersection of cross-sectional regions 56 (shown, by way of example, in FIGS. 4A-D), if the cross-sectional regions were to be superimposed while preserving orientation and position of the cross-sectional regions with respect to cuff 24, would define a combined inner cross-sectional region 58 (shown, by way of example, in FIG. 5B). Combined inner cross-sectional region 58, if extended along the entire length of cuff 24, would define the combined innermost volume. In addition, a periphery of combined inner cross-sectional region 58 defines combined innermost closed curve 60.

Typically, combined innermost closed curve 60 and/or the combined inner cross-sectional region is shaped to correspond to and/or accommodate the shape of the tubular body tissue, such as the nerve, around which cuff 24 is placed. For some applications, combined innermost closed curve 60 and/or the combined inner cross-sectional region is elliptical, such as circular (as shown in the figures). For other applications, the combined innermost closed curve and/or the combined inner cross-sectional region has another non-elliptical shape, such as a shape chosen to correspond to the anatomical perpendicular cross section of the tubular body tissue, e.g., the nerve, to which the cuff is applied. Combined innermost closed curve 60, if extended along the entire length of cuff 24, would define a combined innermost volume (combined inner cross-sectional region 58, if extended along the entire length of cuff 24, would also define the combined innermost volume). For example, for applications in which combined innermost closed curve 60 is elliptical, the combined innermost volume is an elliptical cylinder, and, for applications in which combined innermost closed curve 60 is elliptical, the combined innermost volume is a circular cylinder. (As used herein, including in the claims, the term “elliptical” includes, but is not limited to, “circular” within its scope.) For some applications in which the combined innermost volume is an elliptical (e.g., circular) cylinder, the cylinder has a major axis that is at least 1 mm, no more than 8 mm, and/or between 1 and 8 mm and a minor axis that is at least 0.5 mm, no more than 6 mm, and/or between 0.5 and 6 mm.

Generally, the cuff is placeable around an elliptical (e.g., circular) cylinder. The cuff is also placeable around tubular body tissue, such as a nerve, which is not perfectly elliptical, but may be generally elliptical. It is to be understood that the cylinder and the tubular body tissue are not elements of apparatus of the present invention, and are described, and recited in a portion of the claims, for purposes of helping to define the structure of the actual elements of the apparatus. The cylinder may be considered an abstraction of the tubular body tissue, which may be helpful, in some cases, for defining with definiteness the structure of the cuff without reference to parts of the human body.

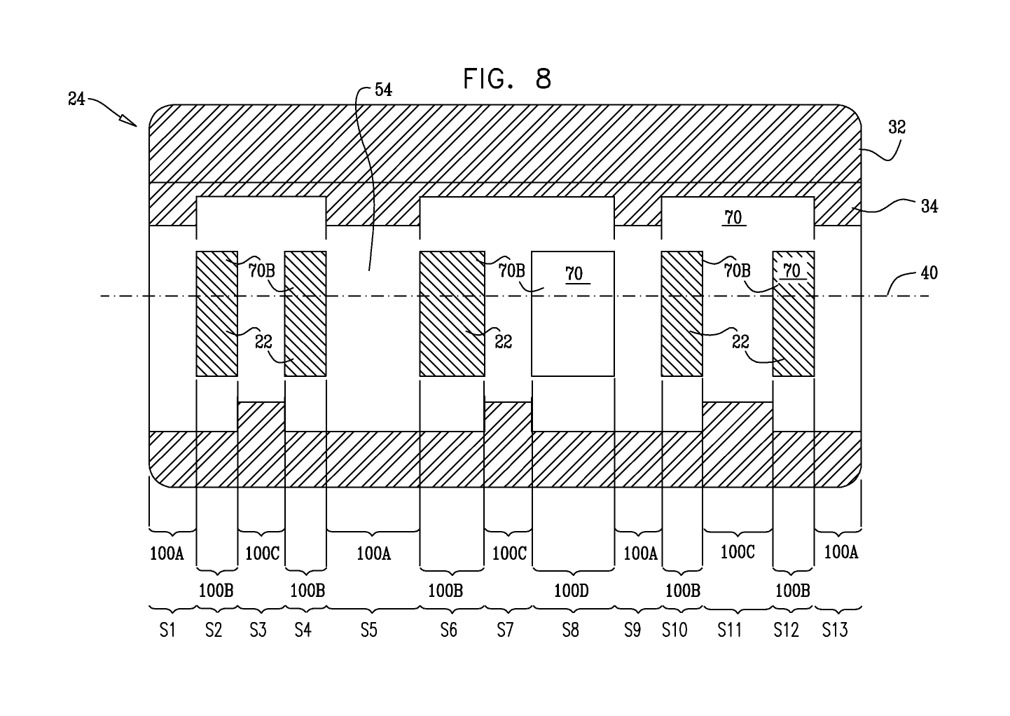

Cuff 24 is shaped so as to define a plurality of recesses 70 that extend and are recessed radially outwardly from the combined innermost volume. (The recesses also extend radially outwardly from the tubular body tissue if the cuff is placed therearound, and/or from the elliptical cylinder, if the cuff is placed therearound.) At any given angle around longitudinal axis 40 in any given planar cross section perpendicular to axis 40 that includes a recess, the surface of the recess facing longitudinal axis 40 is further from the axis than is the combined innermost closed curve at given the angle and cross section. The recesses extend along the longitudinal axis of the cuff. (In FIGS. 3 and 8, the recesses labeled 70B extend in a direction perpendicular to the plane of the page, into the page.)

For some applications, cuff 24 is shaped such that every one of the planar cross sections perpendicular to axis 40 along entire length L_{C} of the cuff partially defines at least one of recesses 70, such that cuff 24 is recessed at every longitudinal location along the entire length L_{C} of the cuff, and at least one of the recesses is at every longitudinal location along the entire length L_{C} of the cuff. (Any given perpendicular planar cross section only partially, rather than fully, defines at least one of the recesses, because the cross section defines the at least one of the recesses in combination with other cross sections.) In other words, at every longitudinal location along its entire length L, cuff 24 is recessed in at least one radially outward direction (the radially outward directions typically differ at at least some of the longitudinal locations). As a result, inner closed curves 52 respectively defined by the perpendicular cross sections enclose respective areas, each of which areas is greater than an area enclosed by combined innermost closed curve 60. It is noted that, for these applications, cuff 24 is recessed even at its longitudinal ends. In other words, even at its ends, cuff 24 is not shaped so as to define an inner surface that coincides with combined innermost closed curve 60. For applications in which cuff 24 is applied to a nerve, recesses 70 may serve to prevent damage to the nerve by allowing the nerve to swell in at least one radial direction into at least one of the recesses, along entire length L_{C} of the cuff.

For some applications, such as shown in FIGS. 4A and 4C, at least one segment of the cuff is shaped so as to define a single recess 70. Alternatively or additionally, at least one segment of the cuff is shaped so as to define more than one recess 70, such as at least two recesses 70 (e.g., exactly two recesses 70), at least three recesses 70 (e.g., exactly three recesses 70), or at least four recesses 70 (e.g., exactly four recesses 70, as shown in FIGS. 4B and 4D).

For some applications, each of recesses 70 has a length L_{R} along the cuff that is less than entire length L_{C} of the cuff, e.g., less than 50%, 40%, 25%, or 15% of length L_{C}. This design generally prevents migration of the tubular body tissue, such as the nerve, over time into the recesses, away from the center of the cuff, as might occur if any of the recesses extended along the entire length, or even most of the length, of the cuff. Holding the cuff in position around the nerve helps maintain good electrical contact between the electrical contact surfaces and the tubular body tissue, such as the nerve. In addition, the recesses thus do not provide a continuous path for current applied by the electrode contact surfaces to pass through the cuff without entering the tubular body tissue, such as the nerve.

Typically, one or more portions of each of inner closed curves 52 coincides with combined innermost closed curve 60, such that each of the inner closed curves coincides with the combined innermost closed curve at a portion of, but not all, angles with respect to axis 40, such that the cuff comes in contact with the tubular body tissue at a portion of, but not all, angles with respect to axis 40. Such contact of these non-recessed portions may help hold the cuff in position around the tubular body structure, thereby aiding in maintaining good electrical contact between the electrical contact surfaces and the tissue.

For some applications, each of the recesses has a length, measured in parallel with longitudinal axis 40, of at least 0.1 mm, no more than 15 mm, and/or between 0.1 and 15 mm. For some applications in which cuff 24 defines two or more longitudinal segments 100, as described hereinbelow with reference to FIG. 8, each of the recesses that longitudinally spans a single segment 100 has a length of at least 0.1 mm, no more than 10 mm, and/or between 0.1 and 10 mm, and/or a length of at least 2% of the entire length of the cuff (e.g., at least 5%), no more than 50% of the entire length (e.g., no more than 20%), and/or between 2% and 50% of the entire length (e.g., between 5% and 20%), while each of the recesses that longitudinally spans more than one segment 100 has a length of at least 0.3 mm, no more than 15 mm, and/or between 0.3 and 15 mm, and/or a length of at least 5% of the entire length of the cuff (e.g., at least 10%), no more than 50% of the entire length (e.g., no more than 40%), and/or between 5% and 50% of the entire length (e.g., between 10% and 40%). For some applications, each of the recesses has a length of at least 0.1 mm, such as at least 0.3 mm. Typically, recesses have a plurality of different respective lengths. For example, the cuff may be shaped so as to define recesses having all or a portion of the following respective ranges of lengths and exemplary lengths:

TABLE 1

| | Range of lengths | Exemplary length |
|---|---|---|
| L_{R1} | 0.3 mm-15 mm | 1.5 mm |
| L_{R2} | 0.3 mm-15 mm | 2.2 mm |
| L_{R3} | 0.3 mm-15 mm | 3.4 mm |
| L_{R4} | 0.3 mm-15 mm | 3.3 mm |
| L_{R5} | 0.3 mm-15 mm | 2.9 mm |
| L_{R6} | 0.3 mm-15 mm | 2.6 mm |
| L_{R7} | 0.3 mm-10 mm | 1.5 mm |
| L_{R8} | 0.1 mm-10 mm | 0.7 mm |
| L_{R9} | 0.1 mm-10 mm | 0.7 mm |
| L_{R10} | 0.1 mm-10 mm | 1.1 mm |
| L_{R11} | 0.1 mm-10 mm | 1.4 mm |
| L_{R12} | 0.1 mm-10 mm | 0.7 mm |
| L_{R13} | 0.1 mm-10 mm | 0.7 mm |

For some applications, the recesses having these lengths are arranged as shown in FIG. 3. For other applications, the recesses are otherwise arranged.

Typically, at least some of recesses 70 overlap one another lengthwise along the cuff (i.e., along axis 40), either partially or completely, without overlapping anglewise with respect to axis 40 (i.e., the recesses are recessed radially outward from the axis 40 at different, non-overlapping angles with respect to the axis, so that the recesses do not intersect one another). As a result, at least one of the perpendicular cross sections partially defines at least two of the recesses. For example, as shown in FIG. 3, a first recess having length L_{R1} partially overlaps lengthwise a second recess having length L_{R2} (with an overlap length of 0.7 mm). The first recess is recessed in an upward direction in the figure, while the second recess is recessed in a downward direction in the figure, such that the first and second recesses do not overlap each other anglewise with respect to axis 40. The second recess (having length L_{R2}) additionally overlaps lengthwise a third recess having length L_{R3} (by 0.7 mm). For some applications, a first one of recesses 70 partially overlaps lengthwise a second one of recesses 70 with an overlap length of at least 0.1 mm, no more than 15 mm, and/or between 0.1 and 15 mm, and/or an overlap length equal to at least 10%, no more than 60%, and/or between 10% and 60% of the length of the first one of the recesses. In addition, for example, as shown in FIG. 3, the recess having length L_{R2} partially overlaps the recesses having lengths L_{R7} and L_{R8} (by the entire lengths of L_{R7} and L_{R8}). For some applications, recesses 70 that partially overlap lengthwise each other do not overlap each other anglewise; in other words, the recesses extend in different, non-overlapping radial directions.

Reference is still made to FIGS. 3 and 4A-D. Cuff 24 is typically shaped such that each planar cross section thereof perpendicular to axis 40 includes one or more non-recessed portions 72 that coincide with combined innermost closed curve 60. These non-recessed portions serve in part to hold the cuff in position around the tubular body tissue, such as a nerve.

Reference is made to FIGS. 4A-D. For some applications, at least two of recesses 70 extend radially outwardly in different radial directions, such as in opposite radial directions. For example, the recess shown in FIG. 4A extends radially outwardly in the opposite radial direction of the recess shown in FIG. 4C. For some applications, the at least two of the recesses that extend radially outwardly in different radial directions are defined at least in part by a common perpendicular cross section. For example, perpendicular cross section IVB-IVB, shown in FIG. 4B, defines a first recess that extends upward in the figure, and a second recess that extends downward in the figure, i.e., in opposite radial directions.

Reference is again made to FIGS. 4A, 4C, and 5B. For some applications, at least two of the perpendicular cross sections of cuff 24 define respective inner closed curves 52 that have different shapes, and not merely different sizes, when orientation and position of the perpendicular cross sections and inner closed curves with respect to the cuff are preserved. For example, assume that a first perpendicular cross section has the circular shape of combined innermost closed curve 60, shown in FIG. 5B, and a second perpendicular cross section has the shape of inner closed curve 52A, shown in FIG. 4A. These two perpendicular cross sections have different shapes. Likewise, two perpendicular cross sections having the shapes of inner closed curves 52A and 52C, shown in FIGS. 4A and 4C, respectively, would also have different shapes, when orientation and position of the perpendicular cross sections and inner closed curves with respect to the cuff are preserved (even though inner closed curves 52A and 52C would have the same shape if orientation were not preserved). However, two circular perpendicular cross sections having different radii would not have different shapes, but merely different sizes.

As used in the present application, including in the claims, “preserving orientation” of the perpendicular cross sections and/or inner closed curves with respect to the cuff means not rotating the perpendicular cross sections or and inner closed curves, such as not rotating the perpendicular cross sections or inner closed curves around longitudinal axis 40. For example, inner closed curves 52A and 52C, shown in FIGS. 4A and 4C, respectively, are considered to have different shapes when preserving orientation of the perpendicular cross sections and inner closed curves with respect to the cuff. This is the case even though inner closed curves 52A and 52C would have the same shape if one of the inner closed curves were to be rotated 180 degrees around longitudinal axis 40, i.e., if the orientation of the inner closed curves with the cuff were not preserved. As used in the present application, including in the claims, “preserving position” of the perpendicular cross sections and/or inner closed curves with respect to the cuff means not translating the perpendicular cross sections and/or inner closed curves within their respective planes, e.g., changing offsets of the perpendicular cross sections and/or inner closed curves with respect to longitudinal axis 40. For example, assume that two inner closed curves were both circles of the same size. These two circular inner closed curves would be considered to have the same shape, even though they would cross each other if one of the inner closed curves were translated in any direction in its plane.

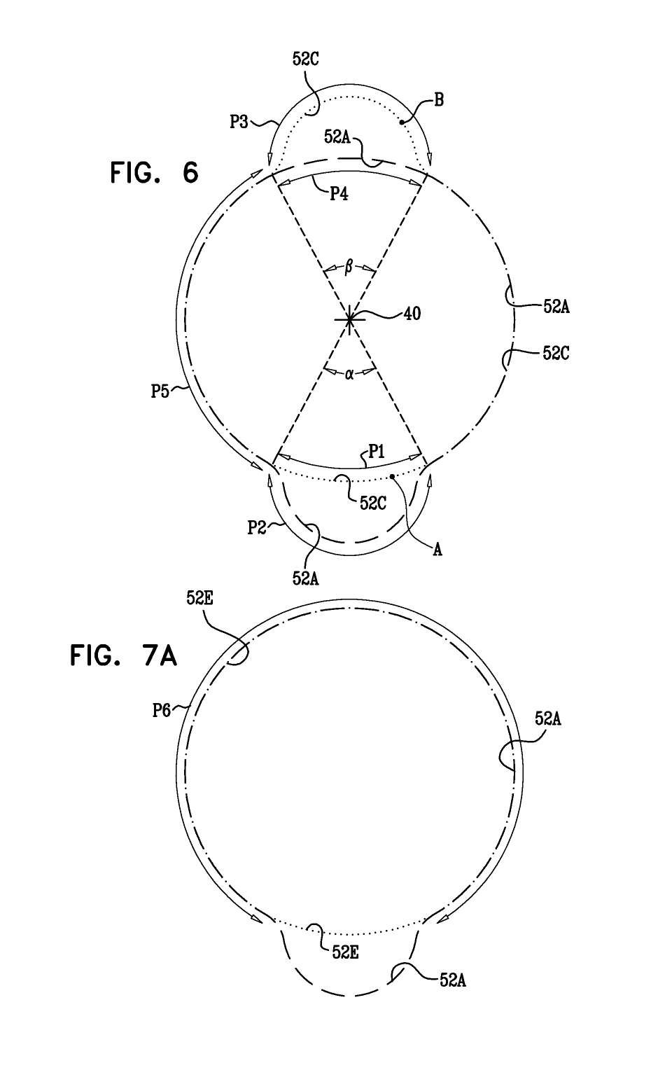

Reference is now made to FIG. 6, which is a schematic illustration of the crossing and intersection of two of the inner closed curves of FIGS. 4A-D, in accordance with an application of the present invention. In this application, inner closed curves 52 of the at least two of the perpendicular cross sections would cross, and not merely intersect, one another if superimposed while preserving orientation and position of the perpendicular cross sections and inner closed curves with respect to the cuff. For example, in FIG. 6 inner closed curve 52A (of FIG. 4A) and inner closed curve 52C (of FIG. 4C) are shown superimposed. As can be seen, inner closed curves 52A and 52C cross each other, and do not merely intersect. For example, a portion P1 of inner closed curve 52C is closer to longitudinal axis 40 of the combined perpendicular cross section than is a portion P2 of inner closed curve 52A at the same first range of angles α from axis 40, while a portion P3 of inner closed curve 52C is further from axis 40 than is a portion P4 of inner closed curve 52A at the same second range of angles β from axis 40. This is only possible because the inner closed curves cross each other. In other words, any path along inner closed curve 52C from a point A on portion P1 to a point B on portion P3 must cross inner closed curve 52A, i.e., go from one side of inner closed curve 52A with respect to axis 40 (the inner side) to the other side of inner closed curve 52A with respect to axis 40 (the outer side). It is noted that inner closed curves 52A and 52C cross one another, even though they may coincide (such as along a portion P5) for a certain range of angles with respect to axis 40 while crossing.

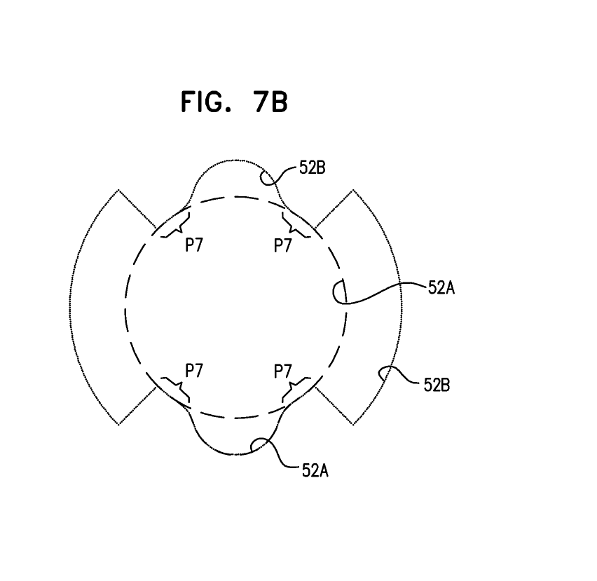

FIGS. 7A and 7B are schematic illustrations of the intersection, but not crossing, of two sets of inner closed curves. In FIG. 7A inner closed curve 52A (of FIG. 4A) (shown as dashed in FIG. 7A) and a circular inner closed curve 52E (shown as dotted) are shown superimposed. As can be seen, inner closed curves 52A and 52E merely intersect (along a portion P6), but do not cross each other. In FIG. 7B inner closed curve 52A (of FIG. 4A) (shown as dashed in FIG. 7B) and inner closed curve 52B (shown as dotted) are shown superimposed. As can be seen, inner closed curves 52A and 52B merely intersect (along portions P7), but do not cross each other.

For some applications, inner closed curve 52 of at least one of the perpendicular cross sections is rotationally non-symmetric for all rotational angles. For example, inner closed curve 52A, shown in FIG. 4A, is rotationally non-symmetric for all rotational angles. Optionally, inner closed curves 52 of at least two of the perpendicular cross sections having different shapes (when orientation and position of the perpendicular cross sections and inner closed curves with respect to the cuff are preserved) are rotationally non-symmetric for all rotational angles. For example, inner closed curves 52A and 52C, shown in FIGS. 4A and 4C, respectively, have different shapes (when orientation and position of the perpendicular cross sections and inner closed curves with respect to the cuff are preserved), and are rotationally non-symmetric for all rotational angles. Optionally, inner closed curves 52 of all of the perpendicular cross sections may be rotationally non-symmetric (configuration not shown). Optionally, for each of these different degrees of rotational non-symmetry, combined innermost closed curve 60, described hereinabove with reference to FIGS. 5A-B, may be circular.

Reference is again made to FIGS. 4A-D. For some applications, such as shown in FIG. 4B, one or more of recesses 70 have respective electrode contact surfaces 22 fixed therein, such that the electrode contact surfaces are recessed radially outward from combined innermost closed curve 60. As a result, when cuff 24 is placed around the tubular body tissue, such as the nerve, the electrode contact surfaces are not in physical contact with the nerve when the cuff is placed around the tissue. In addition, one or more of recesses 70 may not have an electrode contact surface coupled therein, such as shown in FIGS. 1A, 1B, 4A, 4C, and 4D.

Alternatively or additionally, one or more of electrode contact surfaces 22 are coupled to non-recessed portions 72 of cuff 24 that coincide with combined innermost closed curve 60, which are described hereinabove with reference to FIGS. 3 and 4A-D. As a result, when cuff 24 is placed around the tubular body tissue, such as the nerve, the electrode contact surfaces are in physical contact with the tissue.

Reference is made to FIG. 8, which is a schematic longitudinal cut-away illustration of cuff 24, in accordance with an application of the present invention. In FIG. 8, the recesses labeled 70B extend in a direction perpendicular to the plane of the page, into the page. In this application, cuff 24 is constructed so as to define two or more longitudinal segments 100, distributed continuously along the entire length of the cuff. The segments typically do not overlap one another lengthwise along the cuff. The segments are differentiated from one another by their perpendicular cross-sectional shapes and/or by whether they include electrode contact surfaces 22. The segments have respective planar cross sections perpendicular to longitudinal axis 40, which perpendicular cross sections define respective inner closed curves surrounding the longitudinal axis, such that the inner closed curve of a given segment 100 is of uniform shape along the entire given segment, when orientation and position of the perpendicular cross sections and inner closed curves with respect to the cuff are preserved. For some applications, the inner closed curve of each of at least a portion, e.g., all, of the segments is of uniform size along the segment. Alternatively or additionally, the inner closed curve of each of at least a portion, e.g., all, of the segments is of non-uniform size along the segment (for example, the size of inner closed curve may monotonically increase along the segment).

For some applications, inner curves of at least three (such as at least four, at least five, at least ten, or all) longitudinally-adjacent segments 100 have different shapes, and not merely different sizes, when orientation and position of the segments with respect to the cuff are preserved. For some applications, the segments have respective lengths, measured in parallel with longitudinal axis 40, each of which is at least 0.1 mm (e.g., at least 0.5 mm), no more than 50% of the entire length of the cuff (e.g., no more than 20%), and/or between 0.1 mm (e.g., 0.5 mm) and 50% of the entire length of the cuff (e.g., 20%). For some applications, two of segments 100 that include respective electrode contact surfaces 22 are separated by at least one of segments 100 that does not include any electrode contact surfaces.

During manufacture, inner insulating tube 34 of cuff 24 is typically molded as a single piece that is shaped so as to define the segments. The segments are typically not made as separate pieces and subsequently affixed to one another.

For some applications, cuff 24 defines at least three segments 100, such as at least four, at least five, at least six, at least 10, at least 13, or at least 15 segments 100. For some applications, at least two of segments 100 that are not longitudinally adjacent to each other have the same shape, when orientation and position of the segments with respect to the cuff are preserved. For example, segments 100 may have a total of two, three, four, or five different shapes, such that a portion of the segments share a common one of these shapes.

For some applications, segments 100 include segments types 100A, 100B, 100C, and 100D, some of which repeat along the cuff more than once. Each of these instances of a segment type has the same shape (when orientation and position of the segments with respect to the cuff are preserved) as the other instances of the segment type, but may have a different length (along axis 40 of the cuff) from the other instances of the segment type. For some applications, the recess defined by a first segment type (e.g., segment type 100A) extends radially outward beyond innermost closed curve 60 generally in a first radial direction (e.g., downward in FIG. 8), while the recess defined by a second segment type (e.g., segment type 100C) extends radially outward beyond innermost closed curve 60 generally in a second radial direction different from the first radial direction (e.g., upward in FIG. 8), such as generally opposite to the first radial direction, e.g., between 120 and 180 degrees from the first radial direction, such as 180 degrees from the first radial direction.

For some applications, first and second ones of segment types 100 have the same shape (when orientation and position of the segments with respect to the cuff are preserved), but differ in that the first segment type (e.g., segment type 100B) includes one of electrode contact surfaces 22, while the second segment type (e.g., segment type 100D) does not include any of electrode contact surfaces 22.

Typically, each of segments 100A, 100B, 100C, and 100D has a longitudinal length along the cuff of at least 0.2 mm, such as at least 0.5 mm. For some applications, the length of each segment is at least 0.2 mm, no more than 20 mm, and/or between 0.2 and 20 mm, such as at least 0.5 mm, no more than 4 mm, and/or between 0.5 and 4 mm.

For some applications, segment types 100A, 100B, 100C, and 100D have the shapes of perpendicular cross sections IVA-IVA, IVB-IVB, IVC-IVC, and IVD-IVD, respectively, indicated in FIGS. 1C and 3, and shown in FIGS. 4A-D.

In one particular configuration of cuff 24 illustrated in FIG. 8, segments 100 comprise 13 segments S1-S13. For example, the segments may have the segment types (shapes) and ranges of lengths or exemplary lengths shown in the following table:

TABLE 2

| | Segment | Range of | Exemplary |
|---|---|---|---|
| Segment | type | lengths | length |
| S1 | 100A | 0.1 mm-10 mm | 0.8 mm |
| S2 | 100B | 0.1 mm-10 mm | 0.7 mm |
| S3 | 100C | 0.1 mm-10 mm | 0.8 mm |
| S4 | 100B | 0.1 mm-10 mm | 0.7 mm |
| S5 | 100A | 0.1 mm-10 mm | 1.6 mm |
| S6 | 100B | 0.1 mm-10 mm | 1.1 mm |
| S7 | 100C | 0.1 mm-10 mm | 0.8 mm |
| S8 | 100D | 0.1 mm-10 mm | 1.4 mm |
| S9 | 100A | 0.1 mm-10 mm | 0.8 mm |
| S10 | 100B | 0.1 mm-10 mm | 0.7 mm |
| S11 | 100C | 0.1 mm-10 mm | 1.2 mm |
| S12 | 100B | 0.1 mm-10 mm | 0.7 mm |
| S13 | 100A | 0.1 mm-10 mm | 0.8 mm |

Thus, for example, segments S1 and S5 have the same perpendicular cross-sectional shape as each other (when orientation and position of the segments with respect to the cuff are preserved), but may have different lengths from each other. For some applications, a recess 70 defined by a first segment type (e.g., segment type 100A) extends generally in a first radial direction, while a recess 70 defined by a second segment type (e.g., segment type 100C) extends generally in a second radial direction different from the first radial direction, such as generally opposite to the first radial direction, e.g., between 120 and 180 degrees from the first radial direction, such as 180 degrees from the first radial direction.

As mentioned above, for some applications, a portion of segments 100 include electrode contact surfaces 22, in one or more of the recesses defined by the segment. The following tables set forth two exemplary distributions of the electrode contact surfaces in the segments. The tables also indicate, by way of example, which of the surfaces are configured by control unit 38 (FIG. 1A) to function as cathode(s), which as anode(s), and which as passive electrode(s). Each of the passive electrodes is coupled to at least one other passive electrode, and is electrically device-coupled to neither (a) any circuitry that is electrically device-coupled to at least one cathode or at least one anode, nor (b) an energy source. The passive electrodes may be implemented using techniques described in U.S. Pat. No. 7,627,384 to Ayal et al., which is incorporated herein by reference.

TABLE 3

| Segment | Electrode type |
|---|---|
| S2 | Passive electrode |
| S4 | Anode |
| S6 | Cathode |
| S10 | Cathode |
| S12 | Passive electrode |

TABLE 4

| Segment | Electrode type |
|---|---|
| S2 | Passive electrode |
| S4 | Cathode |
| S6 | Anode |
| S10 | Anode |
| S12 | Passive electrode |

For some applications, segment S8 does not include an electrode contact surface. For some applications, each of the segments that includes an electrode contact surface includes two or more electrode contact surfaces, fixed within respective recesses of the segment that extend in different radial directions. For example, the segments containing electrode contact surfaces may have cross sections shaped as shown in FIG. 4B, and may contain exactly two electrode contact surfaces 22 in respective recesses 70 that extend in opposite direction (e.g., in FIG. 4B, to the right and to the left).

For some applications, segment types 100A, 100B, 100C, and 100D of Table 2 have the shapes of perpendicular cross sections IVA-IVA, IVB-IVB, IVC-IVC, and IVD-IVD, respectively, indicated in FIGS. 1C and 3, and shown in FIGS. 4A-D.

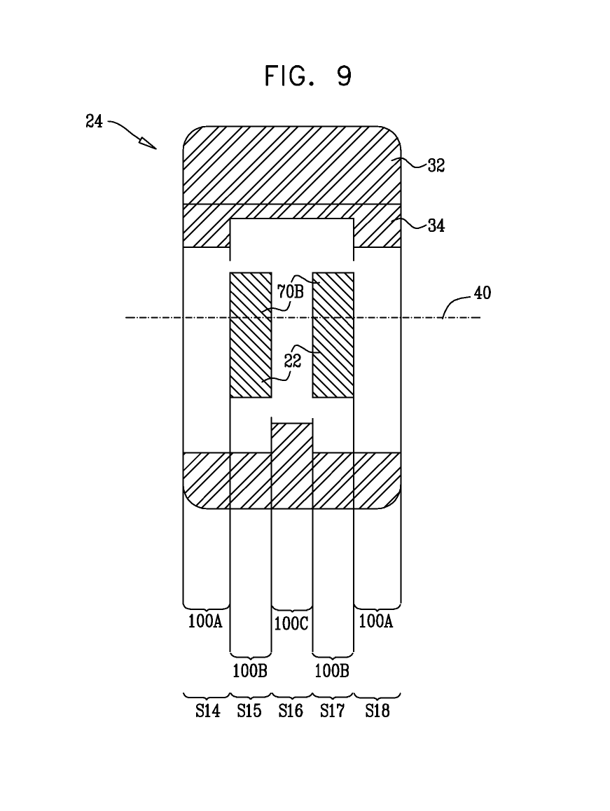

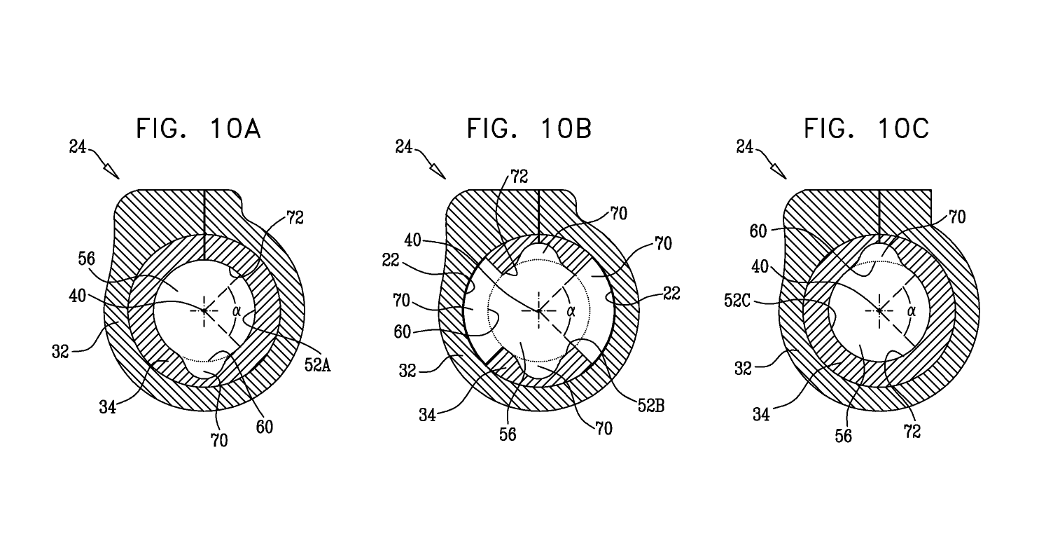

Reference is made to FIG. 9, which is a schematic longitudinal cut-away illustration of an alternative configuration of cuff 24, in accordance with an application of the present invention. In FIG. 9, the recesses labeled 70B extend in a direction perpendicular to the plane of the page, into the page. In this particular configuration, cuff 24 is constructed so as to define five longitudinal segments 100, distributed continuously along the entire length of the cuff. Two of the longitudinal segments include respective electrode contact surfaces 22. The segments are differentiated from one another by their perpendicular cross-sectional shapes and/or by whether they include electrode contact surfaces 22. The segments have respective planar cross sections perpendicular to longitudinal axis 40, which perpendicular cross sections define respective inner closed curves surrounding the longitudinal axis, such that the inner closed curve of a given segment 100 is of uniform shape and size along the entire given segment, when orientation and position of the perpendicular cross sections and inner closed curves with respect to the cuff are preserved. For some applications, inner curves of longitudinally-adjacent segments 100 have different shapes, and not merely different sizes, when orientation and position of the segments with respect to the cuff are preserved. The two of segments 100 that include respective electrode contact surfaces 22 are separated by at least one of segments 100 that does not include any electrode contact surfaces.

During manufacture, inner insulating tube 34 of cuff 24 is typically molded as a single piece that is shaped so as to define the segments. The segments are typically not made as separate pieces and subsequently affixed to one another.

In the particular configuration shown in FIG. 9, cuff 24 defines five segments 100, which include segment types 100A, 100B, and 100C, some of which repeat along the cuff more than once. Each of these instances of a segment type has the same shape (when orientation and position of the segments with respect to the cuff are preserved) as the other instances of the segment type, but may have a different length (along axis 40 of the cuff) from the other instances of the segment type. For some applications, the recess defined by a first segment type (e.g., segment type 100A) extends radially outward beyond innermost closed curve 60 generally in a first radial direction (e.g., downward in FIG. 9), while the recess defined by a second segment type (e.g., segment type 100C) extends radially outward beyond innermost closed curve 60 generally in a second radial direction different from the first radial direction (e.g., upward in FIG. 9), such as generally opposite to the first radial direction, e.g., between 120 and 180 degrees from the first radial direction, such as 180 degrees from the first radial direction.

Typically, each of segments 100A, 100B, and 100C has a longitudinal length along the cuff of at least 0.2 mm, such as at least 0.5 mm. For some applications, the length of each segment is at least 0.2 mm, no more than 20 mm, and/or between 0.2 and 20 mm, such as at least 0.5 mm, no more than 4 mm, and/or between 0.5 and 4 mm. For some applications, segment types 100A, 100B, and 100C have the shapes of perpendicular cross sections IVA-IVA, IVB-IVB, and IVC-IVC, respectively, indicated in FIGS. 1C and 3, and shown in FIGS. 4A-C.

In the particular configuration of cuff 24 illustrated in FIG. 9, segments 100 comprise five segments S14-S18. For example, the segments may have the segment types (shapes) and ranges of lengths or exemplary lengths shown in the following table:

TABLE 5

| | Segment | Range of | Exemplary |
|---|---|---|---|
| Segment | type | lengths | length |
| S14 | 100A | 0.1 mm-10 mm | 0.8 mm |
| S15 | 100B | 0.1 mm-10 mm | 0.7 mm |
| S16 | 100C | 0.1 mm-10 mm | 0.8 mm |
| S17 | 100B | 0.1 mm-10 mm | 0.7 mm |
| S18 | 100A | 0.1 mm-10 mm | 1.6 mm |

Thus, for example, segments S14 and S18 have the same perpendicular cross-sectional shape as each other (when orientation and position of the segments with respect to the cuff are preserved), but may have different lengths from each other. For some applications, a recess 70 defined by a first segment type (e.g., segment type 100A) extends generally in a first radial direction, while a recess 70 defined by a second segment type (e.g., segment type 100C) extends generally in a second radial direction different from the first radial direction, such as generally opposite to the first radial direction, e.g., between 120 and 180 degrees from the first radial direction, such as 180 degrees from the first radial direction.

As mentioned above, in this particular configuration, two of segments 100 include electrode contact surfaces 22, in one or more of the recesses defined by the segment. The following tables set forth two exemplary distributions of the electrode contact surfaces in the segments. The tables also indicate, by way of example, which of the surfaces are configured by control unit 38 (FIG. 1A) to function as a cathode, and which as an anode.

TABLE 6

| Segment | Electrode type |
|---|---|
| S15 | Cathode |
| S17 | Anode |

TABLE 7

| Segment | Electrode type |
|---|---|
| S15 | Anode |
| S17 | Cathode |

For some applications, each of the segments that includes an electrode contact surface includes two or more electrode contact surfaces, fixed within respective recesses of the segment that extend in different radial directions. For example, the segments containing electrode contact surfaces may have cross sections shaped as shown in FIG. 4B, and may contain exactly two electrode contact surfaces 22 in respective recesses 70 that extend in opposite direction (e.g., in FIG. 4B, to the right and to the left).

For some applications, segment types 100A, 100B, and 100C of Table 5 have the shapes of perpendicular cross sections IVA-IVA, IVB-IVB, and IVC-IVC, respectively, indicated in FIGS. 1C and 3, and shown in FIGS. 4A-C.

Reference is made to FIGS. 10A-C, which are perpendicular cross-sectional illustrations of the cuff of FIGS. 1A-C, in accordance with an application of the present invention. For some applications, cuff 24 is configured to include at least first and second segments 100 that include respective first and second electrode contact surfaces 22, fixed within first and second recesses 70, respectively (each of the segments may further include additional electrode surfaces in other respective recesses defined by the segment, and/or additional recesses without electrode surfaces). The first and second segments are longitudinally separated by one or more third segments 100, which typically do not include electrode contact surfaces. The first, second, and third segments are configured such that the one or more third segments electrically isolates the first electrode contact surface from the second electrode contact surface when cuff 24 is placed around the tubular body tissue, such as the nerve. As a result, current driven by control unit 38 (FIG. 1A) between the first and second electrode contact surfaces passes substantially through the tubular body tissue, rather than through the at least a third segment, or between an inner surface of the third segment and the tubular body tissue. In other words, all conductive paths between the first and second electrode contact surfaces pass through the tubular body tissue, and not between the tubular body tissue and the inner surface of the at least a third segment.

For some applications, the first recess is recessed radially outward from the combined innermost volume at a first range of angles with respect to axis 40, and the second recess is recessed radially outward from the combined innermost volume at a second range of angles with respect to axis 40. Typically, the first and second ranges of angles coincide. For example, the first and second segments may both have the cross-sectional shape shown in FIG. 10B, and the first and second ranges of angles may extend from about 45 degrees to 135 degrees, as indicated by an angle α (alpha) in FIG. 10B (assuming that 0 degrees is upwards in the figure). The one or more third segments (which longitudinally separate the first segment from the second segment) have respective inner closed curves 52 that coincide with combined innermost closed curve 60 at both the first and second range of angles.

For example, one or more of the third segments may have the cross-sectional shape shown in FIG. 10A, such that inner closed curve 52A coincides with innermost closed curve 60 at the range of angles indicated by angle α (alpha) in FIG. 10A (about 45 degrees to about 135 degrees).

In an alternative example, the third segments may include two segments, one of which has the cross-sectional shape shown in FIG. 10A, and the other the cross-sectional shape shown in FIG. 10C, in which inner closed curve 52C coincides with innermost closed curve 60 at the range of angles indicated by angle α (alpha) in FIG. 10C (about 45 degrees to about 135 degrees).

Alternatively or additionally, for some applications, cuff 24 surrounds a volume (which corresponds generally to the tubular body tissue for applications in which the cuff is applied to the tubular body tissue) that is defined by extending combined innermost closed curve 60 (described hereinabove with reference to FIGS. 5A-B) along the entire length of the cuff. Any current driven by the control unit between the first and second electrode contact surfaces must pass through this volume, rather than through the at least a third segment, or between an inner surface of the third segment and the volume. In other words, all conductive paths between the first and second electrode contact surfaces pass through the volume, and not between the volume and the inner surfaces of the at least a third segment.

Typically, the perpendicular cross-sectional area enclosed by the at least a third segment is greater than the perpendicular cross-sectional area of the tubular body tissue and/or volume surrounded by the at least a third segment. The at least a third segment nevertheless provides electrical isolation between the first and second segments, because the at least a third segment comes in physical contact with the tubular body tissue and/or volume in the radial direction(s) of electrode contact surfaces. One or more recesses 70 defined by the at least a third segment are recessed in one or more radial directions different from the one or more directions of electrode contact surfaces 22.

For example, as shown in FIG. 8, the first and second segments may be of segment type 100B (e.g., segments S2 and S4), and the at least a third segment may be of segment type 100C (e.g., segment S3). Segment types 100B and 100C may correspond to the perpendicular cross sections shown in FIGS. 4B and 4C, respectively. Segment S3, having the shape of FIG. 4C, includes insulating material on its right and left sides in FIG. 4C, which isolates electrode contact surfaces 22 of segments S2 and S4 from each other. Recess 70 of segment S3, because it is recessed in another direction (upward in FIG. 4C), does not prevent this electrical isolation.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. Provisional Patent Application 60/383,157 to Ayal et al., filed May 23, 2002, entitled, “Inverse recruitment for autonomic nerve systems,”
International Patent Application PCT/IL02/00068 to Cohen et al., filed Jan. 23, 2002, entitled, “Treatment of disorders by unidirectional nerve stimulation,” which published as PCT Publication WO 03/018113, and U.S. patent application Ser. No. 10/488,334, in the national stage thereof, which published as US Patent Application Publication 2004/0243182,
U.S. patent application Ser. No. 09/944,913 to Cohen and Gross, filed Aug. 31, 2001, entitled, “Treatment of disorders by unidirectional nerve stimulation,” which issued as U.S. Pat. No. 6,684,105,
U.S. patent application Ser. No. 09/824,682 to Cohen and Ayal, filed Apr. 4, 2001, entitled “Method and apparatus for selective control of nerve fibers,” which issued as U.S. Pat. No. 6,600,954,
U.S. patent application Ser. No. 10/205,475 to Gross et al., filed Jul. 24, 2002, entitled, “Selective nerve fiber stimulation for treating heart conditions,” which published as US Patent Application Publication 2003/0045909,
U.S. patent application Ser. No. 10/205,474 to Gross et al., filed Jul. 24, 2002, entitled, “Electrode assembly for nerve control,” which issued as U.S. Pat. No. 6,907,295,
International Patent Application PCT/IL03/00431 to Ayal et al., filed May 23, 2003, entitled, “Selective nerve fiber stimulation for treating heart conditions,” which published as PCT Publication WO 03/099377 to Ayal et al.,
International Patent Application PCT/IL03/00430 to Ayal et al., filed May 23, 2003, entitled, “Electrode assembly for nerve control,” which published as PCT Publication WO 03/099373 to Ayal et al., and U.S. patent application Ser. No. 10/529,149, in the national stage thereof, which published as US Patent Application Publication 2006/0116739,
U.S. patent application Ser. No. 10/719,659 to Ben David et al., filed Nov. 20, 2003, entitled, “Selective nerve fiber stimulation for treating heart conditions,” which published as US Patent Application Publication 2004/0193231,
U.S. patent application Ser. No. 11/022,011 to Cohen et al., filed Dec. 22, 2004, entitled, “Construction of electrode assembly for nerve control,” which issued as U.S. Pat. No. 7,561,922,
U.S. patent application Ser. No. 11/234,877 to Ben-David et al., filed Sep. 22, 2005, entitled, “Selective nerve fiber stimulation,” which published as US Patent Application Publication 2006/0100668,
U.S. patent application Ser. No. 11/280,884 to Ayal et al., filed Nov. 15, 2005, entitled, “Techniques for nerve stimulation,” which issued as U.S. Pat. No. 7,627,384,
U.S. patent application Ser. No. 11/517,888 to Ben-Ezra et al., filed Sep. 7, 2006, entitled, “Techniques for reducing pain associated with nerve stimulation,” which published as US Patent Application Publication 2008/0065158,
U.S. patent application Ser. No. 12/217,930 to Ben-David et al., filed Jul. 9, 2008, entitled, “Electrode cuffs,” which published as US Patent Application Publication 2010/0010603,
U.S. patent application Ser. No. 11/347,120, filed Feb. 2, 2006, which published as US Patent Application Publication 2006/0195170,
U.S. patent application Ser. No. 12/228,630 to Ben-David et al., filed Aug. 13, 2008, entitled, “Electrode devices for nerve stimulation and cardiac sensing,” which published as US Patent Application Publication 2010/0042186, and/or
U.S. patent application Ser. No. 12/947,608, filed Nov. 16, 2010, which published as US Patent Application Publication 2011/0098796.

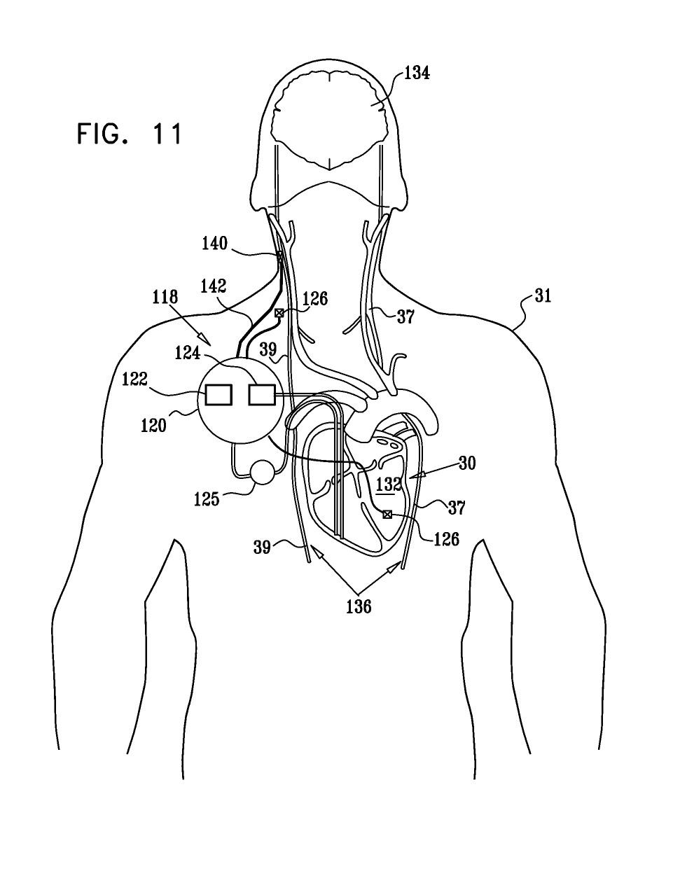

FIG. 11 is a block diagram that schematically illustrates a vagal stimulation system 118 comprising a multipolar electrode device 140, in accordance with an embodiment of the present invention. Electrode device 140 is applied to a portion of a vagus nerve 136 (either a left vagus nerve 37 or a right vagus nerve 39), which innervates a heart 30 of a patient 31. Typically, system 118 is utilized for treating a heart condition such as heart failure and/or cardiac arrhythmia. Vagal stimulation system 118 further comprises an implanted or external control unit 120, which typically communicates with electrode device 140 over a set of leads 142. For some applications, control unit 120 drives electrode device 140 to (i) apply signals to induce the propagation of efferent nerve impulses towards heart 30, and (ii) suppress artificially-induced afferent nerve impulses towards a brain 134 of the patient, in order to minimize unintended side effects of the signal application. Alternatively, control unit 120 drives electrode device 140 to apply signals that induce symmetric or asymmetric bi-directional propagation of nerve impulses. For some applications, the efferent nerve pulses in vagus nerve 136 are induced by electrode device 140 in order to regulate the heart rate of the patient.

For some applications, control unit 120 is adapted to receive feedback from one or more of the electrodes in electrode device 140, and to regulate the signals applied to the electrode device responsive thereto.

Control unit 120 is typically adapted to receive and analyze one or more sensed physiological parameters or other parameters of the patient, such as heart rate, electrocardiogram (ECG), blood pressure, indicators of decreased cardiac contractility, cardiac output, norepinephrine concentration, or motion of the patient. In order to receive these sensed parameters, control unit 120 may comprise, for example, an ECG monitor 124, connected to a site on the patient's body such as heart 30, for example using one or more subcutaneous sensors or ventricular and/or atrial intracardiac sensors. The control unit may also comprise an accelerometer 122 for detecting motion of the patient. Alternatively, ECG monitor 124 and/or accelerometer 122 comprise separate implanted devices placed external to control unit 120, and, optionally, external to the patient's body. Alternatively or additionally, control unit 120 receives signals from one or more physiological sensors 126, such as blood pressure sensors or a copeptin sensor. Sensors 126 are typically implanted in the patient, for example in a left ventricle 32 of heart 30. In an embodiment, control unit 120 comprises or is coupled to an implanted device 125 for monitoring and correcting the heart rate, such as an implantable cardioverter defibrillator (ICD) or a pacemaker (e.g., a bi-ventricular or standard pacemaker). For example, implanted device 125 may be incorporated into a control loop executed by control unit 120, in order to increase the heart rate when the heart rate for any reason is too low.

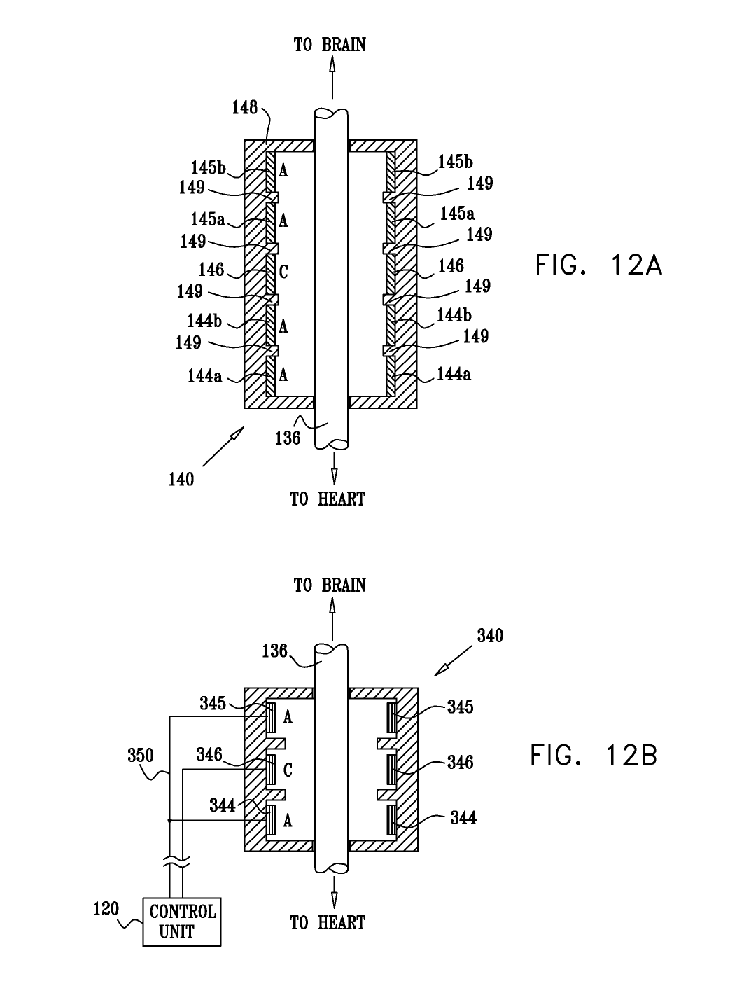

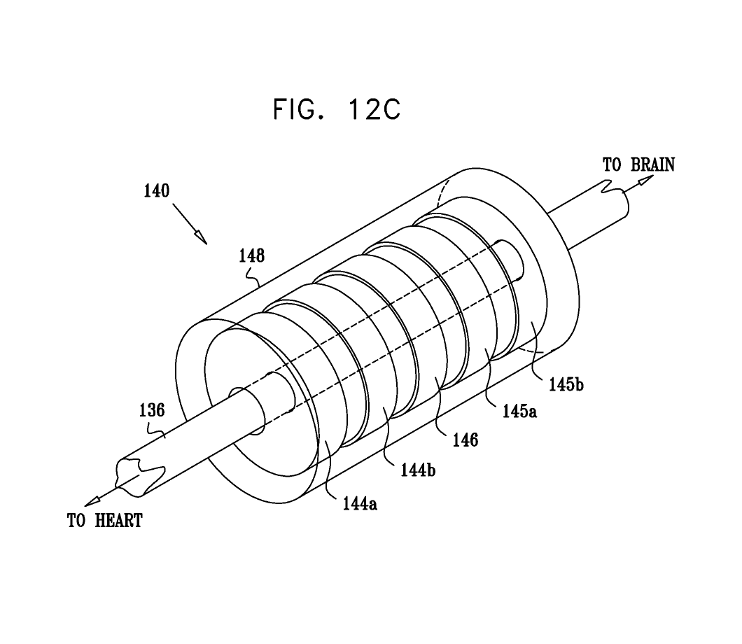

FIG. 12A is a simplified cross-sectional illustration of a generally-cylindrical electrode device 140 applied to vagus nerve 136, in accordance with an embodiment of the present invention. Electrode device 140 comprises a central cathode 146 for applying a negative current (“cathodic current”) in order to stimulate vagus nerve 136, as described below. Electrode device 140 additionally comprises a set of one or more anodes 144 (144a, 144b, herein: “efferent anode set 144”), placed between cathode 146 and the edge of electrode device 140 closer to heart 30 (the “efferent edge”). Efferent anode set 144 applies a positive current (“efferent anodal current”) to vagus nerve 136, for blocking action potential conduction in vagus nerve 136 induced by the cathodic current, as described below. Typically, electrode device 140 comprises an additional set of one or more anodes 145 (145a, 145b, herein: “afferent anode set 145”), placed between cathode 146 and the edge of electrode device 140 closer to brain 134. Afferent anode set 145 applies a positive current (“afferent anodal current”) to vagus nerve 136, in order to block propagation of action potentials in the direction of the brain during application of the cathodic current.

For some applications, the one or more anodes of efferent anode set 144 are directly electrically coupled to the one or more anodes of afferent anode set 145, such as by a common wire or shorted wires providing current to both anode sets substantially without any intermediary elements. Typically, coatings on the anodes, shapes of the anodes, positions of the anodes, sizes of the anodes and/or distances of the various anodes from the nerve are regulated so as to produce desired ratios of currents and/or desired activation functions delivered through or caused by the various anodes. For example, by varying one or more of these characteristics, the relative impedance between the respective anodes and central cathode 146 is regulated, whereupon more anodal current is driven through the one or more anodes having lower relative impedance. In these applications, central cathode 146 is typically placed closer to one of the anode sets than to the other, for example, so as to induce asymmetric stimulation (i.e., not necessarily unidirectional in all fibers) between the two sides of the electrode device. The closer anode set typically induces a stronger blockade of the cathodic stimulation.

Reference is now made to FIG. 12B, which is a simplified cross-sectional illustration of a generally-cylindrical electrode device 340 applied to vagus nerve 136, in accordance with an embodiment of the present invention. Electrode device 340 comprises exactly one efferent anode 344 and exactly one afferent anode 345, which are electrically coupled to each other, such as by a common wire 350 or shorted wires providing current to both anodes 344 and 345, substantially without any intermediary elements. The cathodic current is applied by a cathode 346 with an amplitude sufficient to induce action potentials in large- and medium-diameter fibers (e.g., A- and B-fibers), but insufficient to induce action potentials in small-diameter fibers (e.g., C-fibers).

Reference is again made to FIG. 12A. Cathodes 146 and anode sets 144 and 145 (collectively, “electrodes”) are typically mounted in an electrically-insulating cuff 148 and separated from one another by insulating elements such as protrusions 149 of the cuff. Typically, the width of the electrodes is between about 0.5 and about 2 millimeters, or is equal to approximately one-half the radius of the vagus nerve. The electrodes are typically recessed so as not to come in direct contact with vagus nerve 136. For some applications, such recessing enables the electrodes to achieve generally uniform field distributions of the generated currents and/or generally uniform values of the activation function defined by the electric potential field in the vicinity of vagus nerve 136. Alternatively or additionally, protrusions 149 allow vagus nerve 136 to swell into the canals defined by the protrusions, while still holding the vagus nerve centered within cuff 148 and maintaining a rigid electrode geometry. For some applications, cuff 148 comprises additional recesses separated by protrusions, which recesses do not contain active electrodes. Such additional recesses accommodate swelling of vagus nerve 136 without increasing the contact area between the vagus nerve and the electrodes. For some applications, the distance between the electrodes and the axis of the vagus nerve is between about 1 and about 4 millimeters, and is greater than the closest distance from the ends of the protrusions to the axis of the vagus nerve. Typically, protrusions 149 are relatively short (as shown). For some applications, the distance between the ends of protrusions 149 and the center of the vagus nerve is between about 1 and 3 millimeters. (Generally, the diameter of the vagus nerve is between about 2 and 3 millimeters.) Alternatively, for some applications, protrusions 149 are longer and/or the electrodes are placed closer to the vagus nerve in order to reduce the energy consumption of electrode device 140.

In an embodiment of the present invention, efferent anode set 144 comprises a plurality of anodes 144, typically two anodes 144a and 144b, spaced approximately 0.5 to 2.0 millimeters apart. Application of the efferent anodal current in appropriate ratios from a plurality of anodes generally minimizes the “virtual cathode effect,” whereby application of too large an anodal current stimulates rather than blocks fibers. In an embodiment, anode 144a applies a current with an amplitude equal to about 0.5 to about 5 milliamps (typically one-third of the amplitude of the current applied by anode 144b). When such techniques are not used, the virtual cathode effect generally hinders blocking of smaller-diameter fibers, as described below, because a relatively large anodal current is generally necessary to block such fibers.

Anode 144a is typically positioned in cuff 148 to apply current at the location on vagus nerve 136 where the virtual cathode effect is maximally generated by anode 144b. For applications in which the blocking current through anode 144b is expected to vary substantially, efferent anode set 144 typically comprises a plurality of virtual-cathode-inhibiting anodes 144a, one or more of which is activated at any time based on the expected magnitude and location of the virtual cathode effect.

Likewise, afferent anode set 145 typically comprises a plurality of anodes 145, typically two anodes 145a and 145b, in order to minimize the virtual cathode effect in the direction of the brain. In certain electrode configurations, cathode 146 comprises a plurality of cathodes in order to minimize the “virtual anode effect,” which is analogous to the virtual cathode effect.

As appropriate, techniques described herein are practiced in conjunction with methods and apparatus described in U.S. patent application Ser. No. 10/205,474 to Gross et al., filed Jul. 24, 2002, entitled, “Electrode assembly for nerve control,” which published as US Patent Application Publication 2003/0050677, is assigned to the assignee of the present patent application, and is incorporated herein by reference. Alternatively or additionally, techniques described herein are practiced in conjunction with methods and apparatus described in U.S. patent application Ser. No. 10/205,475 to Gross et al., filed Jul. 24, 2002, entitled, “Selective nerve fiber stimulation for treating heart conditions,” which published as US Patent Application Publication 2003/0045909, is assigned to the assignee of the present patent application, and is incorporated herein by reference. Further alternatively or additionally, techniques described herein are practiced in conjunction with methods and apparatus described in U.S. Provisional Patent Application 60/383,157 to Ayal et al., filed May 23, 2002, entitled, “Inverse recruitment for autonomic nerve systems,” which is assigned to the assignee of the present patent application and is incorporated herein by reference.

FIG. 12C is a simplified perspective illustration of electrode device 140 (FIG. 12A), in accordance with an embodiment of the present invention. When applied to vagus nerve 136, electrode device 140 typically encompasses the nerve. As described, control unit 120 typically drives electrode device 140 to (i) apply signals to vagus nerve 136 in order to induce the propagation of efferent action potentials towards heart 30, and (ii) suppress artificially-induced afferent action potentials towards brain 134. The electrodes typically comprise ring electrodes adapted to apply a generally uniform current around the circumference of the nerve, as best shown in FIG. 12C.

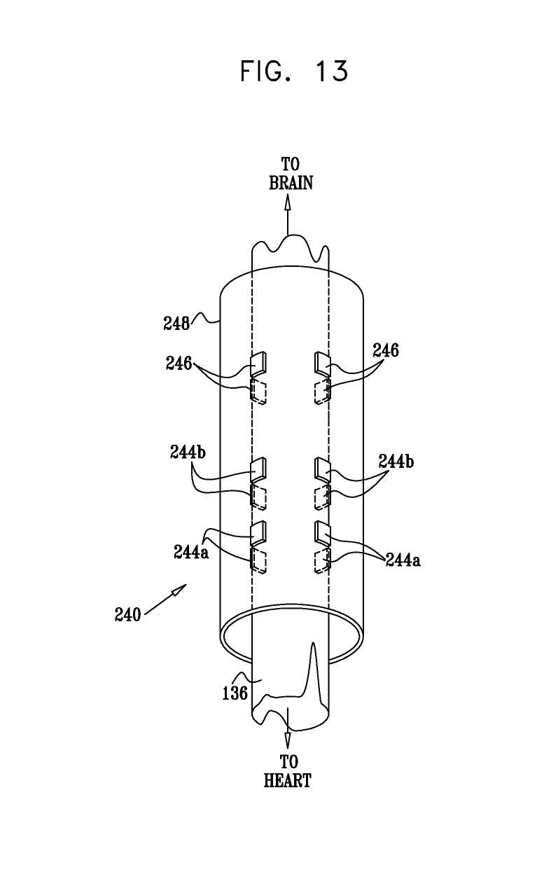

FIG. 13 is a simplified perspective illustration of a multipolar point electrode device 240 applied to vagus nerve 136, in accordance with an embodiment of the present invention. In this embodiment, anodes 244a and 244b and a cathode 246 typically comprise point electrodes (typically 2 to 100), fixed inside an insulating cuff 248 and arranged around vagus nerve 136 so as to selectively stimulate nerve fibers according to their positions inside the nerve. In this case, techniques described in the above-cited articles by Grill et al., Goodall et al., and/or Veraart et al. are typically used. The point electrodes typically have a surface area between about 0.01 mm² and 1 mm². In some applications, the point electrodes are in contact with vagus nerve 136, as shown, while in other applications the point electrodes are recessed in cuff 248, so as not to come in direct contact with vagus nerve 136, similar to the recessed ring electrode arrangement described above with reference to FIG. 12A. For some applications, one or more of the electrodes, such as cathode 246 or anode 244a, comprise a ring electrode, as described with reference to FIG. 12C, such that electrode device 240 comprises both ring electrode(s) and point electrodes (configuration not shown). Additionally, electrode device 240 optionally comprises an afferent anode set (positioned like anodes 145a and 145b in FIG. 12A), the anodes of which comprise point electrodes and/or ring electrodes.

Alternatively, ordinary, non-cuff electrodes are used, such as when the electrodes are placed on the epicardial fat pads instead of on the vagus nerve.

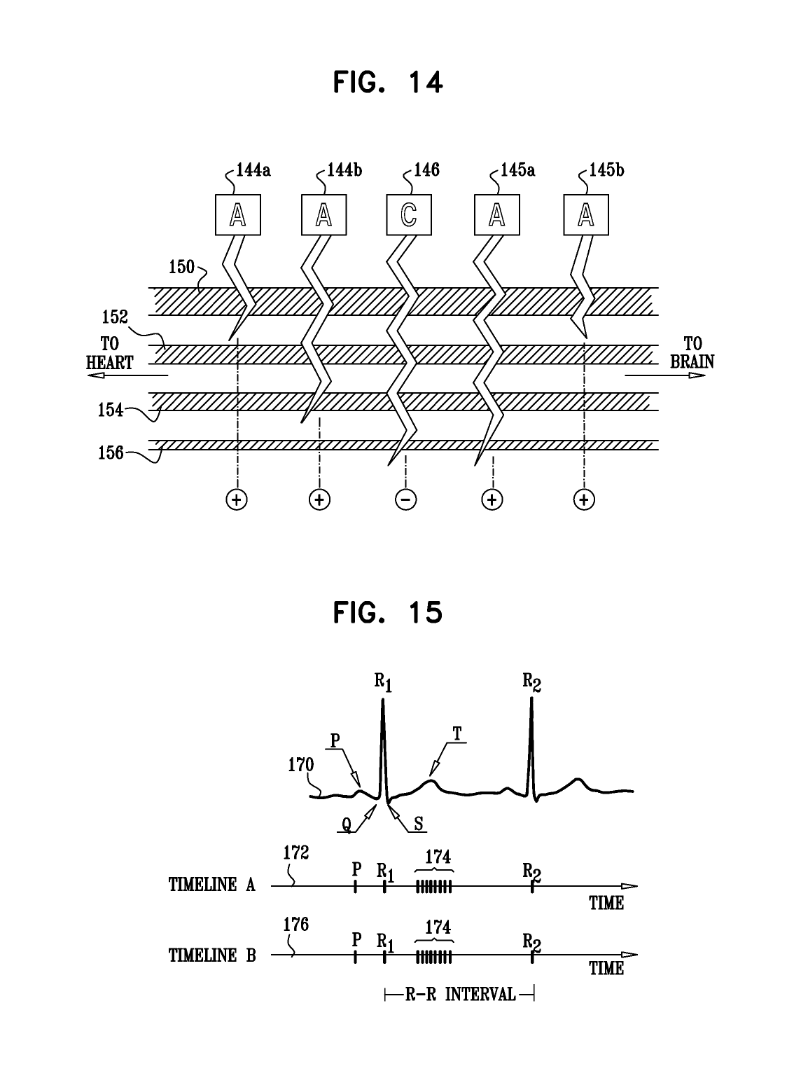

FIG. 14 is a conceptual illustration of the application of current to vagus nerve 136 in order to achieve smaller-to-larger diameter fiber recruitment, in accordance with an embodiment of the present invention. When inducing efferent action potentials towards heart 30, control unit 120 drives electrode device 140 to selectively recruit nerve fibers beginning with smaller-diameter fibers and to progressively recruit larger-diameter fibers as the desired stimulation level increases. This smaller-to-larger diameter recruitment order mimics the body's natural order of recruitment.

Typically, in order to achieve this recruitment order, the control unit stimulates myelinated fibers essentially of all diameters using cathodic current from cathode 46, while simultaneously inhibiting fibers in a larger-to-smaller diameter order using efferent anodal current from efferent anode set 144. For example, FIG. 14 illustrates the recruitment of a single, smallest nerve fiber 156, without the recruitment of any larger fibers 150, 152 and 154. The depolarizations generated by cathode 146 stimulate all of the nerve fibers shown, producing action potentials in both directions along all the nerve fibers. Efferent anode set 144 generates a hyperpolarization effect sufficiently strong to block only the three largest nerve fibers 150, 152 and 154, but not fiber 156. This blocking order of larger-to-smaller diameter fibers is achieved because larger nerve fibers are inhibited by weaker anodal currents than are smaller nerve fibers. Stronger anodal currents inhibit progressively smaller nerve fibers. When the action potentials induced by cathode 146 in larger fibers 150, 152 and 154 reach the hyperpolarized region in the larger fibers adjacent to efferent anode set 144, these action potentials are blocked. On the other hand, the action potentials induced by cathode 146 in smallest fiber 156 are not blocked, and continue traveling unimpeded toward heart 30. Anode pole 144a is shown generating less current than anode pole 144b in order to minimize the virtual cathode effect in the direction of the heart, as described above.

When desired, in order to increase the parasympathetic stimulation delivered to the heart, the number of fibers not blocked is progressively increased by decreasing the amplitude of the current applied by efferent anode set 144. The action potentials induced by cathode 146 in the fibers now not blocked travel unimpeded towards the heart. As a result, the parasympathetic stimulation delivered to the heart is progressively increased in a smaller-to-larger diameter fiber order, mimicking the body's natural method of increasing stimulation. Alternatively or additionally, in order to increase the number of fibers stimulated, while simultaneously decreasing the average diameter of fibers stimulated, the amplitudes of the currents applied by cathode 146 and efferent anode set 144 are both increased (thereby increasing both the number of fibers stimulated and blocked). In addition, for any given number of fibers stimulated (and not blocked), the amount of stimulation delivered to the heart can be increased by increasing the PPT, frequency, and/or pulse width of the current applied to vagus nerve 136.

In order to suppress artificially-induced afferent action potentials from traveling towards the brain in response to the cathodic stimulation, control unit 120 typically drives electrode device 140 to inhibit fibers 150, 152, 154 and 156 using afferent anodal current from afferent anode set 145. When the afferent-directed action potentials induced by cathode 146 in all of the fibers reach the hyperpolarized region in all of the fibers adjacent to afferent anode set 145, the action potentials are blocked. Blocking these afferent action potentials generally minimizes any unintended side effects, such as undesired or counterproductive feedback to the brain, that might be caused by these action potentials. Anode 145b is shown generating less current than anode 145a in order to minimize the virtual cathode effect in the direction of the brain, as described above.

In an embodiment of the present invention, the amplitude of the cathodic current applied in the vicinity of the vagus nerve is between about 2 milliamps and about 10 milliamps. Such a current is typically used in embodiments that employ techniques for achieving generally uniform stimulation of the vagus nerve, i.e., stimulation in which the stimulation applied to fibers on or near the surface of the vagus nerve is generally no more than about 400% greater than stimulation applied to fibers situated more deeply in the nerve. This corresponds to stimulation in which the value of the activation function at fibers on or near the surface of the vagus nerve is generally no more than about four times greater than the value of the activation function at fibers situated more deeply in the nerve. For example, as described hereinabove with reference to FIG. 12A, the electrodes may be recessed so as not to come in direct contact with vagus nerve 136, in order to achieve generally uniform values of the activation function. Typically, but not necessarily, embodiments using approximately 5 mA of cathodic current have the various electrodes disposed approximately 0.5 to 2.5 mm from the axis of the vagus nerve. Alternatively, larger cathodic currents (e.g., 10-30 mA) are used in combination with electrode distances from the axis of the vagus nerve of greater than 2.5 mm (e.g., 2.5-4.0 mm), so as to achieve an even greater level of uniformity of stimulation of fibers in the vagus nerve.

In an embodiment of the present invention, the cathodic current is applied by cathode 146 with an amplitude sufficient to induce action potentials in large- and medium-diameter fibers 150, 152, and 154 (e.g., A- and B-fibers), but insufficient to induce action potentials in small-diameter fibers 156 (e.g., C-fibers). Simultaneously, an anodal current is applied by anode 144b in order to inhibit action potentials induced by the cathodic current in the large-diameter fibers (e.g., A-fibers). This combination of cathodic and anodal current generally results in the stimulation of medium-diameter fibers (e.g., B-fibers) only. At the same time, a portion of the afferent action potentials induced by the cathodic current are blocked by anode 145a, as described above. Alternatively, the afferent anodal current is configured to not fully block afferent action potentials, or is simply not applied. In these cases, artificial afferent action potentials are nevertheless generally not generated in C-fibers, because the applied cathodic current is not strong enough to generate action potentials in these fibers.

These techniques for efferent stimulation of only B-fibers are typically used in combination with techniques described hereinabove for achieving generally uniform stimulation of the vagus nerve. Such generally uniform stimulation enables the use of a cathodic current sufficiently weak to avoid stimulation of C-fibers near the surface of the nerve, while still sufficiently strong to stimulate B-fibers, including B-fibers situated more deeply in the nerve, i.e., near the center of the nerve. For some applications, when employing such techniques for achieving generally uniform stimulation of the vagus nerve, the amplitude of the cathodic current applied by cathode 146 may be between about 3 and about 10 milliamps, and the amplitude of the anodal current applied by anode 144b may be between about 1 and about 7 milliamps. (Current applied at a different site and/or a different time is used to achieve a net current injection of zero.)

In an embodiment of the present invention, stimulation of the vagus nerve is applied responsive to one or more sensed parameters. Control unit 120 is typically configured to commence or halt stimulation, or to vary the amount and/or timing of stimulation in order to achieve a desired target heart rate, typically based on configuration values and on parameters including one or more of the following:
Heart rate—the control unit can be configured to drive electrode device 140 to stimulate the vagus nerve only when the heart rate exceeds a certain value.
ECG readings—the control unit can be configured to drive electrode device 140 to stimulate the vagus nerve based on certain ECG readings, such as readings indicative of designated forms of arrhythmia. Additionally, ECG readings are typically used for achieving a desire heart rate, as described below with reference to FIG. 15.
Blood pressure—the control unit can be configured to regulate the current applied by electrode device 140 to the vagus nerve when blood pressure exceeds a certain threshold or falls below a certain threshold.
Indicators of decreased cardiac contractility—these indicators include left ventricular pressure (LVP). When LVP and/or d(LVP)/dt exceeds a certain threshold or falls below a certain threshold, control unit 120 can drive electrode device 140 to regulate the current applied by electrode device 140 to the vagus nerve.
Motion of the patient—the control unit can be configured to interpret motion of the patient as an indicator of increased exertion by the patient, and appropriately reduce parasympathetic stimulation of the heart in order to allow the heart to naturally increase its rate.
Heart rate variability—the control unit can be configured to drive electrode device 140 to stimulate the vagus nerve based on heart rate variability, which is typically calculated based on certain ECG readings.
Norepinephrine concentration—the control unit can be configured to drive electrode device 140 to stimulate the vagus nerve based on norepinephrine concentration.
Cardiac output—the control unit can be configured to drive electrode device 140 to stimulate the vagus nerve based on cardiac output, which is typically determined using impedance cardiography.
Baroreflex sensitivity—the control unit can be configured to drive electrode device 140 to stimulate the vagus nerve based on baroreflex sensitivity.

The parameters and behaviors included in this list are for illustrative purposes only, and other possible parameters and/or behaviors will readily present themselves to those skilled in the art, having read the disclosure of the present patent application.

In an embodiment of the present invention, control unit 120 is configured to drive electrode device 140 to stimulate the vagus nerve so as to reduce the heart rate of the subject towards a target heart rate. The target heart rate is typically (a) programmable or configurable, (b) determined responsive to one or more sensed physiological values, such as those described hereinabove (e.g., motion, blood pressure, etc.), and/or (c) determined responsive to a time of day or circadian cycle of the subject. Parameters of stimulation are varied in real time in order to vary the heart-rate-lowering effects of the stimulation. For example, such parameters may include the amplitude of the applied current. Alternatively or additionally, in an embodiment of the present invention, the stimulation is applied in a series of pulses that are synchronized or are not synchronized with the cardiac cycle of the subject, such as described hereinbelow with reference to FIG. 15. Parameters of such pulse series typically include, but are not limited to:
Timing of the stimulation within the cardiac cycle. Delivery of the series of pulses typically begins after a fixed or variable delay following an ECG feature, such as each R- or P-wave. For some applications, the delay is between about 20 ms and about 300 ms from the R-wave, or between about 100 and about 500 ms from the P-wave.
Pulse duration (width). Longer pulse durations typically result in a greater heart-rate-lowering effect. For some applications, the pulse duration is between about 0.2 and about 4 ms.
Pulse repetition interval. Maintaining a pulse repetition interval (the time from the initiation of a pulse to the initiation of the following pulse) greater than about 3 ms generally results in maximal stimulation effectiveness for multiple pulses within a burst.
Pulses per trigger (PPT). A greater PPT (the number of pulses in each series of pulses after a trigger such as an R-wave) typically results in a greater heart-rate-lowering effect. For some applications, PPT is between about 0 and about 8.
Amplitude. A greater amplitude of the signal applied typically results in a greater heart-rate-lowering effect. The amplitude is typically less than about 10 milliamps, e.g., between about 2 and about 10 milliamps. For some applications, the amplitude is between about 2 and about 6 milliamps.
Duty cycle. Application of stimulation every heartbeat typically results in a greater heart-rate-lowering effect. For less heart rate reduction, stimulation is applied only once every several heartbeats.
Choice of vagus nerve. Stimulation of the right vagus nerve typically results in greater heart rate reduction than stimulation of the left vagus nerve.
“On”/“off” ratio and timing. For some applications, the device operates intermittently, alternating between “on” and “off” states, the length of each state typically between 0 and about 300 seconds (with a 0-length “off” state equivalent to always “on”). Greater heart rate reduction is typically achieved if the device is “on” a greater portion of the time.

For some applications, values of the “on”/“off” parameter are determined in real time, responsive to one or more inputs, such as sensed physiological values. Such inputs typically include motion or activity of the subject (e.g., detected using an accelerometer), the average heart rate of the subject when the device is in “off” mode, and/or the time of day. For example, the device may operate in continuous “on” mode when the subject is exercising and therefore has a high heart rate, and the device may alternate between “on” and “off” when the subject is at rest. As a result, the heart-rate-lowering effect is concentrated during periods of high heart rate, and the nerve is allowed to rest when the heart rate is generally naturally lower.

For some applications, heart rate regulation is achieved by setting two or more parameters in combination. For example, if it is desired to apply 5.2 pulses of stimulation, the control unit may apply 5 pulses of 1 ms duration each, followed by a single pulse of 0.2 ms duration. For other applications, the control unit switches between two values of PPT, so that the desired PPT is achieved by averaging the applied PPTs. For example, a sequence of PPTs may be 5, 5, 5, 5, 6, 5, 5, 5, 5, 6, . . . , in order to achieve an effective PPT of 5.2.

In an embodiment of the present invention, control unit 120 uses a slow-reacting heart rate regulation algorithm to modify heart-rate-controlling parameters of the stimulation, i.e., the algorithm varies stimulation parameters slowly in reaction to changes in heart rate. For example, in response to a sudden increase in heart rate, e.g., an increase from a target heart rate of 60 beats per minute (BPM) to 100 BPM over a period of only a few seconds, the algorithm slowly increases the stimulation level over a period of minutes. If the heart rate naturally returns to the target rate over this period, the stimulation levels generally do not change substantially before returning to baseline levels.

For example, the heart of a subject is regulated while the subject is inactive, such as while sitting. When the subject suddenly increases his activity level, such as by standing up or climbing stairs, the subject's heart rate increases suddenly. In response, the control unit adjusts the stimulation parameters slowly to reduce the subject's heart rate. Such a gradual modification of stimulation parameters allows the subject to engage in relatively stressful activities for a short period of time before his heart rate is substantially regulated, generally resulting in an improved quality of life.

In an embodiment of the present invention, control unit 120 is adapted to detect bradycardia (i.e., that an average detected R-R interval exceeds a preset bradycardia limit), and to terminate heart rate regulation substantially immediately upon such detection, such as by ceasing vagal stimulation. Alternatively or additionally, the control unit uses an algorithm that reacts quickly to regulate heart rate when the heart rate crosses limits that are predefined (e.g., a low limit of 40 beats per minute (BPM) and a high limit of 140 BPM), or determined in real time, such as responsive to sensed physiological values.

In an embodiment of the present invention, control unit 120 is configured to operate intermittently. Typically, upon each resumption of operation, control unit 120 sets the stimulation parameters to those in effect immediately prior to the most recent cessation of operation. For some applications, such parameters applied upon resumption of operation are maintained without adjustment for a certain number of heartbeats (e.g., between about one and about ten), in order to allow the heart rate to stabilize after resumption of operation.

For some applications, control unit 120 is configured to operate intermittently with gradual changes in stimulation. For example, the control unit may operate according to the following “on”/“off” pattern: (a) “off” mode for 30 minutes, (b) a two-minute “on” period characterized by a gradual increase in stimulation so as to achieve a target heart rate, (c) a six-minute “on” period of feedback-controlled stimulation to maintain the target heart rate, and (d) a two-minute “on” period characterized by a gradual decrease in stimulation to return the heart rate to baseline. The control unit then repeats the cycle, beginning with another 30-minute “off” period.

In an embodiment of the present invention, control unit 120 is configured to operate in an adaptive intermittent mode. The control unit sets the target heart rate for the “on” period equal to a fixed or configurable fraction of the average heart rate during the previous “off” period, typically bounded by a preset minimum. For example, assume that for a certain subject the average heart rates during sleep and during exercise are 70 and 150 BPM, respectively. Further assume that the target heart rate for the “on” period is set at 70% of the average heart rate during the previous “off” period, with a minimum of 60 BPM. During sleep, stimulation is applied so as to produce a heart rate of MAX(60 BPM, 70% of 70 BPM)=60 BPM, and is thus applied with parameters similar to those that would be used in the simple intermittent mode described hereinabove. Correspondingly, during exercise, stimulation is applied so as to produce a heart rate of MAX(60 BPM, 70% of 150 BPM)=105 BPM.

In an embodiment of the present invention, a heart rate regulation algorithm used by control unit 120 has as an input a time derivative of the sensed heart rate. The algorithm typically directs the control unit to respond slowly to increases in heart rate and quickly to decreases in heart rate.

In an embodiment of the present invention, the heart rate regulation algorithm utilizes sensed physiological parameters for feedback. For some applications, the feedback is updated periodically by inputting the current heart rate. For some applications, such updating occurs at equally-spaced intervals. Alternatively, the feedback is updated by inputting the current heart rate upon each detection of a feature of the ECG, such as an R-wave. In order to convert non-fixed R-R intervals into a form similar to canonical fixed intervals, the algorithm adds the square of each R-R interval, thus taking into account the non-uniformity of the update interval, e.g., in order to properly analyze feedback stability using standard tools and methods developed for canonical feedback.

In an embodiment of the present invention, control unit 120 implements a detection blanking period, during which the control unit does not detect heart beats. In some instances, such non-detection may reduce false detections of heart beats. One or both of the following techniques are typically implemented:
Absolute blanking. An expected maximal heart rate is used to determine a minimum interval between expected heart beats. During this interval, the control unit does not detect heart beats, thereby generally reducing false detections. For example, the expected maximal heart rate may be 200 BPM, resulting in a minimal detection interval of 300 milliseconds. After detection of a beat, the control unit disregards any signals indicative of a beat during the next 300 milliseconds.
Stimulation blanking. During application of a stimulation burst, and for an interval thereafter, the control unit does not detect heart beats, thereby generally reducing false detections of stimulation artifacts as beats. For example, assume stimulation is applied with the following parameters: a PPT of 5 pulses, a pulse width of 1 ms, and a pulse repetition interval of 5 ms. The control unit disregards any signals indicative of a beat during the entire 25 ms duration of the burst and for an additional interval thereafter, e.g., 50 ms, resulting in a total blanking period of 75 ms beginning with the start of the burst.

In an embodiment of the present invention, the heart rate regulation algorithm is implemented using only integer arithmetic. For example, division is implemented as integer division by a power of two, and multiplication is always of two 8-bit numbers. For some applications, time is measured in units of 1/128 of a second.

In an embodiment of the present invention, control unit 120 implements an integral feedback controller, which can most generally be described by:

K=K_{I}*∫edt

in which K represents the strength of the feedback, K_{I} is a coefficient, and ∫e dt represents the cumulative error. It is to be understood that such an integral feedback controller can be implemented in hardware, or in software running in control unit 120.

In an embodiment of such an integral controller, heart rate is typically expressed as an R-R interval (the inverse of heart rate). Parameters of the integral controller typically include TargetRR (the target R-R interval) and TimeCoeff (which determines the overall feedback reaction time).

Typically, following the detection of each R-wave, the previous R-R interval is calculated and assigned to a variable (LastRR). e (i.e., the difference between the target R-R interval and the last measured R-R interval) is then calculated as:

e=TargetRR−LastRR

e is typically limited by control unit 120 to a certain range, such as between −0.25 and +0.25 seconds, by reducing values outside the range to the endpoint values of the range. Similarly, LastRR is typically limited, such as to 255/128 seconds. The error is then calculated by multiplying LastRR by e:

Error=e*LastRR

A cumulative error (representing the integral in the above generalized equation) is then calculated by dividing the error by TimeCoeff and adding the result to the cumulative error, as follows:

Integral=Integral+Error/2^{TimeCoeff}

The integral is limited to positive values less than, e.g., 36,863. The number of pulses applied in the next series of pulses (pulses per trigger, or PPT) is equal to the integral/4096.

The following table illustrates example calculations using a heart rate regulation algorithm that implements an integral controller, in accordance with an embodiment of the present invention. In this example, the parameter TargetRR (the target heart rate) is set to 1 second (128/128 seconds), and the parameter TimeCoeff is set to 0. The initial value of Integral is 0. As can be seen in the table, the number of pulses per trigger (PPT) increases from 0 during the first heart beat, to 2 during the fourth heart beat of the example.

| | Heart Beat Number | | | |
|---|---|---|---|---|
| | 1 | 2 | 3 | 4 |
| Heart rate (BPM) | 100 | 98 | 96 | 102 |
| R-R interval (ms) | 600 | 610 | 620 | 590 |
| R-R (1/128 sec) | 76 | 78 | 79 | 75 |
| e (1/128 sec) | 52 | 50 | 49 | 53 |
| Limited e | 32 | 32 | 32 | 32 |
| Error | 2432 | 2496 | 2528 | 2400 |
| Integral | 2432 | 4928 | 7456 | 9856 |
| PPT | 0 | 1 | 1 | 2 |

In an embodiment of the present invention, the heart rate regulation algorithm corrects for missed heart beats (either of physiological origin or because of a failure to detect a beat). Typically, to perform this correction, any R-R interval which is about twice as long as the immediately preceding R-R interval is interpreted as two R-R intervals, each having a length equal to half the measured interval. For example, the R-R interval sequence (measured in seconds) 1, 1, 1, 2.2 is interpreted by the algorithm as the sequence 1, 1, 1, 1.1, 1.1. Alternatively or additionally, the algorithm corrects for premature beats, typically by adjusting the timing of beats that do not occur approximately halfway between the preceding and following beats. For example, the R-R interval sequence (measured in seconds) 1, 1, 0.5, 1.5 is interpreted as 1, 1, 1, 1, using the assumption that the third beat was premature.

In an embodiment of the present invention, control unit 120 is configured to operate in one of the following modes:
vagal stimulation is not applied when the heart rate of the subject is lower than the low end of the normal range of a heart rate of the subject and/or of a typical human subject;
vagal stimulation is not applied when the heart rate of the subject is lower than a threshold value equal to the current low end of the range of the heart rate of the subject, i.e., the threshold value is variable over time as the low end generally decreases as a result of chronic vagal stimulation treatment;
vagal stimulation is applied only when the heart rate of the subject is within the normal of range of a heart rate of the subject and/or of a typical human subjects;
vagal stimulation is applied only when the heart rate of the subject is greater than a programmable threshold value, such as a rate higher than a normal rate of the subject and/or a normal rate of a typical human subject. This mode generally removes peaks in heart rate; or
vagal stimulation is applied using fixed programmable parameters, i.e., not in response to any feedback, target heart rate, or target heart rate range. These parameters may be externally updated from time to time, for example by a physician.

In an embodiment of the present invention, the amplitude of the applied stimulation current is calibrated by fixing a number of pulses in the series of pulses (per cardiac cycle), and then increasing the applied current until a desired predetermined heart rate reduction is achieved. Alternatively, the current is calibrated by fixing the number of pulses per series of pulses, and then increasing the current to achieve a substantial reduction in heart rate, e.g., 40%.

In embodiments of the present invention in which vagal stimulation system 118 comprises implanted device 125 for monitoring and correcting the heart rate, control unit 120 typically uses measured parameters received from device 125 as additional inputs for determining the level and/or type of stimulation to apply. Control unit 120 typically coordinates its behavior with the behavior of device 125. Control unit 120 and device 125 typically share sensors 126 in order to avoid redundancy in the combined system.

Optionally, vagal stimulation system 118 comprises a patient override, such as a switch that can be activated by the patient using an external magnet. The override typically can be used by the patient to activate vagal stimulation, for example in the event of arrhythmia apparently undetected by the system, or to deactivate vagal stimulation, for example in the event of apparently undetected physical exertion.

FIG. 15 is a simplified illustration of an ECG recording 170 and example timelines 172 and 176 showing the timing of the application of a burst of stimulation pulses 174, in accordance with an embodiment of the present invention. Stimulation is typically applied to vagus nerve 136 in a closed-loop system in order to achieve and maintain the desired target heart rate, determined as described above. Precise graded slowing of the heart beat is typically achieved by varying the number of nerve fibers stimulated, in a smaller-to-larger diameter order, and/or the intensity of vagus nerve stimulation, such as by changing the stimulation amplitude, pulse width, PPT, and/or delay. Stimulation with blocking, as described herein, is typically applied during each cardiac cycle in burst of pulses 174, typically containing between about 1 and about 20 pulses, each of about 1-3 milliseconds duration, over a period of about 1-200 milliseconds. Advantageously, such short pulse durations generally do not substantially block or interfere with the natural efferent or afferent action potentials traveling along the vagus nerve. Additionally, the number of pulses and/or their duration is sometimes varied in order to facilitate achievement of precise graded slowing of the heart beat.

In an embodiment of the present invention (e.g., when the heart rate regulation algorithm described hereinabove is not implemented), to apply the closed-loop system, the target heart rate is expressed as a ventricular R-R interval (shown as the interval between R₁ and R₂ in FIG. 15). The actual R-R interval is measured in real time and compared with the target R-R interval. The difference between the two intervals is defined as a control error. Control unit 120 calculates the change in stimulation necessary to move the actual R-R towards the target R-R, and drives electrode device 140 to apply the new calculated stimulation. Intermittently, e.g., every 1, 10, or 100 beats, measured R-R intervals or average R-R intervals are evaluated, and stimulation of the vagus nerve is modified accordingly.

In an embodiment, vagal stimulation system 118 is further configured to apply stimulation responsive to pre-set time parameters, such as intermittently, constantly, or based on the time of day.

Alternatively or additionally, one or more of the techniques of smaller-to-larger diameter fiber recruitment, selective fiber population stimulation and blocking, and varying the intensity of vagus nerve stimulation by changing the stimulation amplitude, pulse width, PPT, and/or delay, are applied in conjunction with methods and apparatus described in one or more of the patents, patent applications, articles and books cited herein.

In an embodiment of the present invention, control unit 120 is configured to stimulate vagus nerve 136 so as to suppress the adrenergic system, in order to treat a subject suffering from a heart condition. For example, such vagal stimulation may be applied for treating a subject suffering from heart failure. In heart failure, hyper-activation of the adrenergic system damages the heart. This damage causes further activation of the adrenergic system, resulting in a vicious cycle. High adrenergic tone is harmful because it often results in excessive release of angiotensin and epinephrine, which increase vascular resistance (blood pressure), reduce heart rest time (accelerated heart rate), and cause direct toxic damage to myocardial muscles through oxygen free radicals and DNA damage. Artificial stimulation of the vagus nerve causes a down regulation of the adrenergic system, with reduced release of catecholamines. The natural effects of vagal stimulation, applied using the techniques described herein, typically reduces the release of catecholamines in the heart, thereby lowering the adrenergic tone at its source.

In an embodiment of the present invention, control unit 120 is configured to stimulate vagus nerve 136 so as to modulate atrial and ventricular contractility, in order to treat a subject suffering from a heart condition. Vagal stimulation generally reduces both atrial and ventricular contractility (see, for example, the above-cited article by Levy M N et al., entitled “Parasympathetic Control of the Heart”). Vagal stimulation, using the techniques described herein, typically (a) reduces the contractility of the atria, thereby reducing the pressure in the venous system, and (b) reduces the ventricular contractile force of the atria, which may reduce oxygen consumption, such as in cases of ischemia. For some applications, vagal stimulation, as described herein, is applied in order to reduce the contractile force of the ventricles in cases of hypertrophic cardiopathy. The vagal stimulation is typically applied with a current of at least about 4 mA.

In an embodiment of the present invention, control unit 120 is configured to stimulate vagus nerve 136 so as to improve coronary blood flow, in order to treat a subject suffering from a heart condition. Improving coronary blood flow by administering acetylcholine is a well known technique. For example, during Percutaneous Transluminal Coronary Angioplasty (PTCA), when maximal coronary dilation is needed, direct infusion of acetylcholine is often used to dilate the coronary arteries (see, for example, the above-cited article by Feliciano L et al.). For some applications, the vagal stimulation techniques described herein are used to improve coronary blood flow in subjects suffering from myocardial ischemia, ischemic heart disease, heart failure, and/or variant angina (spastic coronary arteries). It is hypothesized that such vagal stimulation simulates the effect of acetylcholine administration.

In an embodiment of the present invention, control unit 120 is configured to drive electrode device 140 to stimulate vagus nerve 136 so as to modify heart rate variability of the subject. For some applications, control unit 120 is configured to apply the stimulation having a duty cycle, which typically produces heart rate variability at the corresponding frequency. For example, such duty cycles may be in the range of once per every several heartbeats. For other applications, control unit 120 is configured to apply generally continuous stimulation (e.g., in a manner that produces a prolonged reduced level of heart rate variability).

For some applications, control unit 120 synchronizes the stimulation with the cardiac cycle of the subject, while for other applications, the control unit does not synchronize the stimulation with the cardiac cycle. For example, the stimulation may be applied in a series of pulses that are not synchronized with the cardiac cycle of the subject. Alternatively, the stimulation may be applied in a series of pulses that are synchronized with the cardiac cycle of the subject, such as described hereinabove with reference to FIG. 15.

For some applications, control unit 120 is configured to apply stimulation with parameters selected to reduce heart rate variability, while for other applications parameters are selected that increase variability. For example, when the stimulation is applied as a series of pulses, values of parameters that reduce heart variability may include one or more of the following:
Timing of the stimulation within the cardiac cycle: a delay of between about 50 ms and about 150 ms from the R-wave, or between about 100 and about 500 ms from the P-wave.
Pulse duration (width) of between about 0.5 and about 1.5 ms.
Pulse repetition interval (the time from the initiation of a pulse to the initiation of the following pulse) of between about 2 and about 8 ms.
Pulses per trigger (PPT), e.g., pulses per cardiac cycle, of between about 0 and about 8.
Amplitude of between about 5 and about 10 milliamps.

For some applications, the parameters of the stimulation are selected to both reduce the heart rate of the subject and heart rate variability of the subject. For other applications, the parameters are selected to reduce heart rate variability while substantially not reducing the average heart rate of the subject. In this context, a non-substantial heart rate reduction may be less than about 10%. For some applications, to achieve such a reduction in variability without a reduction in average rate, stimulation is applied using the feedback techniques described hereinabove, with a target heart rate greater than the normal average heart rate of the subject. Such stimulation typically does not substantially change the average heart rate, yet reduces heart rate variability (however, the instantaneous (but not average) heart rate may sometimes be reduced).

For some applications, in order to additionally reduce the heart rate, stimulation is applied using a target heart rate lower than the normal average heart rate of the subject. The magnitude of the change in average heart rate as well as the percentage of time during which reduced heart rate variability occurs in these applications are controlled by varying the difference between the target heart rate and the normal average heart rate.

For some applications, control unit 120 is configured to apply stimulation only when the subject is awake. Reducing heart variability when the subject is awake offsets natural increases in heart rate variability during this phase of the circadian cycle. Alternatively or additionally, control unit 120 is configured to apply or apply greater stimulation at times of exertion by the subject, in order to offset the increase in heart rate variability typically caused by exertion. For example, control unit 120 may determine that the subject is experiencing exertion responsive to an increase in heart rate, or responsive to a signal generated by an accelerometer. Alternatively, the control unit uses other techniques known in the art for detecting exertion.

In an embodiment of the present invention, control unit 120 is configured to drive electrode device 140 to stimulate vagus nerve 136 so as to modify heart rate variability in order to treat a condition of the subject. For some applications, the control unit is configured to additionally modify heart rate to treat the condition, while for other applications, the control unit is configured to modify heart rate variability while substantially not modifying average heart rate.

Therapeutic effects of reduction in heart rate variability include, but are not limited to:
Narrowing of the heart rate range, thereby eliminating very slow heart rates and very fast heart rates, both of which are inefficient for a subject suffering from heart failure. For this therapeutic application, control unit 120 is typically configured to reduce low-frequency heart rate variability, and to adjust the level of stimulation applied based on the circadian and activity cycles of the subject.
Stabilizing the heart rate, thereby reducing the occurrence of arrhythmia. For this therapeutic application, control unit 120 is typically configured to reduce heart rate variability at all frequencies.
Maximizing the mechanical efficiency of the heart by maintaining relatively constant ventricular filling times and pressures. For example, this therapeutic effect may be beneficial for subjects suffering from atrial fibrillation, in which fluctuations in heart filling times and pressure reduce cardiac efficiency.
Eliminating the normal cardiac response to changes in the breathing cycle (i.e., respiratory sinus arrhythmia). Although generally beneficial in young and efficient hearts, respiratory sinus arrhythmia may be harmful to subjects suffering from heart failure, because respiratory sinus arrhythmia causes unwanted accelerations and decelerations in the heart rate. For this therapeutic application, control unit 120 is typically configured to reduce heart rate variability at high frequencies.

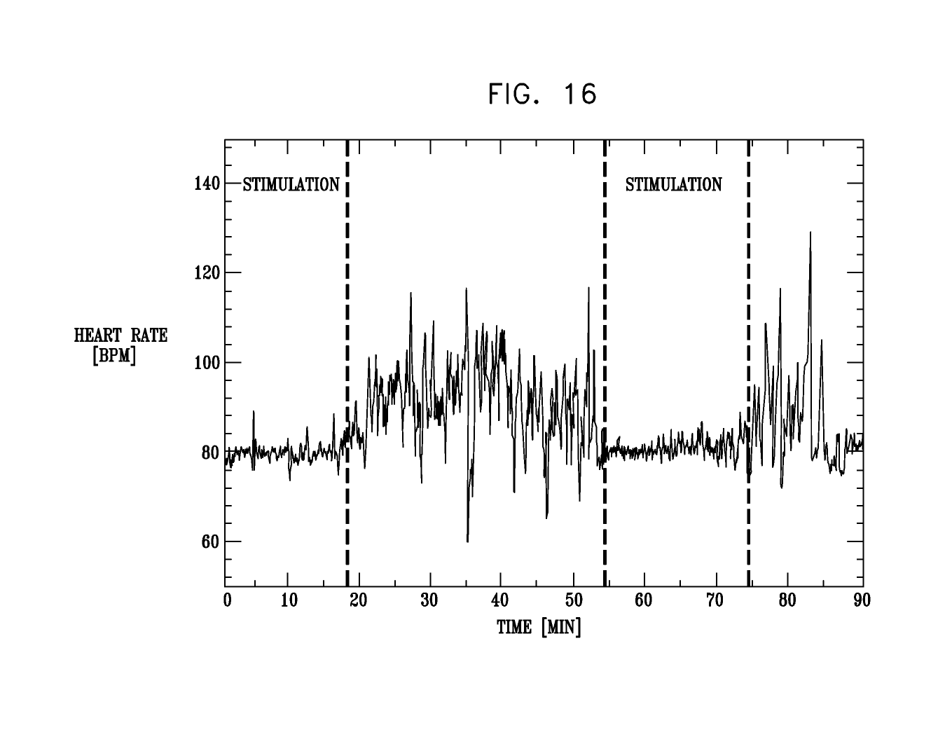

Reference is now made to FIG. 16, which is a graph showing in vivo experimental results, measured in accordance with an embodiment of the present invention. A dog was anesthetized, and cuff electrodes, similar to those described hereinabove with reference to FIG. 12B, were implanted in the right cervical vagus nerve. After a recovery period of two weeks, experimental vagal stimulation was applied to the dog while the dog was awake and allowed to move freely within its cage.

A control unit, similar to control unit 120, was programmed to apply vagal stimulation in a series of pulses, having the following parameters:
Stimulation synchronized with the intracardiac R-wave signal, with a delay from the R-wave of 60 ms;
Stimulation amplitude of 8 mA;
Stimulation pulse duration of 1 ms; and
Time between pulses within a burst of 5 ms.
   The control unit implemented an integral feedback controller, similar to the integral feedback controller described hereinabove, in order to vary the number of pulses within a burst. The integral feedback controller used a target heart rate of 80 beats per minute. After 2 minutes of stimulation, the number of pulses within each burst was typically between about 1 and about 8.

During a first period and a third period from 0 to 18 minutes and 54 to 74 minutes, respectively, the control unit applied stimulation to the vagus nerve. Heart rate variability was substantially reduced, while an average heart rate of 80 beats per minute was maintained. (Baseline heart rate, without stimulation, was approximately 95 beats per minute.) During a second period and a fourth period from 18 to 54 minutes and 74 to 90 minutes, respectively, stimulation was discontinued, and, as a result, heart rate variability increased substantially, returning to normal values. Average heart rate during these non-stimulation periods increased to approximately 95 beats per minute (approximately baseline value). Thus, these experimental results demonstrate that the application of vagal stimulation using some of the techniques described herein results in a substantial reduction in heart rate variability.

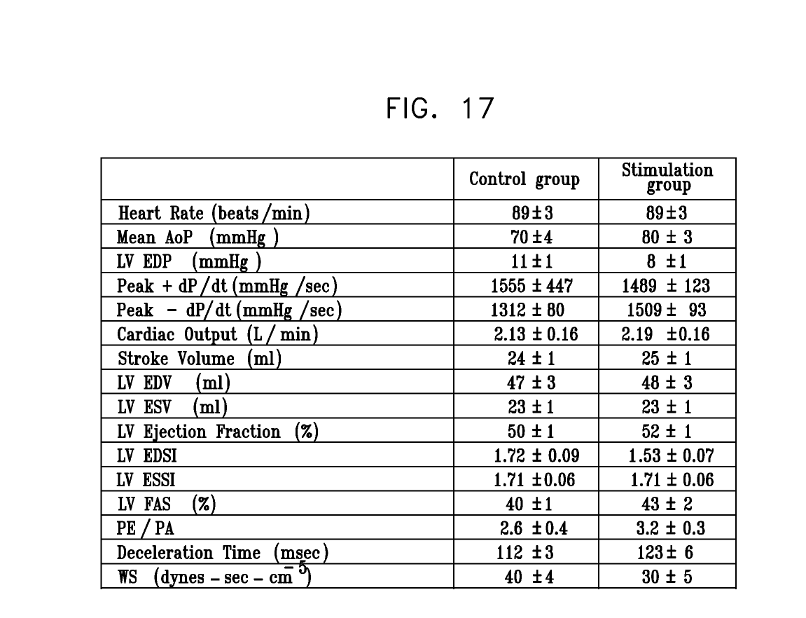

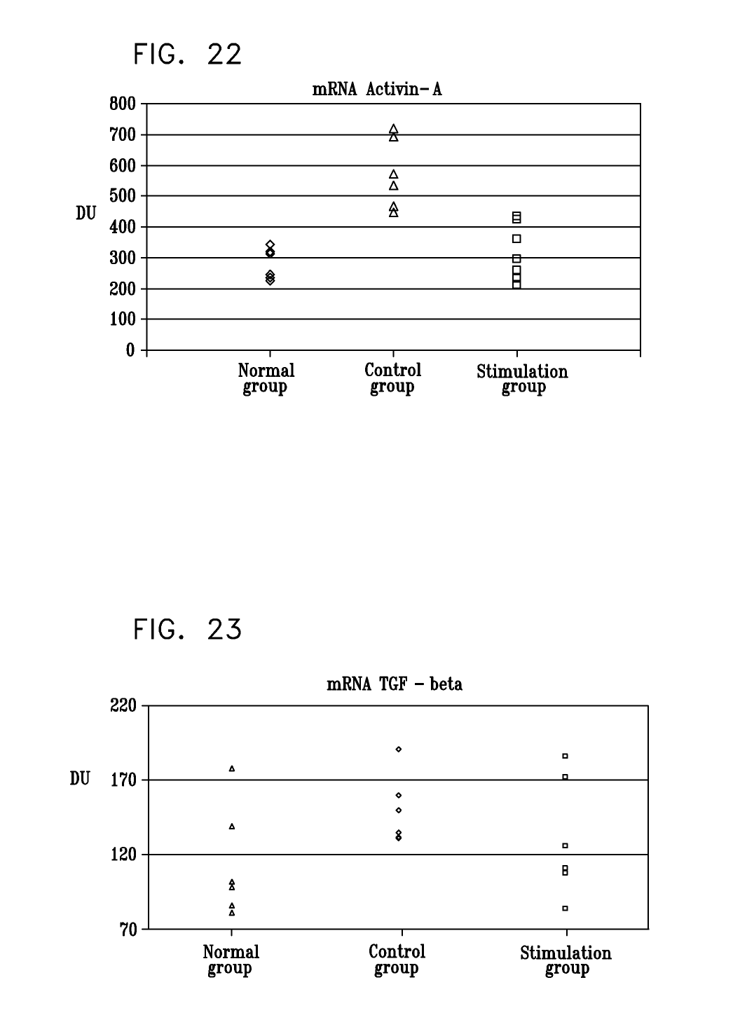

Reference is made to FIGS. 17-25, which are graphs showing in vivo experimental results, measured in accordance with respective embodiments of the present invention. The objective of the study was to assess the efficacy of chronic vagus nerve stimulation therapy, using techniques described herein, in dogs with advanced chronic heart failure. Chronic heart failure was produced by multiple sequential intracoronary microembolizations.

A total of 19 healthy, conditioned purpose-bred mongrel dogs were entered into the study. Six of the dogs served as a non-sham-operated “normal” control group. These dogs, which underwent neither surgical implantations nor induced heart failure, were used in several of the analyses described hereinbelow with reference to FIGS. 19-25. The remaining 13 dogs underwent multiple sequential intracoronary microembolizations in order to produce chronic compensated heart failure (see the above-referenced articles by Sabbah HN et al. (1991 and 1994)). Embolizations were performed during cardiac catheterizations and were discontinued when left ventricular (LV) ejection fraction, determined angiographically, was approximately 35%. Cardiac catheterizations were preformed under general anesthesia and in sterile conditions. Anesthesia was induced using a combination of intravenous injections of hydromorphone (0.22 mg/kg) and diazepam (0.2-0.6 mg/kg), and a plane of anesthesia was maintained throughout the procedure with 1% to 2% isoflurane. During cardiac catheterizations, dogs were intubated and ventilated with room air.

Following the third coronary embolization and before the target ejection was reached, the 13 dogs were implanted with a system similar to vagal stimulation system 118, described hereinabove with reference to FIG. 11. The system comprised a control unit, which was implanted in the neck; a tripolar cuff electrode, which was positioned around the mid-right cervical vagus nerve; and an intracardiac electrode, which was positioned in the right ventricle and used for ECG and heart rate monitoring. An anterior longitudinal cervical incision was made at the midclavicular line. The right carotid sheath and right vagus nerve were exposed. The stimulation electrode was then placed around the vagus nerve and secured by tightening pre-existing tightening strings. A bend and a loop were created to avoid tension on the electrode due to head and neck movements. At least two more sutures were used to secure the electrode to the adjacent fascia. An active fixation ventricular lead was introduced into the right jugular vein and placed in the right ventricle apex using fluoroscopy. A subcutaneous tunnel between the cervical operational wound and the left side of the neck was made with a tunneling tool. The electrode wires and lead were passed through the tunnel and connected to the implantable generator placed in a previously created pocket on the left side of the neck.

A standard pacemaker unipolar ventricular electrode was used for sensing an intracardiac electrocardiogram. A tripolar vagus nerve cuff electrode was used, similar to that described with reference to FIG. 12B. The stimulation lead was connected to the nerve stimulator via two IS-1-like connectors. Adjustments were made while a programming wand was placed over the implanted nerve stimulator.

Two weeks after the last embolization (i.e., after the target ejection fraction was achieved), the 13 dogs underwent a left and right heart catheterization to evaluate LV function. The electrostimulation system was activated in 7 dogs. In the remaining 6 dogs, the system was not activated, such that these dogs served as a concurrent sham-operated placebo control group. In the control group the generators were implanted, but not activated.

The electrostimulation system was configured to adjust the impulse rate and intensity to keep the heart rate within a desired range. The control unit was programmed to apply vagal stimulation in a series of pulses, controlling the heart rate with the feedback algorithm described hereinabove, using the following parameters:
stimulation synchronized with the intracardiac R-wave signal, with a delay from detection of the R-wave of 100 ms;
stimulation current in the range of 4 to 8 mA;
stimulation pulse width of 1 ms;
each stimulation burst included between 0 and 8 pulses;
time between pulses within a burst of 5 ms; and
target heart rate was between 0 and 30 beats per minute above average heart rate.

All dogs were followed for 3 months. Hemodynamic, angiographic, echocardiographic, and electrocardiographic studies were performed just prior to activation of the system, and were repeated at the end of the 3 months of follow-up. After completing the final cardiac catheterization, and while under general anesthesia, the chest and abdomen were opened and examined for evidence of pleural effusion, pericardial effusion, and ascites. The heart was removed and LV tissue was prepared for histological and biochemical examination. Tissue samples were also obtained from lung, kidney, skeletal muscle, major blood vessels, and liver, and stored at −70° C. for future evaluation. Blood samples were collected at all study time points, and plasma samples were stored in cryotubes at −20° C. for future evaluation.

The primary endpoints of the study were:
prevention or attenuation of progressive LV dysfunction assessed by angiographic LV ejection fraction; and
prevention or attenuation of progressive LV remodeling assessed by measurements of LV end diastolic volume, LV end systolic volume, and LV chamber shape (sphericity), also determined from LV angiographic silhouettes.

The secondary endpoints of the study were:
prevention or attenuation of progressive LV diastolic dysfunction assessed by measuring: (1) LV peak −dP/dt, (2) LV deceleration time, (3) mitral valve velocity PE/PA ratio, and (4) LV end-diastolic circumferential wall stress;
extent of attenuation of cardiomyocyte hypertrophy, volume fraction of replacement fibrosis, and volume fraction of interstitial fibrosis;
capillary density and oxygen diffusion distance;
LV end-diastolic pressure determined by catheterization; and
presence and severity of functional mitral regurgitation.

All hemodynamic measurements were performed during cardiac catheterizations in anesthetized dogs. Measurements were made: (a) at baseline, prior to any embolizations (referred to as “baseline” hereinbelow), (b) at two weeks after the last embolization and prior to activation of the stimulation system and initiation of follow-up (referred to as “pre-treatment” hereinbelow), and (c) at 3 months after the initiation of therapy (referred to as “post-treatment” hereinbelow). The following parameters were evaluated in all dogs at all three study time periods: (1) aortic and LV pressures using catheter tip micromanometers (Millar Instruments), (2) peak rate of change of LV pressure during isovolumic contraction (peak +dP/dt) and relaxation (peak −dP/dt), and (3) LV end-diastolic pressure.

Left ventriculograms were performed during cardiac catheterization after completion of the hemodynamic measurements. Ventriculograms were performed with the dog placed on its right side, and were recorded on 35 mm cine at 30 frames/sec during a power injection of 15-20 ml of contrast material (RENO M 60, Squibb Diagnostics). Correction for image magnification was made using a radiopaque grid placed at the level of the LV. LV end-systolic (ESV) and end-diastolic (EDV) volumes were calculated from angiographic silhouettes using the area-length method (see the above-mentioned article by Dodge HT et al.). Premature beats and postextrasystolic beats were excluded from the analysis. LV ejection fraction was calculated as 100*(EDV−ESV)/EDV. Stroke volume was calculated as the difference between LV EDV and ESV, and cardiac output was calculated as the product of stroke volume and heart rate.

Global LV shape, a measure of LV sphericity, was quantified from angiographic silhouettes based upon the ratio of the major to minor axis at end-systole and end-diastole (see the above-mentioned article by Sabbah HN et al. (1992)). The major axis was drawn from the apex of the LV to the midpoint of the plane of the aortic valve. The minor axis was drawn perpendicular to the major axis at its midpoint. As this ratio decreases (i.e., approaches unity), the shape of the LV chamber approaches that of a sphere.

Echocardiographic and Doppler studies were performed in all dogs at all specified study time points, using a 77030A ultrasound system (Hewlett Packard) with a 3.5 MHZ transducer. All echocardiographic measurements were made with the dog placed in the right lateral decubitus position and recorded on a Panasonic 6300 VHS recorder for subsequent off-line analysis. LV fractional area shortening (FAS), a measure of LV systolic function, was measured from the short axis view at the level of the papillary muscles. LV thickness of the posterior wall and interventricular septum were measured, summed and divided by 2 to arrive at average LV wall thickness (h) to be used for calculating wall stress. LV major and minor semiaxes were measured and used for calculation of LV end-diastolic circumferential wall stress. Wall stress was calculated as described in Grossman W., Cardiac Catheterization and Angiography, 3rd ed., Philadelphia, Pa.: Lea & Febiger (1986), which is incorporated herein by reference, on p. 293.

Mitral inflow velocity was measured by pulsed-wave Doppler echocardiography. The velocity waveforms were used to calculate: (1) peak mitral flow velocity in early diastole (PE), peak mitral inflow velocity during LA contraction (PA), (3) the ratio of PE to PA, and (4) a deceleration time (DT) of the early rapid mitral inflow velocity waveform, a measure of LV relaxation. The presence or absence of functional mitral regurgitation (MR) was determined with Doppler color flow mapping (Hewlett Packard model 77020A Ultrasound System) using both apical two chamber and apical four chamber views. When present, the severity of functional MR was quantified based on the ratio of the regurgitant jet area to the area of the left atrium times 100. The ratios calculated from both views were then averaged to obtain a single representative measure of the severity of functional MR.

At the end of the protocol, after completion of all hemodynamic and angiographic studies, the chest and abdomen of the dogs were opened and examined grossly, as described above. Once the gross examination was completed, the heart was rapidly removed and placed in ice cold Tris Buffer (pH 7.4). Three 2 mm thick transverse slices were obtained from the LV (one from the basal third, one from the middle third, and one from the apical third), and were placed in 10% formalin. Transmural blocks were also obtained and rapidly frozen in isopentane cooled to −160° C. by liquid nitrogen, and stored at −70° C. until needed.

Formalin-fixed LV tissue slices were cut into smaller blocks (approximately 6). Each block was labeled for anatomical site, and embedded in paraffin blocks. Five micron thick sections were prepared and stained with Masson trichrome for quantification of replacement fibrosis. The extent of replacement fibrosis was calculated as the percent total surface area occupied by fibrous tissue. This measurement was made for each LV slice. The percent replacement fibrosis for each LV section was calculated as the average of all three slices (basal, middle, and apical). To quantify interstitial fibrosis, sections were stained with lectin. The volume fraction of interstitial collagen in regions remote from any infarcts were quantified as the percent total area occupied by collagen. For this morphometric analysis, 10 microscopic fields were selected at random from noninfarcted regions of each of 6 blocks. The overall volume fraction of interstitial collagen was calculated as the average value of all LV regions combined. Cardiomyocyte cross-sectional area, a measure of cardiomyocyte hypertrophy, was assessed from sections stained with lectin to delineate the myocyte border. Ten radially-oriented, scar free, microscopic fields (X 40) were selected at random from each section and used to measure myocyte cross-sectional area by computer-assisted planimetry. Capillary density was measured also in sections stained with lectin-I. Capillary density was calculated as the number of capillaries per square millimeter and as the index capillary per fiber ratio (C/F). Oxygen diffusion distance was calculated as half the distance between two adjoining capillaries. For histological studies, LV tissue from the six dogs of the normal group was used.

Intragroup comparisons of hemodynamic, angiographic, echocardiographic, and Doppler variables within each of the two study groups were made between measurements obtained just before initiation of therapy and measurements made after completion of 3 months of therapy. For these comparisons, a Student's paired t-test was used, and a probability value <0.05 was considered significant. Study measurements were tested at baseline before any embolizations and at the time of assignment to study arms before initiation of therapy. Intergroup comparisons were made using a t-statistic for two means.

As can be seen in FIG. 17, there were no significant differences at baseline between the stimulation and control groups with respect to any of the hemodynamic, angiographic, echocardiographic and Doppler measurements. The p-values shown in this table are for the control group vs. the stimulation group. (The following abbreviations are used in the table: LV=left ventricular; AoP=aortic pressure; EDP=end-diastolic pressure; EDV=end-diastolic volume; ESV=end-systolic volume; EDSI=end-diastolic sphericity index; ESSI=end-systolic sphericity index; FAS=fractional area of shortening; and WS=wall stress.)

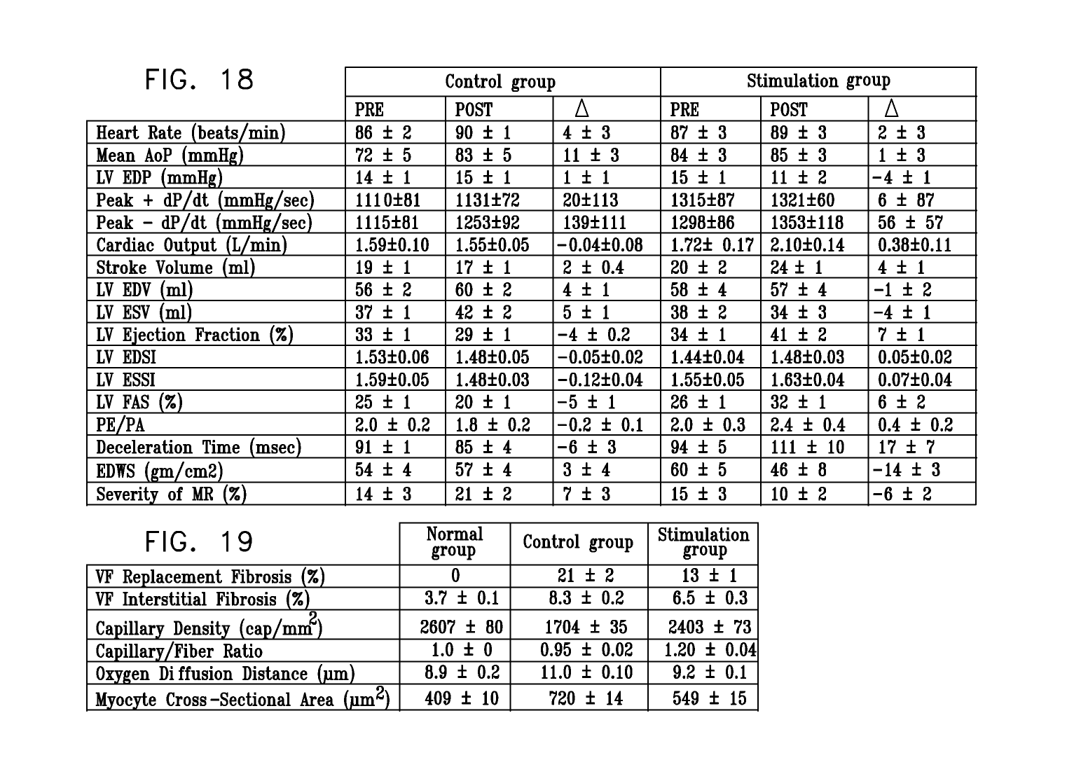

Similarly, as can be seen in FIG. 18, there were no significant differences between the two groups at pre-treatment except for mean aortic pressure, which was modestly but significantly lower in the control group than in the stimulation group. (The following abbreviations are used in the table: LV=left ventricular; AoP=aortic pressure; EDP=end-diastolic pressure; EDV=end-diastolic volume; ESV=end-systolic volume; EDSI=end-diastolic sphericity index; ESSI=end-systolic sphericity index; FAS=fractional area of shortening; WS=wall stress; and MR=functional mitral regurgitation.)

There were no differences between pre-treatment and post-treatment in the sham-operated control group with respect to heart rate, LV end-diastolic pressure, LV peak +dP/dt, LV peak −dP/dt, cardiac output, stroke volume, PE/PA ratio, DT, wall stress, or severity of functional MR. In the control group, however, there was a significant increase in mean aortic pressure, LV end-diastolic volume, and LV end-systolic volume. This was accompanied by a significant decline in LV ejection fraction and FAS. At the same time, ventricular sphericity increased, as evidenced by a significant reduction in LV end-systolic and end-diastolic major-to minor axis ratios, and by a significant increase in LV wall stress.

In the post-treatment analysis, comparisons were made between the sham-operated control group and the stimulation group, as shown in FIG. 18. Treatment with the stimulation system had no effect on heart rate or mean aortic pressure or on LV peak +dP/dt and peak −dP/dt. Treatment with the stimulation system did, however, significantly increase LV ejection fraction, LV FAS, cardiac output, stroke volume, sphericity indices, PE/PA ratio, and DT, while significantly decreasing LV end-diastolic pressure, EDV, ESV wall stress, and functional MR.

FIG. 19 shows the histomorphometric measurements from the six dogs of the normal group, the heart failure sham-operated dogs, and the heart failure dogs treated with the stimulation system. (In the table, VF=Volume Fraction.) Chronic stimulation using the stimulation system was associated with a significant reduction of volume fraction of replacement and interstitial fibrosis, a significant increase in capillary density, a significant decrease in myocyte cross-sectional area (a measure of myocyte hypertrophy), and a significant decrease in oxygen diffusion distance.

The results of this study indicate that chronic (3-month) therapy with the stimulation system in dogs with heart failure improves LV systolic and diastolic function. The improvement in systolic function is evidenced by increased LV ejection fraction, FAS, and stroke volume. The improvement in diastolic function is evidenced by reductions in LV preload, an increase in PE/PA ratio and DT, and a decrease in end-diastolic wall stress. At the global level, chronic therapy attenuated progressive LV remodeling, as evidenced by decreased LV chamber sphericity as well as LV size. At the cellular level, chronic therapy with the stimulation system attenuated remodeling, as evidenced by reduction of replacement and interstitial fibrosis, enhancing capillary density, shortening oxygen diffusion distance, and a decrease in myocyte hypertrophy.

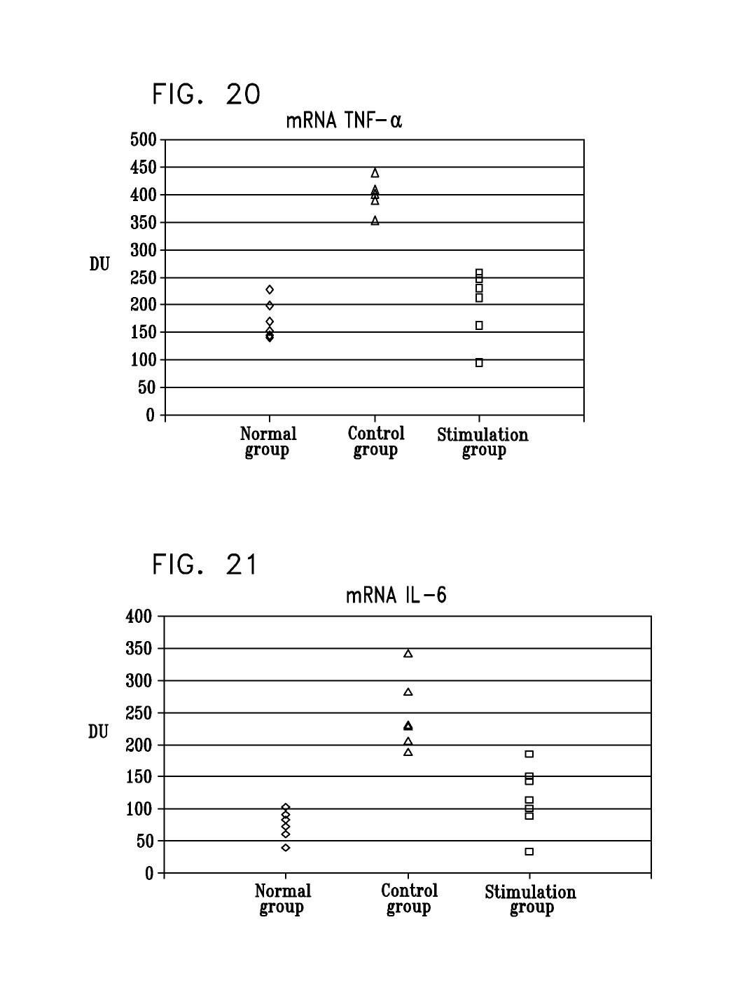

LV tissue from all 13 dogs of the sham-operated control group and stimulation group, and from the six dogs of the normal group, was used to extract RNA. mRNA expression for TNF-alpha, IL-6, Activin-A, and TGF-beta was measured using reverse transcriptase polymerase chain reaction (RT-PCR), and the bands obtained after gel electrophoresis were quantified in densitometric units (du). As can be seen in FIGS. 20-23, mRNA expression for all four cytokines was significantly higher in the sham-control group than in the normal group, and vagal stimulation therapy reduced mRNA expression of all four cytokines in the stimulation group compared to the sham-control group.

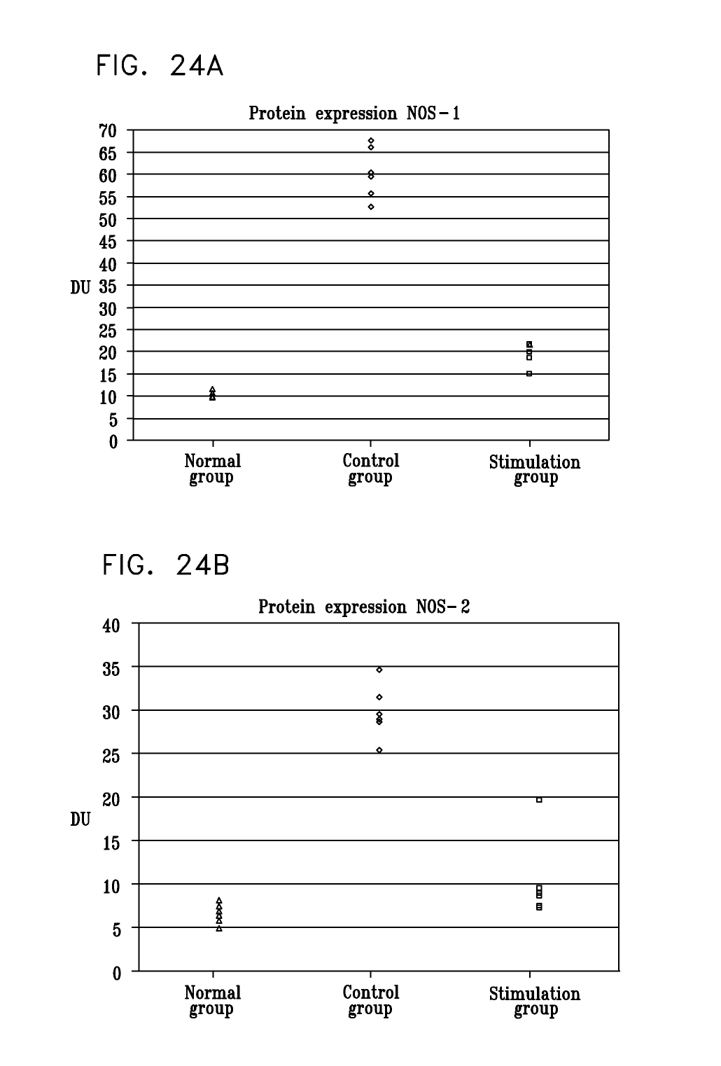

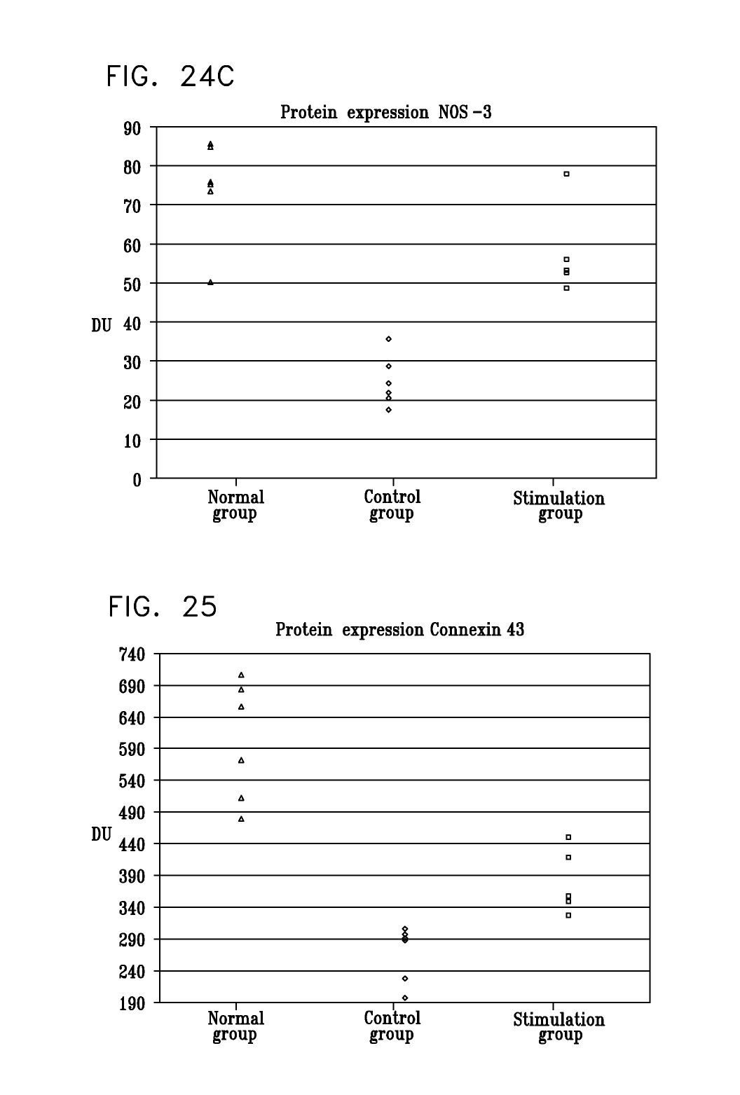

FIGS. 24A, 24B, and 24C are graphs showing the levels of protein expression of NOS-1, NOS-2, and NOS-3, respectively, in each dog of the normal group, the heart failure sham-operated group, and the stimulation group treated with the stimulation system. Protein expression was measured in tissue homogenate using Western blots, and the bands were quantified in densitometric units (du).

As can be seen in FIGS. 24A-C, protein expression of NOS-3 decreased, and NOS-1 and NOS-2 were significantly higher in the sham-operated control group than in the normal group. Three months' treatment with the stimulation system statistically significantly reduced mRNA and protein expression of NOS-1 and NOS-2, and statistically significantly increased mRNA and protein expression of NOS-3, thereby normalizing mRNA and protein expression of NOS-1, NOS-2, and NOS-3. The inventors believe that such normalization of mRNA and protein expression of NOS-1, NOS-2, and NOS-3 in LV myocardium explains, in part, the improvement in global LV function observed when dogs with heart failure received long-term treatment with the electrical stimulation therapy described herein. In an embodiment of the present invention, vagal stimulation applied using techniques described herein is configured to reduce expression of NOS-1 and/or NOS-2, and/or to increase expression of NOS-3.

FIG. 25 is a graph showing the densitometry levels of Connexin 43 in LV tissue of each dog of the normal group, the heart failure sham-operated group, and the stimulation group treated with the stimulation system. As can be seen in the graph, stimulation with the system caused a statistically significant increase in levels of Connexin 43 protein. The ventricular Connexin 43 protein level is substantially reduced in ischemia and heart failure. In a mouse model, reduced expression of Connexin 43 increases the incidence of ventricular tachyarrhythmias and causes a significant reduction in conduction velocity. These results suggest that reduction of Connexin 43 in ventricular tissue promotes conditions such as heart failure. In an embodiment of the present invention, vagal stimulation applied using techniques described herein is configured to increase Connexin 43 levels sufficiently to treat a cardiac condition of the subject, such as heart failure.

FIG. 26 is a graph showing N-terminal pro-brain natriuretic peptide (NT-pro-BNP) serum levels in two human subjects, measured in accordance with an embodiment of the present invention. Two human heart failure subjects (NYHA class III) were implanted, under general anesthesia, with a system similar to vagal stimulation system 18, described hereinabove with reference to FIG. 11. The system comprised a control unit, which was implanted in the subject's chest; a tripolar cuff electrode, which was positioned around the mid-right cervical vagus nerve; and an intracardiac electrode, which was positioned in the right ventricle and used for ECG and heart rate monitoring. The control unit was programmed to apply vagal stimulation in a series of pulses, having the following parameters:
stimulation synchronized with the intracardiac R-wave signal, with a delay from detection of the R-wave of 100 ms;
stimulation current in the range of 2 to 4 mA;
stimulation pulse width of 1 ms;
each stimulation burst included between 0 and 3 pulses; and
time between pulses within a burst of 5 ms.

The stimulation system was activated two weeks after implantation. Blood samples were taken before activation (baseline) and three months after activation. NT-pro-BNP serum levels, a standard diagnostic indicator of the severity of heart failure, were measured using a standard ELISA procedure. As can be seen in the graph, the NT-pro-BNP levels decreased in one subject from 705 to 275, and in a second subject from 1337 to 990. These results demonstrate that vagal stimulation using techniques described herein resulted in improved cardiac function in two human subjects.

In an embodiment of the present invention, vagal stimulation performed using the techniques described herein affects one or more of the following physiological parameters:

### Hemodynamic and Cardiac Geometry Parameters

mean aortic pressure (mmHg)
left ventricular end-diastolic pressure (mmHg)
peak +dP/dt (mmHg/sec)
peak −dP/dt (mmHg/sec)
cardiac output (L/min)
stroke volume (ml)
left ventricular end-diastolic volume (ml)
left ventricular end-systolic volume (ml)
left ventricular Ejection Fraction (%)
left ventricular end-diastolic sphericity index
left ventricular end-systolic sphericity index
left ventricular fractional area of shortening (%)
ratio of peak mitral flow velocity in early diastole (PE) to peak mitral inflow velocity during left atrial contraction (PA)
deceleration time of the early rapid mitral inflow velocity waveform (msec)
left ventricular end-diastolic circumferential wall stress (gm/cm2)
severity of mitral regurgitation (%)
systemic vascular resistance
pulmonary vascular resistance
coronary blood flow
vagal tone
heart rate variability
baroreceptor sensitivity
pulmonary residual volumes and pressures (which facilitate gas exchange and prevent pulmonary edema)
VO2 Max
intracardiac conduction
AV delay
atrial contractility (improvements of which cause less backflow into the lungs, less stress on the myocardium, smaller ventricular volumes and reduced volume overload on the LV)

### Myocardial Cellular Anatomy Parameters

volume fraction replacement fibrosis (%)
volume fraction interstitial fibrosis (%)
capillary density (cap/mm2)
capillary/fiber ratio
oxygen diffusion distance (μm)
myocyte cross-sectional area (μm2)
apoptosis
level of homogeneity of the myocardium
activation of alpha-adrenergic receptors.

### Inflammatory Markers

tumor necrosis factor alpha
interleukin 6
activin A
transforming growth factor
interferon
interleukin 1 beta
interleukin 18
interleukin 12
C-reactive protein

### Neurohormone Peptide

brain natriuretic peptide (BNP), e.g., N-terminal pro-BNP (NT-pro-BNP)
a catecholamine

### NO Synthases (NOSs)

neural NOS (nNOS, or NOS-1)
inducible NOS (iNOS, or NOS-2)
endothelial NOS (eNOS, or NOS-3)

### Gap Junction Proteins

Connexin, e.g., Connexin 43

In an embodiment of the present invention, vagal stimulation is performed using the techniques described herein to treat one or more of the following cardiac pathologies: heart failure, congestive heart failure, diastolic heart failure, atrial fibrillation, atherosclerosis, restenosis, myocarditis, cardiomyopathy, myocardial infarction, post-myocardial infarct remodeling, angina, hypertension, arrhythmia, endocarditis, arteritis, thrombophlebitis, pericarditis, myocardial ischemia, sick sinus syndrome, cardiogenic shock, and cardiac arrest.

In an embodiment of the present invention, vagal stimulation is performed using the techniques described herein to treat a “stimulation-treatable condition.” A “stimulation-treatable condition,” as used in the present application, including in the claims, means a condition selected from the list consisting of: meningitis, encephalitis, multiple sclerosis, cerebral infarction, a cerebral embolism, Guillaume-Barre syndrome, neuritis, neuralgia, spinal cord injury, paralysis, Alzheimer's disease, Parkinson's disease, depression, psychosis, schizophrenia, anxiety, autism, an attention disorder, trauma, spinal cord trauma, CNS trauma, a headache, a migraine headache, back pain, neck pain, syncope, faintness, dizziness, vertigo, memory loss, sleep disorders, insomnia, hypersomnia, dementia, glaucoma, appendicitis, a peptic ulcer, a gastric ulcer, a duodenal ulcer, peritonitis, pancreatitis, ulcerative colitis, pseudomembranous colitis, acute colitis, ischemic colitis, diverticulitis, epiglottitis, achalasia, cholangitis, cholecystitis, hepatitis, Crohn's disease, cirrhosis, inflammatory bowel disease (IBD), dysphagia, nausea, constipation, obesity, an eating disorder, gastrointestinal bleeding, acute renal failure, chronic renal failure, a glomerular disease, cystitis, incontinence, a urinary tract infection and pyelonephritis, enteritis, Whipple's disease, asthma, an allergy, anaphylactic shock, an immune complex disease, organ ischemia, a reperfusion injury, organ necrosis, hay fever, sepsis, septicemia, septic shock, cachexia, hyperpyrexia, eosinophilic granuloma, granulomatosis, sarcoidosis, septic abortion, epididymitis, vaginitis, prostatitis, eczema, urethritis, proteinuria, bronchitis, emphysema, rhinitis, cystic fibrosis, chronic obstructive pulmonary disease, sleep apnea, pneumonitis, pneumoultramicro-scopicsilicovolcanoconiosis, alveolitis, bronchiolitis, an infection of the upper respiratory tract, pulmonary edema, edema, pharyngitis, pleurisy, sinusitis, influenza, respiratory syncytial virus infection, herpes infection, HIV infection, hepatitis B virus infection, hepatitis C virus infection, disseminated bacteremia, tuberculosis, an Epstein-Ban virus infection, Dengue fever, candidiasis, malaria, filariasis, amebiasis, a hydatid cyst, a burn, dermatitis, dermatomyositis, sunburn, urticaria, a wart, a wheal, periarteritis nodosa, rheumatic fever, coeliac disease, adult respiratory distress syndrome, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosus, sarcoidosis, amyloidosis, osteoarthritis, fibromyalgia, chronic fatigue syndrome, Goodpasture's syndrome, Behcet's syndrome, Familial Mediterranean Fever, Sjogren syndrome, allograft rejection, graft-versus-host disease, Type I diabetes, Type II diabetes, ankylosing spondylitis, Berger's disease, sexual dysfunction, impotence, a neoplastic disorder, vasculitis, osteoporosis, a disorder of the pituitary, a disorder of the adrenal cortex, a seizure, epilepsy, an ataxic disorder, a prion disease, autism, a cerebrovascular disease, peripheral neuropathy, an addiction, an alcohol addiction, a nicotine addiction, a drug addiction, an autoimmune disease, a neurological disorder, pain, a psychiatric disorder, a skin disease, an infectious disease, a vascular disease, a kidney disorder, and a urinary tract disorder.

In an embodiment of the present invention, vagal stimulation is performed using the techniques described herein to treat one or more of the following organs or other portions of the body: a heart, a brain, lungs and/or other organs of the respiratory system, a liver, a kidney, a stomach, a small intestine, a large intestine, a muscle of a limb, a central nervous system, a peripheral nervous system, a pancreas, a bladder, skin, a urinary tract, a thyroid gland, a pituitary gland, and an adrenal cortex.

In an embodiment of the present invention, vagal stimulation using the techniques described herein attenuates muscle contractility.

In an embodiment of the present invention, vagal stimulation is performed using the techniques described herein to treat one or more of the following non-cardiac pathologies related to Connexin 43 (for each condition, an article cited hereinabove is indicated that describes the relationship between Connexin 43 and the condition): tuberous sclerosis (Mak B C et al.), breast cancer (Gould V E et al.), carcinoma (Gould V E et al.), melanoma (Haass N K et al.), osteoarthritis (Marino A A et al.), a wound (Brandner J M et al.), a seizure (Gajda Z et al.), bladder overactivity (Christ G J et al.), bladder outlet obstruction (Haefliger J A et al.), Huntington's disease (Vis J C et al.), and Alzheimer's disease (Nagy J I et al.).

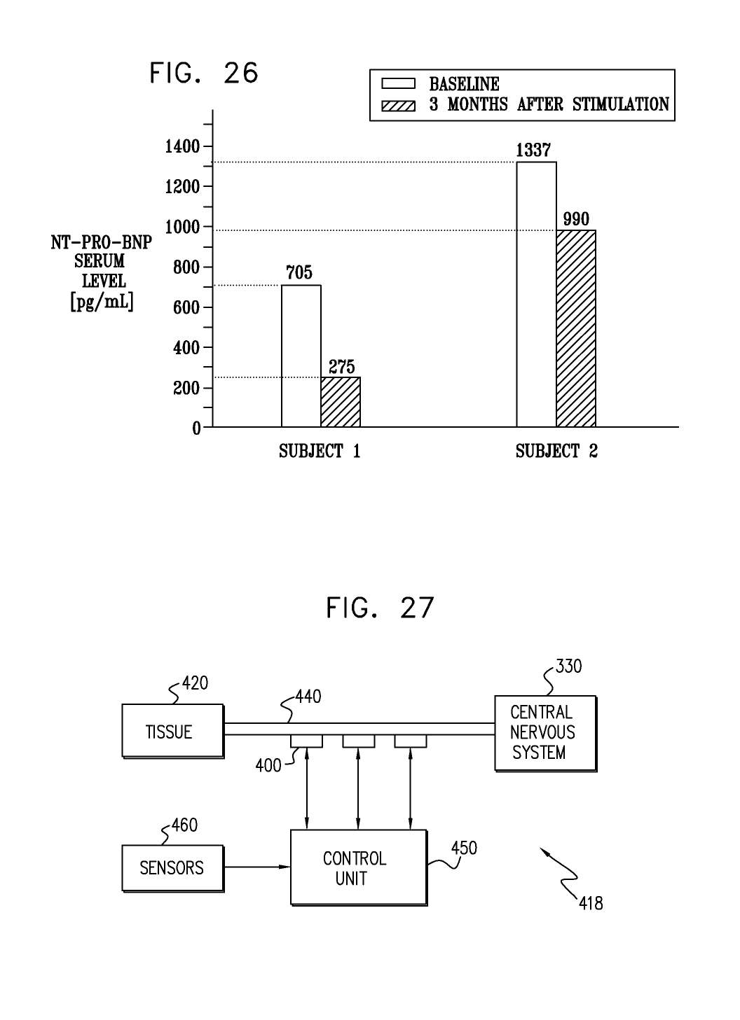

FIG. 27 is a schematic illustration of nerve stimulation apparatus 418, for applying electrical energy to induce propagation of impulses in one direction in a nerve 440, in order to treat a condition, while suppressing action potential propagation in the other direction, in accordance with a preferred embodiment of the present invention. For illustrative purposes, nerve 440 may be a cranial nerve, such as the vagus nerve, which emanates from the nervous tissue of the central nervous system (CNS) 330 and transmits sensory signals to CNS 330 and motor or other effector signals to tissue 420. Apparatus 418 typically comprises an implantable or external control unit 450, which drives one or more electrode devices 400 to apply an appropriate signal to respective sites on nerve 440. It is to be understood that whereas preferred embodiments of the present invention are described herein with respect to controlling propagation in a nerve, the scope of the present invention includes applying signals to other nervous tissue, such as individual axons or nerve tracts.

Preferably, control unit 450 receives and analyzes signals from sensors 460 located at selected sites in, on, or near the body of the patient. These sensor signals are typically qualitative and/or quantitative measurements of a medical, psychiatric and/or neurological characteristic of a disorder being treated. For example, sensors 460 may comprise electroencephalographic (EEG) apparatus to detect the onset of a seizure, or a user input unit, adapted to receive an indication of a level of discomfort, hunger, or fatigue experienced by the patient. Preferably, the sensor signals are analyzed within control unit 450, which, responsive to the analysis, drives electronic devices 400 to apply current to one or more sites on nerve 440, configured such that application thereof stimulates unidirectional propagation of nerve impulses to treat the specific disorder of the patient.

Alternatively, nerve stimulation apparatus 418 operates without sensors 460. In such a preferred embodiment, control unit 450 is typically preprogrammed to operate continuously, in accordance with a schedule, or under regulation by an external source.

For some applications of the present invention, the signals applied by control unit 450 to electrode devices 400 are configured to induce efferent nerve impulses (i.e., action potentials propagating in the direction of tissue 420), while suppressing nerve impulses traveling in nerve 440 towards CNS 330. For illustrative purposes, tissue 420 may comprise muscle tissue of the gastrointestinal tract, and treatment of motility disorders may be accomplished by inducing propagation of nerve impulses towards the muscle tissue, while suppressing the propagation of nerve impulses to CNS 330. Preferably, methods and apparatus described in U.S. Pat. No. 5,540,730 to Terry et al. are adapted for use with this embodiment of the present invention. In contrast to the outcome of application of the apparatus described in the Terry patent, however, in this embodiment of the present invention, CNS 330 substantially does not receive sensory signals that could potentially generate undesired responses.

Alternatively or additionally, gastroesophageal reflux disease (GERD) is treated by stimulating the vagus nerve unidirectionally, in order to induce constriction of the lower esophageal sphincter. Advantageously, such an application of unidirectional stimulation inhibits or substantially eliminates undesired sensations or other feedback to the central nervous system which would in some cases be induced responsive to stimulation of the vagus nerve. It is noted that this suppression of afferent impulses is typically only applied during the relatively short time periods during which pulses are applied to the vagus nerve, such that normal, physiological afferent impulses are in general able to travel, uninhibited, towards the CNS. For some applications, apparatus and methods described in the above-cited U.S. Pat. No. 5,188,104, 5,716,385 or 5,423,872 are adapted for use with unidirectional stimulation as provided by this embodiment of the present invention.

For some applications of the present invention, electrode devices 400 are configured to induce afferent impulses (i.e., action potentials propagating in the direction of CNS 330), while suppressing impulses in the direction of tissue 420. Typically, conditions such as eating disorders, coma, epilepsy, motor disorders, sleep disorders, hypertension, and neuropsychiatric disorders are treated by adapting techniques described in one or more of the above-cited references for use with therapeutic unidirectional impulse generation as provided by these embodiments of the present invention. Advantageously, this avoids unwanted and not necessarily beneficial outcomes of the prior art technique, such as bradycardia, enhanced gastric acid secretion, or other effects secondary to stimulation of the vagus nerve and communication of unintended nerve impulses to tissue 420. Which specific tissue 420 receives the efferent stimulation unintentionally induced by the prior art techniques depends upon the location on the nerve at which the stimulation is applied. For example, branchial motor efferents of the vagus nerve supply the voluntary muscles of the pharynx and most of the larynx, as well as one muscle of the tongue. The visceral efferents include parasympathetic innervation of the smooth muscle and glands of the pharynx, larynx, and viscera of the thorax and abdomen. Consequently, unintended efferent signal generation may induce undesired or unexpected responses in any of the tissue controlled and regulated by the vagus nerve. In preferred embodiments of the present invention, by contrast, such responses are suppressed while, at the same time, the desired afferent nerve signals are transmitted to CNS 330.

A variety of methods for inducing unidirectional propagation of action potentials are known in the art, some of which are described in the references cited in the Background section of the present patent application and may be adapted for use with preferred embodiments of the present invention.

In a preferred embodiment, unidirectional signal propagation is induced using methods and apparatus disclosed in:
U.S. Provisional Patent Application 60/263,834 to Cohen and Ayal, filed Jan. 25, 2001, entitled “Selective blocking of nerve fibers,” which is assigned to the assignee of the present patent application and is incorporated herein by reference,
U.S. patent application Ser. No. 09/824,682, filed Apr. 4, 2001, entitled “Method and apparatus for selective control of nerve fibers,” to Cohen and Ayal, which issued as U.S. Pat. No. 6,600,954 and is assigned to the assignee of the present patent application and is incorporated herein by reference,
PCT Application PCT/IL2002/000070, filed Jan. 23, 2002, entitled “Method and apparatus for selective control of nerve fibers,” to Cohen and Ayal, which published as PCT Publication WO 2002/058782 and is assigned to the assignee of the present patent application and is incorporated herein by reference, and/or
the above-cited U.S. Pat. Nos. 5,199,430, 4,628,942, and/or 4,649,936.

The Cohen and Ayal regular patent application describes a method for:

(a) selectively suppressing the propagation of naturally-generated action potentials which propagate in a predetermined direction at a first conduction velocity through a first group of nerve fibers in a nerve bundle, while (b) avoiding unduly suppressing the propagation of naturally-generated action potentials propagated in the predetermined direction at a different conduction velocity through a second group of nerve fibers in the nerve bundle.

The method includes applying a plurality of electrode devices to the nerve bundle, spaced at intervals along the bundle. Each electrode device is capable of inducing, when actuated, unidirectional “electrode-generated” action potentials, which produce collision blocks with respect to the naturally-generated action potentials propagated through the second group of nerve fibers. Moreover, each electrode device is actuated in sequence, with inter-device delays timed to generally match the first conduction velocity and to thereby produce a wave of anodal blocks, which: (a) minimize undesired blocking of the naturally-generated action potentials propagated through the first group of nerve fibers, while (b) maximizing the generation rate of the unidirectional electrode-generated action potentials which produce collision blocks of the naturally-generated action potentials propagated through the second group of nerve fibers. Such a method may be used for producing collision blocks in sensory nerve fibers in order to suppress pain, and also in motor nerve fibers to suppress selected muscular or glandular activities.

Alternatively or additionally, embodiments of the present invention induce the propagation of unidirectional action potentials using techniques described in the above-cited U.S. Pat. Nos. 4,649,936 to Ungar et al., and 4,608,985 to Chrish et al., which describe apparatus and methods for selectively blocking action potentials passing along a nerve trunk. In this case, electrode device 400 comprises an asymmetric, single electrode cuff, which includes an electrically non-conductive or dielectric sleeve that defines an axial passage therethrough. The dielectric sheath and axial passage extend from a first end, which is disposed toward the origin of orthodromic pulses, to a second end. The gap between the nerve and the cuff is filled by conductive body tissues and fluids after implantation in the body. A single annular electrode is disposed in the axial passage, which may be mounted on the inner surface of the dielectric sleeve within the axial passage. Other implementation details may be found in the Ungar and Chrish patents.

According to another aspect of the present invention, there is provided a method of selectively suppressing the propagation of body-generated action potentials propagated in a predetermined direction at a first velocity through a first group of nerve fibers in a nerve bundle without unduly suppressing the propagation of body-generated action potentials propagated in the predetermined direction at a different velocity through a second group of nerve fibers in the nerve bundle, comprising: applying a plurality of electrode devices to, and spaced along the length of, the nerve bundle, each electrode device being capable of outputting, when actuated, unidirectional electrode-generated action potentials producing collision blocks with respect to the body-generated action potentials propagated through the second type of nerve fibers; and sequentially actuating the electrode devices with delays timed to the first velocity to produce a “green wave” of anodal blocks minimizing undesired blocking of the body-generated action potentials propagated through the first group of nerve fibers while maximizing the generation rate of said unidirectional electrode-generated action potentials producing collision blocks with respect to the body-generated action potentials propagated through said second type of nerve fibers.

Such a method may be used for producing collision blocks in sensory nerve fibers in order to suppress pain, and also in motor nerve fibers to suppress selected muscular or glandular activities.

According to a further aspect of the invention, there is provided a method of selectively controlling nerve fibers in a nerve bundle having fibers of different diameters propagating action potentials at velocities corresponding to their respective diameters, comprising: applying a plurality of electrode devices to, and spaced along the length of, the nerve bundle, each electrode device being capable of producing, when actuated, unidirectional electrode-generated action potentials; and sequentially actuating the electrode devices with delays timed to the velocity of propagation of action potentials through the fibers of one of the diameters.

In some described preferred embodiments, the electrode devices are sequentially actuated to generate unidirectional action potentials producing collision blocks of the body-generated action potentials propagated through the nerve fibers of another diameter. Such collision blocks may be used for suppressing pain sensations without unduly interfering with normal sensations, or for selectively suppressing certain motor controls without unduly interfering with others.

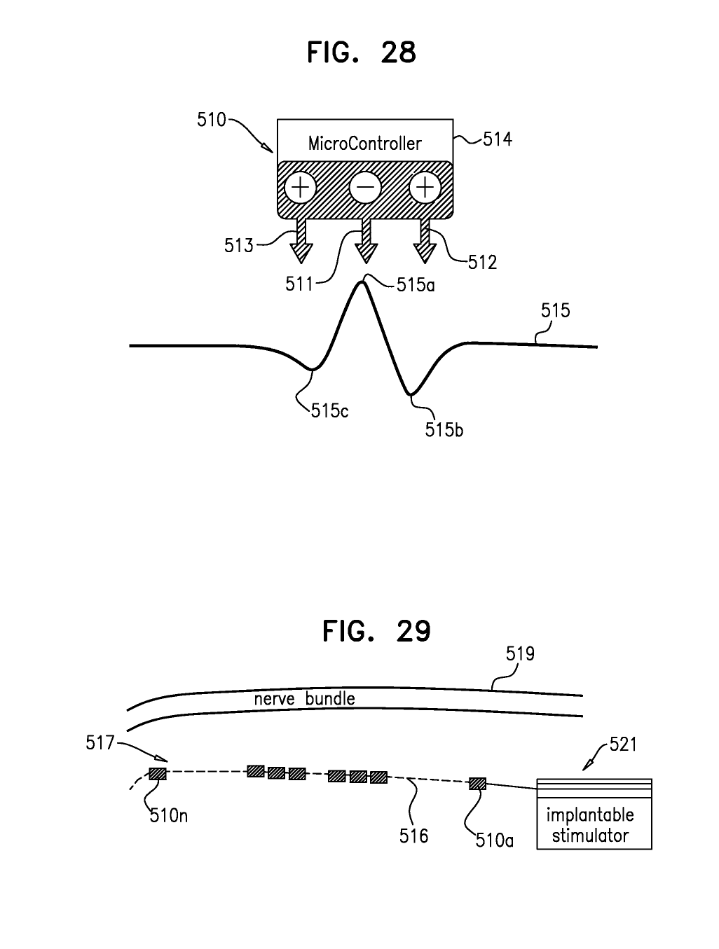

A basic element in the preferred embodiments of the method and apparatus described below is the tripolar electrode device. Its construction and operation are diagrammatically illustrated in FIG. 28.

As shown in FIG. 28, the tripolar electrode device, therein designated 510, includes three electrodes, namely, a central cathode 511, a first anode 512 on one side of the cathode, and a second anode 513 on the opposite side of the cathode. The illustrated tripolar electrode device further includes a microcontroller 514 for controlling the three electrodes 511, 512 and 513, as will be described below.

Curve 515 shown in FIG. 28 illustrates the activation function performed by the tripolar electrode device 510 on the nerve bundle underlying it. As shown in FIG. 28, this activation function includes a sharp positive peak 515a underlying the cathode 511, a relatively deep negative dip 515b underlying the anode 512, and a shallower negative dip 515c underlying the anode 513.

When the tripolar electrode 510 is placed with its cathode 511 and anodes 512, 513 in contact with, or closely adjacent to, a nerve bundle, the energization of the cathode 511 generates, by cathodic stimulation, action potentials in the nerve bundle which are propagated in both directions; the energization of anode 512 produces a complete anodal block to the propagation of the so-generated action potentials in one direction; and the energization of anode 513 produces a selective anodal block to the propagation of the action potentials in the opposite direction.

According to another aspect of the present invention, a plurality of electrode devices, preferably of such tripolar electrodes, are used to generate a sequence of electrode-generated action potentials (EGAPs) for more effectively suppressing the propagation of body-generated action potentials (BGAPs) propagated through sensory nerves towards the central nervous system (CNS) for pain control, as well as for suppressing the propagation of body-generated action potentials propagated through motor nerves from the central nervous system towards the peripheral nervous system (PNS) for muscular or glandular stimulation or suppression. As will be described more particularly below, the plurality of electrode devices are sequentially actuated with delays to produce a “green wave” of unidirectional EGAPs effective to reduce the interference with the BGAPs propagated unhindered, or to reinforce the stimulation of muscular or glandular activities desired to be effected.

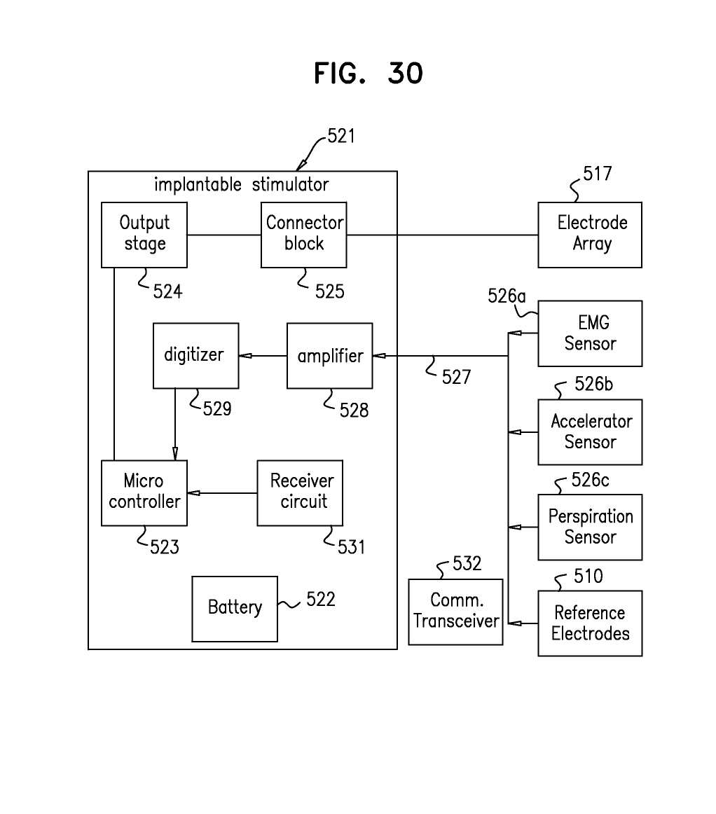

FIGS. 29 and 30 are diagrams illustrating one form of apparatus constructed in accordance with the present invention utilizing a plurality of the tripolar electrode devices, therein designated 510a-510n, shown in FIG. 28. Such electrode devices are interconnected by a bus 516 to form an electrode array 517 to be applied, as by implantation, with the electrode devices spaced along the length of the nerve bundle, shown at 519, and to be selectively actuated, as will be described more particularly below, by a stimulator, generally designated 521. The construction of the stimulator 521 is more particularly illustrated in FIG. 30.

Each of the electrode devices 510a-510n is of the tripolar construction shown in FIG. 28, to include a central cathode 511 flanked on its opposite sides by two anodes 512, 513. Each such electrode device further includes a microcontroller, shown at 514 in FIG. 28, and more particularly described below with respect to FIG. 34, for sequentially controlling the actuation of the electrodes 511-513 of each electrode device in order to produce the “green wave” briefly described above, and to be more particularly described below.

The assembly of electrode devices 510a-510n, and the stimulator 521 for sequentially actuating them, are preferably both implantable in the body of the subject with the electrodes in contact with, or closely adjacent to, the nerve bundle 515. Accordingly, the simulator 521 includes its own power supply, shown at 522 in FIG. 30. The stimulator 521 further includes a microcontroller 523 having output stage 524 connected, via connector block 525, to the plurality of electrode devices 510a-510n for sequentially actuating them, as will be described below.

Stimulator 521 further includes an input circuit for inputting various sensor signals for purposes of calibration and/or control. As shown in FIG. 30, such inputs may be from an EMG (electromyogram) signal sensor 526a and from an accelerator sensor 526b. The EMG sensor 526a may be used for calibration purposes, e.g., to calibrate the apparatus according to EMG signals generated by a subject's muscle during the calibration of the apparatus (described below), or for control purposes, e.g., for automatically actuating the device upon the occurrence of a particular EMG signal. The accelerator sensor 526b may be used for control purposes, e.g., to automatically actuate the device upon the occurrence of tremors or spasms in order to suppress in the tremors by blocking certain motor nerves.

Stimulator 521 may also have an input from a perspiration sensor 526c for automatic control of sweat glands. It may also have an input from one of the electrodes serving as a reference electrode for calibration purposes, as will also be described more particularly below.

The inputs into the stimulator 521 may be by wire or bus, as shown at 527 in FIG. 30. Such inputs are amplified in amplifier 528, and digitized in a digitizer 529, before being inputted into the microcontroller 523.

The inputs to the stimulator 521 may also be by wireless communication, as schematically shown at 532 in FIG. 30, particularly where the device is implanted. For this purpose, stimulator 521 includes a receiver 531 for receiving such inputs. Such inputs are also amplified in amplifier 528 and digitized in digitizer 529 before being inputted into the microcontroller 523.

Operation of the Illustrated Apparatus

The apparatus illustrated in FIGS. 29 and 30, when applied along the length of the nerve bundle 515 as shown in FIG. 29, is capable of suppressing the propagation of body-generated action potentials (BGAPs) propagated through the small-diameter nerve fibers in a nerve bundle without unduly suppressing the propagation of BGAPs propagated through the large-diameter nerve fibers in the nerve bundle. One application of such a device is to reduce pain sensations; and another application of the device is to suppress muscular or glandular activities. The apparatus illustrated in FIGS. 29 and 30 may also be used for generating, by the electrode devices, action potentials (hereinafter frequently referred to as electrode-generated action potentials, or EGAPS) where the body fails to produce the necessary BGAPs to produce a particular muscular or glandular activity. A further application of the apparatus, therefore, is to stimulate a muscular or glandular activity.

As described above, when the cathode 511 of each tripolar electrode device 510 is actuated, it generates an action potential by cathodic stimulation propagated in both directions; whereas when anode 512 of the respective tripolar electrode 510 is energized, it produces a complete anodal block on one side of the cathode, to thereby make the electrode-generated action potential unidirectional and propagated away from the central nervous system. On the other hand, when anode 513 is energized, it produces an anodal block only with respect to the BGAPs propagated through the large-diameter sensory nerves, since they are more sensitive to the anodal current. Accordingly, the EGAPs from the small-diameter sensory nerves are permitted, to a larger extent, to propagate through the anodal block.

The EGAPs outputted by the anodal block may be used as collision blocks with respect to sensory BGAPs to suppress pain, or with respect to motor BGAPs to suppress undesired muscular activity (e.g., tremors, spasms), or glandular activity (e.g., excessive perspiration).

An undesired side effect of this activation scheme, is that at the time when anode 512 of device 510 is actuated to generate an anodal block as described above, all BGAPs in both small and large fibers are blocked and cannot pass the device. Thus every production of an EGAP is accompanied by a brief period in which all BGAPs cannot pass the site of the device 510. In order to minimize the blocking of BGAPs while maximizing the amount of EGAPs produced, the tripolar electrode devices 510a-510n are sequentially actuated, under the control of the stimulator 521. This sequential actuation is timed with the propagation velocity of the action potentials through the nerve fiber not to be blocked. Thus, as well known for controlling vehicular traffic, when stop lights spaced along a thoroughfare are controlled to define a “green wave” travelling at a predetermined velocity, the vehicles travelling at the “green wave” velocity will be less hindered than if the stop lights were not synchronized with their velocity.

The anodal blocks produced by the sequential actuation of the tripolar electrodes are comparable to the stop lights in a thoroughfare, and therefore the action potentials travelling at the velocity of the green wave will be less hindered by such stop lights or anodal blocks.

Thus, where the invention is used for pain control by suppressing the BGAPs in the small-diameter sensory nerves, producing a “green wave” of anodal blocks timed with the conduction velocity through the large-diameter sensory nerves, there will be less interference with the BGAPs representing normal sensations, travelling through the large-diameter sensory nerve fibers, as compared to the BGAPs representing pain sensations travelling through the small-diameter sensory nerve fibers which will be collision blocked by the EGAPs.

The same “green wave” effect can be provided in order to suppress BGAPs propagating through motor nerve fibers in order to block motor controls of selected muscles or glands.

Examples of Use of the Apparatus

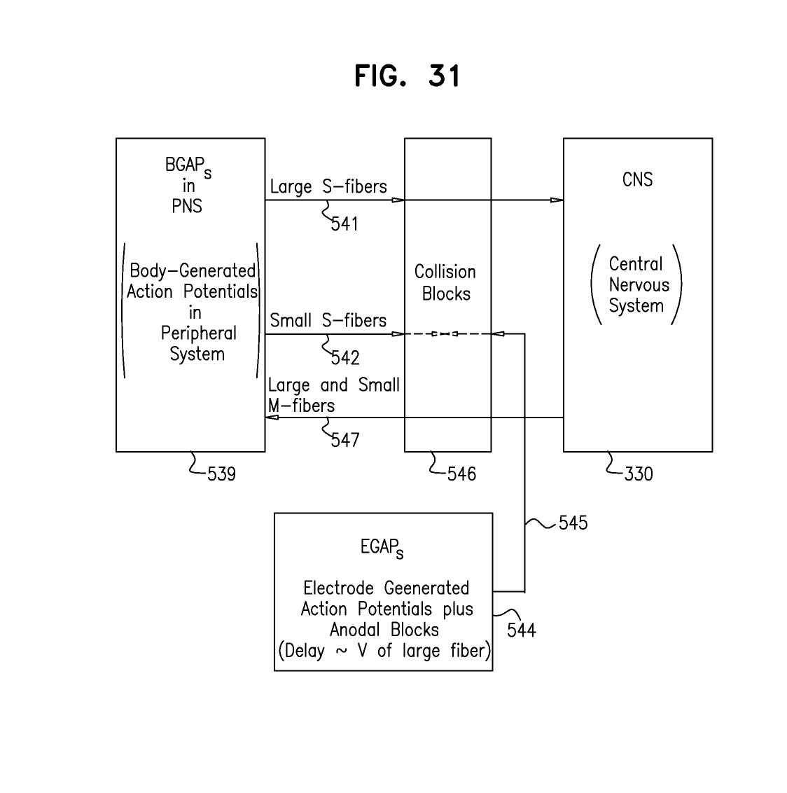

FIG. 31 illustrates an example of use of the described apparatus for reducing pain sensations by suppressing the BGAPs transmitted through the small-diameter sensory fibers without unduly hindering the transmission of the BGAPs through the large-diameter sensory fibers.

Thus, as shown in FIG. 31, the BGAPs in the peripheral nervous system PNS (block 539) generate normal sensations in the large sensory fibers 541 and pain sensations in the small sensory fibers 542. Normally, both types of sensations are propagated through their respective fibers to the central nervous system (CNS, block 443).

However, as shown in FIG. 31, the assembly of electrodes 510a-510n, when sequentially actuated with delays timed to the conduction velocity of the large-diameter fibers 541, generates unidirectional EGAPs (block 544) which are outputted with delays timed to correspond to the velocity of the large sensory fibers (as shown at 545) to produce a collision block (546) with respect to the BGAPs propagated through the small sensory fibers (542) without unduly hindering the BGAPs propagated through the large sensory fibers 541 to the central nervous system 330. Accordingly, the pain sensations normally propagated through the small sensory fibers 542 to the central nervous system 330 will be suppressed, while the normal sensations propagated through the large sensory fibers 541 will continue substantially unhindered to the central nervous system. In addition, as shown by line 547 in FIG. 31, the motor action potentials from the CNS to the PNS are also substantially unhindered.

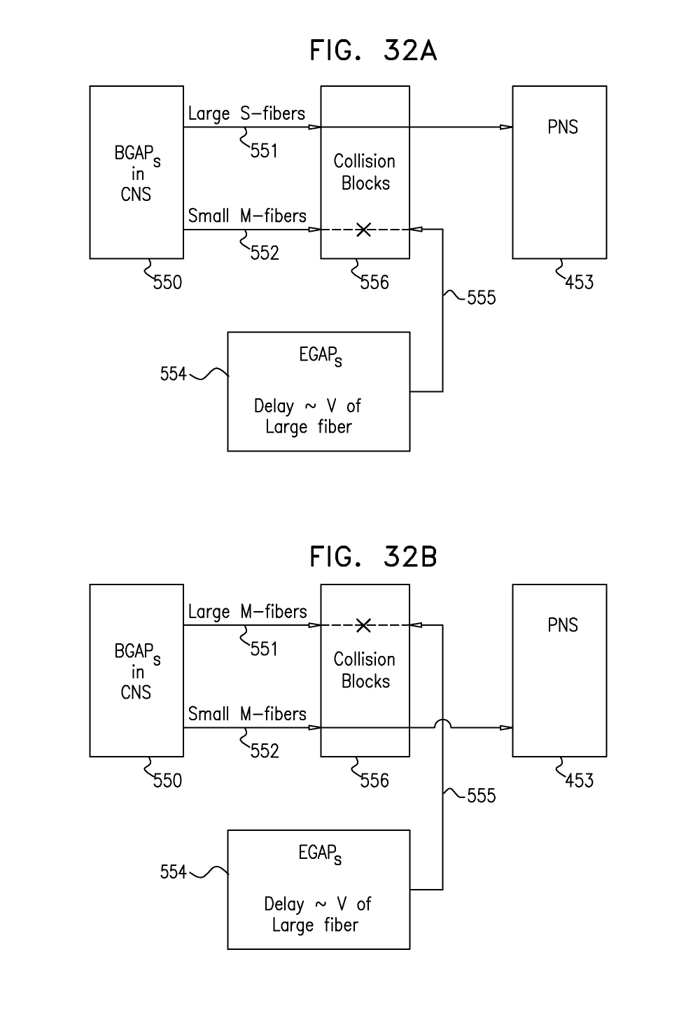

FIGS. 32A and 32B illustrate the application of the apparatus for suppressing certain muscular or glandular activities normally controlled by the BGAPs transmitted through the motor nerve fibers. In this case, as shown in FIG. 32A, the BGAPs are generated in the central nervous system (block 550) and are normally transmitted via large motor fibers 551 and small motor fibers 552 to the peripheral nervous system 553. FIG. 32B illustrates the arrangement wherein the EGAPs are generated at a rate corresponding to the velocity of the large motor fibers, as shown by blocks 554 and 555, so that they produce collision blocks with respect to the small motor fibers 552, and permit the BGAPs to be transmitted through the large motor fibers 551 to the peripheral nervous system 553.

FIG. 32B illustrates the variation wherein the apparatus generates EGAPs at a rate corresponding to the velocity of the small motor fibers (blocks 554, 555), such that the collision blocks (556) block the large motor fibers 551, and permit the BGAPs to be transmitted to the peripheral nervous system 453.

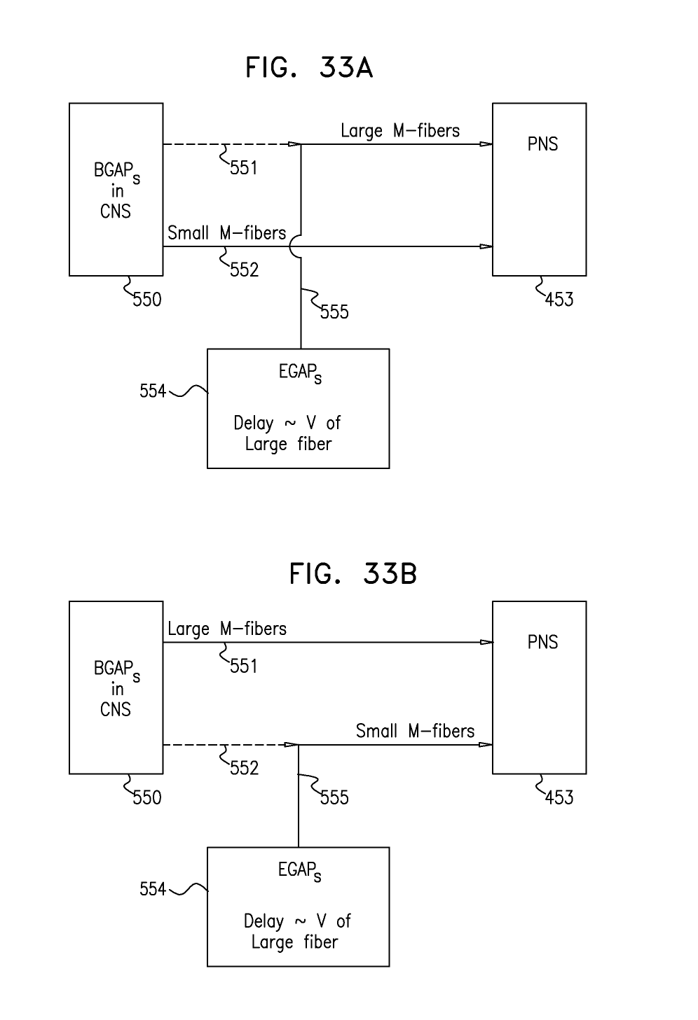

FIGS. 33A and 33B illustrate the applications of the apparatus for stimulating a particular muscle or gland where the body fails to develop adequate BGAPs in the respective motor nerve fiber for the respective muscular or glandular control. In this case, the apparatus generates unidirectional EGAPs selectively for the respective muscle or gland.

FIG. 33A illustrates the application of the invention wherein the body fails to generate in the central nervous system 330 adequate BGAPs for transmission by the large motor fibers to the peripheral nervous system 453, in which case the electrode devices 510a-510n in the electrode assembly would be sequentially energized by the stimulator 554 with delays timed to the velocity of propagation of action potentials through the large motor fibers. The unidirectional EGAPs are thus produced with delays timed to the conductive velocity of the large motor fibers, thereby permitting them to be transmitted via the large motor fibers to the peripheral nervous system.

FIG. 33B, on the other hand, illustrates the case where the electrodes 510a-510n are sequentially energized with delays timed to the velocity of the small motor fibers, thereby permitting the unidirectional EGAPs to be outputted via the small-diameter fibers to the peripheral nervous system 453.

Calibration

For best results, each electrode assembly should be calibrated for each patient and at frequent intervals. Each calibration requires adjustment of the cathodic and anodic currents in each tripolar electrode, and also adjustment of the timing of the sequential actuation of the tripolar electrodes.

To calibrate the cathodic and anodic currents for each electrode, the proximal electrode (510a, FIG. 29) is actuated to produce a unidirectional action potential propagated towards the distal electrode (510n) at the opposite end of the array. The so-produced action potential, after having traversed all the electrodes between electrodes 510a, and 510n, is detected and recorded by the distal electrode 510n. The currents in the electrodes are iteratively adjusted to produce maximum blocking.

FIG. 34A illustrates, at “a”, the signal detected by the distal electrode when the blocking is minimum, and at “b” when the signal detected by the distal electrode when the blocking is maximum.

FIG. 34B illustrates the manner of calibrating the electrode array to produce the proper timing in the sequential actuation of the electrodes for calibrating the sequential timing, the proximate electrode (510a) is again actuated to produce a unidirectional action potential propagated toward the distal electrode (510n). As the so-produced action potential traverses all the electrodes in between, each such in between electrode detects and records the action. This technique thus enables calibrating the electrode array to produce the exact delay between the actuations of adjacent electrodes to time the sequential actuations with the conduction velocity of the respective nerve fiber.

For example, where the sequential actuation is to produce a “green wave” having a velocity corresponding to the conduction velocity of the large sensory nerve fibers for reducing pain sensations, the timing would be adjusted so as to produce the sequential delay shown in FIG. 34B to thereby time the sequential actuations of the electrodes to the conductive velocity in the large sensory fibers.

The EMG sensor 526a shown in FIG. 30 may also be used for calibrating the electrode currents and sequential timing when the apparatus is to be used for providing a stimulation of a muscular or glandular activity where the body fails to provide the necessary BGAPs for this purpose. In this case, the currents and timing would be adjusted to produce a maximum output signal from the EMG sensor 526a for the respective muscle.

The EMG sensor 526a could also be used to automatically actuate the apparatus upon the detection of an undesired EMG signal, e.g., as a result of a tremor or spasm to be suppressed. For example, the accelerator sensor 526b could be attached to a limb of the subject so as to automatically actuate the apparatus in order to suppress tremors in the limb upon detection by the accelerator.

Other sensors could be included, such as an excessive perspiration sensor 526c, FIG. 30. This would also automatically actuate the apparatus to suppress the activity of the sweat glands upon the detection of excessive perspiration.

A method is provided of reducing pain sensations resulting from the propagation of body-generated action potentials towards the central nervous system through small-diameter sensory fibers in a nerve bundle, without unduly reducing other sensations resulting from the propagation of body-generated action potentials towards the central nervous system through large-diameter sensory fibers in the nerve bundle, comprising:

applying to the nerve bundle at least one electrode device capable, upon actuation, of generating unidirectional action potentials to be propagated through both the small-diameter and large-diameter sensory fibers in the nerve bundle away from the central nervous system; and actuating the electrode device to generate the unidirectional action potentials to produce collision blocks with respect to the body-generated action potentials propagated through the small-diameter fibers.

The electrode device may include electrodes which:

(i) generate the electrode-generated action potentials by cathodic stimulation;
(ii) produce a complete anodal block on one side of the cathode to make the electrode-generated action potentials unidirectional; and
(iii) produce a selective anodal block on the opposite side of the cathode to cause the electrode-generated action potentials to produce collision blocks with respect to the body-generated action potentials propagated through the small-diameter sensory fibers.

The electrode device may be a tripolar electrode device which includes a central cathode for producing the cathodic stimulation, a first anode on one side of the cathode for producing the complete anodal block, and a second anode on the opposite side of the cathode for producing the selective anodal block. There may be a plurality of the electrode devices spaced along the length of the nerve bundle; and wherein the electrode devices are sequentially actuated with delays timed to the velocity of propagation of the body-generated action potentials through the large-diameter fibers to produce a “green wave” of electrode-generated anodal blocks, thereby increasing the number of EGAPs in the small diameter fibers producing collision blocks while minimizing anodal blocking of the BGAPs propagated through the large-diameter sensory fibers.

A method is provided of selectively suppressing the propagation of body-generated action potentials propagated in a predetermined direction at a first velocity through a first group of nerve fibers in a nerve bundle without unduly suppressing the propagation of body-generated action potentials propagated in the predetermined direction at a different velocity through a second group of nerve fibers in the nerve bundle, comprising:

applying a plurality of electrode devices to, and spaced along the length of, the nerve bundle, each electrode device being capable of outputting, when actuated, unidirectional electrode-generated action potentials producing collision blocks with respect to the body-generated action potentials propagated through the second type of nerve fibers;

and sequentially actuating the electrode devices with delays timed to the first velocity to produce a “green wave” of anodal blocks minimizing undesired blocking of the body-generated action potentials propagated through the first group of nerve fibers, while maximizing the generation rate of the unidirectional electrode-generated action potentials producing collision blocks with respect to the body-generated action potentials propagated through the second type of nerve fibers.

The first group of nerve fibers may be large-diameter nerve fibers; and the second group of nerve fibers are small-diameter nerve fibers. The nerve fibers may be sensory nerve fibers, in which the predetermined direction of propagation of the body-generated action potentials to be collision blocked is towards the central nervous system, the method being effective for suppressing pain sensations propagated through the small-diameter sensory fibers without unduly suppressing other sensations propagated through the large-diameter sensory fibers.

The nerve fibers may be motor nerve fibers in which the predetermined direction of propagation of the body-generated action potentials to be collision blocked is away from the central nervous system towards a muscle or gland, the method being effective for suppressing motor impulses propagated through the small-diameter motor nerve fibers without unduly suppressing the propagation of the motor impulses through the large-diameter motor nerve fibers.

Each of the electrode devices may be a tripolar electrode which includes a central cathode for producing the electrode-generated action potentials by cathodic stimulation, a first anode on one side of the cathode for making the electrode-generated action potentials unidirectional, and a second anode on the opposite side of the cathode for producing the selective anodal blocking of the electrode-generated action potentials.

A method is provided of selectively controlling nerve fibers in a nerve bundle having fibers of different diameters propagating action potentials at velocities corresponding to their respective diameters, comprising:

applying a plurality of electrode devices to, and spaced along the length of, the nerve bundle, each electrode device being capable of producing, when actuated, unidirectional electrode-generated action potentials;

and sequentially actuating the electrode devices with delays timed to the velocity of propagation of action potentials through the fibers of one of the diameters.

The electrode devices may be sequentially actuated to generate unidirectional action potentials producing collision blocks of the body-generated action potentials propagated through the nerve fibers of a another diameter. The electrode devices may be sequentially actuated with delays timed to the velocity of the larger-diameter nerve fibers to produce a “green-wave” of anodal blocks in order to minimize blocking the body-generated action potentials propagated through the larger-diameter fibers while maximizing the number of EGAPs collision blocking the body-generated action potentials propagated through the small diameter fibers. The fibers may include large-diameter sensory fibers propagating body-generated action potentials representing normal sensations from the peripheral nervous system to the sensor nervous system, and small-diameter sensory fibers propagating body-generated action potentials representing pain sensations from the peripheral nervous system to the central nervous system, which pain sensations in the small-diameter sensory fibers are suppressed by collision block and the “green-wave” of anodal blocks minimizes blocking of the normal sensations in the large-diameter sensory nerves. The nerve fibers may include large-diameter motor fibers propagating body-generated action potentials representing certain motor controls from the central nervous system to the peripheral nervous system, and small-diameter motor nerve fibers representing other motor controls from the central nervous system to the peripheral nervous system, the motor controls in the small-diameter motor fibers being suppressed by collision blocks and the green-wave of anodal blocks minimizes blocking of the motor controls in the large-diameter motor fibers.

The nerve fibers may be motor fibers of different diameters for propagating body-generated action potentials from the central nervous system to the peripheral nervous system, the electrode devices being sequentially actuated to generate unidirectional action potentials to serve as motor action potentials to be propagated from the central nervous system to the peripheral nervous system to replace motor action potentials failed to be generated by the body.

Each of the electrode devices may be a tripolar electrode which includes a central cathode for producing the electrode-generated action potentials by cathodic stimulation, a first anode on one side of the cathode for making the electrode-generated action potentials unidirectional, and a second anode on the opposite side of the cathode for producing the selective anodal blocking of the electrode-generated action potentials.

Apparatus is provided for selectively blocking pain sensations resulting from the propagation of body-generated action potentials towards the central nervous system through small-diameter sensory fibers in a nerve bundle, without unduly reducing other sensations resulting from the propagation of body-generated action potentials towards the central nervous system through large-diameter sensory fibers in the nerve bundle, comprising:

an electrical device to be applied to the nerve bundle and having at least one electrode device capable, upon actuation, of generating unidirectional action potentials to be propagated through both the small-diameter and large-diameter sensory fibers in the nerve bundle away from the central nervous system;

and a stimulator for actuating the electrode device to generate the unidirectional action potentials to produce collision blocks of the body-generated action potentials in the small-diameter sensory fibers.

The electrode device may include electrodes which:

(a) generate the electrode-generated action potentials by cathodic stimulation;
(b) produce a complete anodal block on one side of the cathode to make the electrode-generated action potentials unidirectional; and
(c) produce a selective anodal block on the opposite side of the cathode to block the electrode-generated action potentials propagated through the large-diameter sensory fibers to a greater extent than those propagated through the small-diameter sensory fibers.

The electrode device may be a tripolar electrode which includes a central cathode for producing the cathodic stimulation, a first anode on one side of the cathode for producing the complete anodal block, and a second anode on the opposite side of the cathode for producing the selective anodal block. There may be a plurality of the electrode devices spaced along the length of the nerve bundle; and wherein the electrode devices are sequentially actuated with delays corresponding to the velocity of propagation of the body-generated action potentials through the large-diameter fibers to produce a “green wave” of electrode-generated action potentials collision blocking with the body-generated action potentials propagated through the small-diameter fibers while minimizing anodal blocking of action potentials propagating through the large-diameter fibers.

Apparatus is provided for selectively suppressing the propagation of body-generated action potentials propagated at a first velocity through a first type of nerve fibers in a nerve bundle without unduly suppressing the propagation of body-generated action potentials propagated at a different velocity through a second type of nerve fibers in the nerve bundle, comprising:

spacing a plurality of electrodes to be spaced along the length of the nerve bundle, each capable of producing, when actuated, unidirectional electrode-generated action potentials and a selective anodal block of the latter action potentials propagated through the first type of nerve fibers to a greater extent than those propagated through the second type of nerve fibers;

and a stimulator for sequentially actuating the electrode devices with delays timed to the first velocity to produce a “green wave” of anodal blocks minimizing undesired blocking of the body-generated action potentials propagated through the first group of nerve fibers, while maximizing the generation rate of the unidirectional electrode-generated action potentials producing collision blocks with respect to the body-generated action potentials propagated through the second type of nerve fibers.

Each of the electrode devices may be a tripolar electrode which includes a central cathode for producing the electrode-generated action potentials by cathodic stimulation, a first anode on one side of the cathode for making the electrode-generated action potentials unidirectional, and a second anode on the opposite side of the cathode for producing the selective anodal blocking of the electrode-generated action potentials. The plurality of electrode devices and the stimulator may be constructed to be implanted into the subject's body with the electrodes in contact with or closely adjacent to the nerve bundle.

The stimulator may be connected to the plurality of electrode devices by an asynchronous, serial four-wire bus. The stimulator may communicate with the plurality of electrode devices via a wireless communication link. Each of the tripolar electrode devices may include an insulating base carrying the cathode and two anodes on one face thereof, and control circuitry on the opposite face. The control circuitry may include a microprocessor communicating with the stimulator, and an L-C pulsing network controlled by the microprocessor.

Apparatus is provided for selectively controlling nerve fibers in a nerve bundle having fibers of different diameters propagating action potentials at velocities corresponding to their respective diameters, comprising:

a plurality of electrode devices to be applied to, and spaced along the length of, the nerve bundle, each electrode device being capable of producing, when actuated, unidirectional electrode-generated action potentials;

and a stimulator for sequentially actuating the electrode devices with delays timed to the velocity of propagation of action potentials through the fibers of one of the diameters.

The stimulator may sequentially actuate the electrode devices to generate unidirectional action potentials producing collision blocks of the body-generated action potentials propagated through the nerve fibers of a another diameter. The stimulator may sequentially actuate the electrode devices with delays corresponding to the velocity of larger-diameter nerve fibers to produce a “green-wave” of anodal blocks minimizing undesired blocking of the body-generated action potentials propagated through the large-diameter nerve fibers, while maximizing the generation rate of the unidirectional electrode-generated action potentials producing collision blocks with respect to the body-generated action potentials propagated through the small diameter nerve fibers.

The nerve fibers may be motor fibers of different diameters for propagating body-generated action potentials from the central nervous system to the peripheral nervous system, and the stimulator may sequentially actuate the electrode devices to generate unidirectional action potentials to serve as motor action potentials to be propagated from the central nervous system to the peripheral nervous system to replace motor action potentials failed to be generated by the body.

It is to be understood that whereas preferred embodiments of the present invention are generally described hereinabove with respect to stimulating and inhibiting action potential propagation in the vagus nerve, the scope of the present invention includes applying analogous techniques to other central or peripheral nervous tissue of a patient.

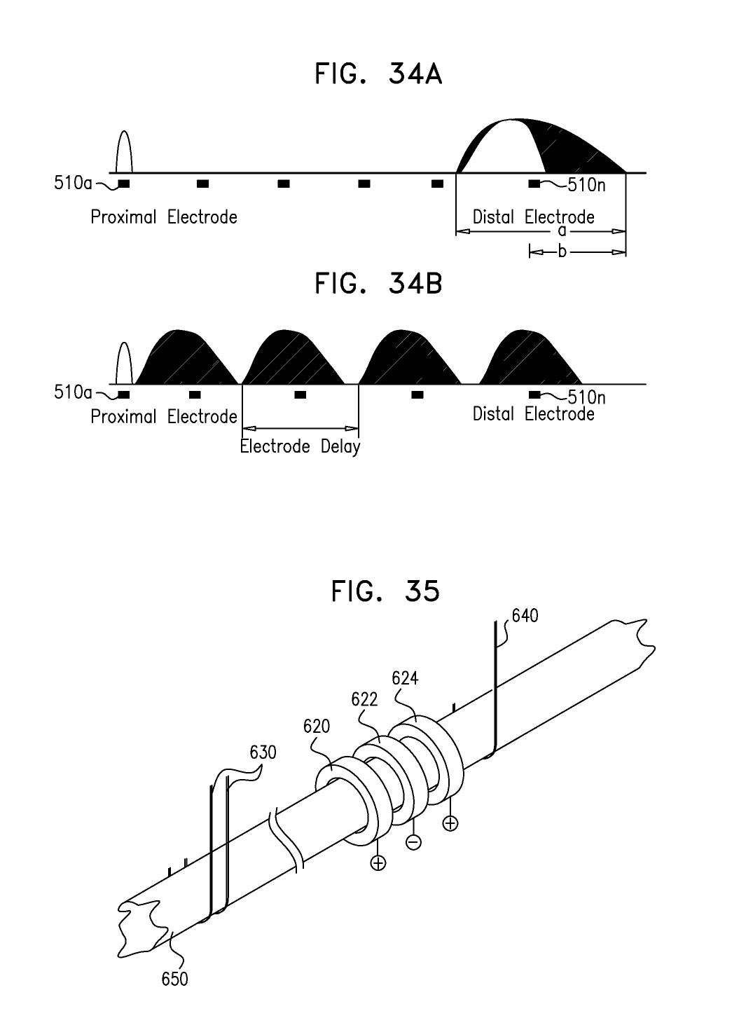

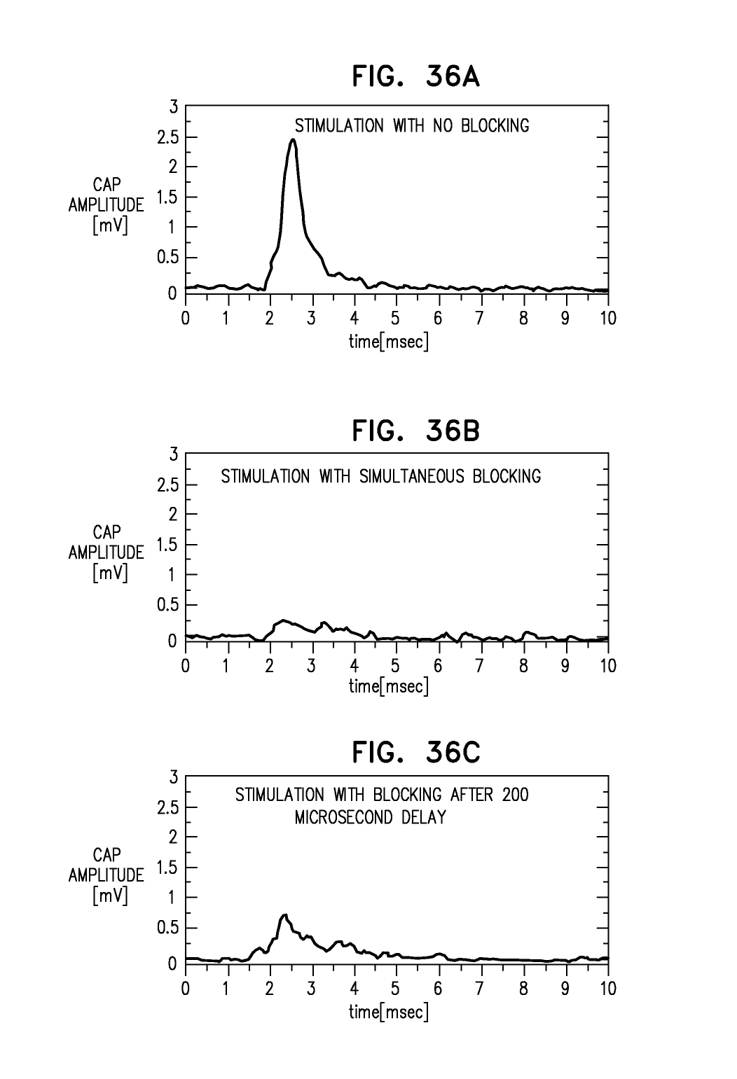

Reference is now made to FIGS. 35, 36A, 36B, and 36C. FIG. 35 is a schematic illustration of experimental apparatus which was applied to a rat sciatic nerve 650, in order to block the propagation of action potentials in A fibers thereof, in accordance with a preferred embodiment of the present invention. FIGS. 36A, 36B, and 36C are graphs showing experimental results attained during the use of the apparatus of FIG. 35, in accordance with a preferred embodiment of the present invention.

Bipolar hook electrodes 630 coupled to a stimulus isolator were placed in contact with nerve 650, and were driven to apply a 20 microsecond, 2 mA square pulse to the nerve. In this experimental preparation, these parameters were found to yield maximal compound action potentials (CAPs), as measured at a recording site by another hook electrode 640.

A tripolar platinum/iridium (Pt/Ir) cuff electrode assembly comprising individual electrodes 620, 622, and 624 was applied to nerve 650 between electrodes 630 and 640. The electrodes in the cuff were separated by gaps of 1 mm from each other, and the overall length of the cuff was 5 mm. (The cuff structure holding electrodes 620, 622, and 624 in place is not shown.) Current was applied to the electrode assembly through two stimulus isolators coupled to a D/A computer card, and was configured such that electrode 622 served as a cathode, and electrodes 620 and 624 served as anodes. A unidirectional action potential was generated by driving through electrode 622 a total cathodic current of 0.8 mA, and controlling electrodes 620 and 624 such that 0.1 mA passed through electrode 620, and 0.7 mA passed through electrode 624. The 0.1 mA was found to be sufficient to generate an action potential traveling towards electrodes 630, which collided with and ended propagation of action potentials generated by hook electrodes 630. Similarly, the 0.7 mA was found to be sufficient to inhibit propagation of action potentials which were generated responsive to the operation of the cuff electrode assembly. In these experiments, the current driven through electrodes 620, 622, and 624 was quasi-trapezoidal in time, having a duration of 200 microseconds and a decay constant of 300 microseconds.

FIG. 36A shows the results of application of a stimulation pulse through electrodes 630, without any blocking applied through the electrode assembly of electrodes 620, 622, and 624. A complete compound action potential, characteristic of this preparation, is seen to peak at approximately T=2.5 milliseconds. In FIG. 36B, stimulation was applied through electrodes 630 at the same time that electrodes 620, 622, and 624 drove blocking currents into nerve 650 as described hereinabove. The CAP is seen to be very significantly reduced, because the action potentials traveling in one direction from electrodes 630 collided with the “blocking” action potentials propagating in the other direction from electrodes 620 and 622. In FIG. 36C, the stimulation through electrodes 630 was followed, after a 200 microsecond delay, by the generation of the blocking currents through electrodes 620, 622, and 624. In this case, it is seen that action potentials propagating through faster fibers had already passed the cuff electrode assembly by the time that the action potentials propagating from electrode 620 had been initiated. Since there was no elimination by collision, the fastest moving action potentials leaving electrodes 630 were detected by electrode 640. However, slower action potentials were eliminated, such that the overall CAP area is seen to be significantly smaller in FIG. 36C than in FIG. 36A. For some applications, the delay between applying the stimulation through electrodes 630 and generating the blocking currents through electrodes 620, 622, and 624 is adjusted based on the CAP detected using electrode 640, so as to maximize blocking (suppression) of the slower action potentials (propagating in the slower fibers), thereby minimizing the CAP of the slow fibers.

For some applications, this technique is utilized to affect action potential propagation in the pelvic nerve, or in another nerve, in order to treat erectile dysfunction. Preferably, the signals applied are configured so as to cause the arterial dilation responsible for erection, e.g., by collision-blocking action potentials propagating in sympathetic C fibers which innervate arteries of the penis that, when constricted, prevent erection. By inhibiting action potential propagation in these fibers, the arteries dilate, and erection is achieved. Preferably, the signal is applied in a unidirectional mode, so as to prevent undesired action potentials from being conveyed to the penis in response to the applied signal.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 11/064,446, filed Feb. 22, 2005, entitled, “Techniques for applying, configuring, and coordinating nerve fiber stimulation,” which issued as U.S. Pat. No. 7,974,693;
U.S. patent application Ser. No. 11/062,324, filed Feb. 18, 2005, entitled, “Techniques for applying, calibrating, and controlling nerve fiber stimulation,” which issued as U.S. Pat. No. 7,634,317;
U.S. patent application Ser. No. 10/719,659, filed Nov. 20, 2003, entitled, “Selective nerve fiber stimulation for treating heart conditions,” which issued as U.S. Pat. No. 7,778,711;
PCT Patent Application PCT/IL03/00431, filed May 23, 2003, entitled, “Selective nerve fiber stimulation for treating heart conditions,” which published as PCT Publication WO 03/099377;
PCT Patent Application PCT/IL03/00430, filed May 23, 2003, entitled, “Electrode assembly for nerve control,” which published as PCT Publication WO 03/099373;
U.S. patent application Ser. No. 10/205,475, filed Jul. 24, 2002, entitled, “Selective nerve fiber stimulation for treating heart conditions,” which issued as U.S. Pat. No. 7,778,703;
U.S. patent application Ser. No. 09/944,913, filed Aug. 31, 2001, entitled, “Treatment of disorders by unidirectional nerve stimulation,” which issued as U.S. Pat. No. 6,684,105;
PCT Patent Application PCT/IL02/00068, filed Jan. 23, 2002, entitled, “Treatment of disorders by unidirectional nerve stimulation,” which published as PCT Publication WO 03/018113, and U.S. patent application Ser. No. 10/488,334 in the national stage thereof, filed Jul. 6, 2004, which issued as U.S. Pat. No. 7,734,355;
U.S. Provisional Patent Application 60/383,157 to Ayal et al., filed May 23, 2002, entitled, “Inverse recruitment for autonomic nerve systems”;
U.S. Provisional Patent Application 60/612,428, filed Sep. 23, 2004, entitled, “Inflammation reduction by vagal stimulation”;
U.S. Provisional Patent Application 60/668,275, filed Apr. 4, 2005, entitled, “Parameter improvement by vagal stimulation”;
U.S. patent application Ser. No. 11/022,011, filed Dec. 22, 2004, entitled, “Construction of electrode assembly for nerve control,” which issued as U.S. Pat. No. 7,561,922;
U.S. Provisional Patent Application 60/628,391, filed Nov. 15, 2004, entitled, “Electrode array for selective unidirectional stimulation”;
U.S. patent application Ser. No. 10/461,696, filed Jun. 13, 2003, entitled, “Vagal stimulation for anti-embolic therapy,” which issued as U.S. Pat. No. 7,321,793;
U.S. Provisional Patent Application 60/478,576, filed Jun. 13, 2003, entitled, “Applications of vagal stimulation”;
PCT Patent Application PCT/IL04/000496, filed Jun. 10, 2004, entitled, “Vagal stimulation for anti-embolic therapy,” which published as PCT Publication WO 04/110550;
PCT Patent Application PCT/IL04/000495, filed Jun. 10, 2004, entitled, “Applications of vagal stimulation,” which published as PCT Publication WO 04/110549;
U.S. Provisional Patent Application 60/655,604 to Ben-David et al., filed Feb. 22, 2005;
U.S. patent application Ser. No. 11/062,324, filed Feb. 18, 2005, entitled, “Techniques for applying, calibrating, and controlling nerve fiber stimulation,” which issued as U.S. Pat. No. 7,634,317;
U.S. patent application Ser. No. 11/064,446, filed Feb. 22, 2005, entitled, “Techniques for applying, configuring, and coordinating nerve fiber stimulation,” which issued as U.S. Pat. No. 7,974,693;
U.S. patent application Ser. No. 10/866,601, filed Jun. 10, 2004, entitled, “Applications of vagal stimulation,” which published as US Patent Application Publication 2005/0065553; and
U.S. patent application Ser. No. 10/205,474, filed Jul. 24, 2002, entitled, “Electrode assembly for nerve control,” which issued as U.S. Pat. No. 6,907,295.

Although embodiments of the invention are generally described herein with respect to electrical transmission of power and electrical stimulation of tissue, other modes of stimulation may also be used, such as magnetic stimulation or chemical stimulation.

The techniques described herein may be performed in combination with other techniques, which are known in the art or which are described in the references cited herein, that stimulate an autonomic nerve, such as the vagus nerve, in order to achieve a desired therapeutic end.

For some applications, techniques described herein are used to apply controlled stimulation to one or more of the following: the lacrimal nerve, the salivary nerve, the vagus nerve, the pelvic splanchnic nerve, or one or more sympathetic or parasympathetic autonomic nerves. Such controlled stimulation may be used, for example, to regulate or treat a condition of the lung, heart, stomach, pancreas, small intestine, liver, spleen, kidney, bladder, rectum, large intestine, reproductive organs, or adrenal gland.

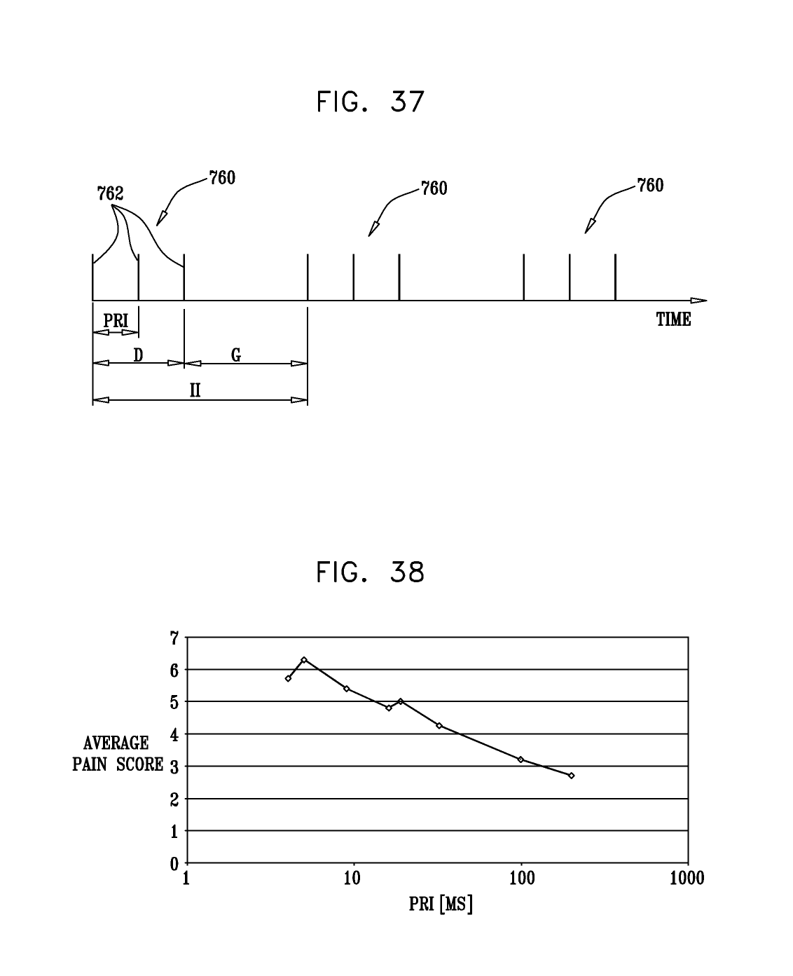

Reference is made to FIG. 37, which is a schematic illustration of a series of bursts 760, in accordance with an embodiment of the present invention. Control unit 120 is configured to drive electrode device 726 to apply stimulation in the series of bursts 760, at least one of which bursts includes a plurality of pulses 762, such as at least three pulses 762. Control unit 120 configures:
(a) a pulse repetition interval (PRI) within each of multi-pulse bursts 760 (i.e., the time from the initiation of a pulse to the initiation of the following pulse within the same burst) to be on average at least 20 ms, such as at least 30 ms, e.g., at least 50 ms or at least 75 ms, and
(b) an interburst interval (II) (i.e., the time from the initiation of a burst to the initiation of the following burst) to be at least a multiple M times the burst duration D. Multiple M is typically at least 1.5 times the burst duration D, such as at least 2 times the burst duration, e.g., at least 3 or 4 times the burst duration. (Burst duration D is the time from the initiation of the first pulse within a burst to the conclusion of the last pulse within the burst.)

In other words, burst duration D is less than a percentage P of interburst interval II, such as less than 75%, e.g., less than 67%, 50%, or 33% of the interval. For some applications, the PRI varies within a given burst, in which case the control unit sets the PRI to be on average at least 20 ms, such as at least 30 ms, e.g., at least 50 ms or at least 75 ms. For other applications, the PRI does not vary within a given burst (it being understood that for these applications, the “average PRI” and the PRI “on average,” including as used in the claims, is equivalent to the PRI; in other words, the terms “average PRI” and the PRI “on average” include within their scope both (a) embodiments with a constant PRI within a given burst, and (b) embodiments with a PRI that varies within a given burst).

Typically, each burst 760 includes between two and 14 pulses 762, e.g., between two and six pulses, and the pulse duration (or average pulse duration) is between about 0.1 and about 4 ms, such as between about 100 microseconds and about 2.5 ms, e.g., about 1 ms. Typically, control unit 120 sets the interburst interval II to be less than 10 seconds. For some applications, control unit 120 is configured to set the interburst interval II to be between 400 ms and 1500 ms, such as between 750 ms and 1500 ms. Typically, control unit 120 sets an interburst gap G between a conclusion of each burst 760 and an initiation of the following burst 760 to have a duration greater than the PRI. For some applications, the duration of the interburst gap G is at least 1.5 times the PRI, such as at least 2 times the PRI, at least 3 times the PRI, or at least 4 times the PRI.

Although the control unit typically withholds applying current during the periods between bursts and between pulses, it is to be understood that the scope of the present invention includes applying a low level of current during such periods, such as less than 50% of the current applied during the “on” periods, e.g., less than 20% or less than 5%. Such a low level of current is hypothesized to have a different, significantly lower, or a minimal physiological effect on the subject. For some applications, control unit 120 is configured to apply an interburst current during at least a portion of interburst gap G, and to set the interburst current on average to be less than 50% (e.g., less than 20%) of the current applied on average during the burst immediately preceding the gap. For some applications, control unit 120 is configured to apply an interpulse current to the site during at least a portion of the time that the pulses of bursts 760 are not being applied, and to set the interpulse current on average to be less than 50% (e.g., less than 20%) of the current applied on average during bursts 760.

For some applications, the control unit is configured to synchronize the bursts with a feature of the cardiac cycle of the subject. For example, each of the bursts may commence after a delay after a detected R-wave, P-wave, or other feature of an ECG. For these applications, one burst is typically applied per heart beat, so that the interburst interval II equals the R-R interval, or a sum of one or more sequential R-R intervals of the subject. Alternatively, for some applications, the control unit is configured to synchronize the bursts with other physiological activity of the subject, such as respiration, muscle contractions, or spontaneous nerve activity.

In an embodiment of the present invention, the control unit sets the PRI to at least 75% of a maximum possible PRI for a given interburst interval II (such as the R-R interval of the subject), desired percentage P, and desired PPT. For some applications, the following equation is used to determine the maximum possible PRI:

PRI=II*P/(PPT−1) (Equation 1)

For example, if the II is 900 ms, percentage P is 33.3%, and the desired PPT is 4 pulses, the maximum possible PRI would be 900 ms*33.3%/(4−1)=100 ms, and the control unit would set the actual PRI to be at least 75 ms. For some applications, control unit 120 uses this equation to determine the PRI, such as in real time or periodically, while for other applications this equation is used to produce a look-up table which is stored in the control unit. For still other applications, this equation is used to configure the control unit. For some applications, multiple M is a constant, which is stored in control unit 120, while for other applications, control unit 120 adjusts M during operation, such as responsively to one or more sensed physiological values, or based on the time of day, for example. It is noted that Equation 1 assumes that the pulse width of the pulses does not contribute meaningfully to burst duration D. Modifications to Equation 1 to accommodate longer pulse widths will be evident to those skilled in the art.

For some applications, when using Equation 1, a maximum value is set for the PRI, such as between 175 and 225, e.g., about 200, and the PRI is not allowed to exceed this maximum value regardless of the result of Equation 1.

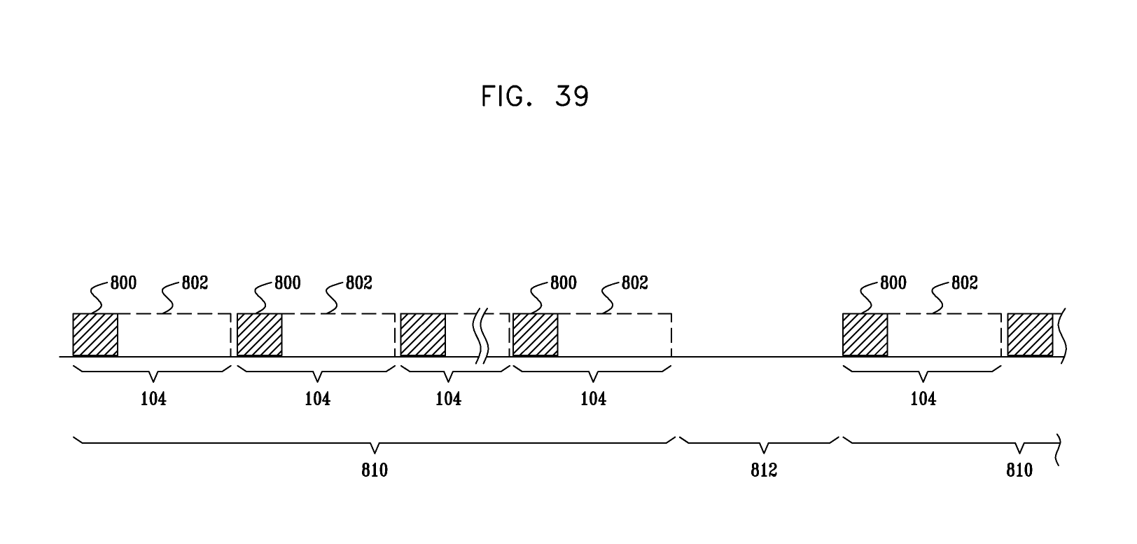

In an experiment conducted on three human subjects, the inventors found that increasing the PRI of the applied stimulation reduced sensations of acute pain experienced by the subjects. In each of the subjects, two stimulation regimens were a applied: (a) stimulation with bursts having a PPT of 3 and a PRI of 6 ms, synchronized with the cardiac cycle, and (b) stimulation with single-pulse (i.e., a PPT of 1) bursts at three times the heart rate, but not synchronized with the cardiac cycle. Regimen (b) had an effective PRI of about 300 ms. The overall number of pulses per minute was thus three times the heart rate in both regimens. Stimulation with the extended PRI of regimen (b) resulted in acute pain that was markedly attenuated compared to stimulation with the shorter PRI of regimen (a). (However, it was observed that stimulation with regimen (b) quickly caused secondary neuropathic pain projecting along the mandible, as described below with reference to FIG. 39. The inventors attribute the occurrence of such secondary pain to the shorter non-stimulation periods between pulses of regimen (b) compared to regimen (a).)

FIG. 38 is a graph showing experimental results obtained in an experiment performed on human subjects, in accordance with an embodiment of the present invention. The digital nerves of five healthy volunteers were stimulated using an external stimulator in several stimulation sessions. During each stimulation session, a single burst was applied, having a PPT of 4, an amplitude of 1 to 5 mA, and a pulse width of 1 ms. Each of the sessions was randomly assigned a PRI, without the knowledge of the subjects, and the subjects scored the pain associated with each session on a scale of 1 to 10, with higher values representing greater perceived acute neuropathic pain. The graph reflects the averaged pain scores for different PRIs across all five subjects. As can be seen in the graph, greater PRIs were strongly correlated with reduced acute pain scores.

In an embodiment, these extended PRI techniques are applied to stimulation of nerves other than the vagus nerve.

Reference is made to FIG. 39, which is a schematic illustration of a stimulation regimen, in accordance with an embodiment of the present invention. Control unit 120 is configured to apply the stimulation during “on” periods 800 alternating with “off” periods 802, during which no stimulation is applied (each set of a single “on” period followed by a single “off” period is referred to hereinbelow as a “cycle” 804). Typically, each of “on” periods 800 has an “on” duration equal to at least 1 second (e.g., between 1 and 10 seconds), and each of “off” periods 802 has an “off” duration equal to at least 50% of the “on” duration, e.g., at least 100% or 200% of the “on” duration. Control unit 120 is further configured to apply such intermittent stimulation during stimulation periods 810 alternating with rest periods 812, during which no stimulation is applied. Each of rest periods 802 typically has a duration equal to at least the duration of one cycle 804, e.g., between one and 50 cycles, such as between two and four cycles, and each of stimulation periods 810 typically has a duration equal to at least 5 times the duration of one of rest periods 812, such as at least 10 times, e.g., at least 15 times. For example, each of stimulation periods 810 may have a duration of at least 30 cycles, e.g., at least 60 cycles or at least 120 cycles, and no greater than 2400 cycles, e.g., no greater than 1200 cycles. Alternatively, the duration of the stimulation and rest periods are expressed in units of time, and each of the rest periods has a duration of at least 30 seconds, e.g., such as at least one minute, at least two minutes, at least five minutes, or at least 25 minutes, and each of the stimulation periods has a duration of at least 10 minutes, e.g., at least 30 minutes, such as at least one hour, and less than 12 hours, e.g., less than six hours, such as less than two hours.

For some applications, low stimulation periods are used in place of “off” periods 802. During these low stimulation periods, the control unit sets the average current applied to be less than 50% of the average current applied during the “on” periods, such as less than 20% or less than 5%. Similarly, for some applications, the control unit is configured to apply a low level of current during the rest periods, rather than no current. For example, the control unit may set the average current applied during the rest periods to be less than 50% of the average current applied during the “on” periods, such as less than 20% or less than 5%. As used in the preset application, including in the claims, the “average current” or “current applied on average” during a given period means the total charge applied during the period (which equals the integral of the current over the period, and may be measured, for example, in coulombs) divided by the duration of the period, such that the average current may be expressed in mA, for example.

For some applications, the copeptin sensor is configured to sense a level of copeptin in blood of the subject. The control unit is configured to set a ratio of (a) an average “on” duration of the “on” periods to (b) an average duration of the low stimulation periods, responsively to the sensed level of copeptin, such that the ratio is positively correlated with the copeptin level. Thus, more stimulation is applied if the copeptin level is higher. For example, to increase the amount of stimulation, the control unit may increase the average “on” duration of the “on” periods, and/or decrease the average duration of the low stimulation periods.

In human experiments conducted by the inventors, it was observed in three subjects that application of continuous intermittent stimulation (i.e., without providing the rest periods described above) for long periods of time (e.g., several hours or several days) caused secondary neuropathic pain projecting along the mandible. Such pain was also observed to commence within several minutes of application of constant stimulation (i.e., non-intermittent stimulation). Providing a rest period of as brief as 30 seconds caused the immediate elimination of this pain. Such pain did not immediately return upon resumption of intermittent stimulation, but did recur after several hours of such stimulation. Providing a longer rest period of several minutes duration once every several hours eliminated this neuropathic pain and prevented its recurrence.

For some applications, these rest period stimulation techniques are combined with the extended PRI techniques described hereinabove with reference to FIG. 37.

In an embodiment, these rest period stimulation techniques are applied to stimulation of nerves other than the vagus nerve.

In an embodiment of the present invention, control unit 120 is configured to apply electrical stimulation to a site, such as the vagus nerve, or one of the other sites described hereinabove, for at least three hours, which at least three hours includes a period having a duration of three hours, which period is divided into a number of equal-duration sub-periods such that each of the sub-periods has a sub-period duration equal to three hours divided by the number of sub-periods, the number between 5 and 10. The control unit configures the stimulation to cause, during at least 20% of each of the sub-periods, an average reduction of at least 5% in a heart rate of the subject compared to a baseline heart rate of the subject. The control unit additionally configures the stimulation to not cause secondary neuropathic pain, such as, by way of non-limiting example, by using one or more techniques described herein. Typically, the control unit additionally configures the stimulation to not cause local pain in a vicinity of the site. For some applications, the control unit configures the stimulation to cause the average reduction during at least 40% of each of the sub-periods. For some applications, the number of sub-periods equals 6 or 9, such that the sub-period duration equals 30 minutes or 20 minutes, respectively.

In an embodiment of the present invention, control unit 120 is configured to apply electrical stimulation to a site, such as a site of the vagus nerve, or another of the sites described hereinabove, for at least three hours, which at least three hours includes a period having a duration of three hours. The control unit configures the stimulation to include at least 3000 pulses during the period, the pulses having on average a pulse duration of at least 0.5 ms (e.g., at least 9 ms), and configures the stimulation to cause, on average during the pulses, at least 3 mA to enter tissue of the vagus nerve. (Depending on the configuration of the electrode device, a portion of the current applied by the device typically does not enter the vagus nerve; the at least 3 mA does not include such current that does not actually enter the vagus nerve.) The control unit additionally configures the stimulation to not cause secondary neuropathic pain, such as, by way of non-limiting example, by using one or more techniques described herein. Typically, the control unit additionally configures the stimulation to not cause local pain in a vicinity of the site. For some applications, the control unit configures the stimulation to cause, on average during the pulses, at least 4 mA to enter the tissue of the vagus nerve.

For some applications, the control unit configures the stimulation to include at least 5000 pulses during the period. For example, if the stimulation were to be applied in a single pulse per second over the three-hour period with a duty cycle of 50% (i.e., the total duration of the “on” periods over the three-hour period equals the total duration of the “off” periods over the three-hour period), a total of 5,400 pulses would be applied (=50%*3 hr*3600 pulses/hr). Without the use of at least one pain reduction technique, such stimulation would generally cause secondary neuropathic pain by the end of the three-hour period. Using techniques described herein, such as, for example, rest periods, a relatively-small portion of the pulses (e.g., up to about 7.5% of the pulses, in this case about 400 of the pulses) are not applied, thereby preventing such secondary neuropathic pain.

In an embodiment of the present invention, control unit 120 is configured to apply the bursts using short “on” periods and, optionally, short “off” periods. Each of the short “on” periods typically has a duration of less than about 10 seconds, e.g., less than about 5 seconds. When short “off” periods are used, each of the “off” periods typically has a duration of between about 5 and about 10 seconds. For example, the “on” periods may have a duration of about 3 seconds, and the “off” periods may have a duration of about 6 seconds. (Stimulation having the configuration described in this paragraph is referred to hereinbelow as “fast intermittent stimulation.”) The use of such short periods generally allows stimulation of any given strength (e.g., as measured by amplitude of the signal, or by PPT of the signal) to be applied as effectively as when using longer “on”/“off” periods, but with fewer potential side effects. In addition, the use of such short “on” periods generally allows side-effect-free application of stimulation at a strength that might increase the risk of side effects if applied for longer “on” periods. It is believed by the inventors that the use of such short periods generally reduces side effects by preventing build-up of sympathetic tone. In general, the parasympathetic reaction to vagal stimulation occurs more quickly than the sympathetic reaction to vagal stimulation. The short “on” periods are sufficiently long to stimulate a desired meaningful parasympathetic reaction, but not sufficiently long to stimulate an undesired, potentially side-effect-causing sympathetic reaction.

For some applications, a desired number of pulses per time period or per heart beat is delivered more effectively and/or with a reduced risk of side effects, by using short “on” periods. For example, assume that it is desired to apply one pulse per trigger. Without the use of short “on” periods, one pulse per trigger could be achieved by applying one PPT constantly. Using short “on” periods, one pulse per trigger could instead be achieved by applying 3 PPT for 3 heart beats (the “on” period), followed by an “off” period of 6 heart beats without stimulation. In both cases, in any given 9-heart-beat period, the same number of pulses (9) are applied. However, the use of short “on” periods generally increases the effectiveness and reduces the potential side effects of the stimulations.

In an embodiment of the present invention, control unit 120 is configured to apply vagal stimulation intermittently using “on”/“off” periods, the durations of which are expressed in heart beats, rather than in units of time. In other words, the control unit alternatingly applies the stimulation for a first number of heart beats, and withholds applying the stimulation for a second number of heart beats. For example, the control unit may alternatingly apply the stimulation for between about 1 and about 30 heart beats, and withhold applying the stimulation for between about 5 and about 300 heart beats. Expressing the duration of the “on”/“off” periods in heart beats results in a constant duty cycle (expressed as “on”/(“on”+“off”)), while expressing the duration in units of time results in a variable duty cycle. In addition, expressing the duration of the “on”/“off” periods in heart beats results in the duration of the “on” and “off” periods varying based on the heart rate (at higher heart rates, the “on” and “off” periods are shorter). Furthermore, expressing the duration of the “on”/“off” periods in heart beats tends to synchronize the stimulation with breathing, which is usually more rapid when the heart rate increases, such as during exercise.

For one particular application, the control unit alternatingly applies the stimulation for exactly one heart beat, and withholds applying the stimulation for exactly one heart beat, i.e., the control unit applies the stimulation every other heart beat. Expressing the duration of “on”/“off” periods in heart beats typically allows precise control of the amount of stimulation applied and the physiological parameter that is being modified, e.g., heart rate.

In an embodiment of the present invention, control unit 120 is configured to apply vagal stimulation intermittently using “on”/“off” periods, the duration of one of which type of periods is expressed in heart beats, and of the other is expressed in units of time. For example, the duration of the “on” periods may be expressed in heart beats (e.g., 2 heart beats), and the duration of the “off” periods may be expressed in seconds (e.g., 2 seconds). In other words, in this example, the control unit alternatingly applies the stimulation for a number of heart beats, and withholds applying the stimulation for a number of seconds. For example, the control unit may alternatingly apply the stimulation for between about 1 and about 100 heart beats, and withhold applying the stimulation for between about 1 and about 100 seconds. Expressing the duration of the “on”/“off” periods in this manner results in an automatic reduction of the duty cycle as the heart rate increases, because, at higher heart rates, more heart beats occur during the “off” periods. As a result, stimulation is automatically reduced at higher rates, which may allow for increased activity and improved quality of life.

In an embodiment of the present invention, control unit 120 operates using feedback, as described hereinabove, and is configured to target a number of pulses applied during each burst of stimulation, responsive to the feedback. Such feedback sometimes results in variations in the average number of pulses per burst. In this embodiment, control unit 120 is configured to monitor the average number of pulses per burst in a given time period. Such monitoring is performed either periodically or substantially continuously. If the average number of pulses per burst exceeds a maximum threshold value over the given time period, the control unit modifies one or more stimulation or feedback parameters, such that the average number of pulses per burst declines below the maximum threshold value. For example, the maximum threshold value may be between about 2 and about 4 pulses per burst, e.g., about 3 pulses per burst. Appropriate parameters for modification include, but are not limited to, (a) one or more of the feedback parameters, such as the target heart rate (e.g., TargetRR), and/or the feedback integral coefficient, and/or (b) one or more stimulation parameters, such as stimulation amplitude, and pulse width, and/or maximum number of pulses within a burst. Alternatively or additionally, for some applications, if the average falls below a minimum threshold value, the control unit modifies one or more stimulation or feedback parameters, such that the average number of pulses per burst increases above the minimum threshold value.

In an embodiment of the present invention, control unit 120 operates using feedback, as described hereinabove, which results in a variable number of bursts per heart beat and/or per unit time. (For example, a burst may be applied every 1-60 heart beats, or every 0.3-60 seconds, as dictated by a feedback algorithm.) Such feedback sometimes results in high- and/or low-frequency variations in the duty cycle. Control unit 120 is configured to monitor the average duty cycle in a given time period. Such monitoring is performed either periodically or substantially continuously. If the average exceeds a maximum threshold value, the control unit modifies one or more stimulation or feedback parameters, such that the average duty cycle declines below the maximum threshold value. Appropriate parameters for modification include, but are not limited to, the target heart rate (e.g., TargetRR), the feedback integral coefficient, stimulation amplitude, pulse width, and maximum number of pulses within a burst. Alternatively or additionally, for some applications, if the average falls below a minimum threshold value, the control unit modifies one or more stimulation or feedback parameters, such that the average duty cycle increases above the maximum threshold value. For some applications, control unit 120 implements the techniques of this embodiment in combination with the techniques for monitoring the average number of pulses per burst described above.

In an embodiment of the present invention, control unit 120 is configured to gradually ramp the commencement and/or termination of stimulation. In order to achieve the gradual ramp, the control unit is typically configured to gradually modify one or more stimulation parameters, such as those described hereinabove, e.g., pulse amplitude, number of pulses, PPT, pulse frequency, pulse width, “on” time, and/or “off” time. Terminating stimulation gradually, rather than suddenly, may reduce the likelihood of a rebound acceleration of heart rate that sometimes occurs upon termination of vagal stimulation. As appropriate, one or more of these parameters is varied by less than 50% of the pre-termination value per heart beat, or less than 5% per heart beat, in order to achieve the gradual ramp.

In an embodiment of the present invention, control unit 120 is configured to gradually increase the strength of stimulation according to a predetermined schedule. Such a gradual increase is typically appropriate during the first several days of use of system 118 by a new subject. Subjects sometimes experience discomfort and/or pain during their initial exposure to stimulation. Such discomfort and/or pain typically ceases after an accommodation period of several days. By gradually increasing stimulation from an initially low level, control unit 120 generally prevents such discomfort and/or pain. For example, the strength of stimulation may be increased less than 50% per hour, or less than 10% per day. The control unit is typically configured to increase the strength of stimulation by adjusting one or more stimulation parameters, such as those described hereinabove, e.g., the amplitude of the applied signal.

For some applications, system 118 is configured to allow the subject to manually control the ramp-up of stimulation, e.g., by selecting when the system proceeds to successive levels of stimulation, and/or by requesting the system to return to a previous level of stimulation.

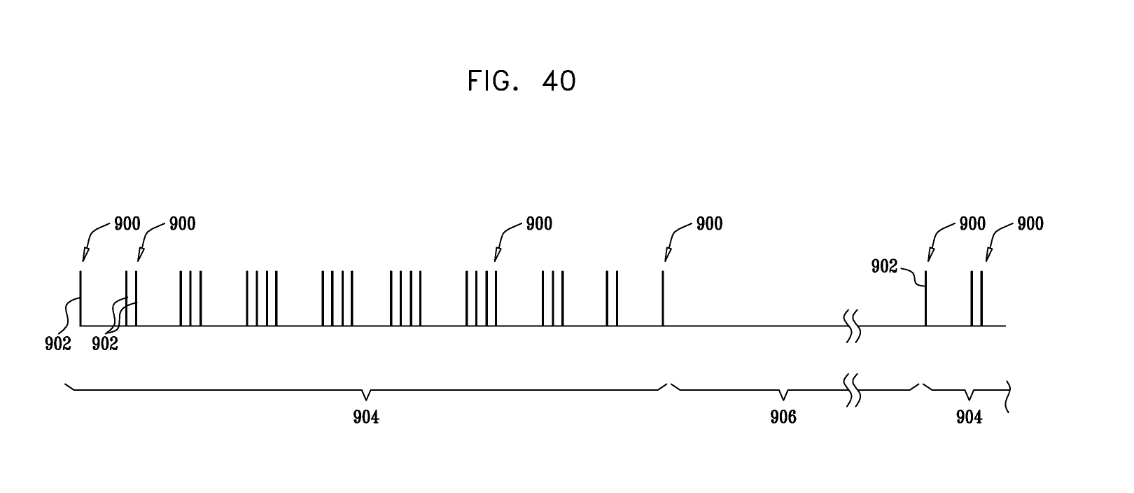

Reference is made to FIG. 40, which is a schematic illustration of a stimulation regimen, in accordance with an embodiment of the present invention. In this embodiment, control unit 120 is configured to apply vagal stimulation in a series of bursts 900, each of which includes one or more pulses 902 (pulses per trigger, or PPT). The control unit is configured to apply the vagal stimulation intermittently during “on” periods 904 alternating with “off” periods 906, during which no stimulation is applied. Each “on” period 904 includes at least 3 bursts 900, such as at least 10 bursts 900, and typically has a duration of between 3 and 20 seconds. At the commencement of each “on” period 904, control unit 120 ramps up the PPT of successive bursts 900, and at the conclusion of each “on” period 904, the control unit ramps down the PPT of successive bursts 900. For example, the first four bursts of an “on” period 904 may have respective PPTs of 1, 2, 3, and 3, or 1, 2, 3, and 4, and the last four bursts of an “on” period 904 may have respective PPTs of 3, 3, 2, and 1, or 4, 3, 2, and 1. Use of such ramping generally prevents or reduces sudden drops and rebounds in heart rate at the beginning and end of each “on” period, respectively. Experimental results are described hereinbelow with reference to FIG. 9 which illustrate the occurrence of such sudden drops and rebounds without the use of the ramping techniques of this embodiment.

Alternatively, rather than increase or decrease the PPT by 1 in successive bursts, control unit 120 increases or decreases the PPT more gradually, such as by 1 in less than every successive burst, e.g., the first bursts of an “on” period may have respective PPTs of 1, 1, 2, 2, 3, 3, and 4, and the last bursts of an “on” period may have respective PPTs of 4, 3, 3, 2, 2, 1, and 1. For some applications, to increase or decrease the PPT by less than 1 in successive bursts, the control unit increases or decreases the PPT by non-integer values, and achieves the non-integer portion of the increase or decrease by setting a parameter of one or more pulses other than PPT, such as pulse duration or amplitude. For example, the first bursts of an “on” period may have respective PPTs of 0.5, 1, 1.5, 2, 2.5, and 3, and the last bursts of an “on” period may have respective PPTs of 3, 2.5, 2, 1.5, 1, and 0.5. To achieve the decimal portion of these PPTs, the control unit may apply a pulse having a pulse duration equal to the decimal portion of these PPTs times the pulse duration of a full pulse. For example, if the pulse duration of a full pulse is 1 ms, a commencement ramp of 0.5, 1, and 1.5 PPT may be achieved by applying a first burst consisting of a single 0.5 ms pulse, a second burst consisting of a single 1 ms pulse, and a third burst consisting of a 1 ms pulse followed by a 0.5 ms pulse. Alternatively, to achieve the decimal portion of these PPTs, the control unit may apply a pulse having a full pulse duration but an amplitude equal to the decimal portion of these PPTs times the amplitude of a full pulse. For example, if the pulse duration and amplitude of a full pulse if 1 ms and 3 mA, respectively, a commencement ramp of 0.5, 1, and 1.5 PPT may be achieved by apply a first burst consisting of a single 1 ms pulse having an amplitude of 1.5 mA, a second burst consisting of a single 1 ms, 3 mA pulse, and a third burst consisting of a 1 ms, 3 mA followed by a 1 ms pulse having an amplitude of 1.5 mA.

For some applications, control unit 120 is configured to synchronize the bursts with a feature of the cardiac cycle of the subject. For example, each of the bursts may commence after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity. For some applications, such ramping is applied only at the commencement of each “on” period 904, or only at the conclusion of each “on” period 904, rather than during both transitional periods.

For some applications, such ramping techniques are combined with the extended PRI techniques described hereinabove with reference to FIG. 37, and/or with the rest period techniques described hereinabove with reference to FIG. 39.

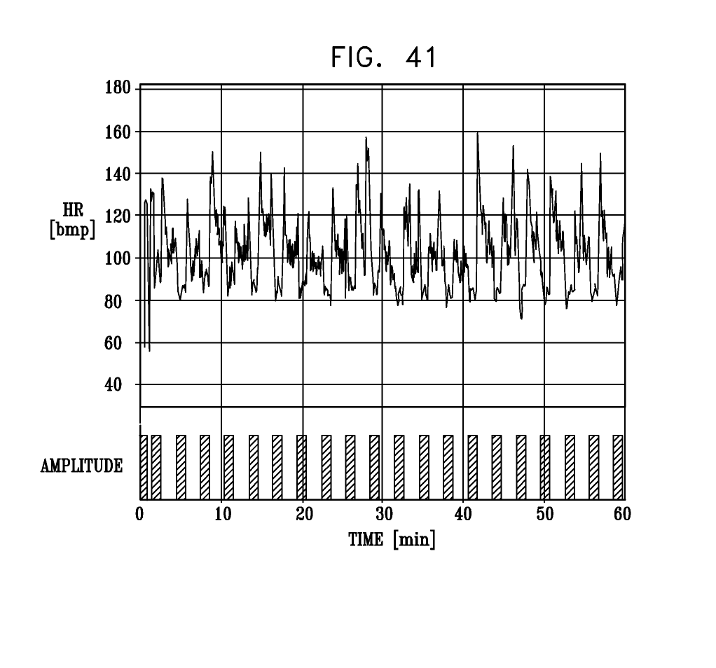

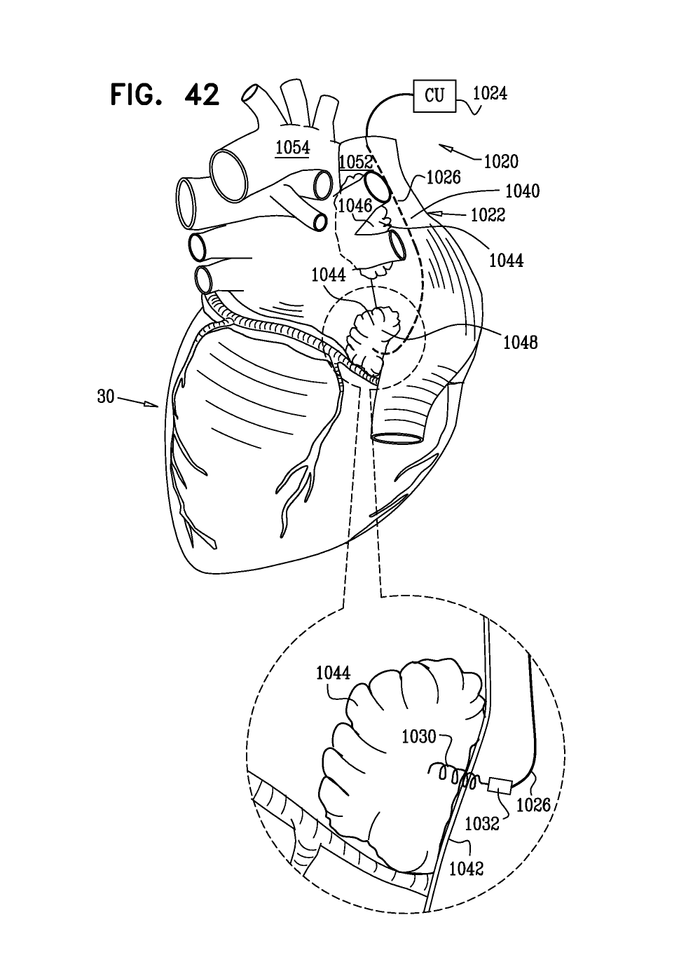

Reference is made to FIG. 41, which is a graph showing experimental results obtained in an animal experiment, in accordance with an embodiment of the present invention. Vagal stimulation was applied to a dog in bursts of pulses during one-minute “on” periods that alternated with two-minute “off” periods. Each of the bursts had a constant PPT of 6, i.e., the stimulation was not ramped, as described hereinabove with reference to FIG. 40. As can be seen in the graph, upon initiation of each “on” period, there was a sudden and strong drop in heart rate, and immediately after the conclusion of each “on” period, there was a strong rebound in heart rate. Such abrupt drops and rebounds are particularly undesirable in patients suffering from heart disease, for whom the abrupt decreases in heart rate may cause a drop in blood pressure, and the abrupt accelerations in heart rate may cause a sensation of palpitation, or increase the risk of arrhythmia.

In an embodiment, these techniques for gradually increasing and/or decreasing the strength of stimulation are applied to stimulation of nerves other than the vagus nerve.

In an embodiment of the present invention, for applications in which control unit 120 is configured to apply vagal stimulation intermittently, as described hereinabove, the control unit begins the stimulation with an “off” period, rather than with an “on” period. As a result, a delay having the duration of an “off” period occurs prior to beginning stimulation. Alternatively or additionally, whether or not configured to apply stimulation intermittently, control unit 120 is configured to delay beginning the application of stimulation for a certain time period (e.g., a pseudo-randomly determined time period, or a predetermined fixed period of time, such as about 5 seconds) after receiving an external command to apply the stimulation. The use of these delaying techniques generally reduces a subject's anticipation of any pain or discomfort that he may associate with stimulation, and disassociates the sensations of stimulation from the physician and/or an external control device such as a wand.

For some applications, the intermittent vagal stimulation is applied with “on” periods having a duration of between about 45 and about 75 seconds each, e.g., about 1 minute each, and “off” periods having a duration of between about 90 and about 150 seconds each, e.g., about 2 minutes each. Alternatively or additionally, the intermittent vagal stimulation is applied with “off” periods having a duration of between about 1.2 and about 3.5 times greater than the “on” periods, e.g., between about 1.5 and about 2.5 times greater than the “on” periods. In order to include the plurality of different naturally-occurring heart rates, the calibration period typically includes at least several hundred “on” and “off” periods. For example, the calibration period may be about 24 hours. Alternatively, the calibration period is shorter, and includes sub-periods of rest, exercise, and recovery from exercise, in order to ensure the inclusion of the plurality of different naturally-occurring heart rates. For example, for at least part of the calibration period the subject may be subjected to an exercise test (e.g., a stress test), such as by using exercise equipment, e.g., a treadmill.

Although embodiments of the present invention are described herein, in some cases, with respect to treating specific heart conditions, it is to be understood that the scope of the present invention generally includes utilizing the techniques described herein to controllably stimulate the vagus nerve to facilitate treatments of, for example, heart failure, atrial fibrillation, and ischemic heart diseases. In particular, the techniques described herein may be performed in combination with other techniques, which are well known in the art or which are described in the references cited herein, that stimulate the vagus nerve in order to achieve a desired therapeutic end.

Although some embodiments of the present invention have been described herein with respect to applying stimulation to parasympathetic autonomic nervous tissue, it is to be understood that the scope of the present invention generally includes utilizing the techniques described herein to apply stimulation to any tissue, such as nervous tissue, muscle tissue, or sensory receptors. For example, the stimulation techniques described herein may be used to stimulate secretion by a gland, such as insulin secretion by the pancreas, or adrenalin by the adrenal gland. For these applications, the stimulation techniques described herein generally maximize the desired effect of stimulation, while generally minimizing any adverse pain, discomfort, or damage that may be caused by the stimulation. For some applications, the stimulation techniques described herein may be used to stimulate sensory receptors (such as coetaneous cold or stretch receptors), in order to activate sensory gateways for chronic pain reduction substantially without inducing pain.

For some applications, stimulation techniques described herein may be used to stimulate a nerve such as the ulnar nerve, in order to cause muscle activity, while minimizing any associated adverse pain, discomfort, or damage that may be caused by the stimulation. For some applications, stimulation techniques described herein may be used to stimulate a sensory nerve, such as the ophthalmic branch of the trigeminal nerve, to induce painless neuromodulation, such as for the treatment of epilepsy, or other disorders treatable by nerve stimulation.

For some applications, stimulation techniques described herein may be used to stimulate skeletal muscles, such as in order to train the muscle, to improve the muscle tone or gait of the subject, or to burn calories, while minimizing any adverse pain, discomfort, or damage that may be caused by such stimulation. For some applications, stimulation techniques described herein may be used to stimulate the detrusor muscle, in order to control urinary symptoms, while minimizing any adverse pain, discomfort, or damage that may be caused by such stimulation.

For some applications, techniques described herein are used to apply controlled stimulation to one or more of the following: the lacrimal nerve, the salivary nerve, the vagus nerve, the pelvic splanchnic nerve, or one or more sympathetic or parasympathetic autonomic nerves. Such controlled stimulation may be applied to such nerves directly, or indirectly, such as by stimulating an adjacent blood vessel or space. Such controlled stimulation may be used, for example, to regulate or treat a condition of the lung, heart, stomach, pancreas, small intestine, liver, spleen, kidney, bladder, rectum, large intestine, reproductive organs, or adrenal gland.

As appropriate, techniques described herein are practiced in conjunction with methods and apparatus described in one or more of the following patent applications, all of which are assigned to the assignee of the present application and are incorporated herein by reference:
U.S. patent application Ser. No. 10/205,474, filed Jul. 24, 2002, entitled, “Electrode assembly for nerve control,” which published as US Patent Publication 2003/0050677
U.S. Provisional Patent Application 60/383,157 to Ayal et al., filed May 23, 2002, entitled, “Inverse recruitment for autonomic nerve systems”
U.S. patent application Ser. No. 10/205,475, filed Jul. 24, 2002, entitled, “Selective nerve fiber stimulation for treating heart conditions,” which published as US Patent Publication 2003/0045909
PCT Patent Application PCT/IL02/00068, filed Jan. 23, 2002, entitled, “Treatment of disorders by unidirectional nerve stimulation,” which published as PCT Publication WO 03/018113, and U.S. Patent application Ser. No. 10/488,334, filed Feb. 27, 2004, in the US National Phase thereof, which issued as U.S. Pat. No. 7,734,355
U.S. patent application Ser. No. 09/944,913, filed Aug. 31, 2001, entitled, “Treatment of disorders by unidirectional nerve stimulation,” which issued as U.S. Pat. No. 6,684,105
U.S. patent application Ser. No. 10/461,696, filed Jun. 13, 2003, entitled, “Vagal stimulation for anti-embolic therapy,” which issued as U.S. Pat. No. 7,321,793
PCT Patent Application PCT/IL03/00430, filed May 23, 2003, entitled, “Electrode assembly for nerve control,” which published as PCT Publication WO 03/099373
PCT Patent Application PCT/IL03/00431, filed May 23, 2003, entitled, “Selective nerve fiber stimulation for treating heart conditions,” which published as PCT Publication WO 03/099377
U.S. patent application Ser. No. 10/719,659, filed Nov. 20, 2003, entitled, “Selective nerve fiber stimulation for treating heart conditions,” which issued as U.S. Pat. No. 7,778,711
PCT Patent Application PCT/IL04/00440, filed May 23, 2004, entitled, “Selective nerve fiber stimulation for treating heart conditions,” which published as PCT Publication WO 04/103455
PCT Patent Application PCT/IL04/000496, filed Jun. 10, 2004, entitled, “Vagal stimulation for anti-embolic therapy,” which published as PCT Publication WO 04/110550
U.S. patent application Ser. No. 10/866,601, filed Jun. 10, 2004, entitled, “Applications of vagal stimulation,” which published as US Patent Application Publication 2005/0065553
PCT Patent Application PCT/IL04/000495, filed Jun. 10, 2004, entitled, “Applications of vagal stimulation,” which published as PCT Publication WO 04/110549
U.S. patent application Ser. No. 11/022,011, filed Dec. 22, 2004, entitled, “Construction of electrode assembly for nerve control,” which published as US Patent Application Publication 2006/0136024
U.S. patent application Ser. No. 11/062,324, filed Feb. 18, 2005, entitled, “Techniques for applying, calibrating, and controlling nerve fiber stimulation,” which published as US Patent Application Publication 2005/0197675
U.S. patent application Ser. No. 11/064,446, filed Feb. 22, 2005, entitled, “Techniques for applying, configuring, and coordinating nerve fiber stimulation,” which published as US Patent Application Publication 2005/0267542
U.S. patent application Ser. No. 11/280,884, filed Nov. 15, 2005, entitled, “Techniques for nerve stimulation,” which published as US Patent Application Publication 2006/0106441
U.S. patent application Ser. No. 11/340,156, filed Jan. 25, 2006, entitled, “Method to enhance progenitor or genetically-modified cell therapy,” which published as US Patent Application Publication 2006/0167501
U.S. patent application Ser. No. 11/359,266, filed Feb. 21, 2006, entitled, “Parasympathetic pacing therapy during and following a medical procedure, clinical trauma or pathology,” which published as US Patent Application Publication 2006/0206155

It is noted that in many embodiments of the present invention, durations of various stimulation and non-stimulation periods are specified, either as actual values or ranges of actual values, or in relation to durations of other periods. It is to be understood that occasional deviations from such durations during application of stimulation are within the scope of the present invention, so long as on average the parameters of the stimulation meet the specified parameters. “Average,” as used herein, including in the claims, is to be understood as meaning an arithmetic mean.

It will be appreciated by persons skilled in the art that current application techniques described herein may be appropriate for application to additional nerves or tissues, such as, for example, cardiac tissue. In addition, techniques described herein may be appropriate for implementation in pacemakers and/or ICDs, mutatis mutandis. For example, techniques described herein for configuring and/or regulating the application of an electrical current may be performed, mutatis mutandis, for applying pacing pulses or anti-arrhythmic energy to the heart.

FIG. 42 is a schematic illustration of a parasympathetic stimulation system 1020 for stimulating autonomic nervous tissue from at least partially within a heart 30, in accordance with an embodiment of the present invention. System 1020 comprises at least one electrode assembly 1022, which is applied to a cardiac site containing parasympathetic nervous tissue, such as an atrial site, and an implantable or external control unit 1024. Electrode assembly 1022 comprises a lead 1026 coupled to one or more electrode contacts 1030 and 1032. Lead 1026 is typically introduced into the heart using an introducer, such as a catheter or sheath.

In an embodiment of the present invention, electrode contacts 1030 and 1032 are configured to be implanted in a right atrium 1040, typically in contact with atrial muscle tissue 1042 in a vicinity of a parasympathetic epicardial fat pad 1044. For some applications, electrode contacts 1030 and 1032 are fixed within atrium 40 using active fixation techniques. For some applications, parasympathetic epicardial fat pad 1044 comprises a sinoatrial (SA) fat pad 1046, while for other applications, parasympathetic epicardial fat pad 1044 comprises an atrioventricular (AV) fat pad 1048. For still other applications, the parasympathetic epicardial fat pad comprises an SVC-AO fat pad located in a vicinity of a junction between a superior vena cava 1052 and an aorta 1054 (SVC-AO fat pad not shown in figure). Alternatively, separate electrode assemblies 1022, or separate electrode contacts of a single electrode assembly 1022, are implanted in the vicinity of both SA node fat pad 1046 and AV node fat pad 1048, for activating both fat pads, such as described hereinbelow.

In an embodiment of the present invention, control unit 1024 applies the parasympathetic stimulation responsively to one or more sensed parameters. Control unit 1024 is typically configured to commence or halt stimulation, or to vary the amount and/or timing of stimulation in order to achieve a desired target heart rate, typically responsively to configuration values and on parameters including one or more of the following:
Heart rate—the control unit can be configured to drive electrode assembly 1022 to stimulate the fat pad(s) only when the heart rate exceeds a certain value.
ECG readings—the control unit can be configured to drive electrode assembly 1022 to stimulate the fat pad(s) responsively to certain ECG readings, such as readings indicative of designated forms of arrhythmia. Additionally, ECG readings are typically used for achieving a desire heart rate.
Blood pressure—the control unit can be configured to regulate the current applied by electrode assembly 1022 to the fat pad(s) when blood pressure exceeds a certain threshold or falls below a certain threshold.
Indicators of decreased cardiac contractility—these indicators include left ventricular pressure (LVP). When LVP and/or d(LVP)/dt exceeds a certain threshold or falls below a certain threshold, control unit 1024 can drive electrode assembly 1022 to regulate the current applied by electrode assembly 1022 to the fat pad(s).
Motion of the subject—the control unit can be configured to interpret motion of the subject as an indicator of increased exertion by the subject, and appropriately reduce parasympathetic stimulation of the heart in order to allow the heart to naturally increase its rate.
Heart rate variability—the control unit can be configured to drive electrode assembly 1022 to stimulate the fat pad(s) responsively to heart rate variability, which is typically calculated responsively to certain ECG readings.
Norepinephrine concentration—the control unit can be configured to drive electrode assembly 1022 to stimulate the fat pad(s) responsively to norepinephrine concentration.
Cardiac output—the control unit can be configured to drive electrode assembly 1022 to stimulate the fat pad(s) responsively to cardiac output, which is typically determined using impedance cardiography.
Baroreflex sensitivity—the control unit can be configured to drive electrode assembly 1022 to stimulate the fat pad(s) responsively to baroreflex sensitivity.
LVEDP—the control unit can be configured to drive electrode assembly 1022 to stimulate the fat pad(s) responsively to LVEDP, which is typically determined using a pressure gauge.

The parameters and behaviors included in this list are for illustrative purposes only, and other possible parameters and/or behaviors will readily present themselves to those skilled in the art, who have read the disclosure of the present patent application.

In an embodiment of the present invention, control unit 1024 is configured to drive electrode assembly 1022 to stimulate the fat pad(s) so as to reduce the heart rate of the subject towards a target heart rate. The target heart rate is typically (a) programmable or configurable, (b) determined responsive to one or more sensed physiological values, such as those described hereinabove (e.g., motion, blood pressure, etc.), and/or (c) determined responsive to a time of day or circadian cycle of the subject. Parameters of stimulation are varied in real time in order to vary the heart-rate-lowering effects of the stimulation. For example, such parameters may include the amplitude of the applied current. Alternatively or additionally, in an embodiment of the present invention, the stimulation is applied in bursts (i.e., series of pulses), which are synchronized or are not synchronized with the cardiac cycle of the subject.

In some embodiments of the present invention, the control unit senses an ECG and/or a local cardiac electrogram, and applies the vagomimetic stimulation during the atrial effective refractory period (AERP) or local refractory period. The stimulation thus causes a vagomimetic effect without causing cardiac capture.

Typical parameters include the following:
Timing of the stimulation within the cardiac cycle. Delivery of each of the bursts typically begins after a fixed or variable delay following an ECG feature, such as each R- or P-wave. For some applications, the delay is between about 0 and about 100 ms after the P-wave.
Pulse duration (width). Longer pulse durations typically result in a greater heart-rate-lowering effect. For some applications, the pulse duration is between about 0.001 ms and about 5 ms, such as between about 0.1 ms and about 2 ms, e.g., about 0.5 ms.
Pulse repetition interval within each burst. Maintaining a pulse repetition interval (the time from the initiation of a pulse to the initiation of the following pulse within the same burst) greater than about 1 ms generally results in better stimulation effectiveness for multiple pulses within a burst. For some applications, the pulse repetition interval is between about 1 and about 20 ms, such as between about 3 and about 10 ms, e.g., about 6 ms.
Pulses per trigger (PPT). A greater PPT (the number of pulses in each burst after a trigger such as an P-wave) typically results in a greater heart-rate-lowering effect. For some applications, PPT is between about 1 and about 20 pulses, such as between about 2 and about 10 pulses, e.g., 5 pulses. For some applications, PPT is varied while pulse repetition interval is kept constant.
Amplitude. A greater amplitude of the signal applied typically results in a greater heart-rate-lowering effect. The amplitude is typically between about 0.1 and about 10 milliamps, e.g., between about 1 and about 3 milliamps, such as about 2 milliamps.
Duty cycle (number of bursts per heart beat). Application of stimulation every heartbeat (i.e., with a duty cycle of 1) typically results in a greater heart-rate-lowering effect. For less heart rate reduction, stimulation is applied less frequently than every heartbeat (e.g., duty cycle=60%-90%), or only once every several heartbeats (e.g., duty cycle=5%-40%).
“On”/“off” ratio and timing. For some applications, the device operates intermittently, alternating between “on” and “off” states, the length of each state typically being between 0 and about 1 day, such as between 0 and about 300 seconds (with a O-length “off” state equivalent to always “on”). No stimulation is applied during the “off” state. Greater heart rate reduction is typically achieved if the device is “on” a greater portion of the time.

In an embodiment of the present invention, control unit 1024 sets the frequency (pulses per second), amplitude, and pulse width of the signal such that the product thereof is less than 12 Hz*mA*ms, e.g., less than 6 Hz*mA*ms. Application of such a signal typically reduces the heart rate by at least 10%, e.g., at least 20%, compared to a baseline heart rate of the subject in the absence of the stimulation. It is noted that the frequency is measured in pulses per second, even for applications in which the pulses are applied in bursts, such that the pulses are not evenly distributed throughout any given second.

For some applications, values of one or more of the parameters are determined in real time, using feedback, i.e., responsive to one or more inputs, such as sensed physiological values. For example, the intermittency (“on”/“off”) parameter may be determined in real time using such feedback. The inputs used for such feedback typically include one or more of the following: (a) motion or activity of the subject (e.g., detected using an accelerometer), (b) the average heart rate of the subject, (c) the average heart rate of the subject when the device is in “off” mode, (d) the average heart rate of the subject when the device is in “on” mode, and/or (e) the time of day. The average heart rate is typically calculated over a period of at least about 10 seconds. For some applications, the average heart rate during an “on” or “off” period is calculated over the entire “on” or “off” period. For example, the device may operate in continuous “on” mode when the subject is exercising and therefore has a high heart rate, and the device may alternate between “on” and “off” when the subject is at rest. As a result, the heart-rate-lowering effect is concentrated during periods of high heart rate, and the nerve is allowed to rest when the heart rate is generally naturally lower. For some applications, the device determines the ratio of “on” to “off” durations, the duration of the “on” periods, and/or the durations of the “off” periods using feedback. Optionally, the device determines the “on”/“off” parameter in real time using the integral feedback techniques described in U.S. application Ser. No. 11/064,446, filed Feb. 22, 2005, which issued as U.S. Pat. No. 7,974,693 and is assigned to the assignee of the present application and is incorporated herein by reference, mutatis mutandis.

For some applications, heart rate regulation is achieved by setting two or more parameters in combination. For example, if it is desired to apply 5.2 pulses of stimulation, the control unit may apply 5 pulses of 1 ms duration each, followed by a single pulse of 0.2 ms duration. For other applications, the control unit switches between two values of PPT, so that the desired PPT is achieved by averaging the applied PPTs. For example, a sequence of PPTs may be 5, 5, 5, 5, 6, 5, 5, 5, 5, 6, . . . , in order to achieve an effective PPT of 5.2.

In an embodiment of the present invention, control unit 1024 is configured to apply the parasympathetic stimulation using feedback, as described hereinabove, wherein a parameter of the feedback is a target heart rate that is a function of an average heart rate of the subject. For some applications, the target heart rate is set equal or approximately equal to the average heart rate of the subject. Alternatively, the target heart rate is set at a rate greater than the average heart rate of the subject, such as a number of beats per minute (BPM) greater than the average heart rate, or a percentage greater than the average heart rate, e.g., about 1% to about 50% greater. Further alternatively, the target heart rate is set at a rate less than the average heart rate of the subject, such as a number of BPM less than the average heart rate, or a percentage less than the average heart rate, e.g., about 1% to about 20% less. For some applications, the target heart rate is set responsively to the duty cycle and the heart rate response of the subject. In an embodiment, control unit 1024 determines the target heart rate in real time, periodically or substantially continuously, by sensing the heart rate of the subject and calculating the average heart rate of the subject. The average heart rate is typically calculated substantially continuously, or periodically. Typically, standard techniques are used for calculating the average, such as moving averages or IIR filters. The number of beats that are averaged typically varies between several beats to all beats during the past week.

For some applications, the techniques described herein are performed in combination with techniques described in above-mentioned U.S. application Ser. No. 11/064,446. In particular, control unit 1024 may use feedback and parameter-setting techniques described therein.

In some embodiments of the present invention, control unit 1024 is configured to test for cardiac capture, and to modify one or more stimulation parameters so as to reduce the probability of cardiac capture. In these embodiments, the stimulation is typically intended to cause parasympathetic stimulation, and causing cardiac capture (i.e., pacing) is thus undesired. For some applications in which the control unit applies one burst per cardiac cycle, the control unit tests for cardiac capture by sensing the ECG signal after completion of each burst within a cardiac cycle, either immediately after completion or after a short blanking period (e.g., less than about 60 ms, such as less than about 30 ms). If the control unit detects additional atrial depolarization waves, the control unit determines that unwanted capture has occurred, and modulates the stimulation accordingly. Typically, to modulate the stimulation, the control unit first withholds stimulation for at least one cardiac cycle (e.g., for one, two, or three cardiac cycles), and then performs stimulation again after re-detecting a P wave. If the control finds that such withholding does not prevent unwanted capture over time, the control unit changes one or more stimulation parameters, typically staging the changes until the disappearance of the capture. For some applications, the control unit first shortens the stimulation duration, such as reduces the burst duration (e.g., from about 80 ms to about 60 ms, and then even lower), until the desired disappearance of capture is achieved. If reduction of the burst duration is insufficient, the control unit then reduces the stimulation current, such as from about 10 mA to about 8 mA, and then even lower, until the desired disappearance of capture is achieved. If neither of these reductions is sufficient, the control unit may reduce the pulse width, such as from about 1 ms to about 0.3 ms.

In some embodiments of the present invention, control unit 1024 senses the cardiac electrogram using a monopolar or bipolar electrode configuration. For some applications, the sensing is bipolar, and the electrode is positioned so as to sense atrial depolarization more strongly than ventricular depolarization.

In an embodiment of the present invention, control unit 1024 is configured to apply respective bursts of pulses in a plurality of cardiac cycles, and to set a strength of the stimulation to be sufficient to generate a vagomimetic response, but insufficient to cause cardiac contraction (and typically insufficient to generate propagating action potentials in the myocardium of the subject). The control unit configures one or more parameters of the stimulation to set a strength thereof (e.g., current, number of pulses per burst, and/or pulse width). For some applications, the bursts are synchronized with a feature of the cardiac cycle, while for other applications, the bursts are not synchronized with a feature of the cardiac cycle.

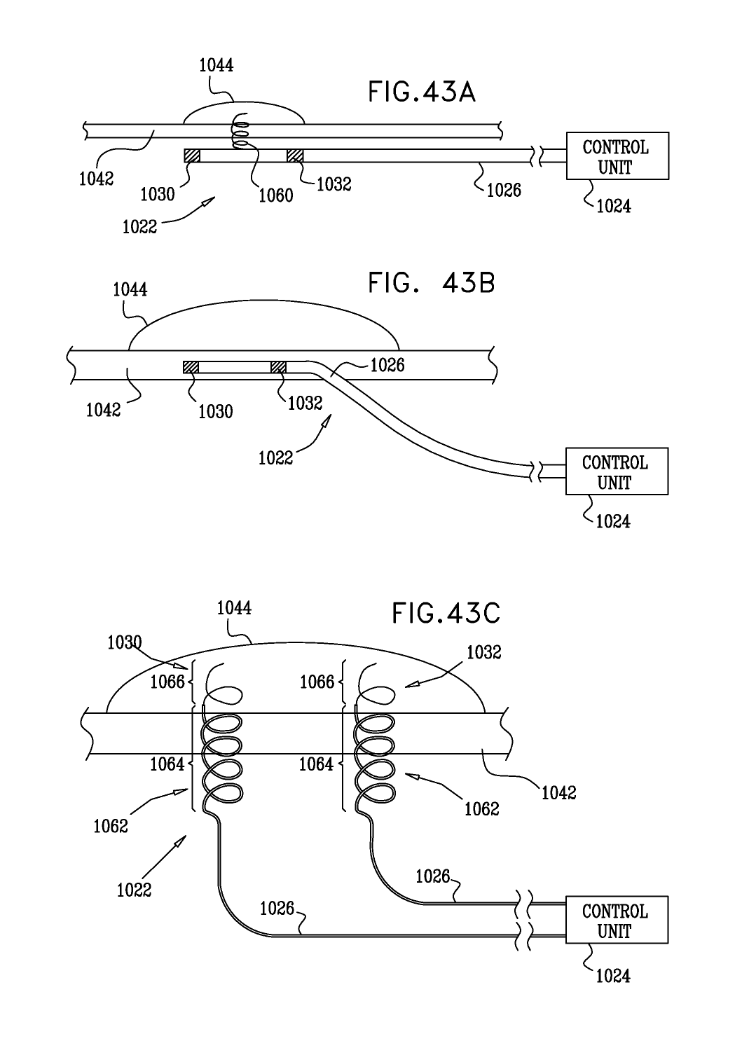

FIGS. 43A-C are schematic illustrations of configurations of electrode assembly 1022, in accordance with respective embodiments of the present invention. In the configuration shown in FIG. 43A, both electrode contacts 1030 and 1032 are configured to be in contact with muscle tissue 1042, or to be at approximately equal distances from the tissue. For some applications, lead 1026 comprises at least one fixation element 1060, such as a screw-in fixation element, positioned along the lead between electrode contacts 1030 and 1032, so as to hold the lead in place with the electrode contacts both in contact with the tissue, or at approximately the same distance therefrom. Alternatively or additionally, the lead comprises a plurality of fixation elements in respective vicinities of the electrode contacts. As used in the present application, including in the claims, “screw-in fixation elements” include, but are not limited to, fixation elements that are shaped as screws, corkscrews, or helices.

In the configuration shown in FIG. 43B, lead 1026 penetrates muscle tissue 1042, such that both electrode contacts 1030 and 1032 penetrate the muscle tissue in a vicinity of fat pad 1044, e.g., in contact therewith or within several millimeters therefrom.

In the configuration shown in FIG. 43C, electrode assembly 1022 comprises a rotational-engagement fixation element 1062, typically a screw-in fixation element. For some applications, fixation element 1062 is sized such that its proximal end extends to the surface of the atrial wall when fully implanted, as shown in FIG. 43C, while for other applications, the fixation mechanism is shorter, such that its proximal end does not reach the surface of the atrial wall when fully implanted, but instead terminates inside the atrial wall. The surface of a proximal portion 1064 of fixation element 1062 is electrically insulated, e.g., comprises a non-conductive coating, such as Teflon or silicone, around a conductive core. A distal portion 1066 of the fixation element is conductive, and serves as electrode contact 1030 or electrode contact 1032. As shown, proximal and distal portions 1064 and 1066 are coaxial, and the conductive core of proximal portion 1064 is continuous with distal portion 1066. Alternatively, electrode assembly 1022 comprises another, non-rotational fixation element. For example, the fixation element may comprise straight electrode contacts, or flexible electrode contacts inserted via a sheath that is later withdrawn.

Insulated portion 1064 is configured to be chronically disposed at least partially within atrial muscle tissue 1042, and electrode contacts 1030 and 1032 are configured to be chronically disposed in contact with parasympathetic epicardial fat pad 1044, typically within the fat pad. Optionally, a portion of insulated portion 1064 penetrates into fat pad 1044. Typically, but not necessarily, electrode contacts 1030 and 1032 are positioned entirely within the fat pad, such that no portion of the electrode contacts are in contact with atrial muscle tissue 1042. A length of insulated portion 1064 is typically greater than a thickness of the atrial wall, e.g., at least 1 mm or at least 2 mm. Avoidance of direct application of current to atrial muscle tissue 1042 generally decreases the risk of undesired cardiac capture.

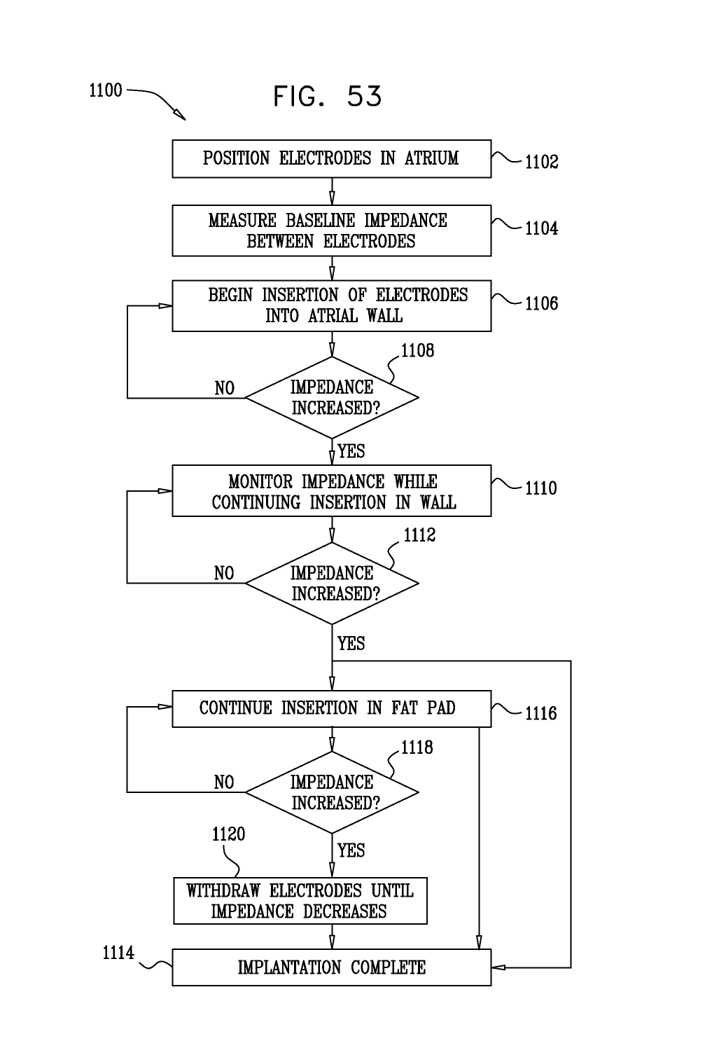

During implantation of the electrode assembly shown in FIG. 43C, distal portions of electrode contacts 1030 and 1032 are advanced entirely through and out the outward site of the cardiac muscle tissue of the atrial wall. The distal tips of electrode contacts 1030 and 1032 are typically positioned in fat pad 1044. For some applications, during an implantation procedure, a check is performed to confirm that the distal portions of the electrode contacts have passed entirely through the cardiac muscle tissue, that the distal tips of the electrode contacts have entered the fat pad, and/or that the distal tips of the electrode contacts have not passed entirely through the fat pad and into the pericardial space. For some applications, techniques for monitoring such accurate positioning are used that are described hereinbelow with reference to FIG. 53. Typically, the distal tips of the electrode contacts are left in position outside the cardiac muscle of the atrial wall for at least one week. For some applications, electrode contacts 1030 and 1032 are inserted through atrial muscle tissue 1042 until they are brought essentially entirely within fat pad 1044. Thus the electrode contacts are positioned entirely within the fat pad, and outside the cardiac muscle.

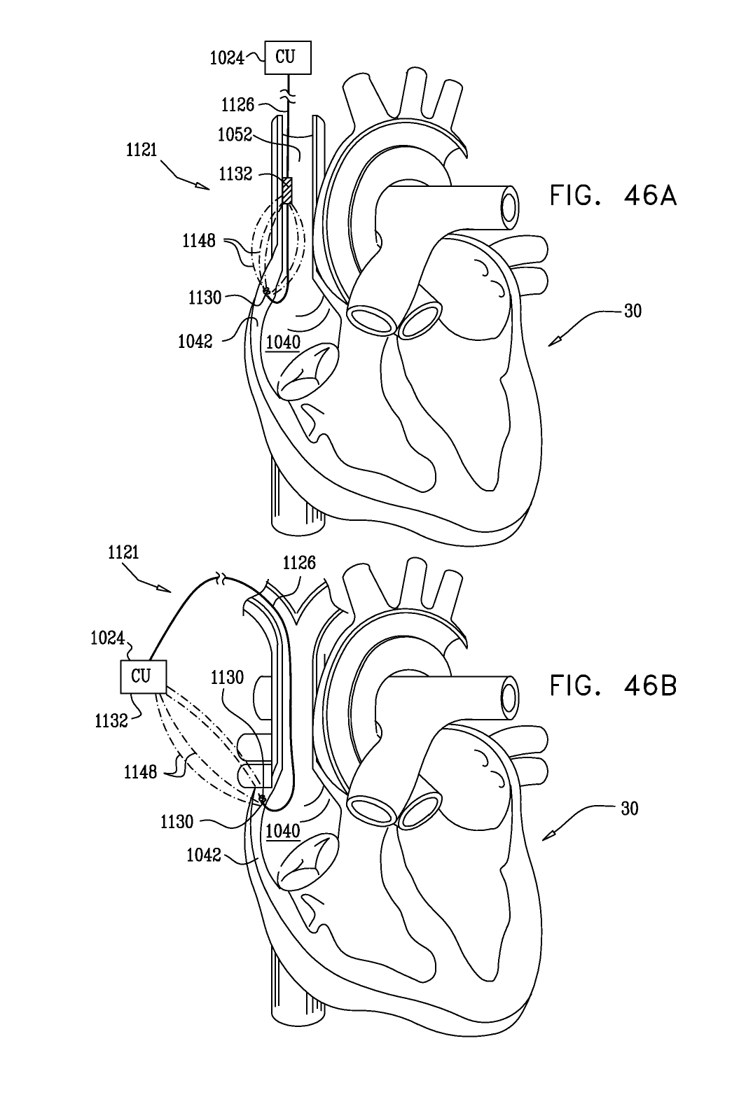

It is noted that although FIGS. 43A-C show two electrode contacts placed in the vicinity of fat pad 1044 (e.g., acting as a cathode and an anode), the scope of the present invention includes using three or more electrode contacts placed in the vicinity of the fat pad (e.g., an anode between two cathodes, or a cathode between two anodes), or using one or more electrode contacts (e.g., one or more cathodes) placed in the vicinity, and another electrode contact disposed remotely from the fat pad (e.g., an anode). The remotely-disposed electrode contact, as appropriate, may be placed within a venous lumen of the subject, such as a coronary sinus (e.g., as described hereinbelow with reference to FIG. 46A, 46B, or 52), or at another site, or may be integrated into an outer conductive surface of control unit 1024.

For some applications, electrode contact 1030 is implanted in atrial muscle tissue 1042 and/or in fat pad 1044, while electrode contact 1032 (e.g., serving as an anode) coupled to lead 1026 remains in right atrium 1040. For some applications, one or more additional electrode contacts (e.g., electrode contact 1032) are also implanted in the atrial tissue and/or fat pad, and/or one or more additional proximal electrode contacts are provided on the lead of electrode contact 1030.

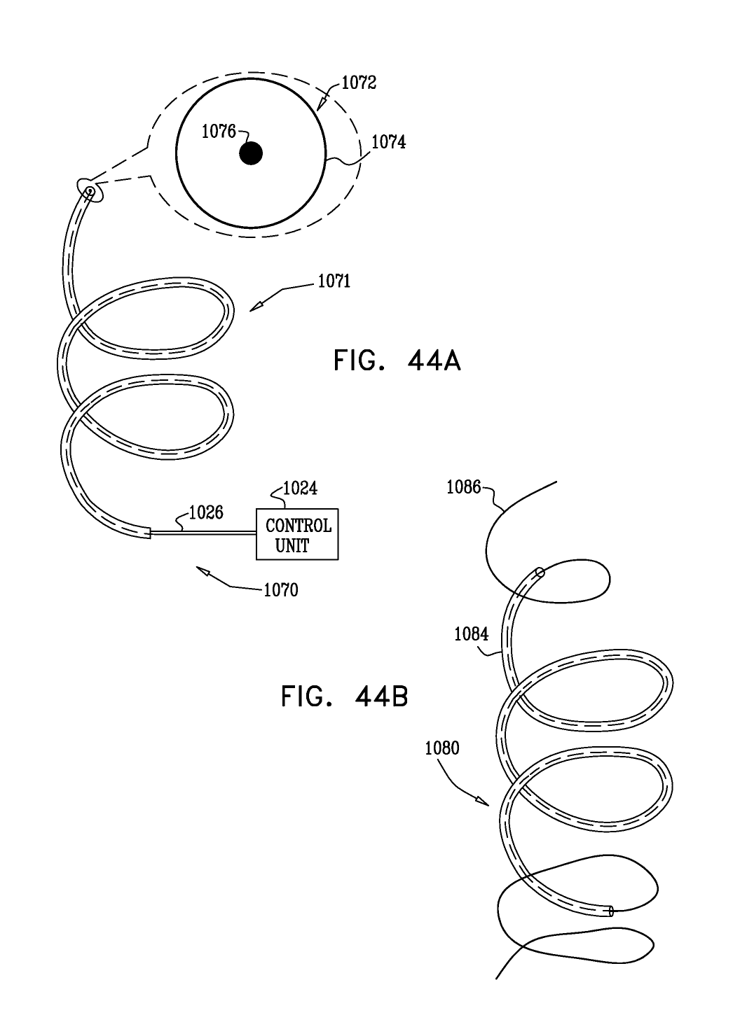

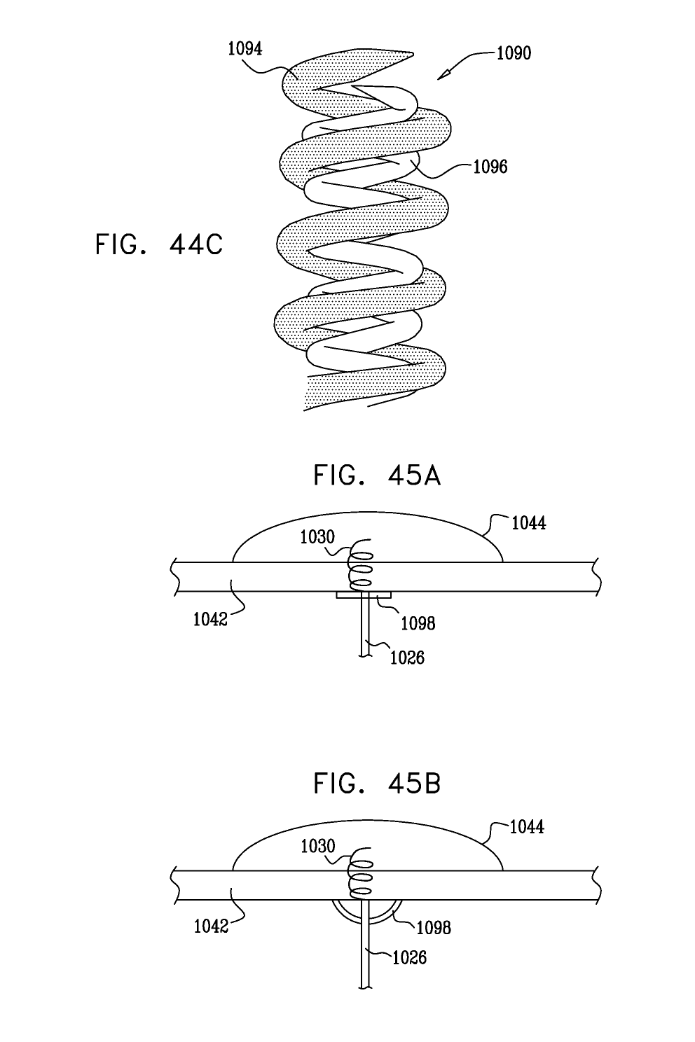

FIG. 44A is a schematic illustration of a screw-in electrode assembly 1070 of system 1020, in accordance with an embodiment of the present invention. Screw-in electrode assembly 1070 comprises a screw-in fixation element 1071 having at its distal tip a concentric bipolar electrode 1072, and a lead 1026. The enlarged portion of FIG. 44A shows a schematic cross-sectional view of bipolar electrode 1072 of screw-in electrode assembly 1070, in accordance with an embodiment of the present invention. Bipolar electrode 1072 comprises an outer electrode contact 1074 and an inner electrode contact 1076, typically having opposite polarities. For example, outer electrode contact 1074 may serve as an anode or a cathode. For some applications, outer electrode contact 1074 extends along the entire length of screw-in fixation element 1071 or a portion thereof, while for other applications the outer electrode contact is limited to only the distal tip of screw-in fixation element 1071, in which case outer electrode contact 1074 and inner electrode contact 1076 are connected to lead 1026 via separate wires.

FIG. 44B is a schematic illustration of a screw-in electrode assembly 1080 of system 1020, in accordance with an embodiment of the present invention. Screw-in electrode assembly 1080 comprises a screw-in fixation element, which comprises an outer helical member 1084 and an inner helical member 1086. Outer helical member 1084 is shaped so as to define a bore through the entire length of the member, and inner helical member 1086 is shaped and sized so as to pass through the bore. All or a distal portion of inner helical member 1086 is configured to serve as a first electrode contact, and all or a distal portion of outer helical member 1084 is configured to serve as a second electrode contact.

During an implantation procedure, inner helical member 1086 is typically rotated with respect to outer helical member 1084 such that the inner helical member partially protrudes from the proximal end of the outer helical element, and substantially does not protrude from the distal end of the outer helical element (i.e., the end that enters the tissue first). After the outer helical element has been screwed into the tissue to a desired depth, the inner helical element is rotated within the outer helical element until the distal end of the inner helical element advances further into the tissue to a desired depth. For some applications, the distal end of the outer helical element is positioned within atrial muscle tissue 1042, and the distal end of the inner helical element is positioned within parasympathetic epicardial fat pad 1044, typically so that the electrode contact of the inner helical element is in direct electrical contact only with the fat pad, and not with the muscle tissue.

FIG. 44C is a schematic illustration of a screw-in electrode assembly 1090 of system 1020, in accordance with an embodiment of the present invention. Screw-in electrode assembly 1090 comprises an outer helical fixation element 1092 having a first radius, and an inner helical fixation element 1094 having a second radius less than the first radius. The inner helical element is positioned within the outer helical element, such that the two helical elements are independently rotatable. All or a distal portion of inner helical member 1092 is configured to serve as a first electrode contact, and all or a distal portion of outer helical member 1094 is configured to serve as a second electrode contact.

During an implantation procedure, the inner and out helical members are independently rotated until each has been screwed into the tissue to a respective desired depth. The two helical members may be rotated together for a portion of the screwing. For some applications, the distal end of the outer helical element is positioned within atrial muscle tissue 1042, and the distal end of the inner helical element is positioned within parasympathetic epicardial fat pad 1044, typically so that the electrode contact of the inner helical element is in direct electrical contact only with the fat pad, and not with the muscle tissue. Alternatively, the distal end of the inner helical element is positioned within atrial muscle tissue 1042, and the distal end of the outer helical element is positioned within parasympathetic epicardial fat pad 1044, typically so that the electrode contact of the outer helical element is in direct electrical contact only with the fat pad, and not with the muscle tissue.

In an embodiment of the present invention, electrode assembly 1022 comprises a first fixation element, which is configured for initial fixation during a first stage of an implantation procedure, and the electrode assembly comprises a second fixation element, which is configured to fix the electrode assembly in place during a second stage of the implantation procedure, with the aid of the initial fixation. For some applications, the first fixation element comprises a thin screw-in element, and the second fixation element comprises a screw-in element having a greater diameter. For some applications, the first fixation element is short and strong for fixation, and the second fixation element is longer and softer. For some applications, the electrode assembly does not comprises the second fixation element, and is held in place entirely or primarily by the first fixation element. For some applications, such electrode assemblies are used for pericardial implantation, while for other application, such electrode assemblies are used for epicardial implantation.

Reference is made to FIGS. 45A-B, which are schematic illustrations of electrode assemblies configured to minimize the risk of bleeding, in accordance with respective embodiments of the present invention. In embodiments of the present invention in which one or more of the electrode assemblies are configured such that at least a portion thereof penetrates the atrial wall and protrudes outside the atrium, the electrode assemblies are configured to minimize the risk of possible bleeding at the site of the penetration. For some applications, one or more of the following techniques are used to minimize this risk:
at least a portion of the electrode assembly that comes in contact with the surface of the cardiac wall has a greater cross-sectional area than a more distal portion of the electrode assembly adjacent thereto. For example, FIG. 45A shows a sealing element 1098 having a cross-sectional area greater than that of lead 1026 where the lead joins the sealing element. Sealing element 1098 typically comprises a flexible material, such as silicone. For some applications, sealing element 1098 is cupulate, such as shown in FIG. 45B;
a portion of the electrode assembly that comes in contact with the cardiac wall is configured to cause fibrosis (configuration not shown). For example, the portion may comprise a rough surface or a mesh, and/or may be coated with a drug for causing fibrosis.

FIG. 46A is a schematic illustration of a parasympathetic stimulation system 1121, in accordance with an embodiment of the present invention. An electrode contact 1130, e.g., part of a screw-in fixation element, is configured to implanted in atrial muscle tissue 1042, either in a vicinity of SA node fat pad 1046 (as shown in FIG. 46A), or in a vicinity of AV node fat pad 1048 (configuration not shown). A second electrode contact 1132 is disposed on a lead 1126 which passes through superior vena cava 1052, such that the second electrode contact is positioned in the superior vena cava. As shown in the figure, an electric field 1148 is generated, the magnitude of which is highest in the area generally between electrode contacts 1130 and 1132. Specifically, a relatively high field strength develops in fat pad 1044 (not visible in the figure) and at areas outside of heart 30, while a relatively low field strength develops in atrial muscle tissue 1042 and the rest of the heart. In this manner, current generated between electrode contacts 1130 and 1132 affects fat pad 1044 to a greater extent than muscle tissue 42. Alternatively, second electrode contact is placed in another blood vessel, such as an inferior vena cava, a coronary sinus, a right pulmonary vein, a left pulmonary vein, or a right ventricular base.

FIG. 46B is a schematic illustration of an alternative configuration of system 1121, in accordance with another embodiment of the present invention. an electrode contact positioned outside of the heart and the circulatory system in a vicinity of the fat pad (but not in physical contact with the heart or the fat pad) serves as electrode contact 1132 as described hereinabove with reference to FIG. 46A. For some applications, an outer surface of control unit 1024 (the “can”) is conductive, and serves as this remote electrode contact. Alternatively, a separate electrode contact is provided for this purpose (configuration not shown). Aside from this difference, the embodiment of FIG. 46B is generally similar to that described with reference to FIG. 46A, with electrode contact 1130 positioned in a vicinity of the fat pad. For some applications, electrode contact 1130 uses a screw-in configuration. For some applications, control unit 1024 is implanted on the lower right side of the subject's chest in a vicinity of heart 30. For some applications, electrode contact 1130 is configured to be implanted subcutaneously. For example, the electrode contact may be implanted on the right side of the chest between the fourth and sixth ribs, typically in the vicinity of the left sides of the ribs, and/or under the ribs. In contrast, conventional pacemaker and ICDs cans are typically implanted on the upper left side of the subject's chest.

In an embodiment of the present invention, during an implantation procedure, the remote electrode contact is implanted before implanting electrode contact 1130. Implanting electrode contact 1130 comprises positioning electrode contact 1130 at a plurality of locations of in the vicinity of the fat pad; while electrode contact 1130 is positioned at each of the locations, driving a current between the remote electrode contact and electrode contact 1130 and sensing a vagomimetic effect; and implanting electrode contact 1130 such that it is positioned at the one of the locations at which a greatest vagomimetic effect was sensed.

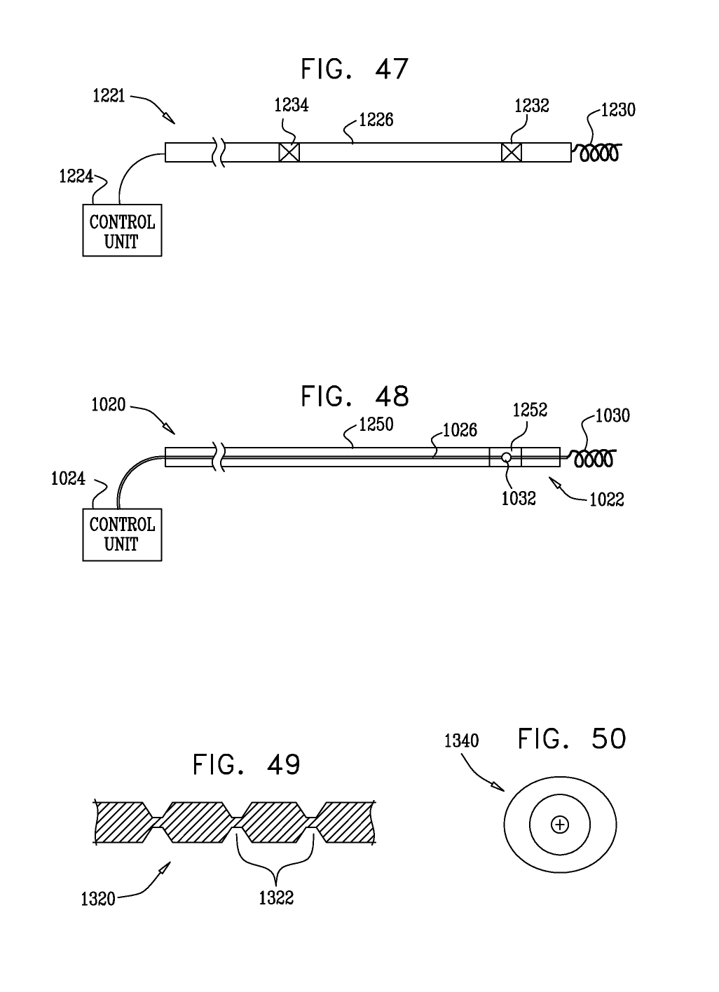

FIG. 47 is a schematic illustration of a parasympathetic stimulation system 1221, in accordance with an embodiment of the present invention. A first electrode contact 1230, e.g., part of a screw-in fixation element, is configured to be positioned in a lower portion of right atrium 1040, typically in a vicinity of AV node fat pad 1048. For some applications, first electrode contact 1230 is configured to be implanted in atrial muscle tissue 1042 in a vicinity of AV node fat pad 1048. A second electrode contact 1232 is disposed on a lead 1226 which passes through superior vena cava 1052, such that the second electrode contact is positioned in right atrium 1040, typically near the first electrode contact, e.g., less than about 2 cm from the first electrode contact, but typically not in contact with the atrial wall. Alternatively, the second electrode contact is configured to be implanted in the atrial muscle tissue near the first electrode contact, such as using one of the electrode contact assemblies described hereinabove with reference to FIGS. 43A-C or 44A-C. During stimulation of SA node fat pad 1046 or AV node fat pad 1048, a control unit 1224 drives a current between first electrode contact 1230 and second electrode contact 1232, typically such that the first electrode contact functions as a cathode and the second electrode contact as an anode.

A third electrode contact 1234 is disposed on lead 1226 such that second electrode contact 1232 is positioned between third electrode contact 1234 and first electrode contact 1230. The third electrode contact is positioned along the lead such that the third electrode contact is positioned in the superior vena cava, or in right atrium 1040 in a vicinity of the superior vena cava.

Prior to driving the first and second electrode contacts to apply stimulation to the fat pad, control unit 1224 uses the first and third electrode contacts, or the second and third electrode contacts, to sense the commencement of a P-wave. As soon as possible after detecting the P-wave (typically within 30 ms, such as within 10 ms), the control unit drives the first and second electrode contacts to stimulate the fat pad. Because the third electrode contact is positioned in the SVC (or near the SVC), the control unit is able to detect early depolarization of the upper portion of right atrium 1040, and thus is able to detect the onset of the atrial depolarization early than would be possible using only the first and second electrode contacts. This enables the control unit to begin stimulation earlier in the atrial refractory period than would otherwise be possible. Such an earlier start typically allows the control unit to apply at least one more pulse during a burst of pulses than would otherwise be possible, without decreasing the interburst period of the pulses within the burst. Alternatively, the early detection of atrial depolarization enables the early detection of atrial capture that may result from the electrical stimulation.

For example, assume the atrial refractory period has a total duration of 100 ms, the pulse duration is 1 ms, pulse repetition interval (PRI) is 7 ms, circuitry of the control unit requires 30 ms to initiate stimulation after detection of the P-wave, and the P-wave arrives near the AV node fat fad about 30 ms after it is generated at the SA node. If the control unit were to detect the P-wave using the first and second electrode contacts, the control unit would have time to apply 10 pulses in the burst (pulses per trigger, or PPT). By instead using the first and third electrode contacts, the control unit is able to apply 14 pulses. This greater PPT enables an increased strength of stimulation without requiring an increase in other stimulation parameters, such as amplitude or pulse duration.

For some applications, system 1221 comprises a fourth electrode contact positioned along lead 1226, typically in a vicinity of second electrode contact 1232 (configuration not shown). The control unit uses the third and fourth electrode contacts to sense the P-wave.

In an embodiment of the present invention, system 1221 is integrated into an implantable cardioverter defibrillator (ICD), and third electrode contact 1234 serves both for detecting the P-wave, as described above, and as the lead of the ICD conventionally positioned in superior vena cava 1052.

Reference is made to FIG. 48, which is a schematic illustration of a sheath 1250, in accordance with an embodiment of the present invention. Sheath 1250 is configured to enable stimulation of the target site during an implantation while at least one the electrode contacts (e.g., electrode contact 1032) of electrode assembly 1022 is still within the sheath. Sheath 1250 includes at least one portion 1252 through which electricity is conductible. For some applications, sheath 1250 is shaped so as to define at least one window that defines the at least one portion 1252. For other applications, sheath 1250 comprises a conductive element that serves as the at least one portion 1252. For some applications, the sheath is configured such that the conducting portion extends from a distal opening of the sheath for at least 1 cm in a proximal direction along the sheath. For some applications, at least a portion of the sheath is deflectable, such as at least a portion of the conducting portion.

Before or during an implantation procedure, lead 1026 is positioned in sheath 1250 such that electrode contact 1032 is aligned with conducting portion 1252. During the implantation procedure, as a distal electrode contact, e.g., electrode contact 1030, is positioned at various potential stimulation sites, system 1020 applies stimulation between electrode contact 1032 and electrode contact 1030 within the sheath. For some applications, the sheath includes a plurality of conducting portions 1252, and stimulation is applied sequentially through each of the portions and the proximal electrode.

Reference is made to FIG. 49, which is a schematic illustration of an electrode lead 1320 shaped so as to define grooves 1322 on an external surface thereof, in accordance with an embodiment of the present invention. The grooved electrode lead is configured for trans-septum placement of an electrode contact at a left-atrial site. The grooves enable better sealing of the opening made in the septum for passage of the lead therethrough.

In some embodiments of the present invention, the stimulation site includes an interatrial groove. For example, the electrode contact may be placed at least partially in a vicinity of the groove, such as within about 2 mm of the groove, or in contact with the groove.

Reference is made to FIG. 50, which is a schematic illustration of tripolar ganglion plexus (GP) electrode assembly 1340, in accordance with an embodiment of the present invention. This configuration generally limits the spread of any depolarization through the atrial tissue. The external anode generally blocks the propagation of atrial depolarization. The internal anode is used as a reference point, and as a means to limit the excitation potential of the external anode (known as an anode-induced virtual cathode), by dividing the current flow between the external and the internal anodes.

The stimulation waveform is typically quasi-trapezoidal, so as to avoid anodal break, for example. Furthermore, the stimulation waveform is typically asymmetrically balanced, with the discharge current spread over a longer period of time than the charging (stimulating) current. For example, for a stimulation with a duration of 0.5 ms, the discharge current may have a duration of at least 1.5 ms.

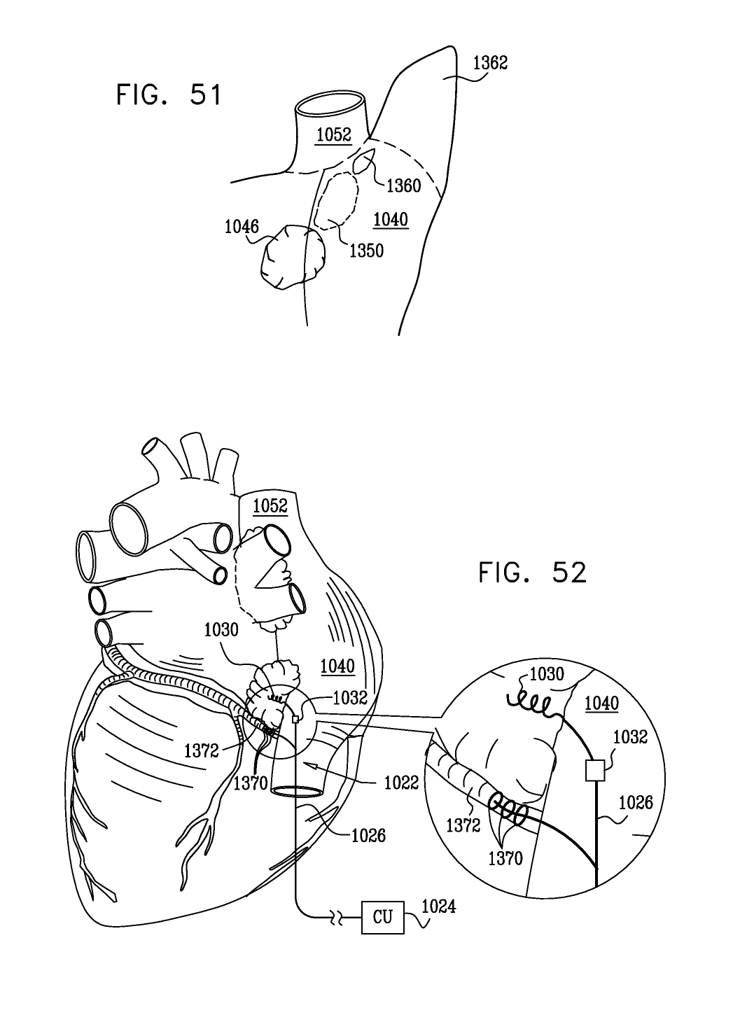

Reference is made to FIG. 51, which is a schematic illustration of an atrial region 1350 for stimulation of postganglionic fibers, in accordance with an embodiment of the present invention. In this embodiment, at least one electrode contact is positioned at atrial region 1350 within an atrium (typically the right atrium, or alternatively in the left atrium) in contact with the atrial wall, within the atrial wall, and/or through the atrial wall, in a vicinity of postganglionic fibers of a parasympathetic epicardial fat pad, such as SA node fat pad 1046 and/or AV node fat pad 1048, but not at or in the fat pad itself (i.e., not in contact with, or within, tissue of the cardiac wall that underlies the fat pad). Typically, but not necessarily, atrial region 1350 is located generally between SA node fat pad 1046 and an SA node 1360, as shown in FIG. 51 (which also shows a right atrial appendage 1362), or generally between AV node fat pad 1048 and an AV node (location not shown). The inventors believe that stimulation of the postganglionic fibers in this region has a greater heart-rate-reduction effect than stimulation at or in the fat pads. The inventors also hypothesize that such postganglionic stimulation generally causes less afferent activation than stimulation of the fat pads or preganglionic fibers, and is thus less likely to cause side effects.

FIG. 52 is a schematic illustration of yet another configuration of stimulation system 1020, in accordance with an embodiment of the present invention. In this embodiment, electrode assembly 1022 comprises one or more electrode contacts 1370 which are configured to be placed in a coronary sinus 1372. For some applications, electrode contacts 1370 comprise ring electrodes, as shown in the figure. Alternatively or additionally, electrode contacts 1370 are incorporated into one or more baskets or coronary stents (configurations not shown). Electrode contact 1030 (which may comprise any of the fixation elements described herein, such as a screw-in fixation element) is configured to be implanted in a vicinity of AV node fat pad 46, in contact with the atrial wall, within the atrial wall, and/or through the atrial wall into the fat pad. Optionally, electrode assembly also comprises an electrode contact 1032 positioned along lead 1026 in a vicinity of electrode contact 1030. Control unit 1024 is configured to drive a current between (a) electrode contact 1030 and (b) one or more of electrode contacts 1370, or, optionally electrode contact 1032.

In an embodiment, control unit 1024 is configured to drive the current in alternation between (a) electrode contact 1030 and (b) each of electrode contacts 1370 or, optionally, electrode contact 1032. For some applications, the control unit configures electrode contact 1030 to be the cathode, and the other contacts to be the anode. The alternation among electrode contacts 1370 and 1032 generally reduces the likelihood of exhausting the ganglia within AV node fat pad 1046. Typically, the alternation has a frequency of between about 1 Hz and about 1000 Hz.

Alternatively, one or more of electrode contacts 1370 are positioned in the inferior vena cava instead of or in addition to in coronary sinus 1372. Further alternatively, electrode contact 1030 is applied to another parasympathetic epicardial fat pad, such as the SA node fat pad, in which case electrode contacts are typically positioned in one or more of the right pulmonary arteries.

In an embodiment of the present invention, the burst length is configured to be as long as possible without extending beyond the conclusion of the AERP. To shorten the time from detection of an atrial depolarization to the initiation of the stimulation, the system charges the stimulation capacitor after each stimulation, and maintains the voltage on the stimulation capacitor until the next stimulation is applied, thus maintaining a “loaded and ready” situation. Each burst thus begins about 30 ms earlier than it otherwise could have if the capacitor had not been already charged, at the expense of battery life.

In an embodiment of the present invention, parasympathetic stimulation is combined with pacing at a location remote from the parasympathetic stimulation site. Application of such pacing allows the control unit to know exactly when the refractory period begins, in order to ensure that the parasympathetic stimulation is applied during the refractory period, and/or to apply the first pulse as early as possible during the refractory period, as described above. For some applications, the control unit applies the parasympathetic stimulation slightly before applying the pacing, such as up to 50 ms before the pacing, e.g., between about 10 ms and about 50 ms before the pacing. Alternatively, for some applications, the control unit applies the parasympathetic stimulation after a delay after application of the pacing, such as a delay equal to the estimated conduction time between the site of the pacing and the site of the parasympathetic stimulation.

In an embodiment of the present invention, system 1020 comprises at least one electrode contact configured to be implanted epicardially (i.e., from outside the heart, rather than transvascularly). Control unit 1024 drives the electrode contact to apply stimulation such that more of the current of the stimulation passes through pericardium than passes through myocardium. For some applications, such epicardial implantation is used for applying stimulation for preventing and/or terminating atrial fibrillation, typically by applying the stimulation to the AV node fat pad. For some applications, techniques are used that are described in U.S. patent application Ser. No. 11/657,784, filed Jan. 24, 2007, which issued as U.S. Pat. No. 8,204,591, and/or U.S. patent application Ser. No. 10/560,654, filed May 1, 2006, which issued as U.S. Pat. No. 7,885,711, both of which are assigned to the assignee of the present application and are incorporated herein by reference. For some applications, such epicardial implantation is used for treating a subject suffering from both heart failure and atrial fibrillation. For some applications, such stimulation is applied only when a sensed heart rate of the subject exceeds a threshold heart rate, such as about 60 BMP.

In an embodiment of the present invention, a parasympathetic epicardial fat pad radiopaque marker is placed during an open chest operation, to facilitate later positioning (e.g., position and/or angle of penetration) of an intra-atrial electrode contact.

In an embodiment of the present invention, system 1020 is configured to detect whether the electrode contact has become dislodged and passed out of the atrium into the ventricle (either from the right atrium to the right ventricle, or the left atrium to the left ventricle). Application of the stimulation to the ventricle may cause ventricular capture, which could be potentially dangerous. Control unit 1024 uses the electrode contact to sense a local electrogram. The control unit analyzes the electrogram to determine whether it is characteristic of atrial electrical activity. Atrial signals have characteristic signatures, such as shape and signal width, that are different from those of ventricle signals, both when the subject is in NSR and in AF, as is known to those skilled in the art. Upon finding that the electrode contact remains at its implantation site in the atrium, the control unit applies parasympathetic stimulation, such as using techniques described herein. Otherwise, the control unit withholds applying the stimulation. Typically, the control unit configures the stimulation to avoid causing capture, such as by setting the signal strength to be too low to cause capture, applying the signal during the atrial refractory period, or using other techniques for avoiding capture described herein.

For some applications, the control unit performs this verification of atrial location generally continuously for application of stimulation. Alternatively, the control unit performs the verification periodically, such as once per minute, or once per hour. For some applications, the control unit periodically withholds applying the parasympathetic stimulation during a sensing period having a duration of at least 2 seconds, e.g., at least 5 seconds, or at least one minute, and performs the sensing during this period. Providing such a non-stimulation period generally provides a cleaner sensed signal because the parasympathetic stimulation is less likely to cause interference. For some applications, the control unit uses known signatures of atrial activity, while for other applications, the control unit performs a characterization of the subject's atrial electrical activity to generate a unique signal fingerprint for the subject, during a calibration procedure performed prior to, during, or soon after implantation of the electrode contact.

For some applications, system 1020 includes a ventricular lead configured to be placed in a ventricle. The control unit periodically compares the signal detected by the electrode contact in the atrium and the signal sensed by the ventricular lead in the ventricle. The control unit interprets a change in the comparison as indicating that the electrode contact or the lead has moved, and thus withholds driving the electrode contact to apply the parasympathetic stimulation.

Reference is made to FIG. 53, which is a flow chart schematically illustrating a method 1100 for implanting an electrode contact at a desired position in parasympathetic epicardial fat pad 1044, in accordance with an embodiment of the present invention. For some applications, method 1100 is used to implant electrode contact 1030 and/or electrode contact 1032, described hereinabove with reference to FIG. 42 and FIGS. 43A-C; the screw-in electrode assemblies described hereinabove with reference to FIGS. 44A-C; or any of the other electrode assemblies described herein or otherwise known in the art.

Method 1100 aids in the positioning of the distal portion of one or more electrode contacts in parasympathetic epicardial fat pad 1044, which for many applications is the optimal positioning. It is generally desirable not to advance the electrode contact entirely through the fat pad and out into the pericardial space, where the presence of an electrode contact may cause pericardial irritation and effusion.

Method 1100 begins with the positioning of the electrode contacts in an atrium, such as right atrium 1040 or a left atrium, in a vicinity of parasympathetic epicardial fat pad 1044, at an electrode contact positioning step 1102. During the implantation procedure, impedance between the electrode contacts is periodically or generally continuously monitored to aid with locating and fixating the electrode contacts in the fat pad. Such monitoring is generally achieved by passing a fixed current pulse between the electrode contacts and measuring the required voltage, or by applying a fixed voltage and measuring the resulting current. Such pulses are typically applied at least once every two seconds, to provide a generally continuous impedance assessment. At a baseline impedance measurement step 1104, baseline impedance is measured while the electrode contacts are still in the atrium. Such impedance is relatively low while the electrode contacts are in contact only with blood.

Insertion of the electrode contacts into atrial muscle tissue 1042 begins at a insertion step 1106. Impedance is monitored during the insertion, and at an impedance check step 1108, it is determined whether an increase in impedance has occurred. Such an increase indicates that insertion into the muscle tissue has begun. Further insertion of the electrode contacts through the muscle tissue while monitoring impedance continues at a continuing insertion step 1110.

At an impedance check step 1112, it is determined whether a further increase in impedance has occurred. Such a further increase indicates that the electrode contacts have entered fatty tissue of fat pad 1044. Optionally, upon detecting this further increase in impedance, insertion is stopped immediately, at an implantation completion step 1114. Alternatively, the electrode contacts are advanced slightly more into the fat pad in order to provide better contact, and a continued insertion step 1116. Impedance is monitored, and at an impedance check step 1118, it is determined whether impedance has decreased. Such a decrease indicates that the electrode contacts have been inserted too far, and have exited the fat pad into the pericardial space. The electrode contacts are thus withdrawn while monitoring impedance until the impedance returns to approximately its previous level, at an electrode contact withdrawal step 1120, upon which the implantation is complete at step 1114.

For some applications of method 1100, two or more electrode contacts are positioned in the fat pad, and impedance is monitored between or among the electrode contacts. For other applications, one or more electrode contacts are positioned in the fat pad, and impedance is monitored between each of the electrode contacts and one or more electrode contacts positioned in the atrium.

In another embodiment of the present invention, method 1100 monitors pressure instead of impedance. The pressure at or near the distal tip of the one or more electrode contacts, or of one or more dedicated guidewires, is monitored during the positioning of the electrode contacts. An increase in pressure detected at check step 1108 indicates that the electrode contacts have entered the atrial wall. A decrease in pressure detected at check step 1112, characterized by sinusoidal periodic changes in pressure, indicates penetration of the electrode contacts into the fat pad tissue. If the distal tip is undesirably further advanced into the pericardial space, a flat or spiked pressure pattern is observed at impedance check step 1118. Corrections to electrode contact position are made accordingly as to position the electrode contact within the fat pad tissue but not in the pericardial space, at withdrawal step 1120.

In another embodiment of the present invention, the positioning of the one or more electrode contacts is achieved under echocardiogram visualization, such as transesophageal echocardiogram visualization, or transthoracic echocardiogram visualization. The active fixation screw is advanced through the atrial wall, but not into the pericardial space.

In an embodiment of the present invention, the one or more electrode contacts are driven to apply parasympathetic stimulation while being advanced into the cardiac muscle and/or fat pad (the stimulation is typically applied either at subthreshold strength, and/or only during the AERPs of each cardiac cycle). Advancement of the electrode contacts is stopped when a desired heart-rate-lowering effect of stimulation is observed, so that the electrode contact is not advanced further than needed. If an insufficient heart-rate-lowering effect is observed at all depths of insertion, the electrode contacts are withdrawn and re-inserted in at a slightly different position and/or angle.

In an embodiment of the present invention, the control unit is configured to drive the electrode contacts to apply to the fat pad a burst of pulses comprising one or more initial pulses followed by one or more subsequent pulses. The control unit sets a strength of the initial pulses to be insufficient to cause parasympathetic activation of the fat pad, and a strength of the subsequent pulses to be sufficient to cause the parasympathetic activation. The initial pulses serve to precondition the fat pad for more effective subsequent parasympathetic activation. For some applications, the strength of the initial and subsequent pulses are set during a calibration procedure, in which the electrode contacts are driven to apply a plurality of calibration bursts at respective calibration strengths, whether the calibration bursts cause a vagomimetic effect is sensed, a minimal strength necessary to cause the vagomimetic effect is found, and the preconditioning strength of the initial pulses is set to be less than the minimal strength, and the activating strength of the subsequent pulses to be at least the minimal strength.

In an embodiment of the present invention, system 1020 comprises at least two electrodes contacts that are configured to be positioned intra-atrially at at least two separate vagomimetic sites (i.e., sites causing a vagal response when stimulated), or at least thee electrode contacts that are configured to be positioned at at least three separate vagomimetic sites. For some applications, a first one of the electrode contacts is positioned in a vicinity of SA node fat pad 1046, and a second one of the electrode contacts AV node fat pad 1048. For some applications, one or more (such as all) of the electrode contacts are coupled to the wall using a screw-in fixation element.

In an embodiment, control unit 1024 is configured to simultaneously drive all of the electrode contacts to apply stimulation to the respective sites. For some applications, the control unit is configured to drive the electrode contacts to apply the stimulation during the atrial absolute refractory period.

In an embodiment, control unit 1024 is configured to drive each of the electrode contacts to apply stimulation to its respective site during a local refractory period at the site. For some applications, the control unit uses each of the electrode contacts to both apply the stimulation and to sense a local electrogram, which the control unit uses to detect the local refractory period.

In an embodiment of the present invention, combined intra-atrial stimulation of the SA and AV fat pad nodes is applied to treat a subject suffering from heart failure and paroxysmal atrial fibrillation (AF). According to one method for such treatment, control unit 1024 detects whether the subject is currently in normal sinus rhythm (NSR) or experiencing an episode of AF. If the subject is experiencing the episode of AF, the control unit drives the electrode contact in the vicinity of the AV node fat pad to apply stimulation to AV node fat pad, in order to reduce the ventricular rate (stimulation of the SA node fat pad has minimal effect on ventricular rate during AF). If, on the other hand, the subject is in NSR, the control unit drives the electrode contact in the vicinity of the SA node fat pad to apply stimulation to the SA node fat pad, in order to reduce the ventricular rate. For some applications, if the subject is in NSR, the control unit measures the heart rate and compares it to a threshold value (e.g., between about 60 and about 150 BPM, such as about 80). The control unit drives the electrode contact in the vicinity of the SA node fat pad to apply the stimulation only if the heart rate is greater than the threshold.

In an embodiment of the present invention, system 1020 comprises a sensor of cardiac activity, configure to generate a cardiac activity signal. Control unit 1024 is configured to receive and analyze the signal, and, upon finding that the subject is in AF, to perform cardioversion by applying simultaneous stimulation of the AV node and SA node fat pads. For some applications, this embodiment is performed in combination with techniques described in U.S. application Ser. No. 11/724,899, filed Mar. 16, 2007, entitled, “Parasympathetic stimulation for termination of non-sinus atrial tachycardia,” which issued as U.S. Pat. No. 8,060,197 and is assigned to the assignee of the present application and is incorporated herein by reference.

In an embodiment of the present invention, a method is provided for combined reduction of heart rate and prolongation of PR interval to obtain optimal cardiac performance, comprising: (a) intra-atrially stimulating the SA node fat pad to cause heart rate reduction, (b) intra-atrially stimulating the AV node fat pad to cause PR prolongation, (c) sensing a measure of cardiac performance (e.g., cardiac contractility, blood pressure, or cardiac output), and (d) responsively to the measure, configuring one or more parameters of the stimulation of the AV node fat pad to improve the sensed measure of cardiac performance.

In an embodiment of the present invention, a method is provided for achieving cardiac arrest, comprising: intra-atrially applying stimulation to both the SA node and AV node fat pads, and configuring the stimulation to achieve the cardiac arrest. This method is typically performed during a cardiac surgical procedure.

In an embodiment of the present invention, a method is provided for delivering rate control therapy while maintaining AF. The control unit detects whether the subject is in AF. When the control unit finds that the subject is in AF, the control unit drives the electrode contact to apply stimulation to the AV node fat pad, and configures the stimulation to reduce the heart rate. When, on the other hand, the control unit finds that the subject is in NSR, the control unit drives the same electrode contact or another electrode contact to apply a pacing signal to the atrium, and configures a rate of the signal to be at least 1.5 Hz (e.g., at least 20 Hz) in order to convert the subject to AF. For some applications, the pacing signal is applied at the same site as the stimulation of the AV node fat pad, for example using at least one common electrode, or, alternatively, using different electrodes.

Such AF maintenance generally reduces the frequency of recurring transitions between AF and NSR, which transitions are common in subjects with AF, particularly in subjects with chronic episodic AF. Such repeated transitions are generally undesirable because: (a) they often cause discomfort for the subject, (b) they may increase the risk of thromboembolic events, and (c) they often make prescribing an appropriate drug regimen difficult. Drug regimens that are beneficial for the subject when in AF are often inappropriate when the subject is in NSR, and vice versa. Knowledge that the subject will generally remain in AF typically helps a physician prescribe a more appropriate and/or lower-dosage drug regimen.

In some embodiments of the present invention, a subject is identified as suffering from a cardiac condition, and intra-atrial stimulation of one or more parasympathetic epicardial fat pads is applied to treat the condition. The condition typically includes chronic heart failure (HF), atrial flutter, chronic atrial fibrillation (AF), chronic AF combined with HF, atrial flutter combined with HF, hypertension, angina pectoris, and/or an inflammatory condition of the heart. Alternatively or additionally, the stimulation is applied to regular the production of nitric oxide (NO) (e.g., by changing the level of at least one NO synthase, e.g., increase a level of eNOS), such as in combination with techniques described in U.S. application Ser. No. 11/234,877, filed Sep. 22, 2005, entitled, “Selective nerve fiber stimulation,” which issued as U.S. Pat. No. 7,885,709 and is assigned to the assignee of the present application and is incorporated herein by reference.

For some applications, the stimulation is configured to stimulate vagal ganglion plexuses (GPs). In other embodiments, the stimulation is applied at a site in the pulmonary veins of the subject, or in the great veins leading to the right atria (vena cava veins and coronary sinus).

In an embodiment of the present invention, stimulation of autonomic sites in heart failure subjects has a therapeutic effect by multiple mechanisms of action, including, but not limited to, control over heart rate, increase in coronary blood flow, attenuation of inflammation and apoptosis, reduction in wall tension, and improved relaxation.

The application of chronic autonomic system stimulation using an intra-atrial electrode for treating heart failure subjects enables separate control of rate (by stimulation of the SA node fat pad) and conduction time (by stimulation of the AV node fat pad). Other autonomic stimulation methods generally have an effect on both rate and conduction time. Furthermore, the implantation of atrial electrodes in heart failure subjects has become recently become more common. The autonomic stimulation techniques described herein can generally be applied using the same atrial electrodes implanted for other purposes, and thus may not require the performance of a separate implantation procedure. In addition, the stimulation of parasympathetic epicardial fat pads, the ganglion plexus (GP), and/or postganglionic fibers is believed by the inventors to cause less afferent activation than stimulation of preganglionic axons, and thus fewer side effects. Also, procedures to implant intra-atrial electrodes generally do not require general anesthesia.

For some applications, the intra-atrial stimulation techniques described herein are used in combination with other techniques for treatment of heart failure known in the art, such as techniques that use cervical and thoracic vagal stimulation, intravascular vagal stimulation, and/or epicardial implantation of electrodes for fat pad stimulation.

In an embodiment of the present invention, the intra-atrial fat pad stimulation techniques described herein are used for treating subjects that suffer from both heart failure (HF) and concurrent atrial fibrillation (AF). In such subjects, the risk of causing inadvertent atrial excitation is minimized, since the atria are fibrillating and cannot generally be excited. For some applications, for the treatment of subjects suffering from both HF and AF, SA node fat pad stimulation is applied alone or in conjunction with AV node fat pad stimulation. For some applications and subjects, when SA node or AV node fat pad stimulation is applied in subjects suffering from both HF and AF, the stimulation elicits the beneficial effects of heart failure therapy, and at the same time delivers the beneficial effects of AF prevention, such as preventing remodeling of the atria and reducing atrial wall tension, such as described in above-mentioned U.S. patent application Ser. No. 11/657,784, filed Jan. 24, 2007, which issued as U.S. Pat. No. 8,204,591, and/or U.S. patent application Ser. No. 10/560,654, filed May 1, 2006, which issued as U.S. Pat. No. 7,885,711. For some applications, the SA node fat pad alone, the AV node fat pad alone, or both fat pads are stimulated to treat heart failure subjects with AF even if the stimulation has no or only a minimal effect on the heart rate. (Control over heart rate is usually not achieved when the SA node fat pad is stimulated alone.)

In some embodiments of the present invention, the control unit is configured to apply the stimulation in a series of bursts, each of which includes one or more pulses. For some applications, the control unit is configured to apply one burst per cardiac cycle, synchronized with a feature of the cardiac cycle of the subject. For example, each of the bursts may commence upon detection of a P-wave, or after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity. Further alternatively, for some applications, the bursts are not synchronized with the cardiac cycle, or with other physiological activity.

In an embodiment of the present invention, the control unit configures the stimulation such that at least one pulse in each burst is applied during the atrial effective refractory period (AERP), such as at least half of the pulses in each burst, or all of the pulses in each burst. For some applications, each burst is initiated upon detection of a naturally-occurring P-wave (e.g., immediately upon detection of the P-wave, or within 1-150 ms of detection of the P-wave, and is applied entirely within the AERP.

In some embodiments of the present invention, the techniques described herein are used for treatment of heart failure and/or atrial fibrillation. Alternatively or additionally, the techniques described herein are used post-myocardial infarct, post heart surgery, post heart transplant, during heart surgery, or during an otherwise indicated catheterization (such as PTCA).

Alternatively or additionally, the techniques described herein are used for classic pacing indications (e.g., bradycardia, sick sinus syndrome, or cardiac resynchronization), where instead of applying a single pacing signal, the device applies a burst of pulses, each burst having a duration that is shorter than the AERP, such as no more than 85% of the AERP. Typically the first pulse of each burst paces the atrium, and the subsequent pulses generate a vagomimetic response, but do not cause additional capture, because they are applied during the AERP.

In some embodiments of the present invention, the apparatus is configured to pace the atria, in addition to applying parasympathetic stimulation. The apparatus is configured to begin application of each of the stimulation bursts when the atria is not refractory, and thus the first one or more pulses (e.g., the first one) of the burst causes atrial depolarization, and the remaining pulses of the burst, falling within the effective refractory period, facilitate the vagomimetic effect.

In some embodiments, the first pulse in the train, which is configured to cause atrial depolarization, has an amplitude or pulse duration that is greater than that of the subsequent pulses. For these embodiments, the rate and timing of the stimulation bursts are set according to the clinical indication for atrial pacing, i.e., according to the desired heart rate. Such indications include, but are not limited to, bradycardia and sick sinus syndrome.

In some embodiments of the present invention, the system further comprises a pacemaker/CRT and/or an ICD. For some applications, the control unit is configured to apply the vagomimetic stimulation in bursts including one or more pulses. For some applications, the control unit synchronizes the bursts with a feature of the cardiac cycle. For some applications, the control unit sets a duration of each of the bursts to be no longer then the atrial effective refractory period at the site of stimulation.

In some embodiments of the present invention, the apparatus comprises a CRT pacemaker-like lead that is positioned at a coronary sinus. The electrode comprises a bipolar stimulation tip, that is advanced along the cardiac veins until the stimulation tip is positioned over the left ventricle, and additional proximal bipolar stimulation rings that are positioned in the coronary sinus. The distance between the two electrode sets is typically between about 2 to about 10 cm. The control unit drives the second, proximal, electrodes to stimulate the local vagomimetic site. This site is stimulated after the distal electrode set is stimulated and ventricular depolarization is initiated. Thus, the vagomimetic stimulation does not to interfere with the CRT signal propagation and ventricular depolarization sequence, even if local cardiac excitation occurs, since it is timed to the wanted timing for excitation of the underlying cardiac tissue. Alternatively, vagomimetic stimulation is applied before the distal signal, according to the desired AV delay and interventricular delay.

In some embodiments of the present invention, a method for pacing a heart comprises delivering a burst of pulses, wherein the burst duration is no longer then the effective refractory period at the site of the pacing. For some applications, this method is used for pacing an atrial site, while for other applications, this method is used for pacing a non-atrial site, such as a ventricular site.

In an embodiment of the present invention, an additional sensing and/or pacing electrode is placed in the right ventricle. For some applications, this electrode is used to pace the heart if the heart rate falls below a certain threshold. For other embodiments, the ventricular electrode is used to sense the ventricular rate for confirmation of the sensing of the P-wave by the atrial electrode. In practice, a depolarization sensed in the ventricle inhibits the detection of a P-wave and/or the stimulation by the atrial electrode, for a period of about 250 ms. Thus, ventricular premature beats that might be accidentally detected also in the atria are not detected, and stimulation outside of the refractory period is avoided.

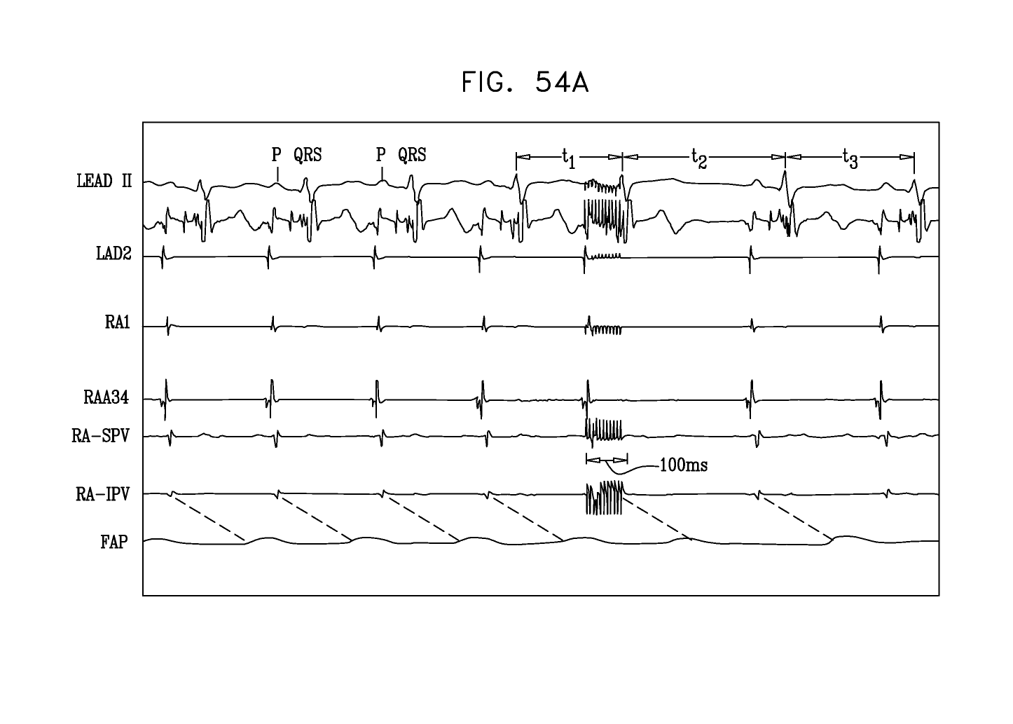

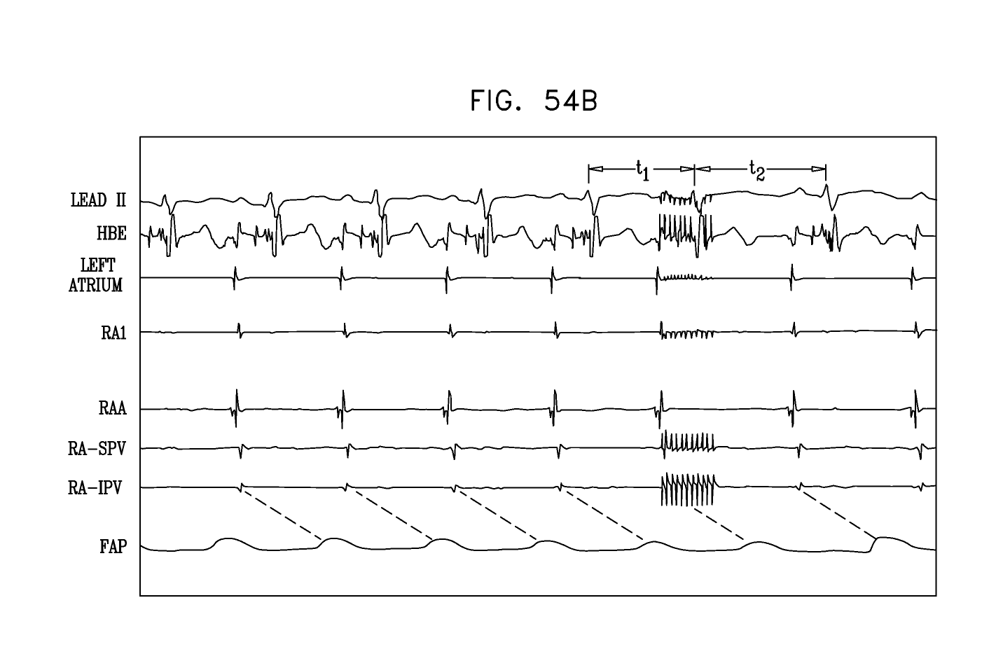

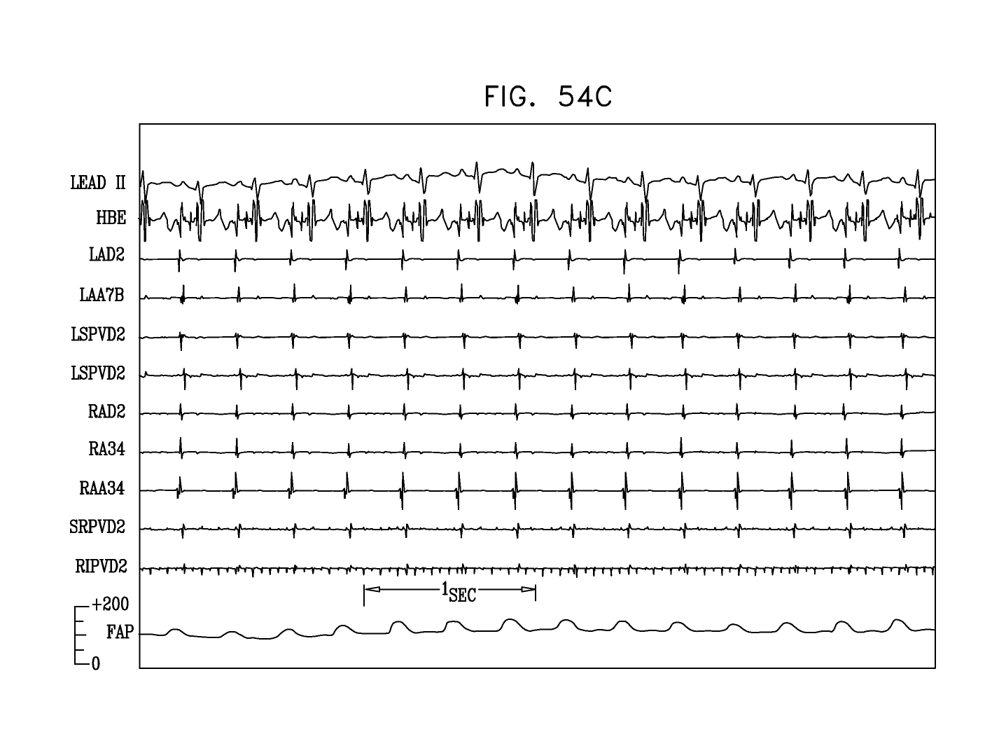

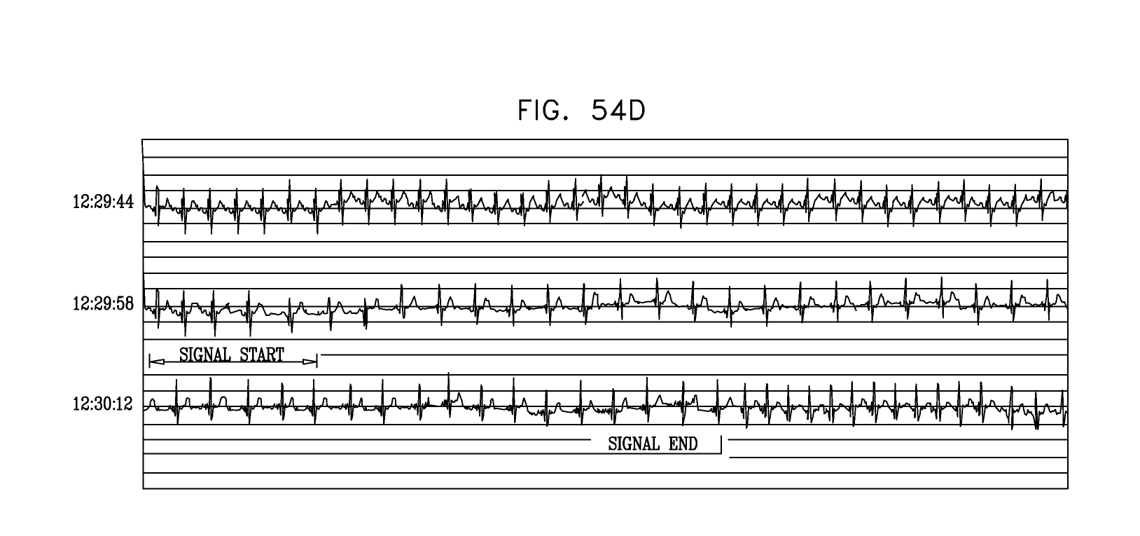

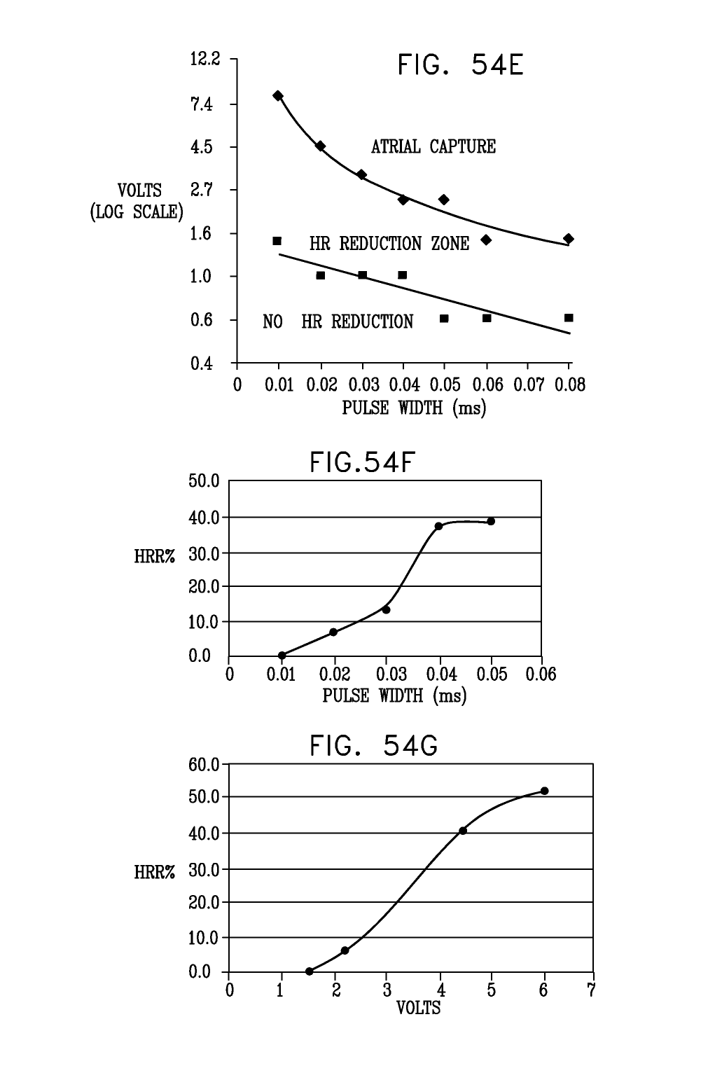

Reference is now made to FIGS. 54A-G, which are graphs showing data recorded in a dog experiment performed in accordance with an embodiment of the present invention. A first intra-atrial active fixation lead was implanted, penetrating through the atrial wall from within the right atrium, to arrive at the vicinity of the sinoatrial (SA) node fat pad, and a second intra-atrial activate fixation lead was implanted in the vicinity of the atrioventricular (AV) node fat pad. Stimulation of the SA and AV node fat pads was achieved using intra-atrial electrode contacts suitable for chronic implantation, in an appropriate medical procedure to chronically implant the electrode contact. Following the experiment, the dog was sacrificed.

Two small canines were utilized (15-20 kg), such that the size of heart chambers and musculature dimensions were approximately 60% of adult human size. Choice of this model enabled the use of “off-the-shelf” electrode contacts marketed for human use, without any adaptations. (For adult human use, some embodiments of the present invention utilize larger electrode contacts than are provided for normal pacing applications.)

Bilateral thoracotomy was performed under general anesthesia using phenobarbital, and the animals were mechanically ventilated. Recording electrodes were placed as described hereinbelow, and direct visualization of the epicardial surfaces was achieved. The pericardium was opened, and recording electrodes were placed on the left atrial roof, left atrial appendage, left superior pulmonary vein, left inferior pulmonary vein, right superior pulmonary vein, and right inferior pulmonary vein. All “vein” electrodes were placed externally to and in contact with the respective vein.

An active fixation lead with a deflectable sheath was used to facilitate placement of the electrode contact at the SA node fat pad. The tip of the sheath was directly viewed, to facilitate placement of the electrode contact. The electrode tip was placed in an open surgical procedure into the right external jugular vein, then passed into the right atrium, midway between the superior vena cava and the inferior vena cava (IVC). The deflectable sheath was deflected toward the free wall (i.e., in an upward direction, since the animal was lying on its left side). The electrode contact was advanced in the dorsal direction, toward the interatrial septum, and was screwed into the muscular part of the right atrium, at a location that was approximately midway between (a) the meeting point of the interatrial septum with the atrial free wall and (b) the crista terminalis, in the vicinity of the SA node fat pad.

In an embodiment of the present invention, all or a portion of this implantation technique is used in a human subject for chronically implanting an electrode contact in a vicinity of a SA node fat pad from within an atrium. For some applications, the electrode contact is inserted into the atrial musculature at a posterior portion of the atrium within about one cm of the interatrial septum. Alternatively, the sheath is pre-shaped. Typically, the sheath is more rigid than the electrode contact. For some applications, the sheath presses (at least in part) against the IVC alternatively or additionally to pressing against the free atrial wall. The applied pressure helps fixate the electrode contact. The sheath also typically helps position the electrode contact at a desired orientation, position, or angle with respect to the tissue to which the electrode contact is fixated.

In order to place an electrode contact at the atrioventricular (AV) fat pad, a second electrode contact was advanced from the right external jugular vein toward the right atrium, until it reached the area of insertion of the inferior vena cava (IVC) into the right atrium. Once the second electrode contact was placed at the most caudal point of the insertion of the IVC, the electrode contact was further advanced approximately 1 cm. The sheath was then deflected and directed towards the dorsolateral wall of the atrium. The inferior atrial wall was then pushed to a perpendicular position in relation to the IVC axis, and pushed towards the electrode contact. The second electrode contact was then screwed through the atrial musculature to the vicinity of the AV node fat pad. In this manner, the second electrode contact was positioned in a vicinity of the AV node fat pad. Data collected during stimulation of the AV node fat pad showed that stimulation of the AV node fat pad also reduced heart rate (data not shown).

In an embodiment of the present invention, all or a portion of this implantation technique is used in a human subject for chronically implanting an electrode contact in a vicinity of an AV node fat pad from within an atrium.

Activation of the SA node fat pads was achieved with signals that were non-excitatory to the atrial muscle tissue, using two different methods. The data shown in FIGS. 54A-G relate to stimulation of the SA node fat pad.

### Atrial Effective Refractory Period (AERP) Stimulation

A human grade muscle stimulator applied symmetric biphasic current pulses to the fat pads (SA node and AV node fat pads) A short burst of pulses was applied within the atrial refractory period, once every several beats, resulting in substantial cycle prolongation. AERP stimulation of the SA node fat pad, which produced the results shown in FIGS. 54A and 54B, was limited to within the effective refractory period. Atrial capture was observed when stimulation extended beyond this period (e.g., for bursts lasting longer than about 130 ms). However, the absolute atrial refractory period was actually shorter than the pulse bursts applied, as demonstrated by applying a burst of relatively long 1.5 ms pulses, where atrial capture could be observed even when the stimulation period was limited to 40 ms. Additionally, 0.02 ms pulses applied outside of the effective refractory period were sufficient to cause capture (data not shown).

Exemplary parameters that produced heart rate reduction included: pulses per trigger (PPT, i.e., pulses applied in one cardiac cycle)=11, pulse repetition interval (PRI)=10 ms and 15 ms, pulse width=0.02-1 ms, current=5-20 mA (e.g., 8 mA). Parameters such as these yielded a heart rate reduction (HRR) from 128 to 95 BPM.

### Asynchronous Stimulation

Stimulation by applying monophasic voltage pulses was performed, without synchronizing the stimulation to the cardiac cycle. Pulse width was manipulated to achieve effective fat pad (SA node and AV node fat pads) stimulation and to avoid atrial capture. In addition, the pulse voltage was also shown to control these effects.

Exemplary parameters that caused heart rate reduction (e.g., from 196 to 160 BPM) were reached while still maintaining a good therapeutic window, i.e., a substantial difference between the minimum voltage that yielded atrial capture and the minimum voltage that yielded effective fat pad stimulation, i.e., heart rate reduction (see FIG. 54E). Heart rate reduction was achieved, for example, using 1.5-8 V (e.g., 2.4 V), 0.01-0.08 ms (e.g., 0.04 ms) pulse width, and 5-20 Hz (e.g., 20 Hz).

Heart rate reduction was found to be correlated with both pulse width and voltage of stimulation, as shown in FIGS. 54F and 54G.

FIG. 54A is a graph showing data recorded in accordance with the AERP stimulation method described hereinabove, in accordance with an embodiment of the present invention. Pulse width was set to 8 mA, PRI was 10 ms, and PPT was 10. Sensing electrodes measured electrical activity on the skin surface (Lead II), at the His bundle, left atrial roof (LAD2), at the right atrial roof (RA1), right atrial appendage (RAA), right superior pulmonary vein (RA-SPV), inferior pulmonary vein (RA-IPV). Femoral arterial pressure (FAP) was also measured.

Dashed lines are shown linking P-waves on the RA-IPV data line with the corresponding pressure pulse on the FAP data line, although it is noted that the pressure pulse is actually caused by the QRS-complex, shown most clearly on the LEAD II data line.

Four normal cardiac cycles are shown in FIG. 54A before the initiation of a 100 ms pulse burst, initiated upon detection of a P-wave. It is seen that the pulse burst did not induce additional atrial electrical activity. Whereas the R-R interval was essentially constant during the four cardiac cycles preceding stimulation, the R-R interval increased by over 50% in the first heartbeat following stimulation (i.e., t2>1.5 t1), and was still elevated by over 20% in the second heartbeat following stimulation (i.e., t3>1.2*t1). It is additionally noted that the femoral arterial pressure (peak-to-peak time) also showed substantial lengthening, indicating that the stimulation provided in this experiment affected both the electrical and the mechanical behavior of the heart.

As can be observed in the graph, the stimulation had an effect on the next beat; not only did the next beat arrive after a longer than usual interval than in the preceding intervals, but the stimulation caused a steeper increase in femoral systolic blood pressure and was conducted through the His bundle in a different way from that seen in the preceding beats.

FIG. 54B is a graph showing data from an experiment performed in accordance with an embodiment of the present invention. The data shown is similar to that described hereinabove with reference to FIG. 54A, except that the PRI was set to 15 ms. In this experiment, the R-R interval increased by approximately 25% in the first heartbeat after stimulation.

FIG. 54C is a graph showing data from an experiment carried out using the asynchronous method described hereinabove, in accordance with an embodiment of the present invention. Pulse width was 0.01 ms, pulse amplitude was 2.4 V, and pulses were applied at 20 Hz, not synchronized to the cardiac cycle. Cardiac electrical and mechanical are seen to not be adversely affected by the stimulation.

FIG. 54D is a graph showing additional data from the experiment described hereinabove with reference to FIG. 54C, in accordance with an embodiment of the present invention. Approximately 15 seconds of baseline data are shown, in which no signal was applied to the heart. Then, at some point during the period marked “signal start,” the same signal as described with reference to FIG. 54C was applied to the SA node fat pad. After about 20 seconds of signal application, the signal was terminated, at the point marked “signal end.” FIG. 54D clearly shows the ability to apply a non-synchronized signal to the fat pads which substantially reduces heart rate, in accordance with an embodiment of the present invention.

FIG. 54E is a graph showing the results of an experiment carried out using the asynchronous method described hereinabove, to determine a therapeutic window which yields heart rate reduction, while avoiding atrial capture, in accordance with an embodiment of the present invention. In this experiment, for a range of pulse widths, signal voltage was increased until heart rate reduction was seen. This voltage was marked with a square. Signal voltage was increased further, until atrial capture was observed. This voltage is marked with a diamond. It is seen that for all of the pulse widths shown in FIG. 54E (0.01-0.08 ms), a window of at least a factor of two exists from the minimum voltage which yields heart rate reduction to the minimum voltage which yields atrial capture. Pulses were applied at 20 Hz, not synchronized to the cardiac cycle.

FIG. 54F is a graph showing the results of an experiment carried out using the asynchronous method described hereinabove, in accordance with an embodiment of the present invention. Pulses of 1.5 V and 20 Hz were applied over a range of pulse widths, from 0.01 to 0.05 ms. Heart rate reduction is seen to occur for pulse widths as low as 0.02 ms (HRR ˜7%), and to increase substantially as pulse width reaches 0.05 ms.

FIG. 54G is a graph showing the results of an experiment carried out using the asynchronous method described hereinabove, in accordance with an embodiment of the present invention. Pulses having a pulse width of 0.01 ms were applied at 20 Hz over a range of voltages. Heart rate reduction is seen for voltages as low as about 2.4 V, and the reduction increases to 40-50% for voltages of 5-6 V.

In an embodiment of the present invention, electrode assembly 1022 comprises two electrode contacts configured to be placed in contact with the atrial wall in a vicinity of a parasympathetic epicardial fat pad. During an implantation procedure, control unit 1024 separately drives each of the electrode contacts to apply stimulation to the wall, and determines respective heart-rate-lowering effects of the stimulation applied by the two electrode contacts. Whichever electrode contact has a great effect on heart rate is left in place, and the other electrode contact is repositioned at one or more addition locations. If stimulation at any of these other locations is found to have a greater heart-rate-lowering effect than at the location at which the first electrode contact remains, the other electrode contact is left at this new location, and the first electrode contact is repositioned at one or more locations. This testing and relocating is repeated until a satisfactory location has been identified, at which point the electrode contact positioned at this location is implanted in the wall. Alternatively, if the heart-rate-lowering effects of the two locations converge, either of the electrodes is implanted. Because an electrode contact is positioned at the identified location, there is no need to attempt to reposition an electrode contact at the location.

In an embodiment of the present invention, control unit 1024 is configured to drive the electrodes to apply low-frequency bursts without synchronizing the bursts with any feature of the cardiac cycle of the subject. Typically, the frequency of the bursts is less than or equal to 2.5 Hz, e.g., less than or equal to 2 Hz (i.e., the number of bursts applied per second, not the number of pulses applied per second). Each of the bursts typically includes between 2 and about 20 pulses, with a pulse repetition interval (PRI) of between about 1 ms to about 30 ms, e.g., between about 3 and about 10 ms, such as about 5 ms. (The PRI is the time from the initiation of a pulse to the initiation of the following pulse within the same burst.) Using this technique, if the system should undesirably cause ventricular capture, the maximum ventricular rate would be no greater than the frequency of the burst. At such low frequencies, such unintended ventricular pacing would not be life-threatening. For some applications, such stimulation is applied when the subject is experiencing atrial fibrillation (AF), while for other applications, the stimulation is applied when the subject is not experiencing AF.

In an embodiment of the present invention, control unit 1024 is configured to apply a signal to tissue in a vicinity of a fat pad, and to configure the signal to both pace the heart (i.e., cause capture) and activate parasympathetic tissue of the fat pad. Typically, an initial portion of the signal causes the pacing. For example, the signal may include bursts each of which include a plurality of pulses, and one or more of the initial pulses of the burst are configured to pace the heart. The control unit senses features of the cardiac cycle of the subject, and applies the signal at a desired portion of the cardiac cycle, as is known in the pacemaker art. Typically, the control unit senses whether the signal has caused capture, and increases the strength of the signal if it has not. At least the pulses configured to cause capture typically have an amplitude of at least 5 mA, and an aggregate duration of at least 2 ms. Typically, the signal is applied in the vicinity of the SA node fat pad; alternatively, the signal is applied in the vicinity of the AV node fat pad. For some applications, one or more electrode contacts are placed in the either the right or left atria.

In an embodiment of the present invention, control unit 1024 is configured to use electrode contacts 1030 and 1032 to both apply fat pad stimulation and sense a local electrogram in the vicinity of the stimulation. The control unit measures a baseline electrogram before beginning application of the stimulation. If, during stimulation, the control unit detects a significant change in the sensed electrogram indicative of the undesired causing of capture by the stimulation, the control unit modifies one or more parameters of the stimulation to reduce the strength of the stimulation, or ceases stimulation.

In an embodiment of the present invention, to aid in the placement of the electrode contact, a CT scan is performed before the implantation, similar to the CT scan sometimes performed before AF ablation. Unlike such a conventional CT scan, in the present embodiment, the area of interest is the right atrium. Therefore, the time from injection of contrast material to triggering of the scan is shorter and the contrast material is less concentrated than conventionally applied for cardiac CT scans (conventional cardiac CT scans aim at the left side of the heart).

In an embodiment of the present invention, to aid in the placement of the electrode contact, prior to implantation a standard bipolar lead is used to find the location with the heart chamber at which application of stimulation causes the greatest heart-rate-lowering effect. The lead is placed at a plurality of locations, and stimulation is applied using the lead at each of the locations in order to determine at which location the stimulation causes the greatest heart-rate-lowering effect. The chronic implantable electrode contact is then positioned at the same location, e.g., using fluoroscopic guidance or a wireless position sensor. Alternatively, for some applications, the location of maximal heart rate reduction is found by applying test stimulation through the implantable electrode contact.

In an embodiment of the present invention, techniques are provided for avoiding inadvertent stimulation of the phrenic nerve. The right phrenic nerve is anatomically close to the SA node fat pad, and stimulation of the SA node fat pad might inadvertently stimulate the phrenic nerve under certain circumstances. To avoid such stimulation, possible stimulation of the phrenic nerve is noted during parameter setting (e.g., by noting irritation of the diaphragm), and the stimulation parameters are configured so as to not activate the phrenic nerve.

In an embodiment of the present invention, a bipolar electrode assembly is provided, comprising two monopolar electrode contacts. For some applications, the electrode assembly comprises more than two electrode contacts. For example, the use of more than two electrode contacts may compensate for post-implantation changes. For some applications, the control unit comprises multiple electrode contacts and switching capabilities, such that external programming can direct the stimulation current to different electrode contacts. For example, if an undesired reduction in stimulation efficacy is observed after the implantation, e.g., due to the development of local fibrosis, the stimulation can be directed through different electrode contacts.

In some embodiments of the present invention, an atrial electrode assembly is provided that hooks around the insertion of the superior vena cava into the right atrium.

In some embodiments of the present invention, the system comprises a first electrode contact, which is configured to be placed in the superior vena cava, and a second electrode contact, which is configured to be placed in the right atrium. For some applications, the control unit drives the first electrode contact to apply a cathodic current, and the second electrode contact to apply an anodal current, thereby limiting the potential of the stimulation to cause atrial depolarization, for example.

In some embodiments, the anode is larger than the cathode (e.g., in length and/or surface area) and/or segmented, such as to further reduce the likelihood of tissue depolarization in the vicinity of the anode.

In some embodiments of the present invention, the system comprises a first electrode contact, which is configured to be placed in the coronary sinus, and a second electrode contact, which is configured to be placed at an atrial site. For some applications, the control unit drives the first electrode contact to apply an anodal current, and the second electrode contact to apply a cathodic current, during the atrial refractory period. Alternatively or additionally, the control unit configures the first electrode contact to apply a cathodic current, and the second electrode contact to apply an anodal current, during the ventricular refractive period. In either case, the refractory periods may be absolute or relative refractory periods.

In an embodiment of the present invention, one or more of the electrode assemblies comprise an active fixation element, including an atrial-wall-penetrating screw-in fixation element that is configured to function as an electrode contact of the electrode assembly. In some embodiments the screw-in fixation element is placed in physical contact with the vagal ganglion plexus within the cardiac fat pads.

In an embodiment of the present invention, the electrode contacts are implanted in a chamber of the heart using a percutaneous approach.

In some embodiments of the present invention, a method for placing electrode contacts at an atrial site comprises testing placement of the electrode contacts by pacing the atrium while increasing vagal tone during a calibration stimulation period, which typically has a duration of between about 2 and about 15 seconds. The control unit paces and increases vagal tone by driving the electrode contacts to apply stimulation bursts that are shorter than the AERP at a rate that is above the basic normal sinus rhythm (NSR) rate, but not so rapid as to induce AF. For example, the rate may be between about 80 and about 140 bursts per minute, such as between about 90 to and about 130 bursts per minute. Upon conclusion of the calibration stimulation period, the atrium naturally returns to its original rate. However, because of the additional pulses applied during the AERP after capture has been achieved, pulses that may cause a vagomimetic effect if positioned correctly, the atrial rate falls below its original rate for several heartbeats, generally between about three and six beats. The control unit measures the R-R interval during at least one of these beats, e.g., during the one, two, or three of these beats. The degree of slowing detected is used to estimate the vagomimetic effect of applying stimulation at the site. If the achieved vagomimetic effect is insufficient, addition location(s) for the electrode contacts are tried until the desired effect is achieved. Alternatively or additionally, the method comprises applying the stimulation bursts as described, and observing the effect on pressure curves in the atria, ventricle, and/or pulmonary system.

Further alternatively or additionally, the method comprises applying stimulation bursts at a fixed rate (such as 120 per minute), with each burst having a duration that is shorter than the AERP, such as less than 90% of the AERP. When the stimulation is positioned at a site that elicits vagomimetic effects, the AERP shortens, resulting in double atrial excitation in each stimulation burst. Such shortening of the AERP can be observed from the atrial or ventricular electrogram.

Further additionally or alternatively, the method of placing the electrode contact includes applying stimulation bursts exclusively within the AERP, without causing atrial excitation. The natural sinus rate is then be observed for slowing that can verify the vagomimetic effect of the stimulation.

Further additionally or alternatively, a temporary pacing lead is positioned within the atria, to provide the atrial electrogram. This lead is removed once the correct position of the stimulating electrode contact is verified.

Further alternatively or additionally, the method comprises selecting a position of the electrode contacts responsively to subject-reported sensations, such as a feeling of warmth in the chest, a radiation of pain to the jaw or neck, or a burning sensation.

Further alternatively or additionally, the method of placing the electrode contact includes sensing the electrogram at the site and searching for irregularity in the ECG signal that is indicative for vagomimetic site. Such irregularity may be fractured ECG signal. Identify such irregularity may indicate that the electrode contact is positioned at a proper vagomimetic site.

Alternatively or additionally, the techniques described herein are used for classic pacing indications (e.g., bradycardia, sick sinus syndrome, or cardiac resynchronization), where instead of applying a single pacing signal, the device applies a burst of pulses, each burst having a duration that is shorter than the AERP, such as no more than 85% of the AERP. Typically the first pulse of each burst paces the atrium, and the subsequent pulses generate a vagomimetic response, but do not cause additional capture, because they are applied during the AERP.

“Heart failure,” as used in the specification and the claims, is to be understood to include all forms of heart failure, including ischemic heart failure, non-ischemic heart failure, and diastolic heart failure. A “screw,” as used in the present application, including in the claims, is to be understood broadly as including a screw, a corkscrew, or any helical element. “Chronically,” as used in the specification and in the claims, means for at least one month.

Techniques described herein for treating atrial fibrillation may also be performed for treating other forms of non-sinus atrial tachycardia, such as atrial flutter.

In some embodiments of the present invention, techniques and/or apparatus described in one or more of the following patents:
U.S. Pat. No. 6,006,134 to Hill et al.;
US Patent RE38,705 to Hill et al.; and/or
U.S. Pat. No. 6,292,695 to Webster, Jr. et al.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 10/205,474, filed Jul. 24, 2002, entitled, “Electrode assembly for nerve control,” which issued as U.S. Pat. No. 6,907,295
U.S. patent application Ser. No. 10/076,869, filed Feb. 15, 2002, entitled, “Low power consumption implantable pressure sensor,” which issued as U.S. Pat. No. 6,712,772
U.S. Provisional Patent Application 60/383,157 to Ayal et al., filed May 23, 2002, entitled, “Inverse recruitment for autonomic nerve systems”
U.S. patent application Ser. No. 10/205,475, filed Jul. 24, 2002, entitled, “Selective nerve fiber stimulation for treating heart conditions,” which published as US Patent Application Publication 2003/0045909
PCT Patent Application PCT/IL02/00068, filed Jan. 23, 2002, entitled, “Treatment of disorders by unidirectional nerve stimulation,” which published as PCT Publication WO 03/018113, and U.S. patent application Ser. No. 10/488,334, filed Feb. 27, 2004, in the US National Phase thereof, which issued as U.S. Pat. No. 7,734,355
U.S. patent application Ser. No. 09/944,913, filed Aug. 31, 2001, entitled, “Treatment of disorders by unidirectional nerve stimulation,” which issued as U.S. Pat. No. 6,684,105
U.S. patent application Ser. No. 10/461,696, filed Jun. 13, 2003, entitled, “Vagal stimulation for anti-embolic therapy,” which issued as U.S. Pat. No. 7,321,793
PCT Patent Application PCT/IL03/00430, filed May 23, 2003, entitled, “Electrode assembly for nerve control,” which published as PCT Publication WO 03/099373
PCT Patent Application PCT/IL03/00431, filed May 23, 2003, entitled, “Selective nerve fiber stimulation for treating heart conditions,” which published as PCT Publication WO 03/099377
U.S. patent application Ser. No. 10/538,521, filed Jan. 11, 2006, entitled, “Efficient dynamic stimulation in implanted device,” which published as US Patent Application Publication 2006/0265027
U.S. patent application Ser. No. 10/719,659, filed Nov. 20, 2003, entitled, “Selective nerve fiber stimulation for treating heart conditions,” which published as US Patent Application Publication 2004/0193231
PCT Patent Application PCT/IL04/00440, filed May 23, 2004, entitled, “Selective nerve fiber stimulation for treating heart conditions,” which published as PCT Publication WO 04/103455
PCT Patent Application PCT/IL04/000496, filed Jun. 10, 2004, entitled, “Vagal stimulation for anti-embolic therapy,” which published as PCT Publication WO 04/110550, and U.S. patent application Ser. No. 10/560,654, filed May 1, 2006, in the US national stage thereof, which issued as U.S. Pat. No. 7,885,711
U.S. patent application Ser. No. 10/866,601, filed Jun. 10, 2004, entitled, “Applications of vagal stimulation,” which published as US Patent Application Publication 2005/0065553
PCT Patent Application PCT/IL04/000495, filed Jun. 10, 2004, entitled, “Applications of vagal stimulation,” which published as PCT Publication WO 04/110549
U.S. patent application Ser. No. 11/022,011, filed Dec. 22, 2004, entitled, “Construction of electrode assembly for nerve control,” which published as US Patent Application Publication 2006/0136024
U.S. patent application Ser. No. 11/062,324, filed Feb. 18, 2005, entitled, “Techniques for applying, calibrating, and controlling nerve fiber stimulation,” which published as US Patent Application Publication 2005/0197675
U.S. patent application Ser. No. 11/064,446, filed Feb. 22, 2005, entitled, “Techniques for applying, configuring, and coordinating nerve fiber stimulation,” which published as US Patent Application Publication 2005/0267542
U.S. patent application Ser. No. 11/280,884, filed Nov. 15, 2005, entitled, “Techniques for nerve stimulation,” which published as US Patent Application Publication 2006/0106441
U.S. patent application Ser. No. 11/340,156, filed Jan. 25, 2006, entitled, “Method to enhance progenitor or genetically-modified cell therapy,” which published as US Patent Application Publication 2006/0167501
PCT Patent Application PCT/IL06/000616, filed May 25, 2006, entitled, “Suture loops for implantable device,” which published as PCT Publication WO 06/126201
U.S. patent application Ser. No. 11/359,266, filed Feb. 21, 2006, entitled, “Parasympathetic pacing therapy during and following a medical procedure, clinical trauma or pathology,” which published as US Patent Application Publication 2006/0206155
U.S. patent application Ser. No. 10/745,514, filed Dec. 29, 2003, entitled, “Nerve-branch-specific action-potential activation, inhibition, and monitoring,” which published as US Patent Application Publication 2005/0149154
U.S. patent application Ser. No. 11/234,877, filed Sep. 22, 2005, entitled, “Selective nerve fiber stimulation,” which published as US Patent Application Publication 2006/0100668
U.S. patent application Ser. No. 11/517,888, filed Sep. 7, 2006, which issued as U.S. Pat. No. 7,904,176, entitled, “Techniques for reducing pain associated with nerve stimulation”
U.S. patent application Ser. No. 11/657,784, filed Jan. 24, 2007, entitled, “Techniques for prevention of atrial fibrillation,” which published as US Patent Application Publication 2007/0179543
U.S. patent application Ser. No. 11/724,899, filed Mar. 16, 2007, entitled, “Parasympathetic stimulation for termination of non-sinus atrial tachycardia,” which issued as U.S. Pat. No. 8,060,197,
U.S. Provisional Patent Application 60/937,351, filed Jun. 26, 2007, entitled, “Intra-atrial parasympathetic stimulation”
U.S. Provisional Patent Application 60/965,731, filed Aug. 21, 2007, entitled, “Intra-atrial parasympathetic stimulation”

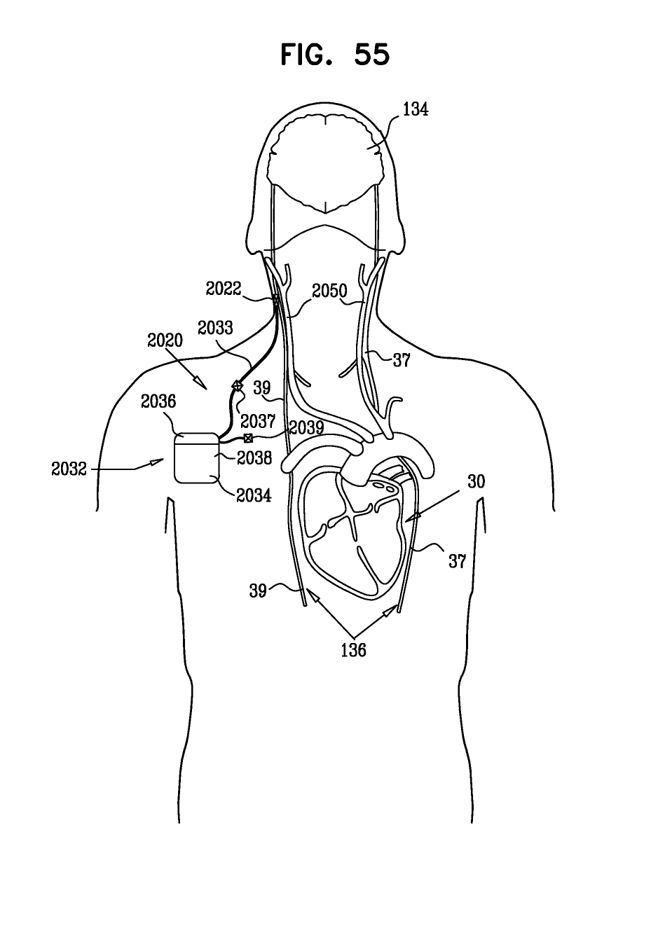

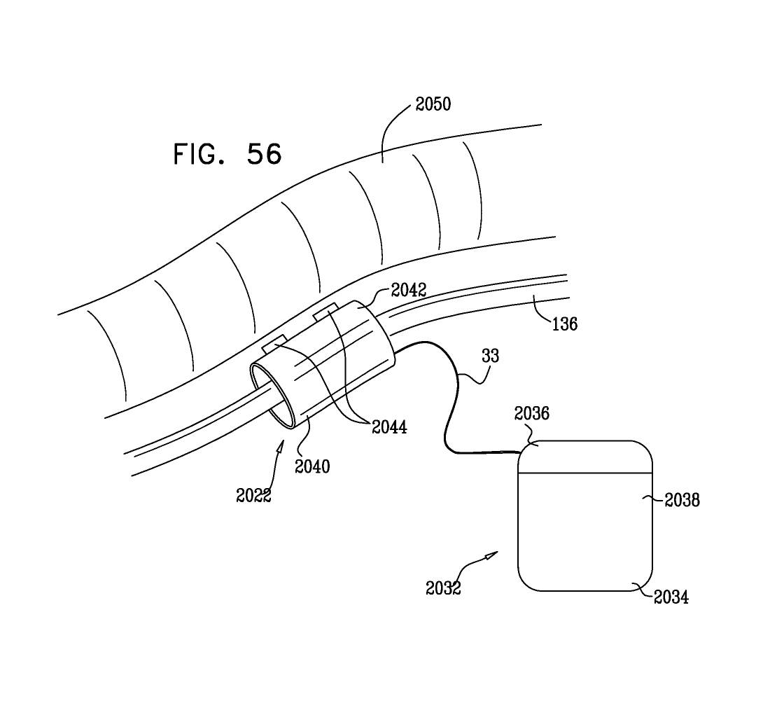

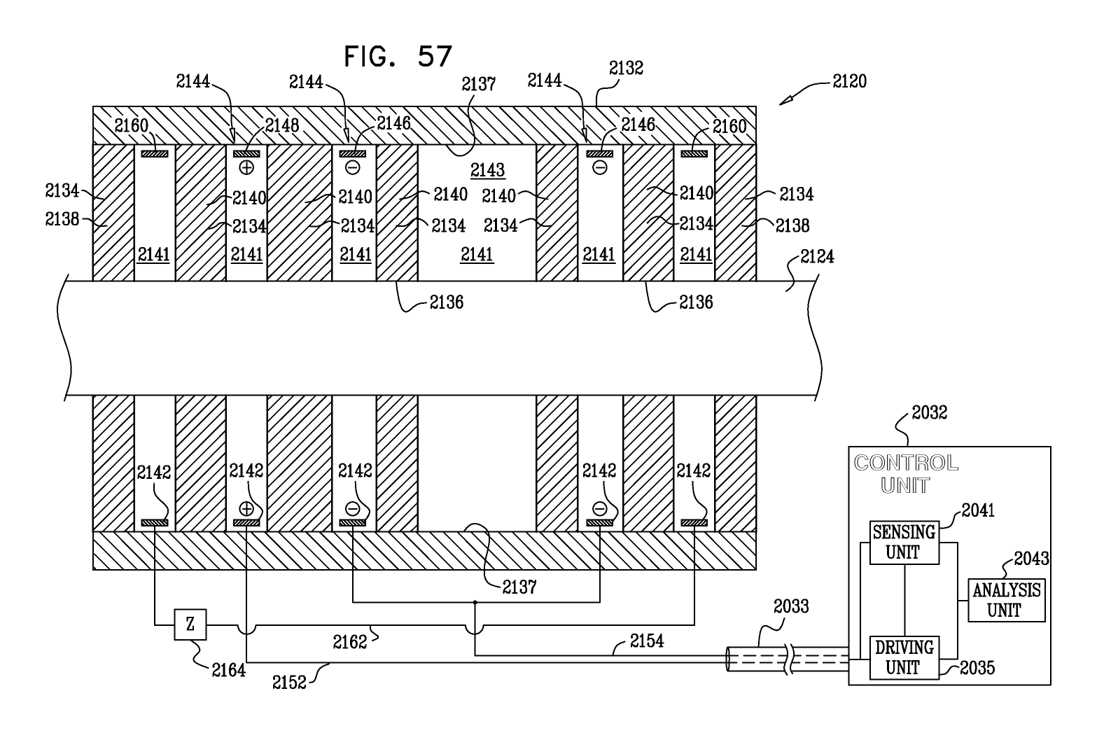

FIG. 55 is a schematic illustration of a nerve stimulation and cardiac sensing system 2020, in accordance with an embodiment of the present invention. System 2020 comprises at least one electrode device 2022, which is configured to be positioned in a vicinity of a nerve of a subject, at a location neither within nor in contact with a heart 30 of the subject (i.e., neither within the heart nor in contact with any tissue of the heart, including tissue of an external surface of the heart). For example, the nerve may be a vagus nerve 136 (either a left vagus nerve 37 or a right vagus nerve 39), which innervates heart 30. For some applications, the electrode device is configured to be placed at least partially around the nerve. For some applications, electrode device 2022 is applied to a cervical vagus nerve of the subject, as shown in FIG. 55. Electrode device 2022 comprises one or more nerve-facing electrode contact surfaces that are configured to be placed in electrical contact with the nerve, such as using techniques described hereinbelow with reference to FIG. 57, described in the references incorporated herein by reference hereinbelow or in the Background section, and/or known in the art.

System 2020 further comprises a control unit 2032, which typically communicates with electrode device 2022 over at least one lead 2033, which comprises one or more elongated conducting elements, such as wires, and an electrically insulating outer layer, comprising, for example, polyurethane or a similar insulation material. Control unit 2032 typically comprises an implantable can, which houses circuitry of the control unit. The can typically comprises a metal body 2034 and a non-metallic header 2036, which provides one or more connection points for lead 2033. For some applications, control unit 2032 comprises one or more of a driving unit 2035, a sensing unit 2041, and an analysis unit 2043, as shown in FIG. 57 and described hereinbelow. For some applications, control unit 2032 further comprises an output unit, which is configured to generate an output signal, as described hereinbelow.

In an embodiment of the present invention, control unit 2032 comprises at least one first sensing electrode contact surface 2038. For some applications, all or a portion of the outer surface of metal body 2034 of the can serves as first sensing electrode contact surface 2038. Alternatively, the control unit comprises a separate conductive element that serves as first sensing electrode contact surface 2038, which is directly mechanically coupled to the outer surface of the can. Alternatively or additionally, system 2020 comprises a second sensing electrode contact surface 2037 directly mechanically coupled to lead 2033 at a point along the lead between control unit 2032 and electrode device 2022. Still further alternatively or additionally, system 2020 comprises a third sensing electrode contact surface 2039, positioned at a location other than in direct mechanical contact with electrode device 2022 or lead 2033, and which is typically configured to positioned in the subject's body elsewhere than in heart 30 (e.g., at a location neither within nor in contact with the heart, or at a location in a vicinity of the heart, such as in contact with an external surface of the heart). For example, as shown in FIG. 55, third sensing electrode contact surface 2039 may be coupled to another lead that is coupled to control unit 2032.

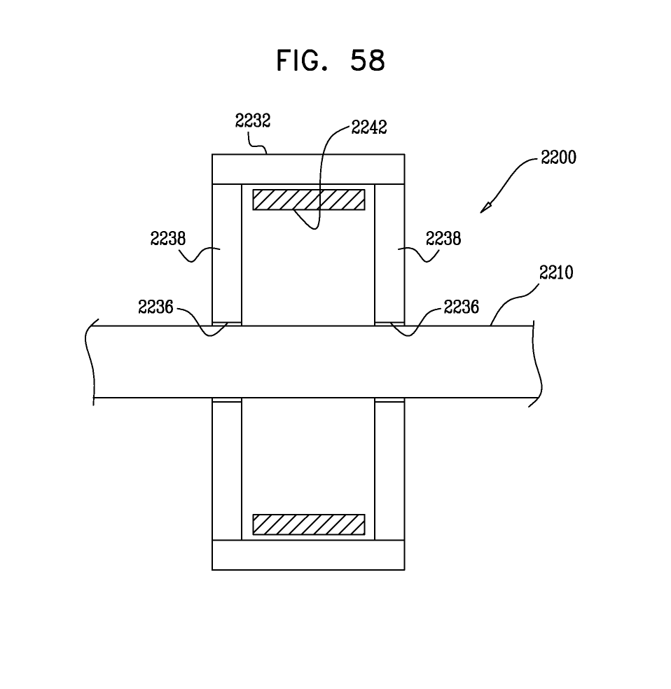

In order to sense a signal indicative of a parameter of a cardiac cycle of the subject, such as one or more components of an electrocardiogram (ECG) of heart 30, sensing unit 2041 of control unit 2032 uses two or more of the following electrodes contact surface:
first sensing electrode contact surface 2038;
second sensing electrode contact surface 2037;
third sensing electrode contact surface 2039;
at least one of the nerve-facing electrode contact surfaces of electrode device 2022, such as at least one of nerve-facing electrode contact surfaces 2142 described hereinbelow with reference to FIG. 57;
at least one of external sensing electrode contact surfaces 2044 of the electrode device, described hereinbelow with reference to FIG. 56; and
at least one blood-vessel-facing electrode contact surface of a blood vessel cuff configured to be placed in a vicinity of or around a blood vessel of the subject, such as a jugular vein or a carotid artery, such as described hereinbelow with reference to FIG. 58.

For example, the sensing unit of the control unit may sense the cardiac signal using one of the following combinations of electrode contact surfaces:
first sensing electrode contact surface 2038 and at least one of the nerve-facing electrode contact surfaces of electrode device 2022;
first sensing electrode contact surface 2038 and second sensing electrode contact surface 2037;
first sensing electrode contact surface 2038 and at least one of external sensing electrode contact surfaces 2044;
first sensing electrode contact surface 2038 and third sensing electrode contact surface 2039;
second sensing electrode contact surface 2037 and at least one of the nerve-facing electrode contact surfaces of electrode device 2022;
second sensing electrode contact surface 2037 and at least one of external sensing electrode contact surfaces 2044;
third sensing electrode contact surface 2039 and at least one of the nerve-facing electrode contact surfaces of electrode device 2022;
third sensing electrode contact surface 2039 and at least one of external sensing electrode contact surfaces 2044;
at least two of external sensing electrode contact surfaces 2044;
at least one of external sensing electrode contact surfaces 2044 and at least of the nerve-facing electrode contact surfaces of electrode device 2022; or
at least one of the blood-vessel-facing electrode contact surface of the blood vessel cuff.

Sensing unit 2041 of control unit 2032 senses an electrical signal, and analysis unit 2043 of control unit 2032 analyzes the sensed signal to identify the parameter of the cardiac cycle. For example, the parameter may be an R-R interval, an average heart rate, the timing of an A wave, or the timing of an R wave. For some applications, the output unit of the control unit is configured to generate an output signal responsively to the parameter of the cardiac cycle.

For some applications, the sensing unit of the control unit senses the cardiac signal using at least one first blood-vessel-facing electrode contact surface of a first blood vessel cuff, and at least one second blood-vessel-facing electrode contact surface of a second blood vessel cuff. For example, the blood vessel cuffs may use techniques described hereinbelow with reference to FIG. 58. Alternatively, the two electrode contact surfaces may be configured to be placed in a vicinity of the blood vessel without being placed around at least a portion of the blood vessel.

Driving unit 2035 of control unit 2032 is typically configured to apply stimulation to the nerve, and/or configure the applied stimulation, at least in part responsively to the sensed cardiac parameter. For example, the driving unit of the control unit may configure the stimulation to regulate a heart rate of the subject, using heart rate regulation techniques described in the art, and/or in the applications incorporated by reference hereinbelow or in the Background section. For example, the parameter may be a R-R interval of the ECG, and the control unit may cease or reduce a strength of the applied stimulation when the R-R interval exceeds a threshold value. For some applications, the cardiac parameter is indicative of ventricular contraction, and the driving unit of the control unit is configured to apply the stimulation during at least one heart beat after a delay from the ventricular contraction. For example, the delay may have a duration of at least 10 ms, such as at least 20 ms or at least 30 ms.

For some applications, electrode device 2022 comprises one or more stimulating nerve-facing electrode contact surfaces and one or more sensing nerve-facing electrode contact surfaces. Driving unit 2035 of control unit 2032 is configured to drive the stimulating nerve-facing electrode contact surfaces, and not the sensing nerve-facing electrode contact surfaces, to apply the stimulation to the nerve. Sensing unit 2041 of control unit 2032 is configured to sense the cardiac signal using the sensing nerve-facing electrode contact surfaces, and not using the stimulating nerve-facing electrode contact surfaces (and, optionally, one or more additional sensing electrodes of system 2020, as described hereinabove). This technique generally allows sensing unit 2041 to begin sensing the cardiac signal soon after the conclusion of the application of stimulation, without waiting for the stimulating nerve-facing electrode contact surfaces to discharge.

FIG. 56 is a schematic illustration of electrode device 2022, in accordance with an embodiment of the present invention. In this embodiment, electrode device 2022 comprises a housing 2040 which defines an outer surface 2042 of the device when the device is placed at least partially around vagus nerve 136, or another nerve of the subject, e.g., an autonomic nerve (either parasympathetic or sympathetic). Typically, electrode device 2022 comprises an electrode cuff. Except as described below, electrode device 2022 may be configured in accordance with any of the embodiments described hereinbelow, in the patent applications incorporated by reference hereinbelow or in the Background section, or otherwise as known in the art of electrode cuffs.

In addition to comprising a plurality of nerve-facing stimulating electrode contact surfaces within the electrode device (for example, nerve-facing electrode contact surfaces 2142, described hereinbelow with reference to FIG. 57), electrode device 2022 comprises one or more external sensing electrode contact surfaces 2044, fixed to outer surface 2042 of housing 2040. Sensing unit 2041 of control unit 2032 uses external sensing electrode contact surfaces 2044 to sense a signal indicative of a parameter of a cardiac cycle of the subject, such as one or more components of an ECG of heart 30. For some applications, in order to sense this property, the electrode device is configured to be implanted in a vicinity (e.g., within 10 mm, such as within 2 mm) of a blood vessel 2050 of the subject, such as an artery, e.g., a carotid artery or a jugular vein. Alternatively or additionally, the electrode device is implanted within a distance of the blood vessel that is no more than twice a distance between two of external sensing electrode contact surfaces 2044. For some applications, the electrode device is implanted around a cervical vagus nerve in a vicinity of the carotid artery or the jugular vein. Alternatively, the electrode device is not configured to be placed in a vicinity of a blood vessel.

Driving unit 2035 of control unit 2032 is typically configured to apply stimulation to the nerve, and/or configure the applied stimulation, at least in part responsively to the sensed cardiac parameter. For example, the control unit may configure the stimulation to regulate a heart rate of the subject, as described hereinabove with reference to FIG. 55.

For some application, sensing unit 2041 of control unit 2032 senses the cardiac property using one or more sensing electrode contact surfaces 2044 and one or more of the electrode contact surfaces within the electrode device. Alternatively or additionally, the sensing unit of the control unit senses the cardiac property using one or more sensing electrode contact surfaces 2044 and sensing electrode contact surface 2038, described hereinabove with reference to FIG. 55.

FIG. 57 is a schematic, cross-sectional illustration of an electrode cuff 2120 for applying current to a nerve 2124, in accordance with an embodiment of the present invention. Electrode cuff 2120 comprises a housing 2132 which defines an outer surface of the cuff when the cuff is placed at least partially around nerve 2124. Housing 2132 typically comprises an elastic, electrically-insulating material such as silicone or polyurethane, which may have, for example, a Shore A of between about 35 and about 70, such as about 40.

Electrode cuff 2120 further comprises a plurality of insulating elements 2134 that are arranged at respective positions along the housing, and are typically fixed to an inner surface 2137 of housing 2132 that faces nerve 2124 when the electrode cuff is placed at least partially around the nerve. Insulating elements 2134 typically comprise an elastic, electrically-insulating material such as silicone or silicone copolymer, which, for some applications, is softer than that of housing 2132, for example, a Shore A of between about 10 and about 30, such as about 10. Electrode cuff 2120 is typically configured such that, after placement of the cuff around the nerve, respective contact surfaces 2136 of insulating elements 2134 come in physical contact with the nerve, or substantially in physical contact with the nerve, e.g., are less than about 0.5 mm from the surface of the nerve. For some applications, a length that at least one of insulating elements 2134 protrudes from housing 2132 toward nerve 2124 is at least 0.5 mm, such as at least 1 mm. For some applications, insulating elements 2134 and housing 2132 are constructed as separate elements that are coupled to one another, while for other applications, the insulating elements and housing are constructed as a single integrated element that is shaped to define the insulating elements and housing.

Insulating elements 2134 typically comprise one or more (such as exactly two) end insulating elements 2138 arranged at or near respective ends of the cuff, and two or more internal insulating elements 2140 arranged at respective positions along the cuff between the end insulating elements. End insulating elements 2138 extend along nerve 2124 in order to electrically isolate a portion of the nerve within electrode cuff 2120 from a portion of the nerve outside the electrode cuff.

Inner surface 2137 of housing 2132 and pairs of insulating elements 2134 define a respective cavities 2141 along the housing. (It is noted that some pairs of the insulating elements may not define a cavity, such as if two or more of the insulating elements are arranged in contact with one another.)

Electrode cuff 2120 comprises a plurality of nerve-facing electrode contact surfaces 2142, fixed within housing 2132 in respective cavities 2141 defined by respective pairs insulating elements 2134 and inner surface 2137 of housing 2132. At least one of cavities 2141 defined by a pair of the insulating elements does not have an electrode contact surface positioned therein. For example, in the embodiment shown in FIG. 57, the insulating elements define six cavities 2141, a fourth one 2143 of which (counting from the left in the figure) does not have an electrode contact surface positioned therein. For some applications, at least two, such as least three, of the cavities do not have electrode contact surfaces positioned therein. Nerve-facing electrode contact surfaces 2142 are typically fixed to inner surface 2137 of housing 2132.

For some applications, at least one of the empty cavities has a length along the cuff of at least 0.5 mm, such as at least 0.7 mm, e.g., at least 1.4 mm or at least 2 mm, and/or no more than 5 mm, e.g., no more than 2 mm. For some applications, a length along the cuff of one of the empty cavities is between about 0.5 and about 5 times a length of one of the cavities that has an electrode contact surface therein, such as between about 1 and about 2 times the length.

For some applications, at least one of the empty cavities is directly adjacent along the cuff to two cavities containing an anode electrode contact surface and a cathode electrode contact surface, respectively. For some applications, at least one of the empty cavities is directly adjacent along the cuff to two cavities containing two respective anode electrode contact surfaces, or to two cavities containing two respective cathode electrode contact surfaces. Alternatively, at least one of the two endmost cavities is empty, e.g., one side of at least one of the empty cavities is defined by one of end insulating elements 2138.

Internal insulating elements 2140 are arranged so as to electrically separate nerve-facing electrode contact surfaces 2142, and to guide current from one of the electrode contact surfaces towards the nerve prior to being taken up by another one of the electrode contact surfaces. Typically (as shown), insulating elements 2134 are closer to nerve 2124 than are the electrode contact surfaces, i.e., the electrode contact surfaces are recessed within the cavities. Alternatively (not shown), insulating elements 2134 are generally flush with the faces of the electrode contact surfaces, such that the inner surfaces of insulating elements and the conductive surfaces of the electrode contact surface are equidistant from the nerve.

Nerve-facing electrode contact surfaces 2142 comprise at least one active, i.e., stimulating and/or sensing, electrode contact surface 2144, such as at least one cathode electrode contact surface 2146 and at least one anode electrode contact surface 2148. Active electrode contact surfaces 2144 are coupled to control unit 2032 by conducting elements 2152 and 2154 of lead 2033. For some applications, active electrode configurations and/or stimulation techniques are used which are described in one or more of the patent applications incorporated by reference hereinbelow. For some applications, two or more of the active electrode contact surfaces are shorted to one another inside or outside of the cuff, such as shown for cathode electrode contact surfaces 2146 in FIG. 57.

In an embodiment of the present invention, electrode cuff 2120 further comprises two or more passive electrode contact surfaces 2160, fixed within housing 2132, and a conducting element 2162, typically a wire, which electrically couples the passive electrode contact surfaces to one another. A “passive electrode contact surface,” as used in the present application including the claims, is an electrode contact surface that is electrically “device-coupled” to neither (a) any circuitry that is electrically device-coupled to any of the cathode electrode contact surfaces or anode electrode contact surfaces, nor (b) an energy source. “Device-coupled” means coupled, directly or indirectly, by components of a device, and excludes coupling via tissue of a subject. (It is noted that the passive electrode contact surfaces may be passive because of a software-controlled setting of the electrode assembly, and that the software may intermittently change the setting such that these electrode contact surfaces are not passive.) To “passively electrically couple,” as used in the present application including the claims, means to couple using at least one passive electrode contact surface and no non-passive electrode contact surfaces. Passive electrode contact surfaces 2160 and conducting element 2162 create an additional electrical path for the current, such as an additional path for the current that would otherwise leak outside electrode cuff 2120 and travel around the outside of the housing through tissue of the subject. For some applications, conducting element 2162 comprises at least one passive element 2164, such as a resistor, capacitor, and/or inductor. In this embodiment, end insulating elements 2138 help direct any current that leaks from active electrode contact surfaces 2144 through the electrical path created by passive electrode contact surfaces 2160 and conducting element 2162. For some applications, active electrode contact surfaces 2144 are positioned within housing 2132 longitudinally between the two or more passive electrode contact surfaces 2160 (as shown in FIG. 57). Alternatively, at least one of the passive electrode contact surfaces is positioned between the at least one cathode electrode contact surface and the at least one anode electrode contact surface (configuration not shown).

In an embodiment of the present invention, electrode cuff 2120 comprises one or more passive electrode contact surfaces 2160 which are not electrically device-coupled to one another. For some applications, the electrode cuff comprises exactly one passive electrode contact surface 2160. A separate conducting element, typically a wire, is coupled to each passive electrode contact surface at a first end of the conducting element. The second end of the conducting element terminates at a relatively-remote location in the body of the subject that is at a distance of at least 1 cm, e.g., at least 2 or 3 cm, from electrode cuff 2120. The remote location in the body thus serves as a ground for the passive electrode contact surface. For some applications, an electrode contact surface is coupled to the remote end of the conducting element, so as to increase electrical contact with tissue at the remote location.

For some applications, housing 2132 has a length of between about 10 and about 14 mm, e.g., about 12.1 mm; an outer radius of between about 4 and about 8 mm, e.g., about 5.9 mm; and an inner radius of between about 3 and about 6 mm, e.g., about 4.5 mm. For some applications, insulating elements 2134 have an outer radius of between about 3 and about 6 mm, e.g., about 4.5 mm (the outer radius of the insulating elements is typically equal to the inner radius of the housing), and an inner radius of between about 2 and about 3.5 mm. For some applications in which cuff 2120 comprises exactly two end insulating elements 2138 and exactly five internal insulating elements 2140, respective edges of insulating elements 2134 are positioned within cuff 2032 at the following distances from one end of the cuff: 0.0 mm, between 1.3 and 1.7 mm (e.g., 1.5 mm), between 2.7 and 3.3 mm (e.g., 3.0 mm), between 5.1 and 6.3 mm (e.g., 5.7 mm), between 7.1 and 8.7 mm (e.g., 7.9 mm), between 8.5 and 10.3 mm (e.g., 9.4 mm), and between 10.2 and 12.4 mm (e.g., 11.3 mm), and the insulating elements having the following respective widths: between 0.7 and 0.9 mm (e.g., 0.8 mm), between 0.7 and 0.9 mm (e.g., 0.8 mm), between 1.4 and 1.8 mm (e.g., 1.6 mm), between 0.7 and 0.9 mm (e.g., 0.8 mm), between 0.7 and 0.9 mm (e.g., 0.8 mm), between 1.1 and 1.3 mm (e.g., 1.2 mm), and between 0.7 and 0.9 mm (e.g., 0.8 mm). For some applications, electrode contact surfaces 2142 comprise Pt/Ir. For some applications, as shown in FIG. 57, electrode contact surfaces 2142 are shaped as rings (e.g., reference numeral 2160 and leftmost reference numeral 2142 in FIG. 57 refer to a single ring electrode contact surface). The rings may have an outer radius that equals, or is slightly greater or less than, the inner radius of housing 2132.

In an embodiment of the present invention, at least some of the electrode contact surfaces do not comprise ring electrode contact surfaces. Instead, each of at least one of non-empty cavities 2141 has fixed therein a plurality of electrode contact surfaces positioned at least partially circumferentially around a central axis of the cuff. In other words, electrode contact surfaces 2142 are first electrode contact surfaces 2142, fixed within housing 2132 in respective cavities 2141, and cuff 2120 comprises at least one second electrode contact surface 2142, fixed within housing 2132 in one of the cavities 2141 in which one of the first electrode contact surfaces 2142 is fixed. For some applications, the plurality of electrode contact surfaces within a single cavity are circumferentially separated from one another by one or more circumferentially arranged insulating elements.

In an embodiment of the present invention, at least one of the one or more of cavities 2141 which are empty in the embodiments described hereinabove, instead has fixed therein one or more electrode contact surfaces that are not electrically device-coupled (as defined hereinabove) to any elements of the device outside of the cavity. These electrode contact surfaces thus do not serve the normal function of electrode contact surfaces in an electrode cuff, i.e., conducting current to and/or from tissue.

In some embodiments of the present invention in which nerve 2124 is vagus nerve 136, electrode cuff 2120 is configured to be placed at least partially around the vagus nerve such that anode electrode contact surface 2148 is more proximal to a brain 134 of patient 31 (FIG. 55) than are cathode electrode contact surfaces 2146.

For some applications, electrode cuff 2120 is configured to selectively stimulate fibers of the nerve having certain diameters, such as by using techniques described in one or more of the patent applications incorporated by reference hereinbelow. For example, control unit 2032 may comprise a driving unit, which is configured to drive cathode electrode contact surface 2146 to apply to nerve 2124 a stimulating current, which is capable of inducing action potentials in a first set and a second set of nerve fibers of the nerve, and drive anode electrode contact surface 2148 to apply to the nerve an inhibiting current, which is capable of inhibiting the induced action potentials traveling in the second set of nerve fibers, the nerve fibers in the second set having generally larger diameters than the nerve fibers in the first set.

For some applications, electrode cuff 2120 is configured to apply unidirectional stimulation to the nerve, such as by using techniques described in one or more of the patent applications incorporated by reference hereinbelow. For example, control unit 2032 may comprise a driving unit, which is configured to drive anode electrode contact surface 2148 to apply an inhibiting current capable of inhibiting device-induced action potentials traveling in a non-therapeutic direction in nerve 2124. For some applications, electrode cuff 2120 comprises primary and secondary anode electrode contact surfaces, the primary anode electrode contact surface located between the secondary anode electrode contact surface and the cathode electrode contact surface. The secondary anode electrode contact surface is typically adapted to apply a current with an amplitude less than about one half an amplitude of a current applied by the primary anode electrode contact surface.

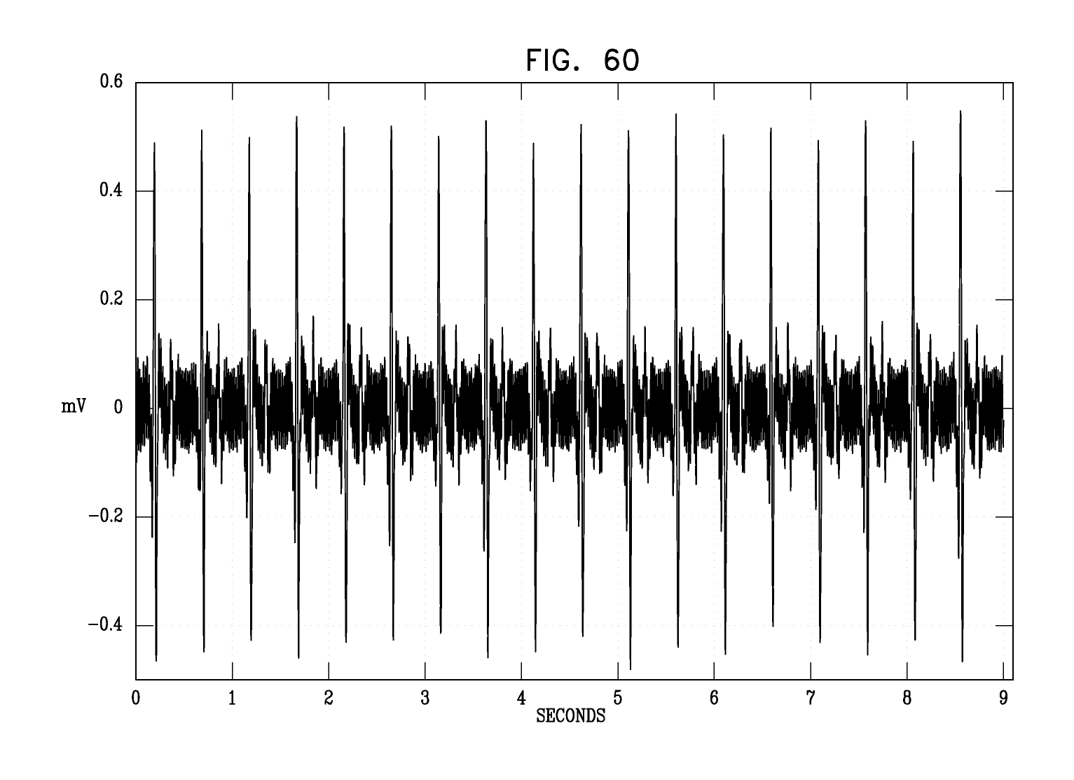

In an embodiment of the present invention, techniques described herein are practiced in combination with techniques described with reference to FIGS. 56, 57, and/or 60 of U.S. patent application Ser. No. 11/280,884 to Ayal et al., filed Nov. 15, 2005, which published as US Patent Application Publication 2006/0106441, and which is assigned to the assignee of the present application and is incorporated herein by reference. For example:
for some applications, a closest distance between cathode electrode contact surfaces 2146 (i.e., the distance between the respective cathode electrode contact surfaces' edges that are closest to one another) is equal to at least a radius R of nerve 2124, e.g., at least 1.5 times the radius of the nerve, as described with reference to FIG. 56 of the '441 publication; and/or
for some applications, end insulating elements 2138 are elongated, as described with reference to FIG. 60 of the '441 publication.

FIG. 58 is a schematic, cross-sectional illustration of an electrode cuff 2200 for sensing a cardiac signal at a blood vessel 2210, in accordance with an embodiment of the present invention. For example, blood vessel 2210 may be a jugular vein or a carotid artery. Electrode cuff 2200 comprises a housing 2232 which defines an outer surface of the cuff when the cuff is placed at least partially around blood vessel 2210. Housing 2232 may comprise an elastic, electrically-insulating material such as silicone or polyurethane.

Electrode cuff 2200 further comprises one or more (such as exactly two) end insulating elements 2238 arranged at or near respective ends of the cuff, which are typically fixed to an inner surface of housing 2232 that faces blood vessel 2210 when the electrode cuff is placed at least partially around the blood vessel. Insulating elements 2238 typically comprise an elastic, electrically-insulating material such as silicone or silicone copolymer, which, for some applications, is softer than that of housing 2232.

Electrode cuff 2200 comprises at least one blood-vessel-facing electrode contact surface 2242 (such as exactly one electrode contact surface 2242) fixed within housing 2232. For some applications, electrode contact surface 2242 is used for sensing an electrical signal, such as described hereinabove with reference to FIG. 55.

It is noted that although electrode cuffs 2120 and 2200 and their elements are generally shown in the figures and described herein in a cylindrical configuration, other geometrical configurations, such as non-rotationally symmetric configurations, are also suitable for applying the principles of the present invention. In particular, housings 2132 or 2232 of the electrode cuffs (and the electrode contact surfaces themselves) may form a complete circle around nerve 2124 or blood vessel 2210, or they may define an arc between approximately 0 and 90 degrees, between 90 and 180 degrees, between 180 and 350 degrees, or between 350 and 359 degrees around the nerve or blood vessel. For some applications, electrode cuff 2120 or 2200 comprise electrode contact surfaces that form rings around the nerve or blood vessel, such that housing 2132 or 2232 surrounds the electrode contact surfaces.

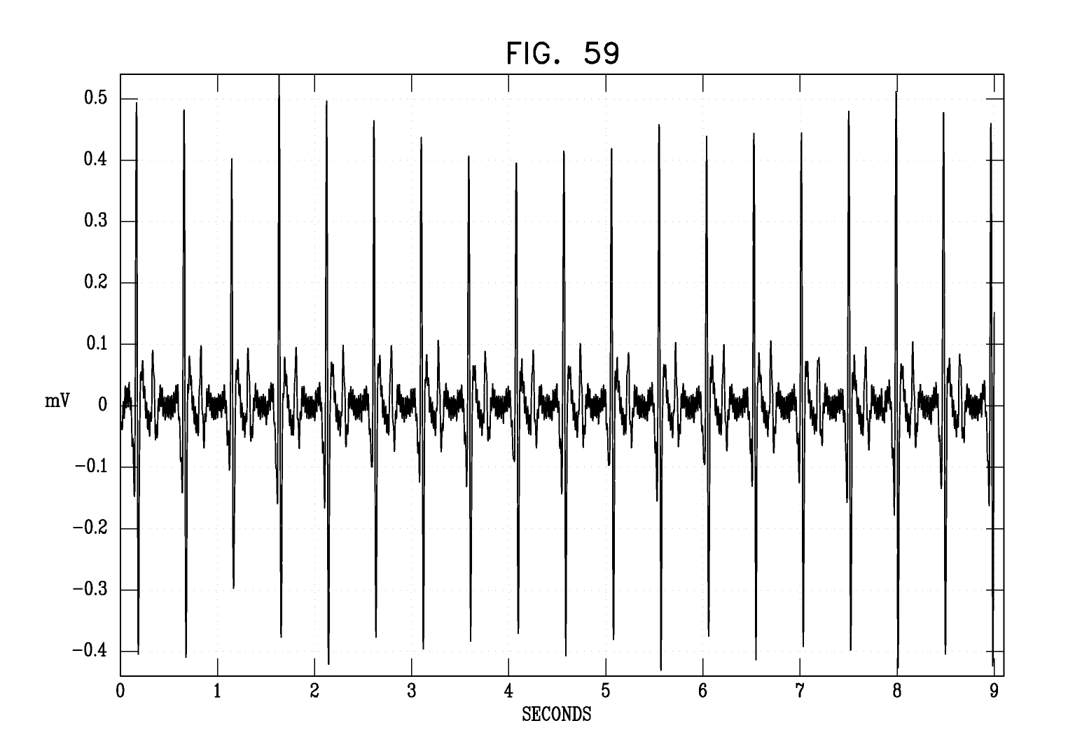

FIGS. 59-61 are graphs illustrating experimental results measured in accordance with respective embodiments of the present invention. These graphs show respective electrocardiograms measured in a single dog while under general anesthesia.

The electrocardiogram shown in FIG. 59 was measured between (a) a can implanted in the chest of the dog on the right thoracic side, over the pectoralis major muscle, inferior to the clavicle bone and (b) a cardiac pacemaker electrode lead placed at a cervical location, in proximity to the right cervical vagus nerve and the right jugular vein, approximately 30 cm from the can. This configuration was similar to the embodiment described hereinabove with reference to FIG. 55, in which the cardiac signal is sensed using (a) the outer surface of metal body 2034 of the can serving as first sensing electrode contact surface 2038, and (b) second sensing electrode contact surface 2037. This configuration was also similar to the embodiment described hereinabove with reference to FIGS. 55 and 57, in which the cardiac signal is sensed using (a) the outer surface of metal body 2034 of the can serving as first sensing electrode contact surface 2038, and (b) external sensing electrode contact surface 2044, described hereinabove with reference to FIG. 56, when not necessarily placed in the vicinity of blood vessel 2050. As can be seen in FIG. 59, the measured electrocardiogram is clear and provides clinically useful information.

The electrocardiogram shown in FIG. 60 was measured between (a) a can implanted in the chest of the dog on the right thoracic side, over the pectoralis major muscle, inferior to the clavicle bone and (b) a nerve-facing ring electrode contact surface of an electrode cuff similar to electrode cuff 2120 described hereinabove with reference to FIG. 57. The electrode cuff was placed around the cervical vagus nerve at a cervical location in proximity to the right jugular vein approximately 30 cm from the can. This configuration was similar to the embodiment described hereinabove with reference to FIG. 55, in which the cardiac signal is sensed using (a) the outer surface of metal body 2034 of the can serving as first sensing electrode contact surface 2038, and (b) one of the nerve-facing electrode contact surfaces of electrode device 2022. As can be seen in FIG. 60, the measured electrocardiogram is clear and provides clinically useful information.

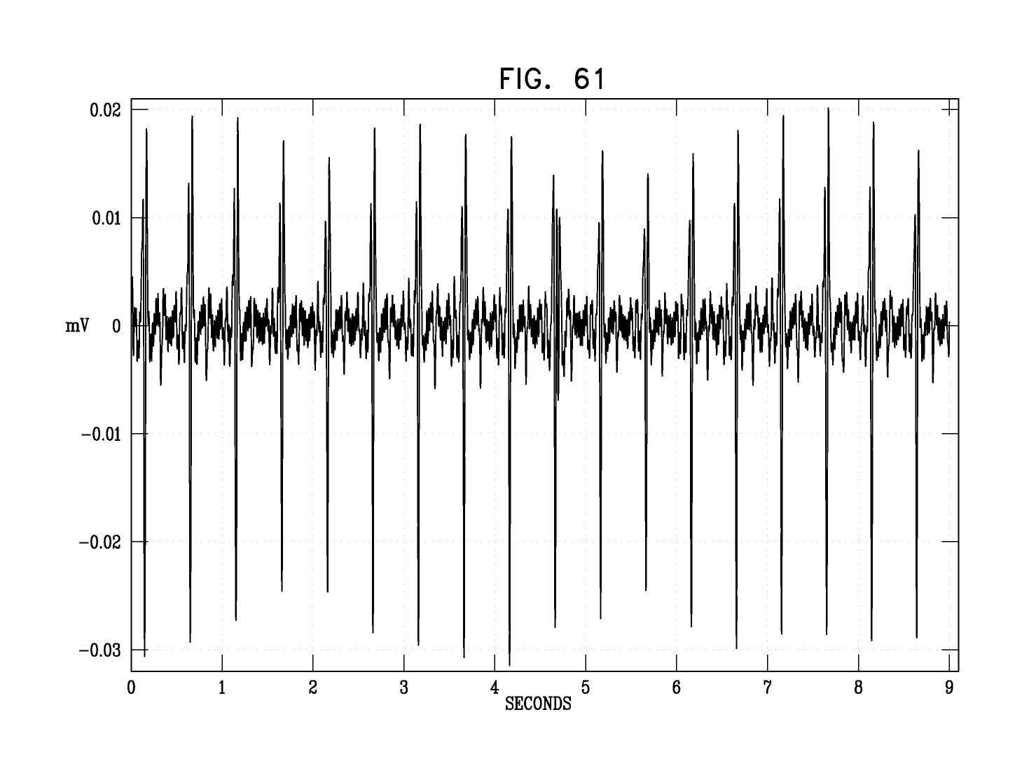

The electrocardiogram shown in FIG. 61 was measured between two electrode contact surfaces within two respective cuffs placed around the cervical jugular vein such that the two electrode contact surfaces were 2 cm apart from one another. The cuffs were similar to the nerve cuffs described hereinabove with reference to FIG. 57, except that the cuffs were applied to a blood vessel rather than a nerve. This configuration was similar to the embodiment described hereinabove with reference to FIG. 55, in which the cardiac signal is sensed using two blood-vessel facing electrode contact surfaces of two respective blood vessel cuffs. As can be seen in FIG. 61, the measured electrocardiogram is clear and provides clinically useful information.

As used in the present patent application, including in the claims, “longitudinal” means along the length of, and does not mean “around” or “circumferential.” For example, “longitudinal positions” along the housing means positions along the length of the housing, rather than positions arranged circumferentially around a longitudinal axis of the housing or the nerve. Such longitudinal positions might be measured in mm from one end of the housing.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. Provisional Patent Application 60/383,157 to Ayal et al., filed May 23, 2002, entitled, “Inverse recruitment for autonomic nerve systems,”
International Patent Application PCT/IL02/00068 to Cohen et al., filed Jan. 23, 2002, entitled, “Treatment of disorders by unidirectional nerve stimulation,” and U.S. patent application Ser. No. 10/488,334, in the national stage thereof, which published as US Patent Application Publication 2004/0243182,
U.S. patent application Ser. No. 09/944,913 to Cohen and Gross, filed Aug. 31, 2001, entitled, “Treatment of disorders by unidirectional nerve stimulation,” which issued as U.S. Pat. No. 6,684,105,
U.S. patent application Ser. No. 09/824,682 to Cohen and Ayal, filed Apr. 4, 2001, entitled “Method and apparatus for selective control of nerve fibers,” which issued as U.S. Pat. No. 6,600,954,
U.S. patent application Ser. No. 10/205,475 to Gross et al., filed Jul. 24, 2002, entitled, “Selective nerve fiber stimulation for treating heart conditions,” which published as US Patent Application Publication 2003/0045909,
U.S. patent application Ser. No. 10/205,474 to Gross et al., filed Jul. 24, 2002, entitled, “Electrode assembly for nerve control,” which issued as U.S. Pat. No. 6,907,295,
International Patent Application PCT/IL03/00431 to Ayal et al., filed May 23, 2003, entitled, “Selective nerve fiber stimulation for treating heart conditions,” which published as PCT Publication WO 03/099377 to Ayal et al.,
International Patent Application PCT/IL03/00430 to Ayal et al., filed May 23, 2003, entitled, “Electrode assembly for nerve control,” which published as PCT Publication WO 03/099373 to Ayal et al., and U.S. patent application Ser. No. 10/529,149, in the national stage thereof, which published as US Patent Application Publication 2006/0116739,
U.S. patent application Ser. No. 10/719,659 to Ben David et al., filed Nov. 20, 2003, entitled, “Selective nerve fiber stimulation for treating heart conditions,” which published as US Patent Application Publication 2004/0193231,
U.S. patent application Ser. No. 11/022,011 to Cohen et al., filed Dec. 22, 2004, entitled, “Construction of electrode assembly for nerve control,” which issued as U.S. Pat. No. 7,561,922,
U.S. patent application Ser. No. 11/234,877 to Ben-David et al., filed Sep. 22, 2005, entitled, “Selective nerve fiber stimulation,” which published as US Patent Application Publication 2006/0100668,
U.S. patent application Ser. No. 11/280,884 to Ayal et al., filed Nov. 15, 2005, entitled, “Techniques for nerve stimulation,” which published as US Patent Application Publication 2006/0106441, and
U.S. patent application Ser. No. 12/217,930 to Ben-David et al., filed Jul. 9, 2008, entitled, “Electrode cuffs,” which published as US Patent Application Publication 2010/0010603.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-168. (canceled)

169. Apparatus comprising:
an electrode device, configured to be coupled to a site of a subject selected from the group consisting of: a vagus nerve, an epicardial fat pad, a pulmonary vein, a carotid artery, a carotid sinus, a coronary sinus, a vena cava vein, a right ventricle, a right atrium, and a jugular vein; and
a control unit, configured to:
drive the electrode device to apply to the site a current in bursts of one or more pulses, during “on” periods that alternate with low stimulation periods, wherein at least one of the “on” periods has an “on” duration of at least three seconds, and includes at least three bursts, and wherein at least one of the low stimulation periods immediately following the at least one of the “on” periods has a low stimulation duration equal to at least 50% of the “on” duration,
set the current applied on average during the low stimulation periods to be less than 20% of the current applied on average during the “on” periods, and
during at least one transitional period of the at least one of the “on” periods, ramp a number of pulses per burst, the at least one transitional period selected from the group consisting of: a commencement of the at least one of the “on” periods, and a conclusion of the at least one of the “on” periods.

170-171. (canceled)

172. The apparatus according to claim 169, further comprising a sensor configured to sense a level of copeptin in blood of the subject, wherein the control unit is configured to set a ratio of (a) an average “on” duration of the “on” periods to (b) an average duration of the low stimulation periods, responsively to the sensed level of copeptin, such that the ratio is positively correlated with the copeptin level.

173-181. (canceled)

182. The apparatus according to claim 169, wherein the at least one transitional period includes the commencement of the at least one of the “on” periods, and wherein the control unit is configured to ramp up the number of pulses per burst during the commencement.

183. The apparatus according to claim 182, wherein the control unit is configured to set the number of pulses of an initial burst of the at least one of the “on” periods and a second burst immediately subsequent to the initial burst to be equal to 1 and 2, respectively.

184. The apparatus according to claim 183, wherein the control unit is configured to set the number of pulses of a third burst of the at least one of the “on” periods immediately subsequent to the second burst to be equal to 3.

185. The apparatus according to claim 169, wherein the at least one transitional period includes the conclusion of the at least one of the “on” periods, and wherein the control unit is configured to ramp down the number of pulses per burst during the conclusion.

186. The apparatus according to claim 185, wherein the control unit is configured to set the number of pulses of last and penultimate bursts of the at least one of the “on” periods to be equal to 1 and 2, respectively.

187. The apparatus according to claim 186, wherein the control unit is configured to set the number of pulses of an antepenultimate burst of the at least one of the “on” periods to be equal to 3.

188. The apparatus according to claim 169, wherein the control unit is configured to set the current applied on average during the low stimulation periods to be less than 20% of the current applied on average during the “on” periods.

189. The apparatus according to claim 188, wherein the control unit is configured to set the current applied on average during the low stimulation periods to be less than 5% of the current applied on average during the “on” periods.

190. The apparatus according to claim 189, wherein the control unit is configured to withhold applying the current during the low stimulation periods.

191-268. (canceled)

269. A method comprising:
applying, to a site of a subject, a current in bursts of one or more pulses, during “on” periods that alternate with low stimulation periods, at least one of the “on” periods having an “on” duration of at least three seconds, and including at least three bursts, and at least one of the low stimulation periods immediately following the at least one of the “on” periods having a low stimulation duration equal to at least 50% of the “on” duration, the site selected from the group consisting of: a vagus nerve, an epicardial fat pad, a pulmonary vein, a carotid artery, a carotid sinus, a coronary sinus, a vena cava vein, a right ventricle, a right atrium, and a jugular vein;
setting the current applied on average during the low stimulation periods to be less than 20% of the current applied on average during the “on” periods; and
during at least one transitional period of the at least one of the “on” periods, ramping a number of pulses per burst, the at least one transitional period selected from the group consisting of: a commencement of the at least one of the “on” periods, and a conclusion of the at least one of the “on” periods.

270-271. (canceled)

272. The method according to claim 269, further comprising sensing a level of copeptin in blood of the subject, wherein applying the current comprises setting a ratio of (a) an average “on” duration of the “on” periods to (b) an average duration of the low stimulation periods, responsively to the sensed level of copeptin, such that the ratio is positively correlated with the copeptin level.

273-281. (canceled)

282. The method according to claim 269, wherein the at least one transitional period includes the commencement of the at least one of the “on” periods, and wherein ramping comprises ramping up the number of pulses per burst during the commencement.

283. The method according to claim 282, wherein ramping comprises setting the number of pulses of an initial burst of the at least one of the “on” periods and a second burst immediately subsequent to the initial burst to be equal to 1 and 2, respectively.

284. The method according to claim 283, wherein ramping comprises setting the number of pulses of a third burst of the at least one of the “on” periods immediately subsequent to the second burst to be equal to 3.

285. The method according to claim 269, wherein the at least one transitional period includes the conclusion of the at least one of the “on” periods, and wherein ramping comprises ramping down the number of pulses per burst during the conclusion.

286. The method according to claim 285, wherein ramping comprises setting the number of pulses of last and penultimate bursts of the at least one of the “on” periods to be equal to 1 and 2, respectively.

287. The method according to claim 286, wherein ramping comprises setting the number of pulses of an antepenultimate burst of the at least one of the “on” periods to be equal to 3.

288. The method according to claim 269, wherein setting the current applied on average during the low stimulation periods comprises setting the current applied on average during the low stimulation periods to be less than 20% of the current applied on average during the “on” periods.

289. The method according to claim 288, wherein setting the current applied on average during the low stimulation periods comprises setting the current applied on average during the low stimulation periods to be less than 5% of the current applied on average during the “on” periods.

290. The method according to claim 289, wherein setting the current applied on average during the low stimulation periods comprises withholding applying the current during the low stimulation periods.

291-460. (canceled)
